(19) United States
(12) Patent Application Publication
(10) Pub. No.: US 2024/0124553 A1
(43) Pub. Date: Apr. 18, 2024

(54) COMPOSITIONS AND METHODS FOR INHIBITION OF ALPHAVIRUS INFECTION

(71) Applicant: Washington University, St. Louis, MO (US)

(72) Inventors: Michael Diamond, St. Louis, MO (US); Daved Fremont, St. Louis, MO (US); Rong Zhang, St. Louis, MO (US); Arthur Kim, St. Louis, MO (US); Larissa Thackray, St. Louis, MO (US); Katherine Basore, St. Louis, MO (US); Christopher A. Nelson, St. Louis, MO (US); Ofer Zimmerman, St. Louis, MO (US); Maxwell Zimmerman, St. Louis, MO (US); Saravanan Raju, St. Louis, MO (US)

(73) Assignee: Washington University, St. Louis, MO (US)

(21) Appl. No.: 18/472,918

(22) Filed: Sep. 22, 2023

Related U.S. Application Data
(63) Continuation-in-part of application No. 16/959,867, filed on Jul. 2, 2020.
(60) Provisional application No. 62/613,658, filed on Jan. 4, 2018.
(60) Provisional application No. 62/671,680, filed on May 15, 2018.
(60) Provisional application No. 62/672,080, filed on May 16, 2018.

(51) Int. Cl. C07K 14/705; A61P 31/14

(52) U.S. Cl. CPC C07K 14/70596; A61P 31/14; A61K 38/00

(57) ABSTRACT

The present disclosure is directed to compositions and methods of treating arthritogenic alphavirus infection. Compositions include a fusion protein comprising an Fc region, a first Mxra8 region, and a second Mxra8 region, wherein the fusion protein reduces inflammation and infection by an arthritogenic alphavirus. Methods include administering the fusion protein to a mammalian or non-mammalian subject to reduce or inhibit alphavirus infection.

### CROSS-REFERENCE TO RELATED APPLICATIONS

This application is a continuation-in-part of and claims the benefit of priority to U.S. National Phase application Ser. No. 16/959,867 filed on 2 Jul. 2020, which claims the benefit of PCT International Application No. PCT/US19/12435 filed on 4 Jan. 2019, which claims the benefit of U.S. Provisional Application Ser. No. 62/613,658 filed on 4 Jan. 2018; 62/671,680 filed on 15 May 2018; and 62/672,080 filed on 16 May 2018, which are incorporated herein by reference in their entireties.

### STATEMENT REGARDING FEDERALLY SPONSORED RESEARCH OR DEVELOPMENT

This invention was made with government support under A1114816 awarded by the National Institutes of Health. The government has certain rights in the invention.

### MATERIAL INCORPORATED-BY-REFERENCE

The Sequence Listing, which is a part of the present disclosure, includes a computer-readable form comprising nucleotide and/or amino acid sequences of the present invention (file name “017583-US-CIP_Sequence_Listing.xml” created on 14 Dec. 2023; 221,608 bytes). The subject matter of the Sequence Listing is incorporated herein by reference in its entirety.

### FIELD OF THE INVENTION

The present disclosure generally relates to compositions and methods for inhibition of arthritogenic alphavirus infections.

### SUMMARY OF THE INVENTION

Among the various aspects of the present disclosure is the provision of compositions and methods of treating arthritogenic alphavirus infection and screening methods.

In one aspect of the present disclosure, a fusion protein is provided. The fusion protein comprising an Fc region; a first Mxra8 region; and a second Mxra8 region; and wherein the fusion protein reduces inflammation and infection by an arthritogenic alphavirus.

In some embodiments, the Fc region is selected from human IgG1, human IgG1 with N297Q mutation, and mouse IgG2b. In some embodiments, at least one of the first Mxra8 region and the second Mxra8 region is a Mxra8 ectodomain region.

In some embodiments, the first Mxra8 region comprises a mammalian Mxra8 region and the second Mxra8 region comprises a non-mammalian Mxra8 region. In some embodiments, the mammalian Mxra8 region comprises a D2 mammalian Mxra8 region, including wherein the D2 mammalian Mxra8 region is a D2 mouse Mxra8 region. In other embodiments, the non-mammalian Mxra8 region comprises a D1 avian Mxra8 region or a D1 reptile Mxra8 region, including wherein the D1 avian Mxra8 region is a D1 duck Mxra8 region.

In another aspect of the present disclosure, a method of reducing Mxra8-associated alphavirus infection in a subject is provided. The method comprises administering to the subject a therapeutically effective amount of a Mxra8 inhibiting agent comprising a fusion protein, wherein the fusion protein comprises: an Fc region; a first Mxra 8 region; and a second Mxra8 region.

In some embodiments, the Fc region is selected from human IgG1, human IgG1 with N297Q mutation, and mouse IgG2b. In some embodiments, the first Mxra8 region comprises a mammalian Mxra8 region and the second Mxra8 region comprises a non-mammalian Mxra8 region.

In some embodiments, the Mxra8-associated alphavirus is an arthritogenic alphavirus selected from Chikungunya Virus (CHIKV), Mayaro Virus (MAYV), Ross River Virus (RRV), O'nyong nyong (ONNV), Barmah Forest Virus (BFV), Semliki Forest Virus (SFV), and Getah virus. In some embodiments, the Mxra8-associated alphavirus is a WEE complex alphavirus selected from Aura Virus (AURAV), Fort Morgan Virus (FMV), Highlands J Virus (HJV), Western Equine Encephalitis Virus (WEEV), Sindbis Virus (SINV), and Whataroa Virus (WHAV).

In some embodiments, the subject is a mammalian subject selected from a human, a non-human primate, a horse, a dog, a cat, a sheep, a pig, a mouse, a rat, a monkey, a hamster, and a guinea pig. In other embodiments, the subject is a non-mammalian subject selected from a reptile, a duck, a turkey, and a chicken.

In a further aspect of the present disclosure, a method of binding at least one of a surface-displayed alphavirus E1 protein and a surface-displayed alphavirus E2 protein in alphavirus-infected cells is provided. The method comprises administering to an alphavirus-infected cell a therapeutically effective amount of a Mxra8 inhibiting agent comprising a fusion protein, wherein the fusion protein comprises: an Fc region; a first Mxra8 region; and a second Mxra8 region.

In some embodiments, the Fc region is selected from human IgG1, human IgG1 with N297Q mutation, and mouse IgG2b. In some embodiments, the first Mxra8 region comprises a mammalian Mxra8 region and the second Mxra8 region comprises a non-mammalian Mxra8 region.

In some embodiments, the alphavirus-infected cell is infected with an arthritogenic alphavirus selected from Chikungunya Virus (CHIKV), Mayaro Virus (MAYV), Ross River Virus (RRV), O'nyong nyong (ONNV), Barmah Forest Virus (BFV), Semliki Forest Virus (SFV), and Getah virus. In some embodiments, the alphavirus-infected cell is infected with a WEE complex alphavirus selected from Aura Virus (AURAV), Fort Morgan Virus (FMV), Highlands J Virus (HJV), Western Equine Encephalitis Virus (WEEV), Sindbis Virus (SINV), and Whataroa Virus (WHAV).

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A-FIG. 1H is an exemplary embodiment showing Mxra8 is required for optimal infection of CHIKV and other alphaviruses in accordance with the present disclosure. FIG. 1A is a bar graph showing ΔMxra8 or control 3T3 or MEF cells were inoculated with CHIKV and stained for E2 protein (3 experiments, n=9; two-tailed t-test with Holm-Sidak correction; mean±s.d.). (b-d) FIG. 1B-FIG. 1D include multi-step growth curves with CHIKV-181/25 (FIG. 1B), CHIKV-AF15561 (FIG. 1C) or CHIKV-LR-2006 (FIG. 1D) in control, ΔMxra8 or Mxra8 trans-complemented ΔMxra8 3T3 cells (3 experiments, n=9; mean±s.d.). FFU, focus forming units. FIG. 1E is a bar graph showing ΔMxra8 or control 3T3 cells were inoculated with alphaviruses and processed for E2 or reporter gene expression (3 or more experiments, n=6 except for Semliki Forest virus (SFV), Sindbis-Western equine encephalitis virus chimaera (WEEV) and Sindbis-Eastern equine encephalitis virus chimaera (EEEV) in which with Holm-Sidak correction; mean±s.d.). FIG. 1F is a bar graph showing ΔMxra8 or control 3T3 cells were inoculated with indicated viruses and processed for viral antigen or reporter gene expression (3 experiments, mean±s.d.). FIG. 1G is a bar graph showing HeLa cells were transduced with control or MXRA8-1, MXRA8-2, MXRA8-3, or MXRA8-4 alleles, inoculated with CHIKV and processed for E2 staining (3 experiments, n=6; one-way ANOVA with Dunnett's test; mean±s.d.). FIG. 1H is a bar graph showing human MRC-5 cells depleted of MXRA8 with two different sgRNA were inoculated with CHIKV, and E2 expression was analyzed (3 experiments, n=9; one-way ANOVA with Dunnett's test; mean±s.d.). EMCV, encephalomyocarditis virus; MAYV; Mayaro virus; ONNV, O'nyong nyong virus; RRV, Ross River virus; RVFV, Rift Valley fever virus; VEEV, Venezuelan equine encephalitis virus; VSV, vesicular stomatitis virus; WNV, West Nile virus. *P<0.05; **P<0.01; ***P<0.001; ****P<0.0001; NS, not significant.

FIG. 2A-FIG. 2H is an exemplary embodiment showing Mxra8 modulates CHIKV attachment and internalization in accordance with the present disclosure. FIG. 2A includes bar graphs showing transfection of CHIKV RNA into control or ΔMxra8 cells. Cells were analyzed for E2 expression: left, percent positive; right, mean fluorescence intensity (MFI) (3 experiments, n=9; two-tailed t-test with Holm-Sidak correction, mean s.d.). FIG. 2B is a bar graph showing murine leukaemia virus (MLV) RNAs encoding GFP and pseudotyped with alphavirus structural genes were added to control or ΔMxra8 cells. Data are from three (MLV-CHIKV, n=9) or four (MLV-WEEV and MLV-EEEV, n=12) experiments (two-tailed t-test with Holm-Sidak correction; mean s.d.). FIG. 2C-FIG. 2D include bar graphs showing CH IKV-AF15561 was incubated with control, ΔMxra8 and Mxra8-overexpressing MEF cells at 4° C. (FIG. 2C left, FIG. 2D) or 37° C. (FIG. 2C right) as described in Methods section of Example 1. Cells were collected, and RNA (CHIKV and Gapdh) was measured by qRT-PCR (FIG. 2C) or surface E2 protein was analyzed by flow cytometry (FIG. 2D) (3 experiments; FIG. 2C, n=9; FIG. 2D, n=5; one-way ANOVA with Dunnett's test; mean±s.d.). FIG. 2E-FIG. 2F include graphs showing Mxra8-Fc, MXRA8-2-Fc or OPG-Fc were mixed with CHIKV-181/25 before infection of 3T3 (FIG. 2E) or MRC-5 (FIG. 2F) cells. Data are from two (MRC-5, n=6) or three (3T3, n=8-12) experiments; mean±s.d.; 50% inhibitory concentrations (EC50) are indicated to the right of each protein. FIG. 2G is a graph showing blockade of CHIKV-181/25 infection in 3T3 cells with hamster anti-Mxra8 or isotype control mAbs (2 experiments, n=6; mean±s.d., EC50 values are indicated to the right of each mAb). FIG. 2H is a bar graph showing trans-complementation of ΔMxra8 cells with vector, Mxra8, Mxra8 ΔC-tail, Mxra8 with GPI anchors (derived from placental alkaline phosphatase (PLAP) or the rodent herpesvirus Peru (RHVP) open reading frame R1 gene) or human MXRA8-2 (3 experiments; n=6; one-way ANOVA with Dunnett's test; mean±s.d.). ****P<0.0001; NS, not significant.

FIG. 3A-FIG. 3F is an exemplary embodiment showing direct binding of Mxra8 to CHIKV in accordance with the present disclosure. FIG. 3A-FIG. 3C include graphs showing purified CHIKV-181/25 (FIG. 3A), CHIKV virus-like particles (FIG. 3B) or chimaeric EEEV virions (FIG. 3C) were captured with anti-CHIKV or anti-EEEV human mAbs. Mxra8-Fc, MXRA8-2-Fc or OPG-Fc, and positive-control mAbs (CHK-11 or EEEV-10), were added. Data are from two (FIG. 3B and FIG. 3C) or three (FIG. 3A) experiments (a, n=8; b, n=4; c, n=6); mean±s.d. FIG. 3D includes sensograms of Mxra8 binding to CHIKV virus-like particles (left) and representative response curve for steady-state analysis, in which binding is plotted versus Mxra8 concentration (right). Experimental curves (black traces) were fit using a 1:1 binding model (red traces). Inset, linear Scatchard plot (4 experiments; mean and s.e.m.). FIG. 3E is a bar graph showing antibody blockade of Mxra8-Fc binding to CHIKV. Virus was incubated with indicated human mAbs against CHIKV, a mAb against WNV (E16) or no mAb before the addition of Mxra8-Fc (4 experiments, n=12; one-way ANOVA with Dunnett's test; mean±s.d.). FIG. 3F is a bar graph showing residues that result in loss of Mxra8-Fc binding to cell-surface-displayed CHIKV E2-E1. Residues are considered involved in the epitope if there is diminished binding without loss of protein integrity as judged by retention of interaction with mAbs (except for those previously mapped to a specific residue) Data are from two experiments. ****P<0.0001.

FIG. 4A-FIG. 4J is an exemplary embodiment showing Mxra8 contributes to alphavirus pathogenesis in accordance with the present disclosure. FIG. 4A includes graphs showing surface expression of MXRA8 on primary human keratinocytes, dermal fibroblasts, synovial fibroblasts, osteoblasts, chondrocytes, and skeletal muscle cells. One experiment of three is shown. FIG. 4B is a bar graph showing primary human cells were pre-incubated with anti-MXRA8 blocking mAbs before addition of CHIKV-AF15561 and processed for E2 staining (3 experiments, n=9; one-way ANOVA with Dunnett's test). FIG. 4C-FIG. 4D include bar graphs showing Mxra8-Fc or JEV-13 mAb were incubated with CHIKV-AF15561 for 30 min before subcutaneous inoculation. (FIG. 4C) At 12, 24 and 72 h, CHIKV was measured in the ipsilateral ankle and calf muscle. (FIG. 4D) At 72 h, foot swelling was measured (2 experiments, n=10; median viral titers: two-tailed Mann-Whitney test; mean foot swelling: two-tailed unpaired t-test). FIG. 4E is a bar graph showing Mxra8-Fc or JEV-13 mAb was mixed with O'nyong nyong virus immediately before subcutaneous inoculation. At 12 h, O'nyong nyong virus was measured in the ipsilateral ankle (2 experiments, n=10; two-tailed unpaired t-test; median values). FIG. 4F-FIG. 4G include bar graphs showing Mxra8-Fc or JEV-13 mAb was administered via an intraperitoneal route 6 h before CHIKV-AF15561 inoculation in the footpad. At 24 h, CHIKV was measured in the ipsilateral ankle (FIG. 4F). At 72 h, foot swelling was measured (FIG. 4G) (2 experiments, n=10; median viral titers: two-tailed Mann-Whitney test; mean foot swelling: two-tailed unpaired t-test). FIG. 4H-FIG. 4J include bar graphs showing pairs of anti-Mxra8 mAbs or isotype control hamster mAbs were administered via an intraperitoneal route 12 h before (FIG. 4H and FIG. 4I) or 8 or 24 h after (FIG. 4J) inoculation of CHIKV-AF15561. At 12 (FIG. 4H) and 72 (FIG. 4H and FIG. 4J) h, CHIKV was measured. At 72 h, foot swelling (FIG. 4I) was measured (2 experiments; FIG. 4H left, FIG. 4I: n=10; one-way ANOVA with Dunnett's test; FIG. 4H middle and right: n=10; Kruskal-Wallis with Dunn's test; FIG. 4J: n=8; two-tailed Mann-Whitney test). Ipsilat, ipsilateral; contra, contralateral. *P<0.05; **P<0.01; ***, P<0.001; ****P<0.0001.

FIG. 5A-FIG. 5C is an exemplary embodiment showing CRISPR-Cas9-based gene editing screen in accordance with the present disclosure. FIG. 5A is a schematic showing mouse 3T3 cells were transduced separately with two half libraries (A+B) comprising 130,209 sgRNAs, selected with puromycin and then inoculated with CHIKV-181/25-mKate2 (MOI of 1). One day later, mKate2-negative cells were sorted and then expanded in the presence of 2 μg ml⁻¹ each of CHK-152 and CHK-166 neutralizing mAbs. Several days later, cells were re-inoculated with CHIKV-181/25-mKate2 without neutralizing mAbs and re-sorted for mKate2-negative cells. This procedure was repeated one additional time. Afterwards, genomic DNA was collected for sgRNA sequencing and compared to the parent library for abundance (see e.g., Tables 1A, 1B, 2A, and 2B in provisional application Nos. 62/671,680 and 62/672,080, incorporated herein by reference). FIG. 5B is a diagram of the mouse Mxra8 and human MXRA8 orthologues. The transcript identification numbers and length of proteins are indicated to the right. Partial deletions in the isoforms 3 and 4 are shown as dashed lines. FIG. 5C is a phylogenetic tree of Mxra8 indicating genetic relationships. The neighbor-joining tree was constructed using MEGA 7. Scale bar shows the branch length. Right, identity (red) and similarity (yellow) matrix indicating the conservation of Mxra8 between species. The matrix was generated using MagGat 1.8.

FIG. 6A-FIG. 6E is an exemplary embodiment showing the efficiency of targeting Mxra8 expression by CRISPR-Cas9 gene editing in accordance with the present disclosure. FIG. 6A is a bar graph showing 3T3 cells were edited with a control or three different Mxra8 sgRNAs. After puromycin selection, bulk cells were inoculated with CHIKV-181/25-mKate2 and processed for marker gene expression by flow cytometry. Data are pooled from three experiments and expressed as mean±s.d. (n=6, one-way ANOVA with a Dunnett's multiple comparison test compared to control). FIG. 6B is a Western blot of Mxra8 in control and ΔMxra8 3T3 or MEF cells using hamster mAb 3G2.F5. One representative of two is shown. FIG. 6C includes graphs showing 3T3 and MEF cells (parent or ΔMxra8) were tested for Mxra8 surface expression by flow cytometry using anti-Mxra8 antibody (4E7.D10) and an isotype control mAb. One representative experiment of two is shown. FIG. 6D shows Sanger sequencing of Mxra8 in control and ΔMxra8 3T3 or MEF cells (SEQ ID NOs: 55-61). Sequencing data shows an alignment and individual out-of-frame deletions. FIG. 6E is a bar graph showing viability of control and ΔMxra8 3T3 and MEF cells. Equal numbers of cells were plated and viability was assessed over a 24-h period using the Cell-Titer Glo assay. The results were normalized to control cells and pooled from two experiments (n=6). Error bars indicate s.d. ****P<0.0001.

FIG. 7A-FIG. 7C is an exemplary embodiment showing CHIKV infectivity in CHO-K1 and CHO-745 cells in the presence or absence of ectopic Mxra8 expression in accordance with the present disclosure. FIG. 7A includes graphs showing surface expression of Mxra8 on CHO-K1 (wild type) and CHO-745 (glycosaminoglycan deficient) cells stably transduced with control (vector-only) or mouse Mxra8 as judged by flow cytometry. FIG. 7B includes graphs showing confirmation of heparan sulfate expression on the surface of CHO-K1 (wild type) and CHO-745 cells. Surface expression of heparan sulfate was evaluated using the R17 protein of rodent herpesvirus Peru, which binds to glycosaminoglycans on the surface of cells. R17GAG 2 is a mutant form of the protein that lacks binding to glycosaminoglycans and served as a negative control. In FIG. 7A and FIG. 7B, data are representative of two experiments. FIG. 7C is a bar graph showing CHO-K1 (wild type) and CHO-745 cells were transduced stably with control (vector) or mouse Mxra8 and inoculated with CHIKV (strains 181/25, AF15561 or LR-2006) and processed for intracellular E2 protein staining by flow cytometry. Data are from three experiments: mean±s.d. (n=6, one-way ANOVA with a Dunnett's multiple comparison test). ****P<0.001.

FIG. 8 shows growth curves of related alphaviruses in ΔMxra8 3T3 cells in accordance with the present disclosure. Control and ΔMxra8 3T3 cells were inoculated with BEBV, BFV, GETV, UNAV, MIDV or SFV at an MOI of 0.01 (except for BEBV, which was at 0.001), and supernatants were collected at the indicated times for focus forming assay. Data are pooled from two (BEBV) or three (all others) experiments and expressed as mean±s.d. (n=6, BEBV; n=9, BFV, GETV, UNAV and MIDV; n=12, SFV; two-way ANOVA with Sidak's multiple comparisons test). ***P<0.001; ****P<0.0001.

FIG. 9 shows surface expression of MXRA8 in different human cell lines in accordance with the present disclosure. Human cell lines were tested for MXRA8 surface expression by flow cytometry: 293T (embryonic kidney), A549 (lung adenocarcinoma), JEG3 (placental choriocarcinoma), U2OS (osteosarcoma), HFF-1 (foreskin fibroblasts), HeLa (cervical carcinoma), Huh7 (hepatocarcinoma), HTR8/SVneo (trophoblast progenitor), MRC-5 (lung carcinoma), hCMEC/D3 (cerebral microvascular endothelial cells), RPE (retinal pigment epithelial cell), Jurkat (T cell lymphoma), Raji (B cell lymphoma), K562 (eryrtholeukaemia), HT1080 (fibrosarcoma) and Hs 633T (fibrosarcoma) cells. Representative data are shown of two independent experiments. Grey histograms, isotype control mAb; red histograms, anti-MXRA8 mAb.

FIG. 10A-FIG. 10C is an exemplary embodiment showing MXRA8 supports enhanced infection of different CHIKV strains in accordance with the present disclosure. FIG. 10A includes graphs showing transduction and expression of different MXRA8 (MXRA8-1, MXRA8-2, MXRA8-3, and MXRA8-4) isoforms in HeLa cells. Representative data are shown from two experiments. Grey histograms, isotype control mAb; red histograms, anti-MXRA8 mAb. FIG. 10B is a bar graph showing the effect of ectopic expression of MXRA8-2 on CHIKV (strains 181/25, AF15561, and LR-2006) infection of A549, HeLa or 293T cells. Cells were collected and stained for CHIKV antigen with an anti-E2 antibody. Data are pooled from three experiments and expressed as mean±s.d. (n=6; two-tailed t-test with Holm-Sidak multiple comparison correction). FIG. 10C includes graphs showing transduction and expression of MXRA8-2 in 293T, A549, and HeLa cells. Representative data are shown from two experiments. ***P<0.001; ****P<0.0001.

FIG. 11A-FIG. 11B is an exemplary embodiment showing gene-editing of MXRA8 in human cell lines in accordance with the present disclosure. FIG. 11A includes graphs showing flow cytometry analysis of MXRA8 expression in human MRC-5, HFF-1, RPE and Hs 633T cells expressing control or two different MXRA8 sgRNAs. Data are representative of two experiments. FIG. 11B includes bar graphs showing gene-edited cells were inoculated with CHIKV (strains 181/25, AF15561 or LR-2006) in HFF-1, RPE, and Hs 633T cells. Cells were stained for viral antigen with an anti-E2 antibody. Data are pooled from two (HFF-1 and Hs 633T) or three (RPE) independent experiments and expressed as mean values±s.d. (n=6; one-way ANOVA with a Dunnett's multiple comparison test compared to the control). ****P<0.0001.

FIG. 12A-FIG. 12E is an exemplary embodiment showing Mxra8-Fc and anti-Mxra8 generation and function in accordance with the present disclosure. FIG. 12A includes a diagram of Mxra8-Fc (left) and SDS-PAGE (non-reducing (NR) and reducing (R) conditions) of purified material (right). Data are representative of three experiments. FIG. 12B is a scheme of anti-Mxra8 generation in Armenian hamsters. FIG. 12C includes bar graphs showing ELISA reactivity of anti-Mxra8 mAbs against Mxra8-Fc, MXRA8-2-Fc or OPG-Fc. Purified proteins (50 μl, 5 μg ml⁻¹) were immobilized overnight at 4° C. on ELISA plates. Anti-Mxra8 and isotype control mAbs were incubated for 1 h at room temperature. Signal was detected at 450 nm after incubation with horseradish peroxide conjugated goat anti-Armenian hamster IgG (H+L) and development with 3,3′-5,5′ tetramethylbenzidine substrate. FIG. 12D is a graph showing blockade of CHIKV-181/25 infection in MRC-5 cells with seven different hamster anti-Mxra8 or isotype control mAbs. mAbs were pre-incubated with cells for 1 h at 37° C. before addition of virus. After infection, cells were processed for E2 staining by flow cytometry. Relative infection was compared to a no mAb condition using flow cytometry and anti-E2 staining. Data in c and d are pooled from two experiments (n=6) and expressed as mean±s.d. FIG. 12E includes bar graphs showing anti-Mxra8 mAbs (1G11+7F1) or isotype control hamster mAbs (300 μg total) were administered via intraperitoneal route 8 or 24 h after inoculation of CHIKV-AF15561 in the footpad. Left, at 72 h after initial infection, CHIKV titers were measured in the ipsilateral and contralateral gastrocnemius (calf) muscles. Right, at 72 h, ipsilateral foot swelling was measured and compared to measurements taken immediately before infection. Data are pooled from two experiments (n=8; two-tailed Mann-Whitney test) and expressed as median values. *P<0.05, n.s., not significant.

FIG. 13A-FIG. 13B is an exemplary embodiment showing expression of truncated forms of Mxra8 mutants in accordance with the present disclosure. FIG. 13A includes graphs showing cell-surface expression of Mxra8 in ΔMxra8 3T3 cells trans-complemented with vector, Mxra8, Mxra8 AC-tail, Mxra8 with GPI anchors (derived from PLAP or RHVP open reading frame R1) or MXRA8-2. Data are representative of two independent experiments. FIG. 13B includes graphs showing effect of PI-PLC treatment on expression of different GPI-anchored forms or Mxra8. Data are representative of two independent experiments.

FIG. 14A-FIG. 14D is an exemplary embodiment showing binding of Mxra8-Fc to surface-displayed E2 protein in virus-infected cells and mapping of Mxra8 binding site on E2 in accordance with the present disclosure. FIG. 14A is a diagram of the cell-based binding assay. After infection, viral structural proteins (for example, E2) traffic to the cell plasma membrane where progeny virion assembles and buds. E2 protein is displayed on the cell surface and is accessible to the binding of Mxra8-Fc and detection with a goat anti-mouse IgG secondary antibody by flow cytometry. FIG. 14B includes graphs showing binding of Mxra8-Fc to virus-infected wild-type 3T3 cells. Cells were infected with the indicated viruses and processed for Mxra8-Fc binding by flow cytometry. Virus-specific anti-E2 antibodies were used as positive controls. Data are representative of two independent experiments. FIG. 14C-FIG. 14D include structures showing mapped residues as magenta spheres (FIG. 14C) or sticks (FIG. 14D) on the CHIKV p62-E1 structure (FIG. 14C, trimer of dimers, top view; FIG. 14D, heterodimer, side view) using PyMOL (Protein Data Bank code: 3N41). The E1 and E2 proteins are colored in grey and cyan, respectively.

FIG. 15A-FIG. 15B is an exemplary embodiment showing most animal orthologs of Mxra8 (with the exception of cow Mxra8) support infection of CHIKV in 3T3 trans-complemented cells in accordance with the present disclosure. FIG. 15A is a bar graph showing surface expression of Mxra8 as determined by flow cytometry and staining with cross-reactive anti-Mxra8 monoclonal antibodies. FIG. 15B is a bar graph showing CHIKV infection in cells trans-complemented with Mxra8 from different species. Note, Cow Mxra8 was expressed strongly on the surface but did not support CHIKV infection as judged by staining for viral antigen. Results are representative of three independent experiments.

FIG. 16A-FIG. 16C is an exemplary embodiment showing a 15-amino acid insertion in cow Mxra8 prevents binding to CHIKV virions in accordance with the present disclosure. FIG. 16A is a diagram of generation of recombinant mouse Mxra8-Fc fusion proteins that lacked or contained the additional 15 residues from cow (mouse Mxra8 and mouse Mxra8+moo) and reciprocally cow Mxra8-Fc fusion protein that lacked or contained the 15 residues (cow Mxra8 and cow Mxra8 Δmoo). FIG. 16B is an alignment of mouse Mxra8, mouse Mxra8+moo, cow Mxra8, and cow Mxra8 Δmoo proteins in the region of the 15-amino acid insertion (SEQ ID NOs:62-65). FIG. 16C is a bar graph showing binding data in an ELISA. CHIKV virions were captured with a human anti-CHIKV mAb and then tested for binding to increasing doses (0.1, 1, and 10 μg/ml) of mouse Mxra8-Fc, mouse Mxra8-Fc+moo, cow Mxra8-Fc, and cow Mxra8-Fc Δmoo. Signal was detected by Optical density. Results are representative of three independent experiments.

FIG. 17 includes bar graphs showing a 15-amino acid insertion in cow Mxra8 prevents alphavirus infection in accordance with the present disclosure. 3T3 cells were trans-complemented with mouse Mxra8, mouse Mxra8+moo, cow Mxra8, or cow Mxra8 Δmoo. Subsequently, cells were inoculated with CHIKV, Ross River virus, or Mayaro virus. One day later, cells were harvested and infection was determined by flow cytometry after staining with monoclonal antibodies against CHIKV, Ross River virus, and Mayaro virus E2 protein. Results are representative of three independent experiments. Red arrows show gain of infection when the 15-amino acid insertion is deleted in cow Mxra8. Note that addition of the 15-amino acid insertion to mouse Mxra8 renders it unable to support alphavirus infection.

FIG. 18A-FIG. 18B is an exemplary embodiment showing the generation of Mxra8^{mut/mut} mice by CRISPR-Cas9 gene targeting in accordance with the present disclosure. FIG. 18A is an alignment showing sequencing of Mxra8 gene of two different founder lines establishes 8 and 97 nucleotide deletions (SEQ ID NOs: 66-69). FIG. 18B is a Western blot of muscle tissue from Mxra8^{+/+}, Mxra8^{Δ8/+} (HET), Mxra8^{Δ8/Δ8} (KO) lines. Data are representative of two experiments.

FIG. 19 includes graphs depicting diminished CHIKV infection and swelling in Mxra8-deficient mice during the acute phase in accordance with the present disclosure. Mxra8^{Δ8/Δ8}, Mxra8^{Δ8/+}, Mxra8^{Δ97/Δ97}, or Mxra8^{+/+} (WT) mice were inoculated subcutaneously in the foot with 10³ FFU of CHIKV-AF15561 (top two rows) or CHIKV-LR2006 (bottom row). At 3 dpi, foot swelling (left panels) was measured (2 experiments, n=10, Mann-Whitney test, ***, P<0.001; ****, P<0.0001), and ipsilateral and contralateral ankles and calf muscles (right panels) were harvested for infectious virus titration by FFU assay (2 experiments, n=10, ANOVA (top row) or Mann-Whitney (bottom two rows) tests, *, P<0.05; **, P<0.01; ***, P<0.001; ****, P<0.0001).

FIG. 20 includes graphs showing diminished infection of other arthritogenic alphaviruses (ONNV, MAYV, and RRV) in Mxra8^{Δ8/Δ8} mice in accordance with the present disclosure. Mxra8^{Δ8/Δ8} and Mxra8^{+/+} mice were inoculated subcutaneously in the foot with 3 FFU of ONNV (top row), MAYV (middle row), or RRV (bottom row). Ipsilateral and contralateral musculoskeletal tissues at 3 dpi were harvested for infectious virus titration by FFU assay (2 experiments, n=10, Mann-Whitney test, *, P<0.05; **, P<0.01; ***, P<0.001; ****, P<0.0001).

FIG. 21A-FIG. 21B is an exemplary embodiment showing Mxra8 crystal structure and topology diagram in accordance with the present disclosure. FIG. 21A is a ribbon model of the mouse Mxra8 protein structure. The β-strands are labeled with a letter for each element followed by a number for the domain. The two Ig-like domains are colored from the N- to C-terminus in a rainbow spectrum of blue to red. The disulfides are shown as ball and stick bonds colored yellow. The cysteine positions forming the interdomain disulfide bond is given. The N- and C-termini are labeled in lowercase. FIG. 21B is a secondary structure diagram of Mxra8. Numbering corresponds to the sequence positions in the x-ray structure. The placement and orientation of β-strands are indicated by magenta arrows. The disulfide bonds are shown as yellow connecting lines with cysteine positions labeled. The dashed line divides the two halves of the Ig-like domain β-sandwich. The strand swaps are visible as connections between the D1 and D2 domains.

FIG. 22A-FIG. 22F is an exemplary embodiment showing two-dimensional cisTEM analysis of CHIKV particles with or without Mxra8 bound in accordance with the present disclosure. FIG. 22A includes raw electron micrographs of CHIKV VLPs. FIG. 22B is a two-dimensional classification scheme for binning of CHIKV VLPs. FIG. 22C-FIG. 22D include two-dimensional equatorial slices of CHIKV VLP alone (FIG. 22C) or CHIKV VLP in complex with Mxra8 (FIG. 22D). Dimensions: the outer radius of the nucleocapsid shell (˜180 Å), lipid bilayer (˜240 Å), E1 protein glycoprotein shell (˜280 Å), E2 protein spike (˜340 Å), and bound Mxra8 (˜350 Å) from the viral center. FIG. 22E-FIG. 22F include graphs showing Fourier shell correlation (FSC) plot versus resolution for CHIKV VLP alone (FIG. 22E) or CHIKV VLP with bound Mxra8 (FIG. 22F).

FIG. 23A-FIG. 23D is an exemplary embodiment showing cryo-EM reconstruction of CHIKV particles with or without Mxra8 binding in accordance with the present disclosure. FIG. 23A-FIG. 23C include paired, colored surface representations (top panel) and equatorial cross-sections (bottom panel) of CHIKV VLP (FIG. 23A), CHIKV VLP+Mxra8 (FIG. 23B), and local resolution of CHIKV VLP+Mxra8 (FIG. 23C). The white triangle indicates one icosahedral asymmetric unit. The 5-fold (i5), 3-fold (i3), and 2-fold (i2) icosahedral axes of symmetry are indicated with a pentagon, triangles, and oval, respectively. Trimeric spikes are labeled “i3” if coincident with the i3 axes, and “q3” if on a quasi 3-fold axes. Black arrows: directions of icosahedral symmetry axes (i2, i3, q3, and i5). FIG. 23D includes zoomed-in views of the geometry of Mxra8 binding to CHIKV E2-E1 proteins in different chemical environments, colored by radial distance (left) and local resolution (right). Radial distance color scheme in Figure: red, electron dense core and RNA; yellow, capsid; green, membrane lipid; cyan, E1; dark blue, E2 spike; and magenta, Mxra8.

FIG. 24A-FIG. 24B is an exemplary embodiment showing the atomic model of Mxra8 interaction with CHIKV in accordance with the present disclosure. FIG. 24A is the refined model of Mxra8 and CHIKV structural proteins (E1, E2, transmembrane (TM) helices, and Capsid) in the electron density map from CHIKV VLP with Mxra8 reconstruction, viewed from the top of the asymmetric unit (left panel) and from the side of a subunit (right panel). Color scheme: grey, E1; cyan, E2; green, capsid; and magenta, Mxra8. FIG. 24B is the surface representation of all E1 and E2 proteins in contact with a molecule of Mxra8 (shown as ribbon diagram), labeled and colored by domain. Prime denotes domains from adjacent intraspike E2-E1 heterodimer. Double prime denotes domains from the neighboring interspike E2-E1 heterodimer. D1 and D2 refer to the Ig-like domains of Mxra8.

FIG. 25A-FIG. 25E is an exemplary embodiment showing the mutagenesis and antibody mapping data supporting the Mxra8-CHIKV model in accordance with the present disclosure. FIG. 25A is a cartoon of the quaternary determinants of Mxra8 (magenta) binding to the asymmetric unit of CHIKV. Key Mxra8 contact regions (orange) on domains of E1 (grey) and E2 (cyan) were identified from the electron density map using COOT. FIG. 25B is a model showing CHIKV E2 residues at the binding interface as determined from the electron density map are labeled and shown as magenta spheres on the CHIKV E2-E1 heterotrimer (PDB 5ANY). FIG. 25C is a model showing CHIKV E2 residues at the CHIKV-Mxra8 interface as defined by previous alanine scanning mutagenesis data (Zhang et al., 2018). Mapped CHIKV E2 residues are labeled and shown as green spheres on the CHIKV E2-E1 heterotrimer. FIG. 25D is a model showing CHIKV E2 residues identified as epitopes for neutralizing human anti-CHIKV mAbs (Long et al., 2015; Smith et al., 2015) that also block binding to Mxra8 (Zhang et al., 2018). Antibody binding residues are labeled and shown as salmon spheres on the CHIKV E2-E1 heterotrimer. FIG. 25E includes a model showing CHIKV E2 residues identified as the epitope for CHK-265 mAb (Fox et al., 2015) are labeled and shown as deep blue spheres on the CHIKV E2-E1 heterotrimer (left panel) and order-of-addition BLI assay traces (right panel). CHIKV VLP was captured on the biosensor with mAb and then incubated sequentially with CHK-265 and Mxra8 (red line and box) or Mxra8 and CHK-265 (blue line and box). Similar binding traces were seen using CHK-265 Fab fragments. Color scheme in figure: gray, E1; cyan, E2; and magenta, Mxra8.

FIG. 26A-FIG. 26E is an exemplary embodiment showing the modes of Mxra8 engagement by CHIKV in accordance with the present disclosure. FIG. 26A includes a two-dimensional equatorial slice (top panel) and Fourier shell correlation (FSC) plot (bottom panel) of CHIKV infectious particles in complex with Mxra8. FIG. 26B-FIG. 26C include views of asymmetric units of CHIKV infectious particles (FIG. 26B) or CHIKV VLP (FIG. 26C) with bound Mxra8 electron density at high contour (left panel) and low contour (middle and right panels), colored by structural proteins if within 6 Å of docked model coordinates. Mxra8 coordinates were removed from the model, and E3 was docked in for (FIG. 26B). The σ value is the standard deviation of density values above the mean. Color scheme: grey, E1; cyan, E2; yellow, E3; capsid, green; Mxra8 and other unexplained density, magenta. FIG. 26D-FIG. 26E include kinetic sensograms and steady-state analysis of murine Mxra8 binding to CHIKV VLPs fit to a 1:1 binding model (FIG. 26D) or two-site model (one high affinity site and three low but equal affinity sites) (FIG. 26E). Raw experimental traces are in black, fit traces are in red. Inset, Scatchard plot (4 experiments; mean, standard error of the mean (SEM), and χ² values).

FIG. 27A-FIG. 27E is an exemplary embodiment showing the biochemical characterization of CHIKV VLPs and soluble Mxra8 protein in accordance with the present disclosure. Related to FIG. 21A-FIG. 21B and FIG. 22A-FIG. 22F. FIG. 27A-FIG. 27B includes images showing Mxra8 characterization. Mxra8-Fc was produced in Expi-293 cells, purified by protein A affinity chromatography, and digested with HRV protease to release the monomeric form of Mxra8 from the Fc region. FIG. 27A is a Coomassie-stained SDS-PAGE under non-reducing and reducing conditions of 5 μg of undigested (left panel) and HRV-digested (right panel) Mxra8. One representative experiment of three is shown. FIG. 27B shows SEC-MALS of soluble Mxra8 showing a monomeric form at ˜38 kDa.C-E. VLP characterization. After transfection of HEK-293 cells with plasmid DNA encoding CHIKV C-E3-E2-6K-E1 (strain 37997), VLPs were harvested from the supernatant and purified by anion exchange chromatography. FIG. 27C is a graph showing Coomassie-stained 4-12% SDS-PAGE of 2 μg of purified VLPs. E2 and E1 co-migrate at ˜52-53 kDa. Capsid migrates at ˜35 kDa. One representative experiment of two is shown. FIG. 27D includes images showing immunoblotting of 0.25 μg of VLPs with a rabbit anti-CHIKV polyclonal antibody. One representative experiment of two is shown. FIG. 27E is a graph showing dynamic light scattering analysis of CHIKV VLPs. A single homogeneous peak of 68 nm (680 Å)+5.3 is shown. Data are representative of six experiments from two different VLP preparations.

FIG. 28 shows the sequence alignment of Mxra8 orthologs in accordance with the present disclosure (SEQ ID NOs: 70-72). Related to FIG. 21A-FIG. 21B. The β-strand secondary structure from the mouse model is shown by arrows above the alignment. Numbering above the alignment corresponds to the sequence positions within the mouse protein. Aligned residues are colored by amino acid type with background boxes colored by conservation. Numbers under the alignment indicate contacts between the Mxra8 and individual E2-E1 heterodimers on the VLP, given as percentage buried surface area as defined by PDBePISA server: 1 represents 10% buried surface area, 2 represents 20% buried surface area, and so on. Wrapped E2-E1 represent the contacts to the E2-E1 heterodimer making the primary contacts to Mxra8. Adjacent E2-E1 denote the contacts to the E2-E1 heterodimer adjacent to the primary E2-E1 dimer but within the same trimeric spike. Neighbor E2-E1 denotes contacts against the E2-E1 pair in the neighboring spike.

FIG. 29 shows the sequence alignment of E1 proteins of arthritogenic and encephalitic alphaviruses. Related to FIG. 25A-FIG. 25E. Amino acid sequence alignment of E1 proteins of arthritogenic (CHIKV, ABX40011, SEQ ID NO: 73; MAYV, AAY45742, SEQ ID NO: 74; RRV, ACV67002, SEQ ID NO: 75; ONNV, AOS52786, SEQ ID NO: 76; SFV, AAM64227, SEQ ID NO: 77; and SINV, AUV65223, SEQ ID NO: 78) and encephalitic alphaviruses (WEEV, AAF60166, SEQ ID NO: 79; EEEV, AAF04796, SEQ ID NO: 80; and VEEV, AAB02517, SEQ ID NO: 81). Alignments were performed between alphaviruses that do (group 1, left margin) or do not (group 2, left margin) require Mxra8 for infection using Cobalt (Papadopoulos and Agarwala, 2007) and the Figure was prepared using ESPript 3.0 (Robert and Gouet, 2014). Domains I (red), II (yellow), Ill (blue), stem (clear), transmembrane (grey), and the fusion loop (orange) are indicated below the sequence. Red boxes, 100% conserved; White boxes and red letters; homologous residues; White boxes and black letters, non-conserved residues. Green numbers indicate disulfide bonds. Stars indicate contact residues for Mxra8 (see TABLE 4).

FIG. 30 shows the sequence alignment of E2 proteins of arthritogenic and encephalitic alphaviruses in accordance with the present disclosure. Related to FIG. 25A-FIG. 25E. Amino acid sequence alignment of E2 proteins of arthritogenic (CHIKV, ABX40011, SEQ ID NO: 82; MAYV, AAY45742, SEQ ID NO: 83; RRV, ACV67002, SEQ ID NO: 84; ONNV, AOS52786, SEQ ID NO: 85; SFV, AAM64227, SEQ ID NO: 86; and SINV, AUV65223, SEQ ID NO: 87) and encephalitic alphaviruses (WEEV, AAF60166, SEQ ID NO: 88; EEEV, AAF04796, SEQ ID NO: 89; and VEEV, AAB02517, SEQ ID NO: 90). Alignments were performed between alphaviruses that do (group 1, left margin) or do not (group 2, left margin) require Mxra8 for infection using Cobalt (Papadopoulos and Agarwala, 2007) and the Figure was prepared using ESPript 3.0 (Robert and Gouet, 2014). Domains A (aqua), B (green), C (red), N-terminal linker and arches (purple), stem (clear), transmembrane (grey), and cytoplasmic tail (clear) are indicated below the sequence. Red boxes, 100% conserved; White boxes and red letters; homologous residues; White boxes and black letters, non-conserved residues. Stars indicate contact residues for Mxra8 (see TABLE 4). Green squares indicate key E2 residues that result in loss of Mxra8 binding identified by alanine-scanning mutagenesis (Zhang et al., 2018). Salmon circles represent residues of epitopes of human anti-CHIKV mAbs that block Mxra8 binding (Smith et al., 2015).

FIG. 31A-FIG. 31C is an exemplary embodiment showing the unique E1 contacts by site 2 Mxra8 in accordance with the present disclosure. Related to FIG. 25A-FIG. 25E. FIG. 25A shows the top view of the asymmetric unit of CHIKV VLP with labeled Mxra8 binding sites. Site 2 is the Mxra8 (magenta) site that makes additional contacts to domain I of E1 directly beneath it (grey). E2, yellow; capsid, green. FIG. 25B-FIG. 25C show side views (top panels) and zoomed-in views (bottom panels) of site 2 (FIG. 25B) and site 3 (FIG. 25C), highlighting the unique residues of E1 (orange) that contact site 2 of Mxra8 binding. Contacts were identified from the atomic model using PDBePISA.

FIG. 32A-FIG. 32E is an exemplary embodiment showing the presence of E3 can sterically hinder Mxra8 binding in accordance with the present disclosure. Related to FIG. 26A-FIG. 26E. FIG. 32A shows a view of asymmetric unit of CHIKV infectious particles with difference map of Mxra8 docked on. E1, E2, E3, and Mxra8 sites are labeled, with the high affinity site 2 labeled with a square. FIG. 32B-FIG. 32D includes zoomed-in views highlight the amount of steric clashing of Mxra8 at site with E3 from the adjacent spike. The surface area of occluded Mxra8 density (zoned within 2 Å of E3 coordinates) was calculated as follows: site 1 (FIG. 32B), 400.3 Å²; site 2 (FIG. 32C), 19.6 Å²; site 3 (FIG. 32D), 218.4 Å²; and site 4 (FIG. 32E), 421.4 Å². Color scheme: grey, E1; cyan, E2; transparent yellow, E3; capsid, green; Mxra8, magenta. Prime labels refer to Mxra8 sites on adjacent spikes.

FIG. 33A-FIG. 33C is an exemplary embodiment showing human Mxra8 binding to CHIKV VLPs in accordance with the present disclosure. Related to FIG. 26A-FIG. 26E. FIG. 33A shows SEC-MALS of cleaved human Mxra8 showing a monomeric form at −34 kDa. FIG. 33B-FIG. 33C include kinetic sensograms and steady-state analysis of human Mxra8 binding to CHIKV VLPs fit to a 1:1 binding model (FIG. 33B) or a two-site binding model (FIG. 33C) (one high affinity site and three low but equal affinity sites). Raw experimental traces are in black, fit traces are in red. Inset, Scatchard plot (2 experiments; mean, standard error of the mean (SEM), and χ² values).

FIG. 34A-FIG. 34L is an exemplary embodiment showing avian MXRA8 is required for optimal infection of SINV and WEEV alphaviruses in accordance with the present disclosure. FIG. 34A is a phylogenetic tree based on alphavirus E1 and E2 protein sequences with complexes clustered in blue (WEEV complex), orange (SFV complex), purple (EEEV complex), and yellow (VEEV complex). Viruses in this study are bolded. FIG. 34B-FIG. 34C include bar graphs showing ΔMxra8 3T3 cells complemented with mouse (pink), chicken (red), duck (blue) or turkey (yellow) MXRA8 or empty vector control (gray) were inoculated with CHIKV 181/25 (FIG. 34B) or SINV TR339 (FIG. 34C) and stained for CHIKV or SINV antigen (4 and 3 experiments in triplicate, respectively). Infection is normalized to levels detected with cells expressing mouse (FIG. 34B) or chicken (FIG. 34C) MXRA8. FIG. 34D includes multi-step growth curves with SINV TR339 in ΔMxra8 3T3 cells complemented with chicken or mouse MXRA8 or empty vector control. Virus was titrated by focus-forming unit (FFU) assay (4 experiments, duplicate). FIG. 34E-FIG. 34G include bar graphs showing ΔMxra8 3T3 cells complemented with mouse, chicken, duck, or turkey MXRA8 or empty vector control were inoculated with SINV-WEEV-GFP (FIG. 34E), SINV-EEEV-GFP (FIG. 34F) or SINV-VEEV-GFP (FIG. 34G). Infection was assessed by GFP expression (4 to 7 experiments, triplicate). FIG. 34H is a bar graph showing CEFs were inoculated with CHIKV 181/25, SINV-WEEV-GFP, or SINV TR339 and stained for viral antigen. FIG. 34I-FIG. 34J include bar graphs showing CEFs were pre-incubated with anti-chicken Mxra8 or an isotype control mAb and inoculated with SINV-WEEV-GFP (FIG. 34I) or SINV TR339 (FIG. 34J). Infection was assessed by GFP expression or E1 staining and compared between infected (red) and non-infected cells (blue) (3 experiments in triplicate). FIG. 34K-FIG. 34L include bar graphs showing ΔMxra8 chicken fibroblasts complemented with chicken or mouse MXRA8 (2 independent clones of each shown) or empty vector control and non-targeted (NT1 and NT2) CEFs were inoculated with SINV TR339 (FIG. 34K), CHIKV 181/25 (FIG. 34L). Infection was assessed by flow cytometry, derived from the percent and mean fluorescence intensity of the infected cells, and normalized to the non-targeting control NT1 (for SINV) or ΔMxra8 (1)+Mouse Mxra8 (for CHIKV) (3 experiments, in triplicate). FIG. 34B, FIG. 34C, FIG. 34E, FIG. 34I, FIG. 34J, FIG. 34K and FIG. 34L: one-way ANOVA with Dunnett's post-test; mean±standard deviation (SD). FIG. 34D: Two-way ANOVA with Tukey post-test; mean±SD. ns, non-significant; *, P<0.05; **, P<0.01; ****, P<0.0001). See also FIG. 41A-FIG. 41J, FIG. 42A-FIG. 42M, FIG. 43A-FIG. 43N, FIG. 44A-FIG. 44D, and TABLE 6.

FIG. 35A-FIG. 35G is an exemplary embodiment showing avian MXRA8 modulates SINV attachment and internalization and binds to WEEV VLPs in accordance with the present disclosure. FIG. 35A-FIG. 35C include bar graphs showing SINV was incubated with ΔMxra8 3T3 cells (control), chicken Mxra8-complemented ΔMxra8 3T3 cells, or mouse Mxra8-complemented ΔMxra8 3T3 cells at 4° C. without (FIG. 35A) or with (FIG. 35B) pre-incubation with mouse-anti-chicken MXRA8 mAbs. Bound virions were quantified by measuring viral RNA levels and Gapdh levels via qRT-PCR. After removal of unbound virus, the temperature was shifted to 37° C. to allow internalization (FIG. 35C). Intracellular RNA (SINV and Gapdh) levels were measured by qRT-PCR. (3 experiments, triplicate). FIG. 35D-FIG. 35E include graphs showing binding of chicken MXRA8-Fc and murine MXRA8-Fc to WEEV (FIG. 35D) or CHIKV (FIG. 35E) VLPs by ELISA (WEEV positive control, WEEV-204; CHIKV positive control, CHK-265; negative control, anti-HCV H77.39; CHIKV VLP ELISA: 2 experiments, triplicate; WEEV VLP ELISA: 2 experiments, duplicate). FIG. 35F-FIG. 35G include graphs showing binding of WEEV VLPs (FIG. 35F) and CHIKV VLPs (FIG. 35G) to duck, sparrow, or mouse MXRA8 by ELISA. SARS-CoV-2 receptor-binding domain served as a negative control (3 experiments, duplicate). FIG. 35A-FIG. 35C: one-way ANOVA with Dunnett's post-test; mean±SD. *, P<0.05; ****, P<0.0001).

FIG. 36A-FIG. 36D is an exemplary embodiment showing cryo-EM reconstruction of duck MXRA8 bound to WEEV in accordance with the present disclosure. FIG. 36A is a cryo-EM density map of WEEV-VLP bound to duck MXRA8 at 4.74 Å resolution. WEEV-E1, WEEV-E2, and MXRA8 are colored as light-blue, dark-blue, and violet, respectively. Rotational symmetries along the 2-fold, 3-fold, and 5-fold axes are displayed with white numbers. FIG. 36B is a map of WEEV-VLP bound to duck MXRA8. Asymmetric unit contains the entire quasi 3-fold spike (q3), and a single icosahedral spike (i3) E1-E2 heterodimer. Shown are two views of the asymmetric unit, a side view (left, parallel with the viral membrane) and a top-down view (right, perpendicular with the viral membrane). Local resolution of map is colored from blue (4.25 Å) to white (5.25 Å) to red (6.25 Å). Density maps are viewed at contour level=0.26 (0.966). FIG. 36C is a surface diagram of MXRA8 at site 3, detailing the three unique viral E1-E2 heterodimer contacts, wrapped (light gray), intraspike (gray), and interspike (dark gray). FIG. 36D is a surface diagram of MXRA8 at site 3 and interacting E1-E2 heterodimers, termed: E1-E2-wrapped, E1-E2-intraspike, or E1-E2-interspike. MXRA8 D1: light magenta; MXRA8 D2: dark magenta; E1 domain I: light gray; E1 domain II: medium-gray; E1 domain III: dark-gray; E1-TM (black); E2 Å domain: light-cyan; E2 β-linker: medium-cyan; E2 B domain: dark-cyan; E2 C domain: medium-blue; E2-TM: dark-blue. See also FIG. 45A-FIG. 45D, FIG. 46, and TABLE 7-TABLE 9.

FIG. 37A-FIG. 37E is an exemplary embodiment showing duck and mouse MXRA8 use a domain-inverted binding paradigm to engage WEEV and CHIKV in accordance with the present disclosure. FIG. 37A is a ribbon and topology diagram of duck MXRA8 with β-strands labeled with standard Ig-like fold conventions. The β-strands are colored by a rainbow and are depicted as a cartoon with residue number start/stop locations. Disulfide bonds between cysteines are depicted with a yellow line. FIG. 37B is a cryo-EM density of the duck MXRA8 domain in the binding-groove, modeled as either D1 or D2. D1 has an extra β-strand (strand H) that better fits the density, whereas D2 lacks this β-strand and leaves this density unfilled. Density maps are viewed at contour level=0.42 (1.56a). FIG. 37C is a cryo-EM map of duck D1 with clear density in the region expected for N-linked glycan on Asn-120. Density map is viewed at contour level=0.42 (1.56a). FIG. 37D is a schematic showing differential binding modes for avian and mammalian MXRA8 binding to WEEV and CHIKV, respectively. Avian MXRA8 D1 is distal to the cell membrane and binds E1-intraspike heterodimer, whereas mammalian MXRA8 D1 is proximal to the cell and binds E2-wrapped heterodimer. FIG. 37E is a multi-sequence alignment of WEE- and SF-complex E1 proteins with E1-intraspike duck MXRA8 D1 contacts (depicted with red circles, SEQ ID NOs: 93-99). N-linked glycosylation sites are shown with dotted red boxes. Sequence similarities are colored from white to black, with black being most similar. E1 β-strands are labeled as described in Voss et al. See also FIG. 47, FIG. 48, and FIG. 49.

FIG. 38A-FIG. 38D is an exemplary embodiment showing assessment of the MXRA8 binding model in accordance with the present disclosure. FIG. 38A is a sequence alignment of D1 of MXRA8 at regions that contact WEEV E1 (SEQ ID NOs: 100-105). Similar sequence identities are colored by most variable (white) to conserved (black). b-strands are labeled based on topology in FIG. 37A. Filled circles above the alignment denote avian MXRA8 contact residues in the C-C′ loop (yellow), C″-D loop (red), D-E loop (cyan), and B-C connector (pale green) are denoted by circles. FIG. 38B is a structure of avian MXRA8 in two possible binding modes, non-flipped (model 1, left) and flipped (model 2, mouse/human-like, right), with contact residues in either D1 or D2 shown as space filling spheres in the C-C′ loop (yellow), C″-D loop (red), D-E loop (cyan), and B-C connector (pale green). FIG. 38C shows binding sites for WEEV and CHIKV on D1 (light purple) and D2 (dark purple) of MXRA8. Structurally defined binding sites and amino acid contact residues are colored according to the viral E2-E1 heterodimer engaged: wrapped (cyan), intraspike (red), or interspike (yellow). Stalk attaching MXRA8 to the cell membrane is represented as a red dashed line and is used to denote mode of binding, with mammalian MXRA8 adopting a flipped orientation relative to avian MXRA8. Avian and mammalian MXRA8 engage E2-E1 heterodimers most proximal to viral membrane with D1 and D2, respectively. FIG. 38D is a bar graph showing SINV-GFP infection in ΔMxra8 3T3 cells complemented with wild-type (blue) and indicated mutants (light blue, yellow, or red) duck MXRA8 (3 experiments, triplicate; normalized to wild-type duck MXRA8 infection. Infection and GFP fluorescence were analyzed by flow cytometry. FIG. 38D: one-way ANOVA with Dunnett's post-test; mean±standard deviation (SD). ****, P<0.0001). See also FIG. 50.

FIG. 39A-FIG. 39S is an exemplary embodiment showing chimeric avian-mammalian MXRA8 interacts with both WEE and SF complex alphaviruses in accordance with the present disclosure. FIG. 39A is a schematic of chimeric MXRA8 proteins used to assess binding modes. In addition to mouse or duck MXRA8, chimeras shown are Du-D1-Mo-D2 and Mo-D1-Du-D2. FIG. 39B-FIG. 39C include bar graphs showing SINV-GFP (FIG. 39B) and SINV-WEEV-GFP (FIG. 39C) infection in ΔMxra8 3T3 cells complemented with wild-type duck (blue) and chimeric (blue/pink) duck-mouse MXRA8 (3 experiments in triplicate; normalized to wild-type duck MXRA8 infection. Infection and GFP fluorescence were analyzed by flow cytometry. FIG. 39D-FIG. 39E include bar graphs showing SINV-CHIKV (LR 2006 strain) (FIG. 39D) and MAYV (FIG. 39E) infection of ΔMxra8 3T3 cells complemented with an empty vector, or wild-type mouse and chimeric duck-mouse MXRA8 (3 experiments in triplicate; normalized to wild-type mouse MXRA8 infection). Infection was analyzed by flow cytometry by evaluating viral antigen staining with mAbs (CHIKV or MAYV). FIG. 39F-FIG. 39I include graphs showing staining of the surface of Vero cells infected with SINV-TR339 (FIG. 39F), SINV-CHIKV (LR 2006 strain), (FIG. 39G) MAYV (FIG. 39H), or SINV-VEEV (FIG. 39I) after incubation with serially diluted mouse MXRA8-Fc, duck MXRA8-Fc, Du-D1-Mo-D2 MXRA8-Fc, LDLRAD3-D1-Fc proteins or cross-reactive anti-E1 mAb (DC2.112, positive control)³⁷) control. Data are expressed as the percentage of infected cells that bound positively to the indicated proteins by flow cytometry (representative of 2 experiments). FIG. 39J-FIG. 39L include graphs showing neutralization of SINV TRR399 (FIG. 39J), SINV-CHIKV (FIG. 39K), or SINV-WEEV (FIG. 39L) (all three viruses expressing eGFP) infection by duck MXRA8-Fc, mouse MXRA8-Fc, Du-D1-Mo D2-MXRA8-Fc, Mo-D1-Du-D2-MXRA8-Fc, or LDLRAD3-D1-Fc control (3 experiments, duplicates). Infection was analyzed by flow cytometry (GFP expression) and normalized to levels after incubation with LDLRAD3-D1-Fc protein. FIG. 39M-FIG. 39N include graphs showing clinical disease (FIG. 39M) and survival (FIG. 39N) of 4-week-old female CD-1 mice inoculated with 10³ PFU of WEEV (McMillan strain) mixed with Du-D1-Mo D2 N66R-MXRA8-Fc or LDLRAD3-D1-Fc. FIG. 39O-FIG. 39S include dot plots showing foot swelling (FIG. 39O) and viral RNA levels in indicated tissues (FIG. 39P-FIG. 39S) of 4-week-old male C57BL/6J mice at 72 h after inoculation with 10³ FFU of CHIKV (La Reunion 2006) mixed with 50 mg of mouse Mxra8-Fc, Du-D1-Mo-D2-N66R-MXRA8-Fc, or LDLRAD3-D1-Fc (FIG. 39O: 2 experiments n=6; FIG. 39P-FIG. 39S: 2 experiments n=10). FIG. 39B-FIG. 39E and FIG. 39O: one-way ANOVA with Dunnett's post-test; mean±standard deviation (SD). FIG. 39N: Log rank test. FIG. 39P-FIG. 39S: Kruskal-Wallis ANOVA with Dunn's post-test. **, P<0.01; ***, P<0.001; ****, P<0.0001. See also FIG. 51A-FIG. 51H, FIG. 52A-FIG. 52C, and TABLE 10.

FIG. 40A-FIG. 40H is an exemplary embodiment showing cryo-EM structure of Du-D1-Mo-D2 MXRA8 chimera bound to CHIKV VLP in accordance with the present disclosure. FIG. 40A-FIG. 40B show cryo-EM density of Du-D1-Mo-D2 bound to CHIKV (FIG. 40A) or WEEV (FIG. 40B) VLPs. Shown are two views of a single asymmetric unit, with E1 (light blue), E2 (dark blue), D1 of Du-D1-Mo-D2 (light purple), and D2 of Du-D1-Mo-D2 (dark purple) at site 1. FIG. 40C-FIG. 40D include atomic models of Du-D1-Mo-D2 MXRA8 with experimental density highlighting the symmetry breaking N-linked glycan at Asn120 and b-strands A and H in D1 when binding to CHIKV (FIG. 40C) or WEEV (FIG. 40D). FIG. 40E includes atomic models of Du-D1-Mo-D2 MXRA8 binding to WEEV (left) or CHIKV (right) highlighting the flipped orientations. Surfaces are shown for the wrapped E1-E2 heterodimer (light gray), intra E1-E2 heterodimer (gray), and inter E1-E2 heterodimer (dark gray). Du-D1-Mo-D2 is depicted as a ribbon and is rainbow-colored from the N- to C-terminus. Stalk attaching MXRA8 to the cell membrane is represented as a red dashed line. FIG. 40F is a bar graph showing MXRA8 site occupancies for duck MXRA8 (light blue), mouse MXRA8 (pink), and Du-D1-Mo-D2 (blue and pink stripes) bound to WEEV (left) or CHIKV (right). FIG. 40G includes molecular models of Du-D1-Mo-D2 and mouse MXRA8 bound to CHIKV. Shown are wrapped E1-E2 heterodimer (light gray, transparent), intraspike E1-E2 heterodimer (gray), interspike E1-E2 heterodimer, MXRA8 D1 (light purple), and MXRA8 D2 (dark purple). Observed contact residues in both Du-D1-Mo-D2 and mouse MXRA8 are colored dark red, and contacts observed only for mouse MXRA8 are colored pink. FIG. 40H includes sequences of Du-D1-Mo-D2, mouse, and duck MXRA8 (SEQ ID NOs: 106-108), highlighting conserved contacts between Du-D1-Mo-D2 and mouse MXRA8 to CHIKV (red circles), contacts only in mouse MXRA8 to CHIKV (pink circles), conserved contacts between Du-D1-Mo-D2 and duck MXRA8 to WEEV (red triangles), contacts only in duck MXRA8 to WEEV (blue triangles), and contacts only in Du-D1-Mo-D2 to WEEV (blue and pink stripped triangles). See also FIG. 45A-FIG. 45D, FIG. 51A-FIG. 51H, and TABLE 7-TABLE 9.

FIG. 41A-FIG. 41J is an exemplary embodiment showing VEEV and EEEV infection are not enhanced by avian or mammalian MXRA8 in accordance with the present disclosure. Related to FIG. 34A-FIG. 34L and TABLE 6. FIG. 41A is a diagram of MXRA8 cDNA expressed in mouse, human and chicken cells. MXRA8 signal peptide was replaced with a b₂-microglobulin signal peptide followed by FLAG tag sequence and a short linker. FIG. 41B includes representative flow cytometry plots showing surface expression of mouse, chicken, turkey, and duck MXRA8 with anti-FLAG mAb. FIG. 41C shows sequence relationships between murine and avian MXRA8. Identity (red) and similarity (yellow) matrix indicating the conservation of MXRA8 between species. FIG. 41D-FIG. 41E include bar graphs showing ΔMxra8 3T3 cells complemented with chicken or mouse MXRA8 or empty vector were inoculated with MAYV (FIG. 41D) or RRV (FIG. 41E). Infection was assessed using anti-E2 mAbs (see Methods; 3 experiments in triplicate). FIG. 41F is a bar graph showing ΔMxra8 3T3 cells complemented with mouse or chicken MXRA8 or empty vector control were inoculated with SINV (Girdwood or Toto) and stained for SINV antigen (3 experiments in triplicate). FIG. 41G includes bar graphs showing Jurkat cells expressing mouse MXRA8, turkey MXRA8, LDLRAD3, or transduced with an empty vector control were inoculated with CHIKV (strain 181/25), SINV TR339-GFP, or SINV-VEEV-GFP (3 experiments in triplicate). Infection was assessed by anti-E1 antibody staining or GFP expression. FIG. 41H is a bar graph showing K562 cells expressing mouse MXRA8, turkey MXRA8, LDLRAD3, VLDLR or transduced with an empty vector control were inoculated with SINV-EEEV-GFP (3 experiments in triplicate). Infection was assessed by GFP expression. FIG. 41I is a representative flow cytometry plot showing surface expression of MXRA8 in CEFs after staining with anti-chicken MXRA8 mAbs (red) or an isotype control (blue). FIG. 41J includes plots showing CEFs were inoculated with CHIKV 181/25, SINV-WEEV-GFP, or SINV TR339. Infection was assessed by GFP expression, CHIKV E2, or SINV antigen staining and is compared between infected (red) and non-infected cells (blue). FIG. 41D-FIG. 41F: one-way ANOVA with Dunnett's post-test; mean±SD: ****, P<0.0001.

FIG. 42A-FIG. 42M is an exemplary embodiment showing chicken MXRA8 is required for optimal infection of WHAV, BBKV and OCKV but not EEEV in murine 3T3 cells and CEFs in accordance with the present disclosure. Related to FIG. 34A-FIG. 34L and TABLE 6. FIG. 42A includes representative flow cytometry plots showing ΔMxra8 CEFs complemented with empty vector (blue histogram), chicken or mouse MXRA8 (red histograms) and stained with mouse-anti-chicken MXRA8 mAbs or hamster anti-mouse MXRA8 mAbs. Wild type CEFs served as a control (gray histogram). FIG. 42B is a bar graph showing ΔMxra8 chicken fibroblasts complemented with chicken or mouse MXRA8 (2 independent clones of each shown) or empty vector control and non-targeted (NT1 and NT2) CEFs were inoculated with SINV-EEEV-GFP. FIG. 42C-FIG. 42F include bar graphs showing ΔMxra8 3T3 cells complemented with mouse or chicken MXRA8 or empty vector control were inoculated with WHAV (FIG. 42C), BBKV (FIG. 42D), OCKV (FIG. 42E), or AURAV (FIG. 42F) and stained for viral antigen expression (3 experiments in triplicate). FIG. 42G-FIG. 42J include bar graphs showing CEFs were pre-incubated with mouse anti-chicken MXRA8 mAbs for one hour and then inoculated with WHAV (FIG. 42G), BBKV (FIG. 42H), OCKV (FIG. 42I) and AURAV (FIG. 42J) and stained for E1 antigen expression (3 to 4 experiments in triplicate). FIG. 42K includes multi-step growth curves with OCKV in ΔMxra8 CEFs complemented with chicken or mouse MXRA8 or empty vector control. Virus was titrated by focus-forming assay (3 experiments in duplicate). FIG. 42L is a bar graph showing ΔMxra8 CEFs complemented with chicken or mouse MXRA8 or empty vector control or non-targeting sgRNA (NT1 and NT2) were inoculated with OCKV. Infection was assessed by viral antigen expression and flow cytometry (3 experiments in triplicate; mean±SD). FIG. 42M is a bar graph showing ΔMxra8 chicken fibroblasts and non-targeted (NT1 and NT2) CEFs were inoculated with AURAV. Infection was assessed with flow cytometry (3 experiments in triplicate; mean±SD). FIG. 42C-FIG. 42E and FIG. 42G-FIG. 42I and FIG. 42L; one-way ANOVA with Dunnett's post-test; mean±SD: *, P<0.05; **, P<0.01; ***, P<0.001; ****, P<0.0001. FIG. 42K: two-way ANOVA with Tukey's post-test; mean±SD: ****, P<0.0001.

FIG. 43A-FIG. 43N is an exemplary embodiment showing alphavirus infection in human 293T and HeLa cells expressing chicken, sparrow, or mouse MXRA8 in accordance with the present disclosure. Related to TABLE 6. FIG. 43A-FIG. 43B include flow cytometry histograms showing surface expression of chicken and murine MXRA8 in human 293T (FIG. 43A) or HeLa (FIG. 43B) cells after staining with mouse anti-chicken or hamster anti-murine MXRA8 mAbs. Red shading indicates cells complemented with chicken or mouse MXRA8, and blue shading indicates cells complemented with empty vector control. FIG. 43C-FIG. 43J include bar graphs showing 293T (FIG. 43C, FIG. 43E, FIG. 43G, FIG. 43I) or HeLa cells (FIG. 43D, FIG. 43F, FIG. 43H, FIG. 43J) transfected with mouse or chicken MXRA8 or empty vector control were inoculated with SINV (FIG. 43C, FIG. 43D), CHIKV (FIG. 43E, FIG. 43F), AURAV (FIG. 43G, FIG. 43H, 12 hpi; FIG. 43I, FIG. 43J, 24 hpi) and stained for viral antigen (3 experiments in triplicate). FIG. 43K is a flow cytometry histogram showing surface expression of sparrow MXRA8 in human 293T cells using anti-FLAG mAb. Red shading indicates cells expressing sparrow MXRA8, and blue shading indicates cells transfected with empty vector control. FIG. 43L-FIG. 43N include bar graphs showing 293T cells were transfected with mouse, chicken, or sparrow MXRA8 or empty vector control and inoculated with SINV (FIG. 43L), CHIKV 181/25 (FIG. 43M), or CHIKV 37997 (FIG. 43N). Cells were stained for viral antigen with specific mAbs (3 experiments in triplicate). FIG. 43C-FIG. 43F, FIG. 43L-FIG. 43N: one-way ANOVA with Dunnett's post-test; mean±SD: *, P<0.05; ****, P<0.0001.

FIG. 44A-FIG. 44D is an exemplary embodiment showing chicken MXRA8 promotes SINV infection in mice in accordance with the present disclosure. Related to FIG. 34A-FIG. 34L. FIG. 44A is a timeline showing five-week-old BALB/c mice were transduced via an intranasal route with 10¹⁰ adenoviral particles encoding chicken MXRA8 gene or no transgene. Four days later, mice were inoculated with 0.5 mg of anti-IFNAR1 mAb followed by 10⁴ FFU of SINV one day later via intranasal route. Tissues were harvested at 40 and 72 h post-infection. FIG. 44B-FIG. 44D include dot plots showing infection in the spleen (FIG. 44B) and lung (FIG. 44C-FIG. 44D) were measured by qRT-PCR (FIG. 44B-FIG. 44C; viral RNA) or focus-forming (FIG. 44D; infectious virus) assays (2 experiments, n=7). FIG. 44B-FIG. 44D: Mann-Whitney test; median values: **, P<0.01; ***, P<0.001.

FIG. 45A-FIG. 45D is an exemplary embodiment showing gold-standard Fourier shell correlation curve resolutions for final maps in accordance with the present disclosure. Related to FIG. 36A-FIG. 36D and FIG. 40A-FIG. 40H. FIG. 45A-FIG. 45D include graphs showing cutoffs of 0.143 yield a final resolution of: 4.35 Å for WEEV-VLP alone (FIG. 45A), 4.74 Å for WEEV-VLP bound to duck MXRA8 (FIG. 45B), 3.96 Å for CHIKV-VLP bound to Du-D1-Mo-D2 MXRA8 (FIG. 45C), and 3.89 Å for WEEV-VLP bound to Du-D1-Mo-D2 MXRA8 (FIG. 45D).

FIG. 46 is a cryo-EM processing flow diagram of duck MXRA8 bound to WEEV-VLP in accordance with the present disclosure. Related to FIG. 36A-FIG. 36D. Arrows track the flow of micrographs/particles, and boxes indicate processing steps. Red dots indicate particle centers determined using crYOLO particle picking (top micrograph) or through subparticle extraction using icosahedral symmetry (bottom micrograph).

FIG. 47 shows an E1 sequence alignment (SEQ ID NOs: 109-115) and contact residues in accordance with the present disclosure. Related to FIG. 37A-FIG. 37E. E1 secondary structure is colored in blue cartoon and domains are colored similarly to FIG. 36D. WEEV contact residues (circles) and CHIKV contact residues (diamonds) are colored based on the mode of contact with MXRA8: cyan for wrapped spike heterodimer, red for intraspike heterodimer, and yellow for interspike heterodimer.

FIG. 48 shows an E2 sequence alignment (SEQ ID NOs: 116-122) and contact residues in accordance with the present disclosure. Related to FIG. 37A-FIG. 37E. E2 secondary structure is colored in dark blue cartoon and domains are colored similarly to FIG. 36D. WEEV contact residues (circles) and CHIKV contact residues (diamonds) are colored based on mode of contact with MXRA8: cyan for wrapped spike heterodimer, red for intraspike heterodimer, and yellow for interspike heterodimer.

FIG. 49 shows a MXRA8 sequence alignment (SEQ ID NOs: 123-128) and contact residues in accordance with the present disclosure. Related to FIG. 37A-FIG. 37E. MXRA8 secondary structure is colored in blue cartoon along with labels for 6-strands. Domains 1 and 2 are colored similarly to FIG. 36C, with the stalk colored red and transmembrane region colored green. Duck (circles) and mouse (diamonds) contact residues are colored based on the mode of contact with E1-E2 heterodimers: cyan for wrapped spike heterodimer, red for intraspike heterodimer, and yellow for interspike heterodimer.

FIG. 50 shows MXRA8 chimeras and mutants used to assess avian MXRA8 binding mode in accordance with the present disclosure. Related to FIG. 38A-FIG. 38D. MXRA8 secondary structure is colored in the blue cartoon along with labels for 6-strands. D1 and D2 are colored similarly to FIG. 36C, with the stalk colored red and transmembrane colored green. Sequences include mouse (control), duck, duck D1 with mouse D2, duck D2 with mouse D1, D1 mutations a-d, and D2 mutations a-d, as described in FIG. 38D. Double-arginine mutations and loop insertions are depicted with solid red boxes and dashed red boxes, respectively.

FIG. 51A-FIG. 51H is an exemplary embodiment showing affinity measurements and stalk length effects of chimeric MXRA8 binding to alphaviruses in accordance with the present disclosure. Related to FIG. 39A-FIG. 39S and FIG. 40A-FIG. 40H. FIG. 51A-FIG. 51D show biolayer interferometry binding analysis of monovalent forms of duck MXRA8 (FIG. 51A), Du-D1-Mo-D2 MXRA8 (FIG. 51B and FIG. 51D), or mouse MXRA8 (FIG. 51C) to immobilized WEEV (FIG. 51A-FIG. 51B) or CHIKV (FIG. 51C-FIG. 51D) VLPs. Representative data are shown in black, with fitted (1:1 Langmuir binding models) in red. Kinetic (K_{D}) affinity constants are displayed (mean±SD pooled from two to three independent experiments). FIG. 51E-FIG. 51F include graphs showing neutralization of SINV-CHIKV (FIG. 51E) or SINV-WEEV (FIG. 51F) infection after pre-treatment with indicated concentrations of duck MXRA8-Fc, mouse MXRA8-Fc, Du-D1-Mo D2 duStalk-MXRA8-Fc [duck stalk], or Du-D1-Mo D2 moStalk-MXRA8-Fc [mouse stalk] (two experiments performed in technical duplicates). Infection was analyzed by flow cytometry (GFP expression) and normalized to levels obtained without a MXRA8 inhibitory decoy protein. FIG. 51G-FIG. 51H include graphs showing neutralization of SINV-CHIKV (FIG. 51G) or SINV-WEEV (FIG. 51H) infection by duck MXRA8-Fc, mouse MXRA8-Fc, Du-D1-Mo D2-MXRA8-Fc, or Du-D1-Mo-D2-N66R-MXRA8-Fc (four experiments, technical duplicates). Infection was analyzed by flow cytometry and normalized to levels obtained without a MXRA8 inhibitory decoy protein.

FIG. 52A-FIG. 52C is an exemplary embodiment showing expansion of host reservoirs for WEE complex members in accordance with the present disclosure. Related to FIG. 39A-FIG. 39S. FIG. 52A is a sequence alignment of D1 of MXRA8 (SEQ ID NOs: 141-162). Sequences are ordered based on similarity to duck MXRA8. Sequence identity of the contacting residues is shown adjacent to the colloquial species name. Duck MXRA8 contacts to WEEV E1 are represented as spheres for wrapped (cyan), intraspike (red), and interspike (yellow) E1-E2 heterodimers. Conserved contacts for each sequence are boxed and colored to represent wrapped (cyan), intraspike (red), and interspike (yellow) contacts. FIG. 52B-FIG. 52C include bar graphs showing ΔMxra8 3T3 cells complemented with mouse (pink), chicken (red), duck (blue), alligator (dark gray) or sea turtle (olive green) MXRA8 or empty vector control (gray) were inoculated with SINV-CHIKV-GFP (FIG. 52B) or SINV TR339-GFP (FIG. 52C). Infection was assessed by GFP expression and is compared between infected and non-infected cells (3 experiments, in triplicate). FIG. 52B-FIG. 52C: One-way ANOVA with Dunnett's post-test; mean±standard deviation (SD). ****, P<0.0001.

### DETAILED DESCRIPTION OF THE INVENTION

The present disclosure is based, at least in part, on the discovery that Mxra8 is a receptor for arthritogenic alphaviruses, including chikungunya (CHIKV), Ross River, Mayaro, and O'nyong nyong (ONNV) viruses.

MXRA8

One aspect of the present disclosure provides for targeting of Mxra8 (also called DICAM, ASP3, or limitrin). The present disclosure provides methods of treating alphaviruses based on the discovery that Mxra8 is critical for an entry step in the virus lifecycle.

As described herein, the mouse protein Mxra8 was identified in a whole genome CRISPR/Cas9 screen looking for host factors that were required for infection of chikungunya virus (CHIKV). Mxra8 was one of the top hits of the screen. Subsequent validation studies revealed that gene editing of mouse Mxra8 via CRISPR/Cas9 resulted in markedly reduced infection of chikungunya, Ross River, Mayaro, and O'nyong nyong viruses but did not directly impact distantly related alphaviruses (Sindbis, EEEV, WEEV) or unrelated flaviviruses. Transcomplementation of KO cells with mouse or human Mxra8 restored infectivity of CHIKV.

As described herein, mechanism of action studies using CHIKV pseudotyped viruses or transfection of genomic RNA revealed that Mxra8 was critical for an entry step in the virus lifecycle. Direct binding and entry assays showed Mxra8 was not an attachment receptor but rather functioned as an entry receptor critical for allowing the virus to gain access to the cytoplasm.

As described herein, using a genome-wide CRISPR/Cas9-based screen, cell adhesion molecule Mxra8 was identified as an entry mediator for multiple emerging arthritogenic alphaviruses including chikungunya (CHIKV), Ross River, Mayaro, and O'nyong nyong (ONNV) viruses.

As described herein, gene editing of mouse Mxra8 or human MXRA8 resulted in reduced viral infection of cells, and reciprocally, ectopic expression resulted in increased infection.

As described herein, Mxra8 bound directly to CHIKV particles and enhanced virus attachment and internalization into cells.

As described herein, Mxra8-Fc protein or anti-Mxra8 monoclonal antibodies blocked CHIKV infection in multiple cell types including primary human synovial fibroblasts, osteoblasts, chondrocytes, and skeletal muscle cells.

As described herein, mutagenesis experiments suggest that Mxra8 binds to a surface-exposed region across the A and B domains of CHIKV E2, a speculated site of attachment.

As described herein, administration of Mxr8a-Fc protein or anti-Mxra8 blocking antibodies reduced CHIKV or ONNV infection and associated foot swelling in mice.

As such, it has been shown that pharmacological targeting of Mxra8 can form a strategy for mitigating infection and disease by multiple arthritogenic alphaviruses.

Consistent with a role in attachment and entry, Mxra8-Fc protein or anti-Mxra8 antibody directly blocked CHIKV infection.

Mxra8 can be an isoform of Mxra8. For example, an isoform of Mxra8 can be selected from one or more of the group selected from MXRA8-1, MXRA8-2, MXRA8-3, and MXRA8-4.

Mxra8-Associated Viruses

As described herein, the Mxra8 receptor was discovered on cells as an entry receptor for viruses (e.g., arthritogenic alphaviruses) that expressed Mxra8 on its surface. A Mxra8-associated virus can be any virus expressing Mxra8 on the surface of the virus and using a Mxra8 receptor on cell as the entry point of infection. As described herein, arthritogenic alphaviruses comprise Mxra8 and cells comprise Mxra8 entry receptors. Inhibition of Mxra8, inhibition of Mxra8 receptors, or knock out of the Mxra8 receptors reduce arthritogenic alphavirus infection.

Arthritogenic alphaviruses comprise a group of enveloped RNA viruses that can be transmitted to humans by mosquitoes and cause debilitating acute and chronic musculoskeletal disease¹. The host factors required for alphavirus entry remain poorly characterized 2.

As described herein, Mxra8 was discovered as an entry receptor for several arthritogenic alphaviruses. For example, a Mxra8-associated alphavirus can be any arthritogenic alphavirus. As another example, the Mxra8-associated alphavirus can be Chikungunya (e.g., CHIKV-181/25, 181/25-mKate2, CHIKV-AF15561, CHIKV-LR 2006, CHIKV-37997 (West African lineage)), Ross River (e.g., RRV (T48)), Mayaro (e.g., MAYV (BeH407)), Barmah Forest, or O'nyong nyong (e.g., ONNV (MP30)) viruses. Other Mxra8-associated alphaviruses can be Semliki Forest virus (SFV) (e.g., SFV (Kumba)) or Getah virus. Mxra8 can also be associated with or play an entry role in additional viruses or alphaviruses that have not yet been tested or discovered.

Alphaviruses that were discovered to not be Mxra8-associated alphaviruses were distantly related alphaviruses (e.g., Sindbis, Bebaru, Una, Middleburg, Eastern equine encephalitis, Western equine encephalitis, or Venezuelan equine encephalitis viruses) or unrelated flaviviruses (e.g., West Nile virus), bunyaviruses (Rift Valley fever virus), rhabdoviruses (e.g., Vesicular stomatitis virus), or picornaviruses (encephalomyocarditis virus). It was also discovered that Mxra8 showed no effect for unrelated positive- or negative-sense RNA viruses.

Mxra8 Inhibiting Agent

As described herein, a Mxra8 inhibiting agent reduces, prevents, or aborts infection of many different alphaviruses (e.g., Mxra8-associated alphaviruses). For example, a Mxra8 inhibiting agent can be an agent that interrupts the interaction of Mxra8 expressed on the virus and the Mxra8 entry receptor on the cell surface. As another example, the Mxra8 inhibiting agent can bind to Mxra8 on the virus or can bind to the Mxra8 receptor on the cell. The Mxra8 inhibiting agent can be an agent that binds to the viral structural protein (e.g., E2) of the viron on the cell surface. As another example, a Mxra8 inhibiting agent can be an inhibitor of Mxra8 or an inhibitor of Mxra8 receptors (e.g., antibodies, fusion proteins, small molecules).

A Mxra8 inhibiting agent can be any agent that can inhibit Mxra8, inhibit a Mxra8 receptor (e.g., Mxra8 fusion proteins, Mxra8, Mxra8 dimers, and functional variants thereof), downregulate Mxra8, or knockdown Mxra8.

As another example, a Mxra8 inhibiting agent can be a mutated or genetically edited Mxra8 to render it unable to bind to the Mxra8 receptor on an alphavirus.

As another example, the Mxra8 inhibiting agent can be a fusion protein. For example, the Mxra8 fusion protein can comprise a fragment or variant of Mxra8 (e.g., Mxra8-Fc, MXRA8-2-Fc). As another example, the fusion protein can comprise a mouse or human Fc antibody domain fused to the ectodomain of Mxra8 or MXRA8-2. Fusion proteins as described herein can comprise a Mxra8 or functional variant thereof.

Mxra8 can comprise a Mxra8 ectodomain (see e.g., SEQ ID NOs: 1-4) or a Mxra8 isoform (see e.g., (SEQ ID NOs: 25-31)) or a functional variant or functional fragment thereof.

As an example, the following fusion proteins were developed: human Mxra8 on mouse Fc (see e.g., SEQ ID NOs: 17-18); mouse Mxra8 on mouse Fc (see e.g., SEQ ID NOs: 19-20); mouse Mxra8 on human Fc (see e.g., SEQ ID NOs: 21-22); and mouse

Mxra8 on human Fc N297Q (see e.g., SEQ ID NOs: 23-24). As another example, the fusion proteins can comprise a signal peptide (e.g., an IL-2 signal peptide), a Mxra isoform fragment, or functional variant thereof (e.g., fragment of Mxra8 comprising the ectodomain of Mxra8); a linker (optionally) (e.g., GS linker), or an Fc region (e.g., mIgG2b Fc region).

Human Mxra8 isoform 1 ectodomain nucleotide sequence
(SEQ ID NO: 1)
GTCCTGTTGCATAGCGGCTCCTCTGTCCCGGCCGCAGCGGGATCAAGCGTAGTGAGCGAGTCAGC
AGTTTCCTGGGAAGCGGGGGCAAGGGCTGTTCTGAGGTGTCAGAGCCCAAGGATGGTGTGGACGC
AAGACCGGCTCCACGACAGGCAAAGAGTGCTTCACTGGGACCTCAGGGGACCCGGTGGTGGCCCA
GCACGCCGGCTCCTGGACCTCTATAGCGCGGGAGAACAACGAGTATACGAAGCTAGGGACAGAGG
AAGACTGGAGCTTTCAGCCAGTGCGTTCGATGATGGAAATTTTTCACTTCTGATCCGGGCTGTGG
AAGAAACTGATGCTGGACTGTATACTTGTAATCTCCACCATCACTACTGCCACCTCTATGAGTCA
TTGGCTGTCAGACTGGAAGTTACCGATGGCCCGCCCGCCACACCAGCTTACTGGGATGGCGAAAA
AGAAGTACTTGCTGTCGCGCGAGGTGCCCCTGCGCTCCTGACATGCGTCAACAGGGGTCACGTTT
GGACTGATCGGCACGTCGAAGAGGCTCAGCAAGTAGTTCACTGGGACCGCCAGCCGCCTGGCGTA
CCTCACGATAGGGCCGACCGCCTCTTGGACCTCTACGCCTCCGGTGAAAGGAGGGCCTATGGGCC
TCTGTTCCTCAGAGATAGGGTTGCGGTGGGCGCAGATGCCTTTGAACGAGGCGATTTCAGCCTCC
GCATTGAGCCCCTGGAGGTAGCCGATGAGGGCACCTACTCCTGTCATCTGCATCATCATTATTGC
GGATTGCACGAGAGGAGGGTCTTCCACTTGACTGTAGCTGAACCGCATGCTGAACCGCCACCTCG
CGGATCACCCGGAAACGGTAGCAGTCATTCCGGGGCTCCCGGGCCGGATCCGACATTGGCACGAG
GGCATAACGTAATAAATGTCATCGTTCCCGAGTCACGCGCTCAT
Human Mxra8 isoform 1 ectodomain polypeptide sequence
(SEQ ID NO: 2)
VLLHSGSSVPAAAGSSVVSESAVSWEAGARAVLRCQSPRMVWTQDRLHDRQRVLHWDLRGPGGGP
ARRLLDLYSAGEQRVYEARDRGRLELSASAFDDGNFSLLIRAVEETDAGLYTCNLHHHYCHLYES
LAVRLEVTDGPPATPAYWDGEKEVLAVARGAPALLICVNRGHVWTDRHVERAQQVVHWDRQPPGV
PHDRADRLLDLYASGERRAYGPLFLRDRVAVGADAFERGDESLRIEPLEVADEGTYSCHLHHHYC
GLHERRVFHLIVAEPHAEPPPRGSPGNGSSHSGAPGPDPTLARGHNVINVIVPESRAH
Mouse Mxra8 ectodomain nucleotide sequence
(SEQ ID NO: 3)
TCTGGCCCGAGCGGTACTGCTGCCGCAAGCTCAAGCCTCGTCTCTGAATCAGTTGTGTCTCTCGC
GGCCGGAACTCAGGCGGTCCTGAGGTGTCAAAGTCCAAGGATGGTTTGGACCCAAGATCGCTTGC
ATGATCGACAAAGAGTTGTGCACTGGGACCTCAGCGGTGGTCCTGGATCACAACGAAGAAGGCTG
GTGGACATGTACTCAGCCGGAGAACAGCGCGTTTACGAGCCACGAGACCGAGATCGACTCCTTTT
GTCACCTTCTGCGTTCCATGACGGCAACTTTTCTTTGTTGATAAGAGCCGTAGATCGCGGAGATG
AAGGAGTTTATACGTGTAACCTTCACCACCATTACTGCCATCTCGATGAAAGTCTCGCCGTACGG
CTGGAAGTTACGGAGGACCCACTTCTTTCACGGGCATATTGGGATGGCGAGAAGGAGGTCCTGGT
CGTTGCTCACGGCGCACCAGCCCTGATGACATGCATCAACAGAGCCCATGTTTGGACCGACAGGC
ACCTTGAAGAAGCACAACAAGTTGTCCATTGGGACCGACAGCTCCCAGGTGTTAGCCATGATCGG
GCCGATCGCTTGCTTGACTTGTATGCCAGCGGGGAACGGAGGGCATACGGCCCGCCTTTTCTTCG
GGATCGAGTTAGCGTAAACACTAACGCCTTTGCACGCGGTGACTTCAGCCTTCGCATAGATGAAC
TCGAACGAGCAGATGAAGGAATATACTCCTGCCATTTGCATCATCATTATTGCGGCCTGCATGAG
CGAAGGGTATTTCACCTCCAAGTGACTGAACCTGCATTCGAGCCACCAGCGAGGGCCTCACCGGG
AAACGGCTCCGGCCACAGCAGCGCACCGTCTCCGGACCCGACTTTGACACGGGGCCATAGTATAA
TAAACGTAATAGTTCCTGAGGACCATACCCAC
Mouse Mxra8 ectodomain polypeptide sequence
(SEQ ID NO: 4)
SGPSGTAAASSSLVSESVVSLAAGTQAVLRCQSPRMVWTQDRLHDRQRVVHWDLSGGPGSQRRRL
VDMYSAGEQRVYEPRDRDRILLSPSAFHDGNESLLIRAVDRGDEGVYTCNLHHHYCHLDESLAVR
LEVTEDPLISRAYWDGEKEVLVVAHGAPALMTCINRAHVWTDRHLEEAQQVVHWDRQLPGVSHDR
ADRLLDLYASGERRAYGPPFLRDRVSVNTNAFARGDFSLRIDELERADEGIYSCHLHHHYCGLHE
RRVFHLQVTEPAFEPPARASPGNGSGHSSAPSPDPTLTRGHSIINVIVPEDHTH
mIgG2b Fc nucleotide sequence
(SEQ ID NO: 5)
CCCAGCGGGCCCATTTCAACAATCAACCCCTGTCCTCCATGCAAGGAGTGTCACAAATGCCCAGG
TAAGTCACTACCAGAGCTCCACTCCCAGGAGAATGGTAAGTGCTGTAAAAATCCCCTTAATGGAG
GATAAGCCATGTACAAATCCATTTCCATCTCTCCTCATCAGCTCCTAACCTCGAGGGTGGACCAT
TGTGTGGTGGTGGATGTGAGCGAGGATGACCCAGACGTCCAGATCAGCTGGTTTGTGAACAACGT
GGAAGTACACACAGCTCAGACACAAACCCATAGAGAGGATTACAACAGTACTATCCGGGTGGTCA
GCACCCTCCCCATCCAGCACCAGGACTGGATGAGTGGCAAGGAGTTCAAATGCAAGGTCAACAAC
AAAGACCTCCCATCACCCATCGAGAGAACCATCTCAAAAATTAAAGGTGGGACCTGCAGGACAAC
TGCATGGGGGCTGGGATGGGCATAAGAATAAATGTCTATGTGGACAGCCTTCCACTTCAGCCATG
ACCTCTATGTATGTTTCTAACCCCACAGGGCTAGTCAGAGCTCCACAAGTATACATCTTGCCGCC
GAGACATCAGTGTGGAGTGGACCAGCAATGGGCATACAGAGGAGAACTACAAGGACACCGCACCA
GTCCTGGACTCTGACGGTTCTTACTTCATATATAGCAAGCTCAATATGAAAACAAGCAAGTGGGA
GAAAACAGATTCCTTCTCATGCAACGTGAGACACGAGGGTCTGAAAAATTACTACCTGAAGAAGA
CCATCTCCCGGTCTCCGGGTAAA
mIgG2b Fc polypeptide sequence
(SEQ ID NO: 6)
PSGPISTINPCPPCKECHKCPAPNLEGGPSVFIPPPNIKDVLMISLTPKVTCVVVDVSEDDPDVQ
ISWFVNNVEVHTAQTQTHREDYNSTIRVVSTLPIQHQDWMSGKEFKCKVNNKDLPSPIERTISKI
KGLVPAPQVYILPPPAEQLSRKDVSLTCLVVGFNPGDISVEWTSNGHTEENYKDTAPVLDSDGSY
FIYSKLNMKTSKWEKTDSFSCNVRHEGLKNYYLKKTISRSPGK
Human IgG1 Fc nucleotide sequence
(SEQ ID NO: 7)

[symbol]

Human IgG1 Fc polypeptide sequence
(SEQ ID NO: 8)

[symbol]

Human IgG1 Fc with N297Q mutation nucleotide sequence
(SEQ ID NO: 9)

[symbol]

Human IgG1 Fc with N297Q mutation polypeptide sequence
(SEQ ID NO: 10)

[symbol]

IL-2 signal peptide nucleic acid sequence
(SEQ ID NO: 11)

[symbol]

IL-2 signal peptide polypeptide sequence
(SEQ ID NO: 12)

[symbol]

GS linker nucleotide sequence
(SEQ ID NO: 13)

[symbol]

GS linker polypeptide sequence
(SEQ ID NO: 14)

[symbol]

Signal peptide nucleic acid sequence
(SEQ ID NO: 15)

[symbol]

Signal peptide polypeptide sequence
(SEQ ID NO: 16)

[symbol]

mIgG2b_human_Mxra8_iso1 nucleotide sequence
(SEQ ID NO: 17)

[symbol]

Orange: IL-2 signal peptide
Blue: Human Mxra8 isoform 1 ectodomain
Red: mIgG2b Fc region
mIgG2b_human_Mxra8_iso1 polypeptide sequence
(SEQ ID NO: 18)

[symbol]

QDRLHDRQRVLHWDLRGPGGGPARRLLDLYSAGEQRVYEARDRGRLELSASAFDDGNESLLIRAV
EETDAGLYTCNLHHHYCHLYESLAVRLEVTDGPPATPAYWDGEKEVLAVARGAPALLTCVNRGHV
WTDRHVEEAQQVVHWDRQPPGVPHDRADRLLDLYASGERRAYGPLFLRDRVAVGADAFERGDFSL
RIEPLEVADEGTYSCHLHHHYCGLHERRVFHLIVAEPHAEPPPRGSPGNGSSHSGAPGPDPTLAR

[symbol]

Orange: IL-2 signal peptide
Blue: Human Mxra8 isoform 1 ectodomain
Red: mIgG2b Fc region
mIgG2b_mouse_Mxra8 nucleotide sequence
(SEQ ID NO: 19)

[symbol]

Orange: IL-2 signal peptide
Blue: Mouse Mxra8 ectodomain
Red: mIgG2b Fc region
mIgG2b_mouse_Mxra8 polypeptide sequence
(SEQ ID NO: 20)

[symbol]

HDRQRVVHWDLSGGPGSQRRRLVDMYSAGEQRVYEPRDRDRILLSPSAFHDGNESLLIRAVDRGD
EGVYTQNLHHHYCHLDESLAVRLEVTEDPLUSRAYWDGEKEVLVVARGAPALMTCINRAHVWIDR
HLEEAQQVVHWDRQLPGVSHDRADRLLDLYASGERRAYGPPFLRDRVSVNTNAFARGDESLRIDE
LERADEGIYSCHLHHHYCGLHERRVFHLQVTEPAFEPPARASPGNGSGHSSAPSPDPTLTRGHSI

[symbol]

Orange: IL-2 signal peptide
Blue: Mouse Mxra8 ectodomain
Red: mIgG2b Fc region
hIgG1_9L_mouse_Mxra8 nucleotide sequence
(SEQ ID NO: 21)

[symbol]

TCGTCTCTGAATCAGTTGTGTCTCTCGCGGCCGGAACTCAGGCGGTCCTGAGGTGTCAAAGTCCA
AGGATGGTTTGGACCCAAGATCGCTTGCATGATCGACAAAGAGTTGTGCACTGGGACCTCAGCGG
TGGTCCTGGATCACAACGAAGAAGGCTGGTGGACATGTACTCAGCCGGAGAACAGCGCGITTACG
AGCCACGAGACCGAGATCGACTCCTTTTGTCACCTTCTGCGTTCCATGACGGCAACTTTTCTTTG
TTGATAAGAGCCGTAGATCGCGGAGATGAAGGAGTTTATACGTGTAACCTTCACCACCATTACTG
CCATCTCGATGAAAGTCTCGCCGTACGGCTGGAAGTTACGGAGGACCCACTICTTTCACGGGCAT
ATTGGGATGGCGAGAAGGAGGTCCTGGTCGTTGCTCACGGCGCACCAGCCCTGATGACATGCATC
AACAGAGCCCATGTTTGGACCGACAGGCACCTIGAAGAAGCACAACAAGTIGTCCATTGGGACCG
ACAGCTCCCAGGTGTTAGCCATGATCGGGCCGATCGCTTGCTTGACTTGTATGCCAGCGGGGAAC
GGAGGGCATACGGCCCGCCTTTTCTTCGGGATCGAGTTAGCGTAAACACTAACGCCTTTGCACGC
GGTGACTTCAGCCTTCGCATAGATGAACTCGAACGAGCAGATGAAGGAATATACTCCTGCCATTT
GCATCATCATTATTGCGGCCTGCATGAGCGAAGGGTATTTCACCTCCAAGTGACTGAACCTGCAT

[symbol]

Orange: Signal peptide
Blue: Mouse Mxra8 ectodomain
Green: GS linker
Red: Human IgG1 Fc region
hIgG1_9L_mouse_Mxra8 polypeptide sequence
(SEQ ID NO: 22)

[symbol]

Orange: Signal peptide
Blue: Mouse Mxra8 ectodomain
Green: GS linker
Red: Human IgG1 Fc region
hIgG1_N297Q_9L_mouse_Mxra8 nucleotide sequence
(SEQ ID NO: 23)

[symbol]

Orange: Signal peptide
Blue: Mouse Mxra8 ectodomain
Green: GS linker
Red: Human IgG1 Fc region [symbol]
: N2970 mutation
hIgG1_N297Q_9L_mouse_Mxra8 polypeptide sequence
(SEQ ID NO: 24)

[symbol]

Orange: Signal peptide
Blue: Mouse Mxra8 ectodomain
Green: GS linker
Red: Human IgG1 Fc region [symbol]
: N297Q mutation

[symbol]

indicates data missing or illegible when filed

As another example, the Mxra8 inhibiting agent can comprise an ectodomain of Mxra8 or any of Mxra8 isoforms SEQ ID NOs: 25-31 (e.g., XP_016857006.1; XP_016857005.1; NP_001269511.1; NP_001269512.1; NP_001269513.1; NP_001269514.1; NP_115724.1) or functional fragments or variants thereof with Mxra8 activity or Mxra8 receptor activity.

As described herein, the Mxra8 inhibiting agent can comprise an Fc region. The Fc region can be chosen from any Fc region known in the art. For example, the Fc region can comprise human IgG1, human IgG1 Fc with N297Q mutation, or mouse mIgG2b (see e.g., Saxena et al. 2016 Front Immunol. 7: 580).

As an example, a Mxra8 inhibiting agent can be an anti-Mxra8 antibody. The anti-Mxra8 antibody can be an anti-Mxra8 antibody that has Mxra8 inhibitory activity. As an example, the anti-Mxra8 antibody can be anti-Mxra8 (mouse) armenian hamster serum, anti-Mxra8 (mouse) hamster monoclonal antibodies, or anti-MXRA8 (human) monoclonal antibodies. As another example, the anti-Mxra8 purified hamster monoclonal antibody clones can be 1G11.E6, 1H1.F5, 3G2.F5, 4E7.D10, 7F1.D8, 8F7.E1, 9G2.D6 or any combination of the aforementioned clones (e.g., 1G11.E6+7F1.D8, or 4E7.D10+8F7.E1).

As another example, Mxra8 inhibiting agent can be SYKi, which can be used to downregulate Mxra8.A Mxra8 inhibiting agent can be a short hairpin RNA (shRNA) or a short interfering RNA (siRNA). A Mxra8 inhibiting agent can be an sgRNA targeting Mxra8 (e.g., SEQ ID NOs: 34-40).

Measuring Vaccine or Diagnostic Antigen Integrity

As described herein, Mxra8 can be used to assess an alphavirus vaccine or a diagnostic antigen integrity.

Because Mxra8 is a receptor for multiple arthritogenic alphaviruses, it can be used to physically assess the integrity of alphavirus vaccine or diagnostic antigens. As such, Mxra8 can be used to define lot-to-lot integrity or variation, for example, in the context of commercial production. Alphavirus vaccine or diagnostic antigens can include, but are not limited to, live-attenuated viruses, virus-like particles, viral structural proteins, or nucleic acids or vectors producing these viral proteins or particles.

The integrity assay can include capturing Mxra8 or Mxra8 fusion proteins (e.g., Mxra8-Fc) on a substrate (e.g., microtiter well plate, chip, or pin) and then incubating the alphavirus vaccine or diagnostic antigen and detection with a monoclonal or polyclonal anti-vaccine/diagnostic antigen antibody or even Mxra8-Fc itself. Alternatively, the alphavirus vaccine or diagnostic antigen can be captured in the solid phase (directly or via a bridging antibody) and then incubated with Mxra8 or Mxra8-Fc fusion proteins for binding. The assay could be performed by ELISA, biolayerinterferometry, surface plasmon resonance, or any other detection based system for binding.

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position+1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A constructs of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

| Conservative Substitutions I | |
|---|---|
| SIDE CHAIN | AMINO |
| CHARACTERISTIC | ACID |
| Non-polar | G A P I L V |
| Polar-uncharged | C S T M N Q |
| Polar-charged | D E K R |
| Aromatic | H F W Y |
| Other | N Q D E |

Conservative Substitutions II

| SIDE CHAIN | |
|---|---|
| CHARACTERISTIC | AMINO ACID |
| Non-polar (hydrophobic) | |
| A. Aliphatic: | ALIVP |
| B. Aromatic: | FW |
| C. Sulfur-containing: | M |
| D. Borderline: | G |
| Uncharged-polar | |
| A. Hydroxyl: | STY |
| B. Amides: | NQ |
| C. Sulfhydryl: | C |
| D. Borderline: | G |
| Positively Charged (Basic): | KRH |
| Negatively Charged (Acidic): | DE |

Conservative Substitutions III

| Original | |
|---|---|
| Residue | Exemplary Substitution |
| Ala (A) | Val (V), Leu (L), Ile (I) |
| Arg (R) | Lys (K), Gln (Q), Asn (N) |
| Asn (N) | Gln (Q), His (H), Lys (K), Arg (R) |
| Asp (D) | Glu (E) |
| Cys (C) | Ser (S) |
| Gln (Q) | Asn (N) |
| Glu (E) | Asp (D) |
| His (H) | Asn (N), Gln (Q), Lys (K), Arg (R) |
| Ile (I) | Leu (L), Val (V), Met (M), Ala (A), |
| | Phe (F) |
| Leu (L) | Ile (D), Val (V), Met (M), Ala (A), |
| | Phe (F) |
| Lys (K) | Arg (R), Gln (Q), Asn (N) |
| Met (M) | Leu (L), Phe (F), Ile (I) |
| Phe (F) | Leu (L), Val (V), Ile (I), Ala (A) |
| Pro (P) | Gly (G) |
| Ser (S) | Thr (T) |
| Thr (T) | Ser (S) |
| Trp (W) | Tyr (T) |
| Tyr (Y) | Trp (W), Phe (F), Thr (T), Ser (S) |
| Val (V) | Ile (I), Leu (L), Met (M), Phe (F), |
| | Ala (A) |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides, protein aptamers, nucleotide aptamers, and RNA interference (RNAi) (e.g., small interfering RNAs (siRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinofrmatics & Research Computing). Traits influential in defining optimal siRNA sequences include G/C content at the termini of the siRNAs, Tₘ of specific internal domains of the siRNA, siRNA length, position of the target sequence within the CDS (coding region), and nucleotide content of the 3′ overhangs.

Formulation

The agents and compositions described herein can be formulated by any conventional manner using one or more pharmaceutically acceptable carriers or excipients as described in, for example, Remington's Pharmaceutical Sciences (A.R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating a Mxra8-associated alphavirus in a subject in need administration of a therapeutically effective amount a Mxra8 inhibiting agent, so as to reduce infection, prevent or block infection, reduce viral load, shorten the duration of the infection, or reduce the symptoms of the infection, including joint or tissue swelling or joint or tissue inflammation.

Methods described herein are generally performed on a subject in need thereof. A subject in need of the therapeutic methods described herein can be a subject having, diagnosed with, suspected of having, or at risk for developing a Mxra8-associated alphavirus. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject. The subject can be an insect subject, such as a mosquito.

Generally, a safe and effective amount of a Mxra8 inhibiting agent is, for example, that amount that would cause the desired therapeutic effect in a subject while minimizing undesired side effects. In various embodiments, an effective amount of a Mxra8 inhibiting agent described herein can substantially inhibit a Mxra8-associated alphavirus infection, slow the progress of a Mxra8-associated alphavirus infection, limit the development of a Mxra8-associated alphavirus infection, reduce infection, prevent or block infection, reduce viral load, shorten the duration of the infection, or reduce the symptoms of the infection, including joint or tissue swelling or joint or tissue inflammation.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of a Mxra8 inhibiting agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to inhibit a Mxra8-associated alphavirus infection, slow the progress of a Mxra8-associated alphavirus infection, limit the development of a Mxra8-associated alphavirus infection, reduce infection, prevent or block infection, reduce viral load, shorten the duration of the infection, or reduce the symptoms of the infection, including joint or tissue swelling or joint or tissue inflammation.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a Mxra8 inhibiting agent can occur as a single event or over a time course of treatment. For example, a Mxra8 inhibiting agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for viral infections (e.g., a Mxra8-associated alphavirus infection).

A Mxra8 inhibiting agent can be administered simultaneously or sequentially with another agent, such as an antiviral, an antibiotic, an anti-inflammatory, or another agent. For example, a Mxra8 inhibiting agent can be administered simultaneously with another agent, such as an antiviral, an antibiotic, or an anti-inflammatory. Simultaneous administration can occur through administration of separate compositions, each containing one or more of a Mxra8 inhibiting agent, an antiviral, an antibiotic, an anti-inflammatory, or another agent. Simultaneous administration can occur through administration of one composition containing two or more of a Mxra8 inhibiting agent, an antiviral, an antibiotic, an anti-inflammatory, or another agent. A Mxra8 inhibiting agent can be administered sequentially with an antiviral, an antibiotic, an anti-inflammatory, or another agent. For example, a Mxra8 inhibiting agent can be administered before or after administration of an antiviral, an antibiotic, an anti-inflammatory, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Screening

Also provided are methods for screening candidate molecules.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical successful if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

In Silico Screening

As described herein, the atomic coordinates for Mxra8 alone have been discovered by x-ray crystallography and its complexes with the Mxra8 entry receptor have been discovered by single particle cryo-EM. These coordinates can be used in any in silico screening method known in the art (e.g., Miteva (2011) In silico Lead Discovery; Kortagere (2013) In Silico Models for Drug Discovery).

As used herein, three-dimensional (3D) structure refers to the Cartesian coordinates corresponding to an atom's spatial relationship to other atoms in a macromolecule, for example a protein macromolecule. In the methods provided herein, an estimated three-dimensional (3D) structure can be determined by computer modeling, nuclear magnetic resonance (MR), X-ray crystallography, electron microscopy, or homology modeling.

Many in-silico methods for structure prediction and modeling can be used in connection with the methods described herein, including, without limitation, comparative protein modeling methods (e.g., homology modeling methods such as those described in Marti-Renom et al. 2000. Annu Rev Biophys Biomol Struct 29: 291-325), protein threading modeling methods (such as those described in Bowie et al. 1991. Science 253: 164-170; Jones et al. 1992. Nature 358: 86-89), ab initio or de novo protein modeling methods (Simons et al. 1999. Genetics 37: 171-176; Baker 2000, Nature 405: 39-42; Wu et al. 2007. BMC Biol 5: 17), physics-based prediction (Duan and Kollman 1998, Science 282: 740-744; Oldziej et al. 2005, Proc Natl Acad Sci USA 102: 7547-7552); or any combination thereof. Comparative modeling methods can be performed using a number of modeling programs, including, but not limited to, Modeller (Fiser and Sali 2003, Methods Enzymol 374: 461-91) or Swiss-Model (Arnold et al. 2006, Bioinformatics 22: 195-201). Protein threading modeling methods can be performed using a number of modeling programs, including, but not limited to, HHsearch (Soding 2005, Bioinformatics 21: 951-960), Phyre (Kelley and Sternberg. 2009, Nature Protocols 4: 363-371), or Raptor (Xu et al. 2003, J Bioinform Comput Biol 1: 95-117). Ab initio or de novo protein modeling methods can be performed using a number of modeling programs including, but not limited to, Rosetta (Simons et al. 1999. Genetics 37: 171-176; Baker 2000, Nature 405: 39-42; Bradley et al. 2003, Proteins 53: 457-468; Rohl 2004, Methods in Enzymology 383: 66-93) and I-TASSER (Wu et al. 2007. BMC Biol 5: 17).

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Mxra8 is an Entry Receptor for Arthritogenic Alpha Viruses

The following example describes the discovery that a receptor for Mxra8 on the surface of arthritogenic alphaviruses is an entry receptor. Furthermore, this example describes inhibition of Mxra8 on the alpha virus or inhibition of the Mxra8 receptor on a cell mitigates infection by alphaviruses.

Arthritogenic alphaviruses comprise a group of enveloped RNA viruses that are transmitted to humans by mosquitoes and cause debilitating acute and chronic musculoskeletal disease¹. The host factors required for alphavirus entry remain poorly characterized². This example describes the use of a genome-wide CRISPR-Cas9-based screen to identify the cell adhesion molecule Mxra8 as an entry mediator for multiple emerging arthritogenic alphaviruses, including chikungunya, Ross River, Mayaro and O'nyong nyong viruses. Gene editing of mouse Mxra8 or human MXRA8 resulted in reduced levels of viral infection of cells and, reciprocally, ectopic expression of these genes resulted in increased infection. Mxra8 bound directly to chikungunya virus particles and enhanced virus attachment and internalization into cells. Consistent with these findings, Mxra8-Fc fusion protein or anti-Mxra8 monoclonal antibodies blocked chikungunya virus infection in multiple cell types, including primary human synovial fibroblasts, osteoblasts, chondrocytes, and skeletal muscle cells. Mutagenesis experiments suggest that Mxra8 binds to a surface-exposed region across the A and B domains of chikungunya virus E2 protein, which are a speculated site of attachment. Finally, administration of the Mxra8-Fc protein or anti-Mxra8 blocking antibodies to mice reduced chikungunya and O'nyong nyong virus infection as well as associated foot swelling. Pharmacological targeting of Mxra8 could form a strategy for mitigating infection and disease by multiple arthritogenic alphaviruses.

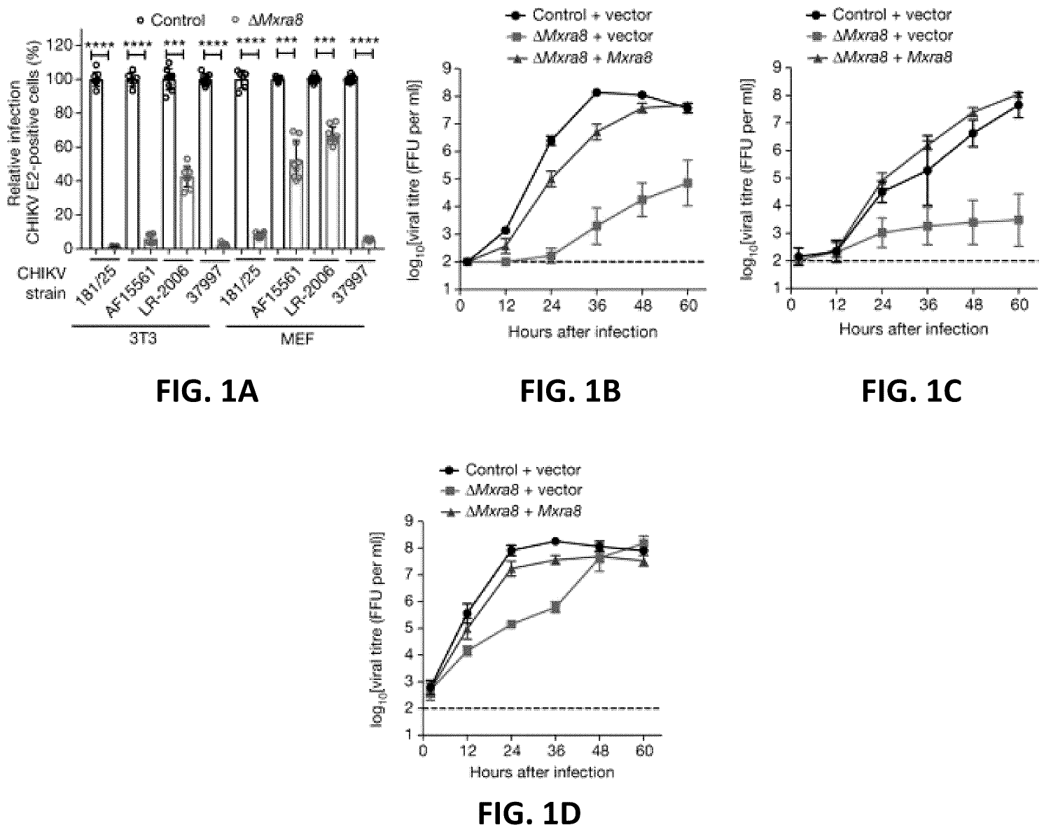

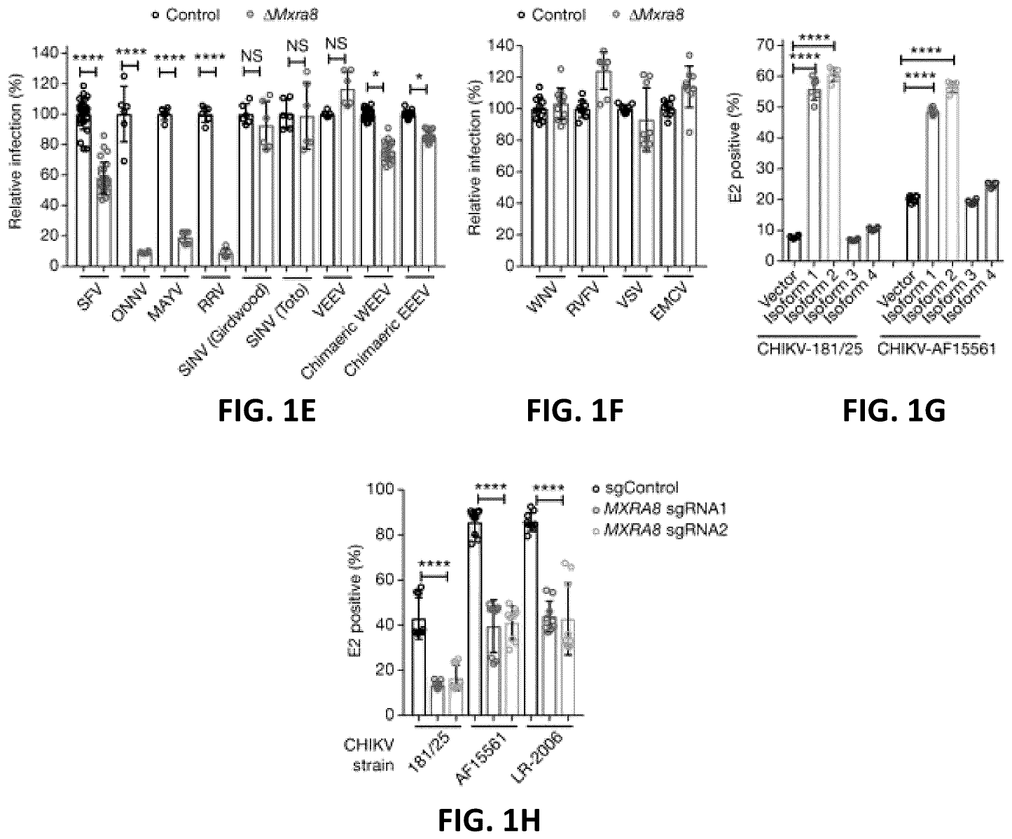

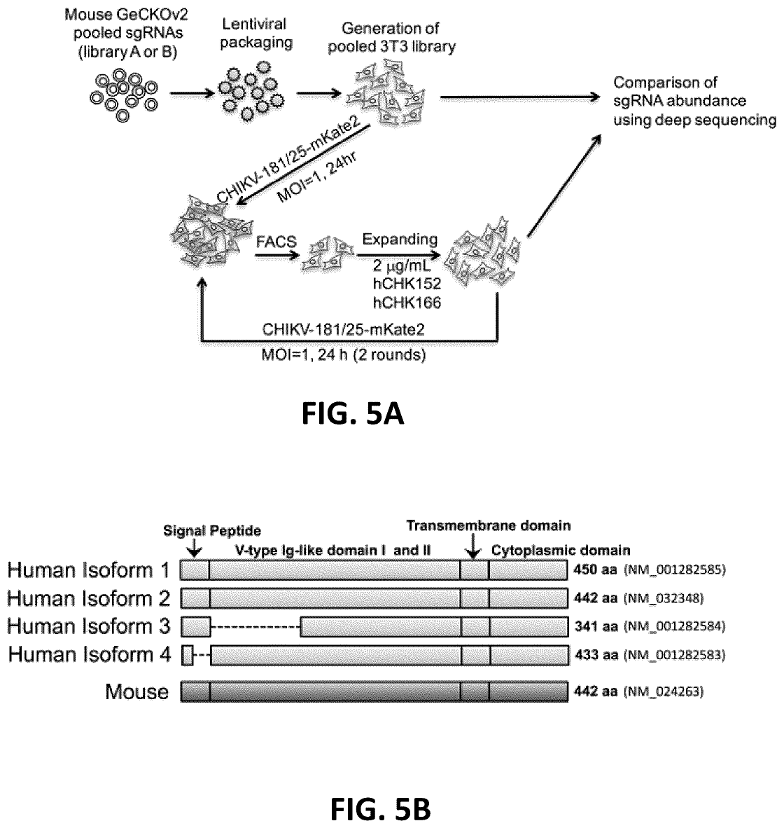

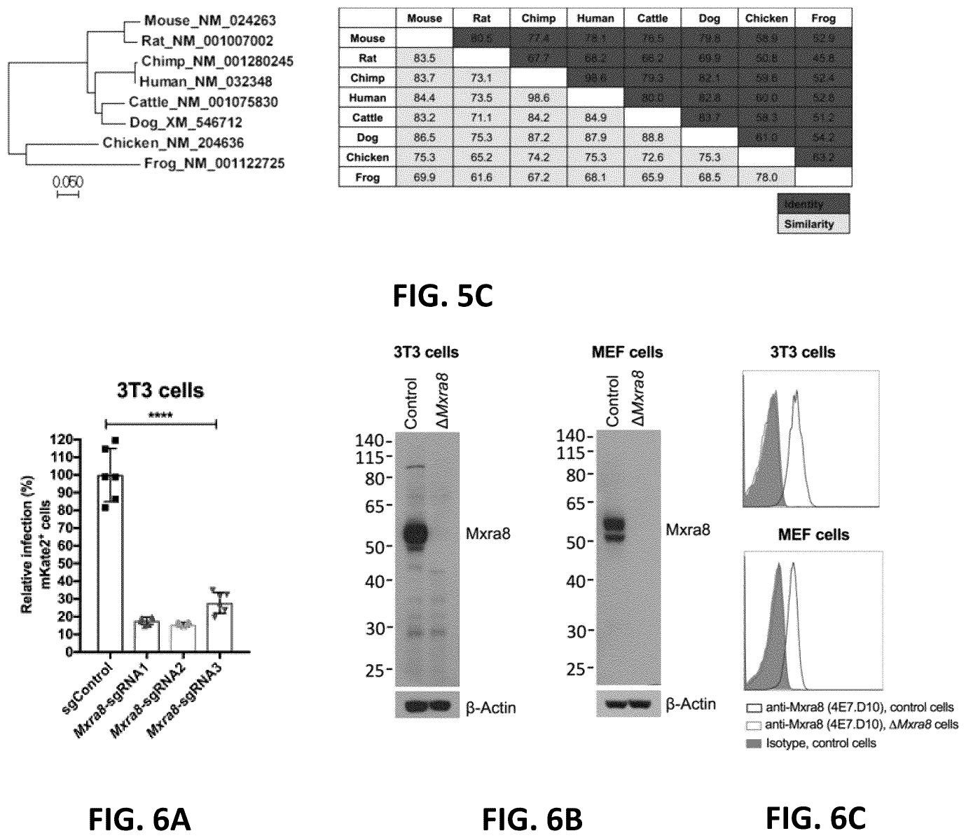

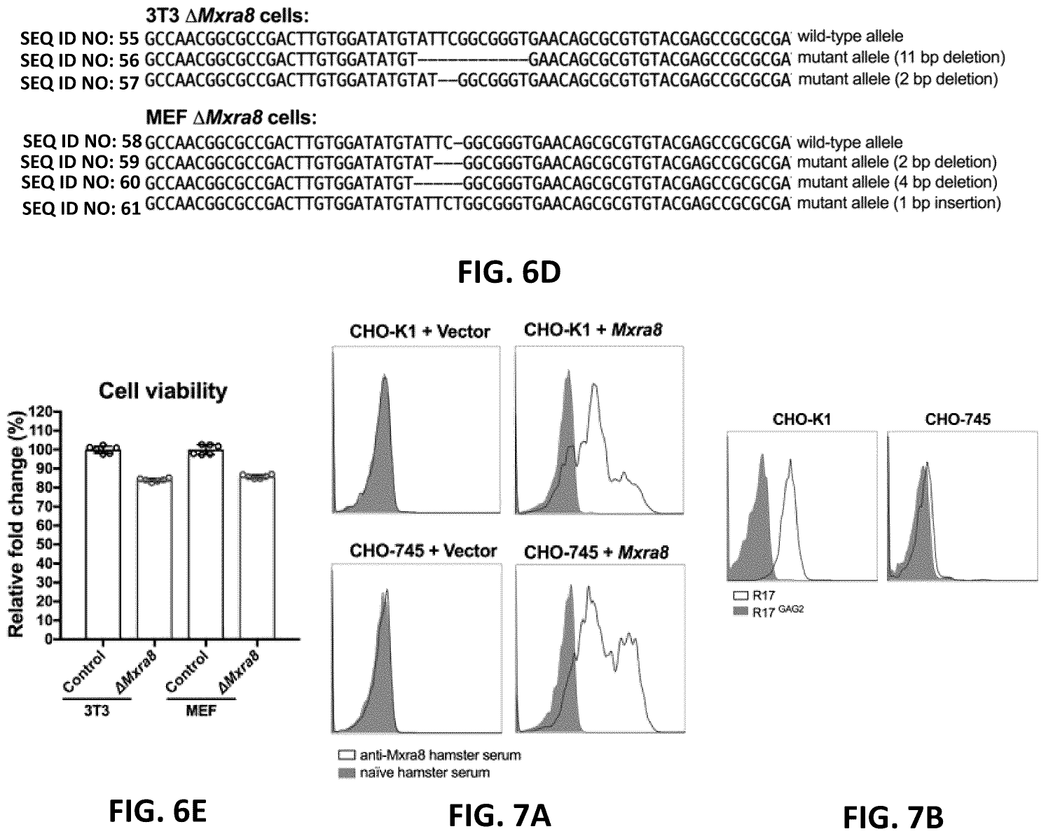

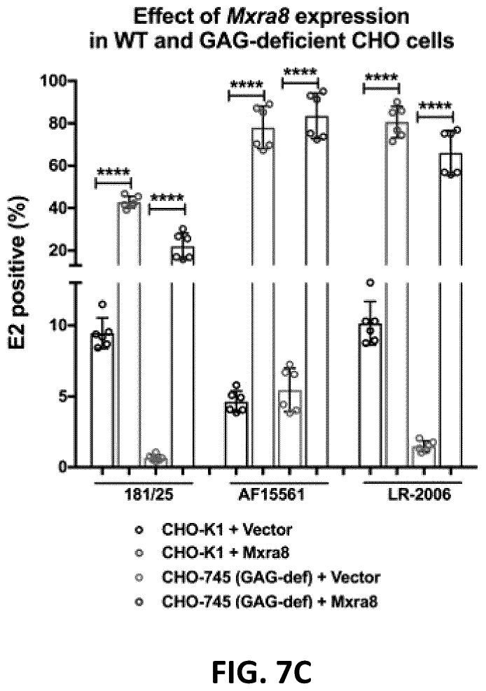

A genome-wide screen was performed for host factors required for chikungunya virus (CHIKV) infection, using the CRISPR-Cas9 system^{3,4} and lentiviruses delivering single-guide RNAs (sgRNA) targeting 20,611 mouse genes (see e.g., FIG. 5A-FIG. 5C). Lentivirus-transduced 3T3 mouse fibroblasts were inoculated with CHIKV strain 181/25 that contained the mKate2 reporter (CHIKV-181/25-mKate2), such that almost all cells expressed the reporter gene by 24 h. The few cells that lacked mKate2 expression were sorted, propagated in the presence of neutralizing anti-CHIKV monoclonal antibodies (mAbs) 5 and then re-inoculated with CH IKV-181/25-m Kate2. After two rounds of infection and sorting, genomic DNA from mKate2-negative cells was collected, sgRNAs were sequenced and then analyzed using MAGeCK⁶ (see e.g., TABLES 1A, 1B, 2A, and 2B in provisional application Nos. 62/671,680 and 62/672,080, incorporated herein by reference describe the frequency of sgRNA deep-sequencing reads). Data was obtained by deep-sequencing of sgRNAs from uninfected or sorted CHIKV-181/25-mKate2-negative cells. The two half libraries (A+B) were used for independent screens. The top candidate was Mxra8 (also known as DICAM, ASP3 or limitrin), an adhesion molecule found in mammals, birds, and amphibians (see e.g., FIG. 5B, FIG. 5C) that is expressed on epithelial, myeloid, and mesenchymal cells and shares homology with junctional adhesion molecule, a reovirus entry receptor. Mxra8 was validated using three different sgRNAs in bulk 3T3 cells, by generating ΔMxra8 single-cell clones in 3T3 and MEF cells, and by confirming gene deletion and cell viability (see e.g., FIG. 6A-FIG. 6E). Infection of CHIKV-181/25 was reduced in ΔMxra8 cells, and trans-complementation of Mxra8 in ΔMxra8 3T3 cells restored infectivity (see e.g., FIG. 1A, FIG. 1B). As CHIKV-181/25 is a cell-culture-adapted vaccine strain 12 that has acquired heparan sulfate binding activity, Mxra8 was evaluated with other CHIKV strains. Infection of CHIKV-AF15561—the parental Asian strain of CHIKV-181/25, which binds poorly to heparan sulfate—and CHIKV-37997, a West African genotype strain, was abolished in ΔMxra8 3T3 cells, reduced in ΔMxra8 MEF cells (see e.g., FIG. 1A) and restored in trans-complemented ΔMxra8 3T3 cells (see e.g., FIG. 1B, FIG. 1C). However, the dependence on Mxra8 was less with CHIKV-LR 2006, a strain of the East/Central/South African genotype (see e.g., FIG. 1A, FIG. 1D). To confirm that CHIKV required Mxra8 independently of heparan sulfate binding, murine Mxra8 was expressed in parental or glycosaminoglycan-deficient Chinese hamster ovary cells 15 (see e.g., FIG. 7A). Expression of Mxra8 enhanced infectivity of CHIKV regardless of whether Chinese hamster ovary cells expressed heparan sulfate or other glycosaminoglycans (see e.g., FIG. 7B, FIG. 7C).

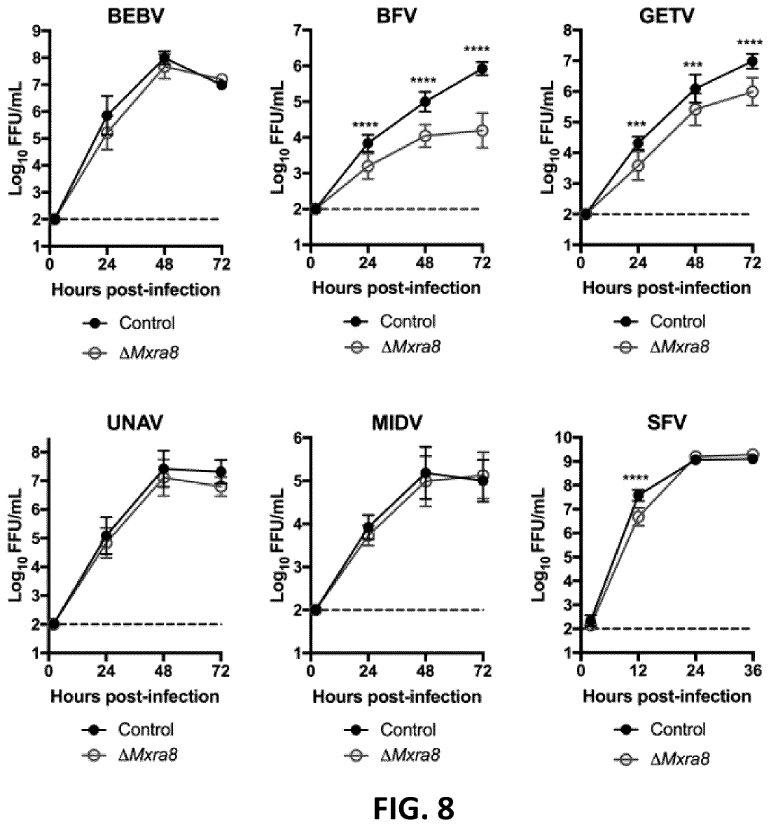

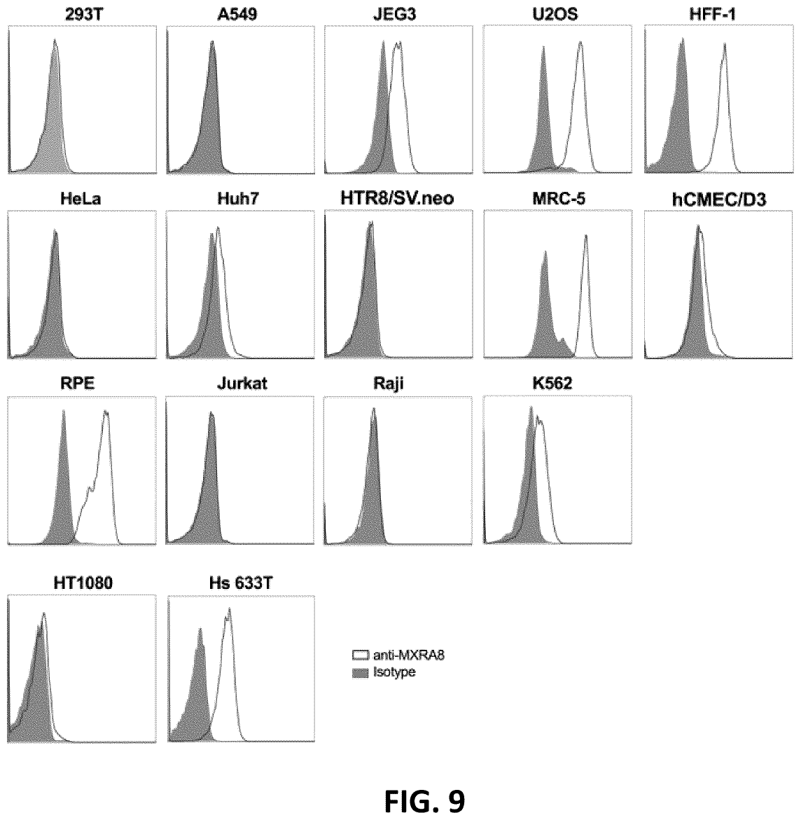

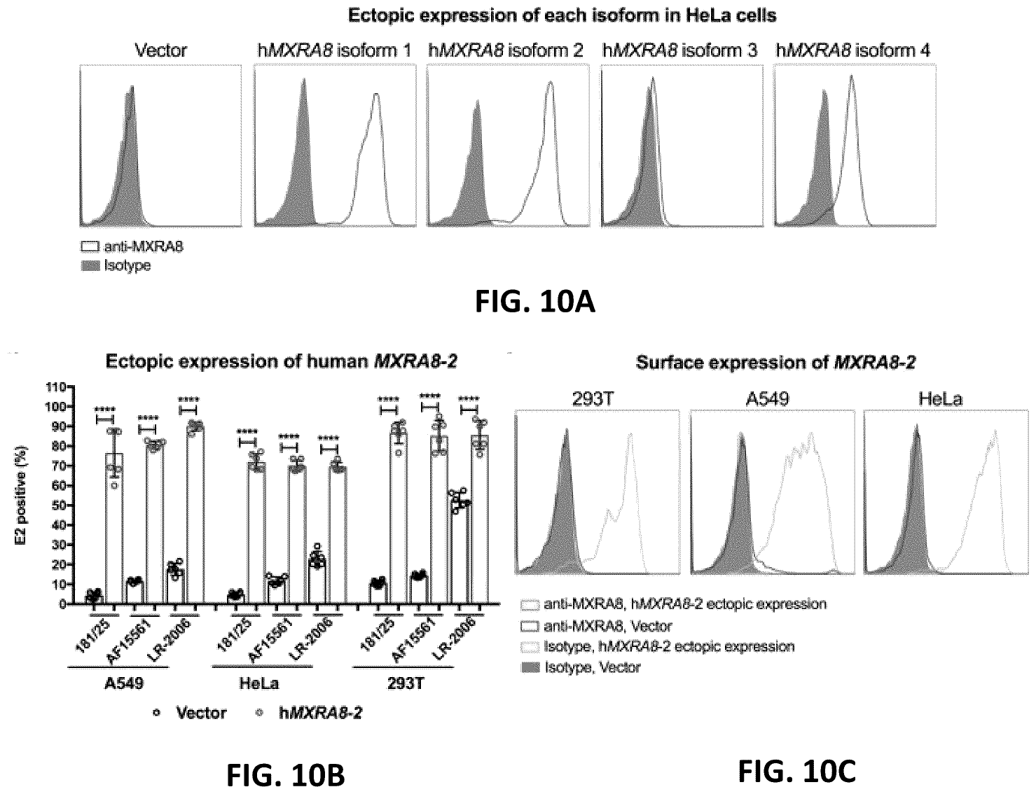

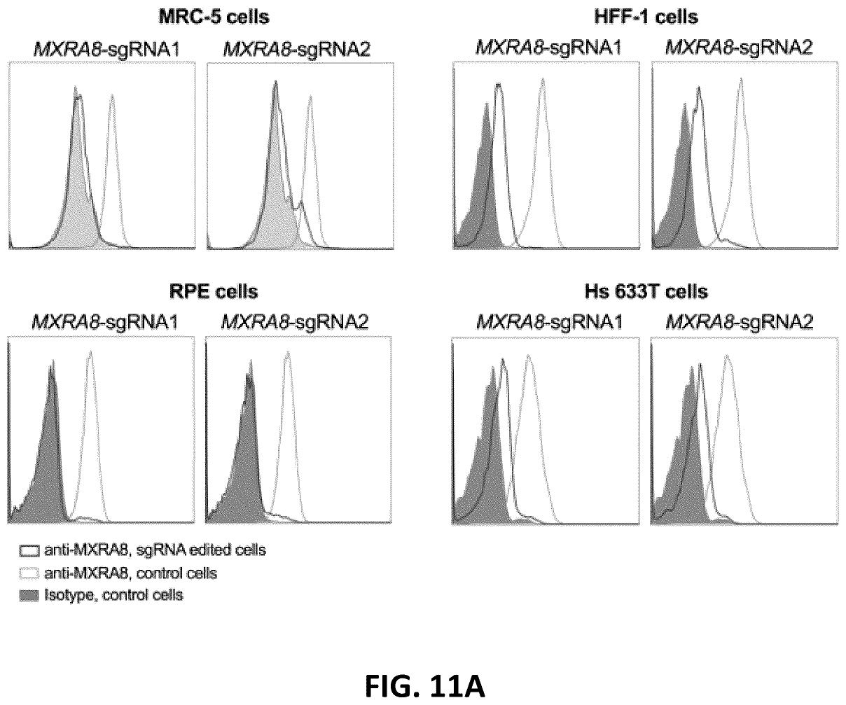

The requirement of Mxra8 for infection by other alphaviruses was tested. Whereas Mayaro, Ross River, O'nyong nyong and Barmah Forest arthritogenic alphaviruses showed reduced infection in ΔMxra8 3T3 cells, Semliki Forest and Getah viruses had partial phenotypes, and other related alphaviruses (Sindbis, Bebaru, Una and Middleburg viruses) showed little dependence on Mxra8 (see e.g., FIG. 1E and FIG. 8). Minimal differences in infection were observed between control and ΔMxra8 3T3 cells with chimaeric Sindbis viruses expressing the structural genes of the Eastern or Western equine encephalitis alphaviruses, or a Venezuelan equine encephalitis virus that expressed GFP (see e.g., FIG. 1E). No effect of Mxra8 was seen on infection of unrelated positive- or negative-sense RNA viruses (see e.g., FIG. 1F). It was next assessed whether any of the four isoforms of the human MXRA8 orthologue (see e.g., FIG. 5B) served a similar function. As HeLa cells did not express MXRA8 (see e.g., FIG. 9), these cells were used for ectopic expression (see e.g., FIG. 10A). MXRA8-1, MXRA8-2, and MXRA8-4—but not MXRA8-3—were detected on the cell surface, and MXRA8-1 and MXRA8-2 enhanced CHIKV infectivity (see e.g., FIG. 1G). Similarly, expression of MXRA8-2 in A549 or 293T cells resulted in greater CHIKV infection (see e.g., FIG. 10B, FIG. 10C). Consistent with this observation, expression of different sgRNAs targeting all isoforms of MXRA8 in MRC-5 human lung fibroblasts, HFF-1 foreskin fibroblasts, RPE retinal pigment epithelial cells and Hs 633T fibrosarcoma cells resulted in less CHIKV infection than in control gene-edited cells (see e.g., FIG. 1H and FIG. 11A, FIG. 11B).

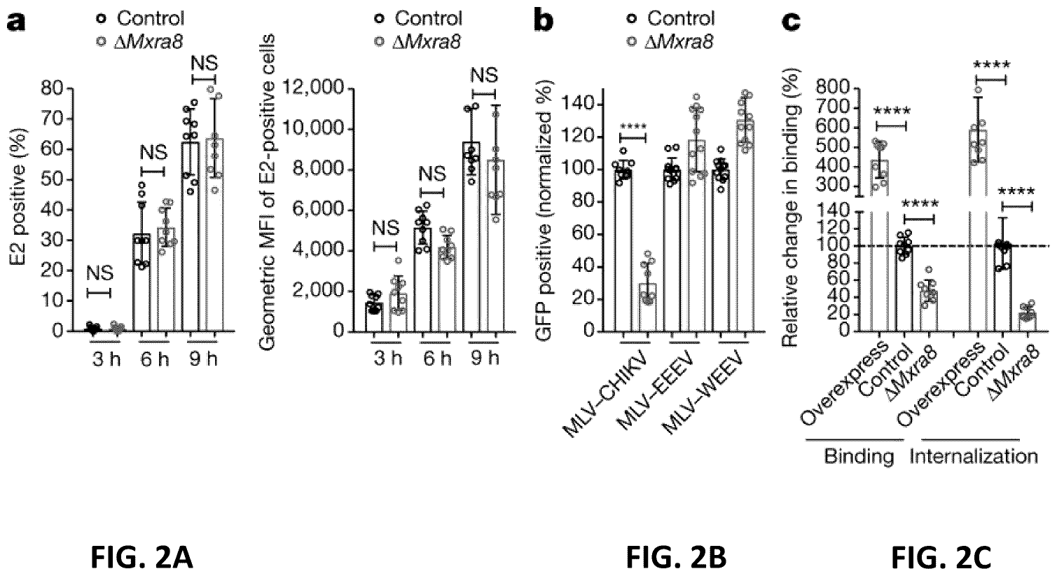

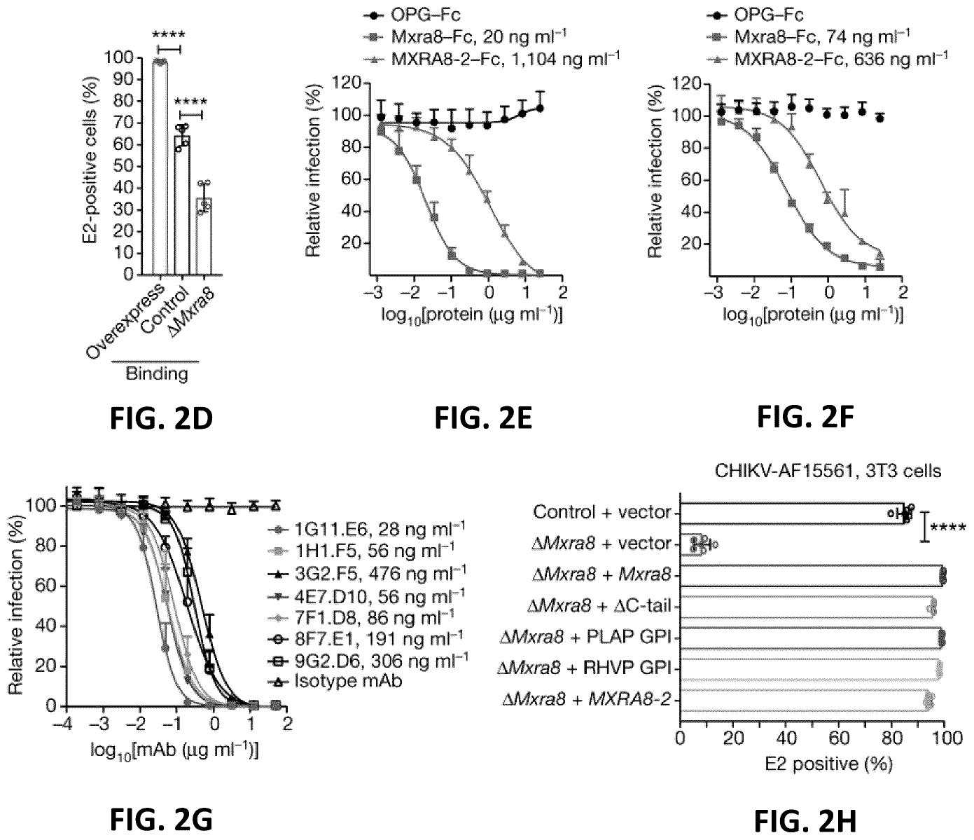

To determine whether Mxra8 is required for replication, CHIKV genomic RNA was transfected into control and ΔMxra8 MEF cells in the presence of NH₄Cl to inhibit virus maturation and further rounds of infection. As no difference in CHIKV gene expression was detected (see e.g., FIG. 2A), Mxra8 does not appear to affect translation or replication. An Mxra8-dependence of the structural proteins using pseudotyped viruses was demonstrated. Whereas infection of CHIKV pseudotyped virions that encapsidated a murine leukaemia virus GFP-reporter RNA was reduced in ΔMxra8 compared to control MEF cells, infection of Eastern or Western equine encephalitis virus pseudotyped virions was not (see e.g., FIG. 2B). Consistent with a role for Mxra8 in the entry pathway, at 4° C. CHIKV-AF15561 showed reduced binding to ΔMxra8 compared to control MEF cells, and increased binding to cells overexpressing Mxra8 (see e.g., FIG. 2C, FIG. 2D). When virus internalization assays were performed at 37° C., less CHIKV RNA was measured within ΔMxra8 MEF cells, and more CHIKV RNA was detected in cells overexpressing Mxra8 (see e.g., FIG. 2C).

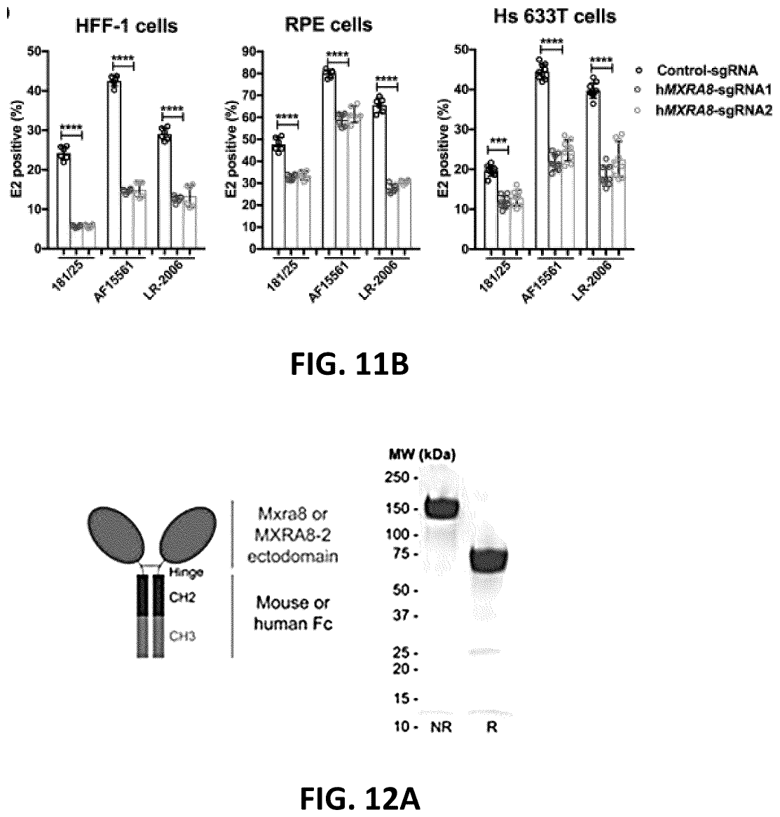

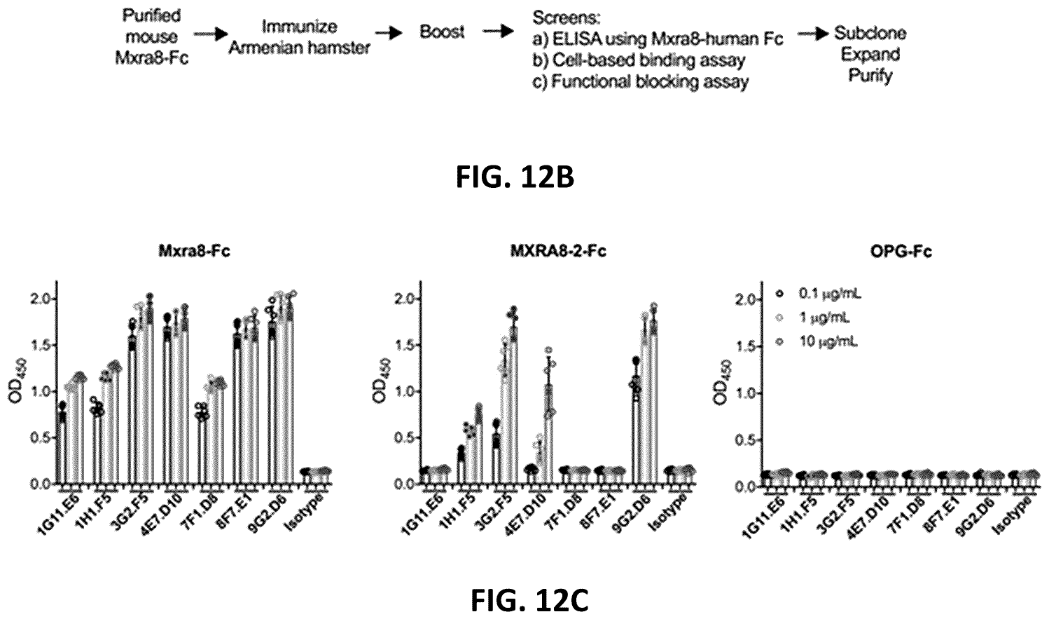

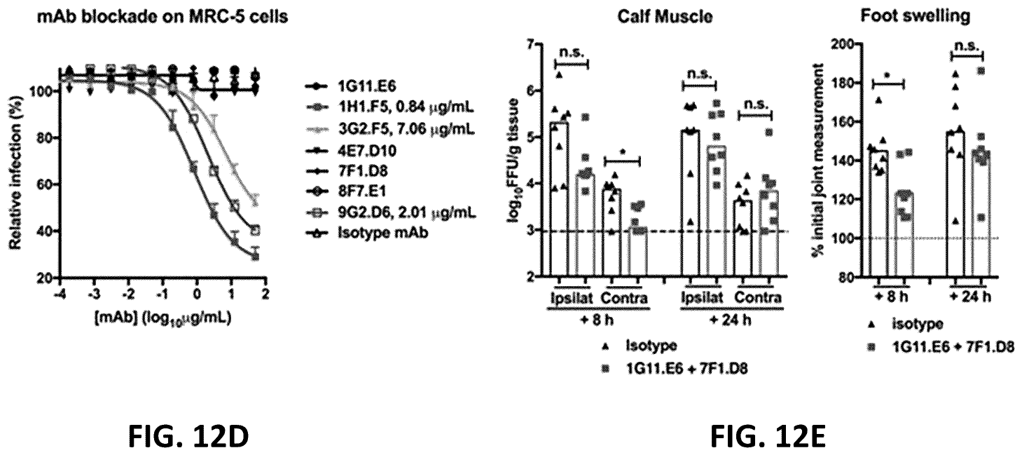

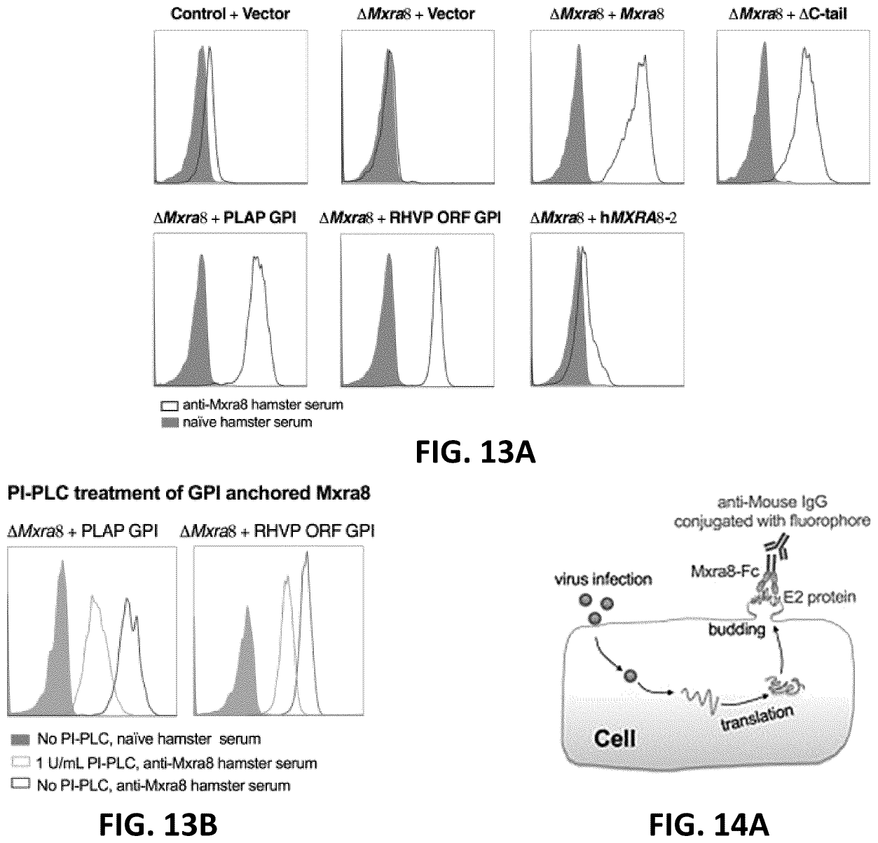

To corroborate an effect of Mxra8 on binding and entry, Fc fusion proteins with the extracellular domains of mouse Mxra8 (Mxra8-Fc) or human MXRA8-2 (MXRA8-2-Fc) were generated, along with a control osteoprotegerin protein (OPG-Fc) (see e.g., FIG. 12A). Pre-incubation with Mxra8-Fc or MXRA8-2-Fc, but not the control OPG-Fc, reduced CHIKV-181/25 infection in 3T3 (see e.g., FIG. 2E) and MRC-5 (see e.g., FIG. 2F) cells. A panel of hamster mAbs were tested against mouse Mxra8 (see e.g., FIG. 12B) for their capacity to inhibit CHIKV infection. Seven mAbs bound to mouse Mxra8-Fc, with four also recognizing human MXRA8-2-Fc (see e.g., FIG. 12C). Pre-treatment of 3T3 cells with anti-Mxra8 mAbs reduced CHIKV infection (see e.g., FIG. 2G). Three of the mAbs that bound human MXRA8-2-Fc also reduced infection in MRC-5 cells (see e.g., FIG. 12D). To establish the importance of the Mxra8 ectodomain, forms with glycophosphatidylinositol anchors (Mxra8-GPI) or lacking a cytoplasmic domain (Mxra8-ΔC-tail) for trans-complementation were engineered (see e.g., FIG. 13A-FIG. 13B). Notably, Mxra8-GPI and Mxra8-AC-tail restored CHIKV infection (see e.g., FIG. 2H). Interaction with the Mxra8 ectodomain may facilitate viral glycoprotein conformational changes that are required for internalization or fusion¹⁶ or potentiate interactions with other host factors that bridge membrane penetration and entry¹⁷. Finally, heterologous expression of human MXRA8-2 in ΔMxra8 mouse cells also restored CHIKV infection (see e.g., FIG. 2H).

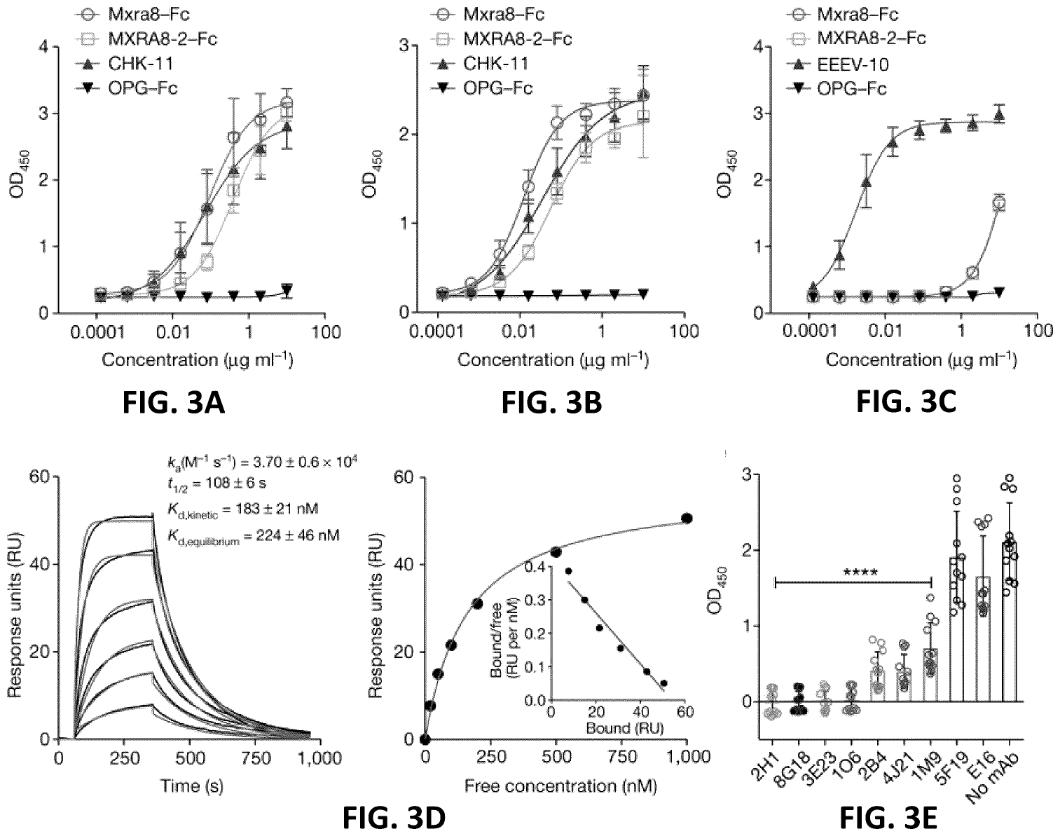

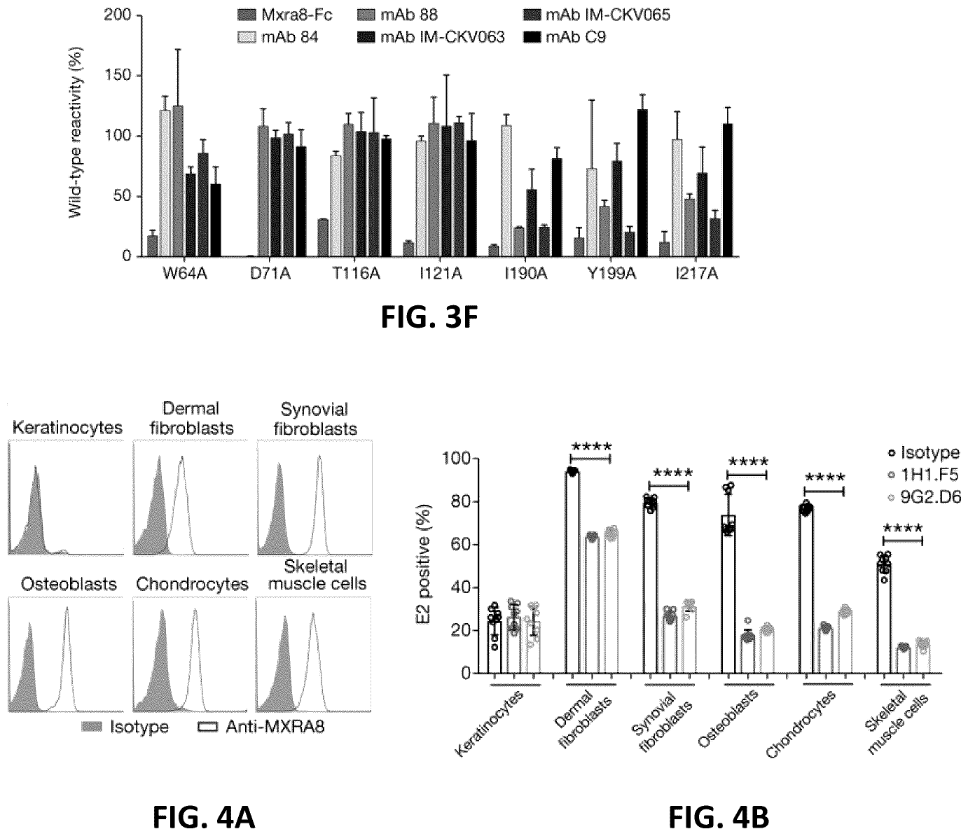

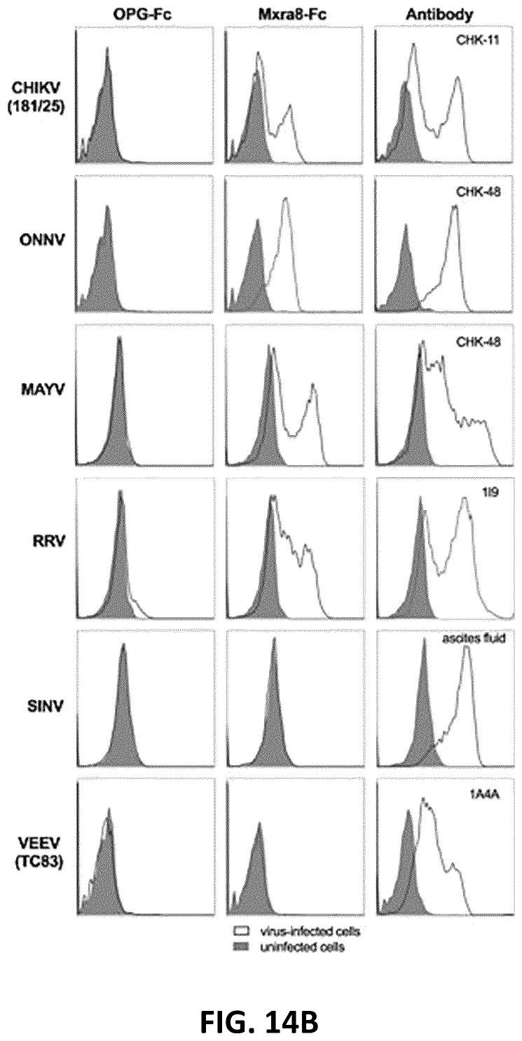

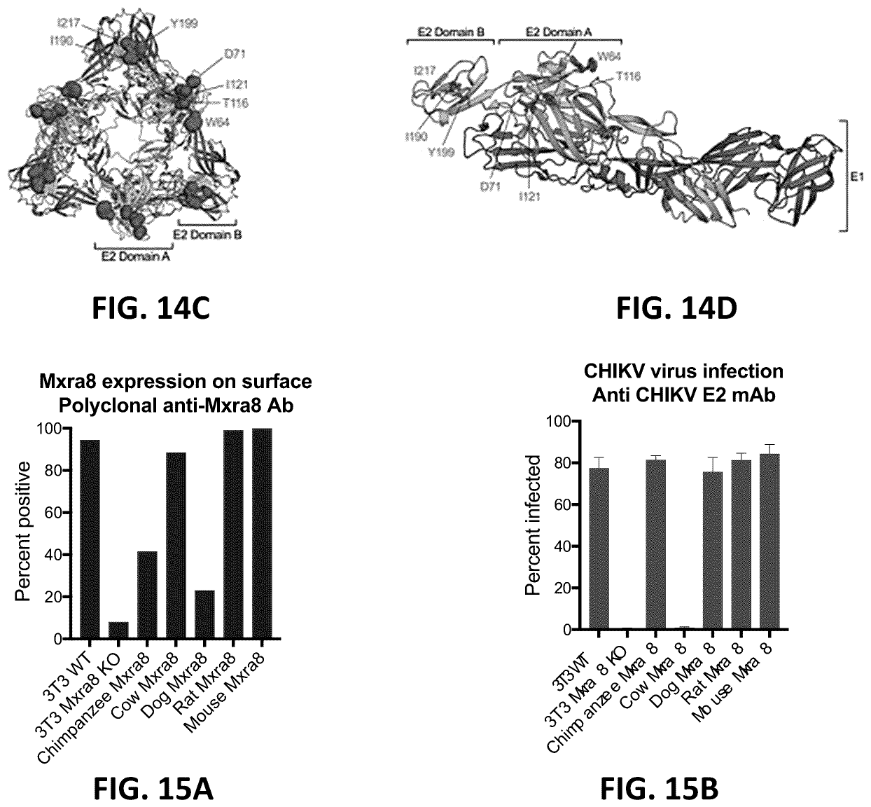

To determine whether Mxra8 directly binds to CHIKV, virions or virus-like particles¹⁸ were captured with a human anti-CHIKV mAb¹⁹, and added Mxra8-Fc or MXRA8-2-Fc in an enzyme-linked immunosorbent assay. Both Mxra8-Fc and MXRA8-2-Fc, but not OPG-Fc, bound to CHIKV virions and virus-like particles (see e.g., FIG. 3A, FIG. 3B). By comparison, Mxra8-Fc and MXRA8-2-Fc did not bind efficiently to Eastern equine encephalitis virus particles derived from a chimaera with Sindbis virus (see e.g., FIG. 3C). In a complementary assay, binding of Mxra8-Fc protein to cell-surface-displayed alphavirus proteins on infected cells^{19,20} was assessed. Mxra8-Fc bound cells infected with CHIKV, O'nyong nyong virus, Mayaro virus and Ross River virus, but not cells infected with Sindbis virus or Venezuelan equine encephalitis virus (see e.g., FIG. 14A, FIG. 14B). Binding of Mxra8 to CHIKV virus-like particles by surface plasmon resonance was analyzed, and a slow association rate, a long half-life, and an affinity of about 200 nM was found (see e.g., FIG. 3D).

It was evaluated whether human anti-CHIKV mAbs that bound epitopes within the E2 protein¹⁹ altered Mxra8-Fc binding to CHIKV. Several mAbs recognizing epitopes in the A domain (2H1, 8G18, 3E23 and 1O6) and mAbs recognizing shared epitopes in the A and B domains (1H12 and 4J14) inhibited binding, whereas other mAbs that localize to distinct sites had less effect (see e.g., FIG. 3E). The binding of Mxra8-Fc to an alanine scanning mutagenesis library of E2 Å and B domains in the context of display on the surface of 293T cells^{19,20} was then tested. Residues W64, D71, T116 and I121 in the A domain, and 1190, Y199 and 1217 in the B domain, of E2 emerged as essential for optimal Mxra8-Fc binding (see e.g., FIG. 3F and TABLE 1) and overlap the binding sites of the blocking mAbs that were tested¹⁹. Mapping of these residues onto the p62 (E2 precursor)—E1 heterodimer or the trimer of heterodimers (see e.g., FIG. 14C, FIG. 14D) on the virion surface²¹ revealed a solvent-accessible epitope across the top of the A and B domains, which is a proposed site of alphavirus receptor engagement^{22,23}.

TABLE 1
Mxra8-Fc binding to 293T cells transfected with alanine scanning mutants of C-E3-E2-6K-E1. Alanine mutations of E2 are indicated in the first column, and binding to Mxra-Fc and several CHIKV mAbs (84, 88, IM-CKV063, IM-CKV065, and C9) was tested in 293T cells and processed by flow cytometry. Percent binding of Mxra-Fc or mAbs to the respective mutant was normalized to cells transfected with wild-type C-E3-E2-6K-E1 plasmid. Data are representative of two independent experiments and parentheses indicate range. Double-underlined rows indicate mutants with loss-of-binding to Mxra-Fc that retain expression and folding. Bolded, italicized rows indicate mutations that affect overall domain/protein folding and expression, and cannot be evaluated with certainty.
Binding Reactivity (% WT (Range))

| | | | | | | |
|---|---|---|---|---|---|---|
| | Fc- | MAb 84 | MAb 88 | IM-CKV063 | IM-CKV065 | MAb C9 |
| Mutation | mMxra8 | Domain B | Domain A | Domain A | A and B | Arch 1 |

DOMAIN A

| | | | | | | | | | | | | |
|---|---|---|---|---|---|---|---|---|---|---|---|---|
| V32A | 47.1 | (10) | 36.6 | (3) | 84.8 | (21) | 44.1 | (4) | 33.3 | (4) | 44 | (0) |
| A33S | 85.3 | (0) | 75.8 | (6) | 122.7 | (26) | 99.4 | (2) | 122.9 | (28) | 86 | (18) |
| L34A | 28.3 | (3) | 23.5 | (0) | 58.3 | (4) | 29.4 | (1) | 16.3 | (1) | 34.4 | (1) |
| E35A | 11 | (2) | 99.1 | (6) | 55.9 | (9) | 19.1 | (2) | 11.7 | (0) | 22.2 | (2) |
| R36A | 68.3 | (3) | 104 | (2) | 83.8 | (16) | 76.5 | (13) | 119.2 | (1) | 75.4 | (4) |
| I37A | 36.2 | (0) | 20.7 | (1) | 63.2 | (15) | 26 | (3) | 12.1 | (3) | 30.4 | (6) |
| R38A | 90.7 | (24) | 153 | (25) | 99.6 | (5) | 94.7 | (14) | 132.9 | (23) | 86.3 | (11) |
| N39A | 91.9 | (18) | 71.5 | (4) | 123.9 | (21) | 119.5 | (4) | 120.1 | (6) | 107.8 | (24) |
| E40A | 86.2 | (12) | 74.9 | (2) | 107.5 | (7) | 106.3 | (37) | 124.1 | (3) | 106.7 | (7) |
| A41S | 4.9 | (2) | 79.9 | (5) | 89.8 | (29) | 51.1 | (23) | 33.8 | (4) | 70.7 | (15) |
| T42A | 95.7 | (5) | 64.3 | (14) | 111.8 | (33) | 110.9 | (52) | 104 | (3) | 102.4 | (30) |
| D43A | 36.2 | (1) | 4.5 | (1) | 37.5 | (5) | 8.3 | (2) | 3.7 | (1) | 11.1 | (1) |
| G44A | 72.2 | (10) | 43.8 | (5) | 98.1 | (7) | 68.2 | (7) | 50.4 | (10) | 72.5 | (8) |
| T45A | 81.2 | (9) | 48 | (4) | 85.7 | (28) | 62.1 | (6) | 62.4 | (4) | 71.7 | (8) |
| L46A | 4.8 | (1) | 3.4 | (3) | 23.6 | (3) | 2.7 | (1) | −2.7 | (1) | 1.7 | (1) |
| K47A | 102.3 | (21) | 66.1 | (18) | 100.4 | (26) | 72.1 | (7) | 85.5 | (16) | 84 | (30) |
| I48A | 11.1 | (2) | 6.4 | (0) | 39.6 | (6) | 7.9 | (0) | 0.3 | (0) | 10.7 | (1) |
| Q49A | 56.8 | (8) | 88.8 | (12) | 87.1 | (5) | 55.4 | (1) | 37.3 | (7) | 47 | (7) |
| V50A | 67.8 | (5) | 43.2 | (3) | 78.6 | (15) | 64.1 | (2) | 48.2 | (4) | 76.7 | (23) |
| S51A | 99.2 | (15) | 97 | (19) | 96.9 | (8) | 89.8 | (0) | 85.6 | (0) | 85.3 | (0) |
| L52A | 86.8 | (3) | 83.6 | (8) | 100.5 | (19) | 106.7 | (33) | 141.6 | (0) | 84.7 | (2) |
| Q53A | 26.3 | (5) | 120.5 | (29) | 85 | (10) | 37.3 | (1) | 43.3 | (4) | 39.6 | (9) |
| I54A | 104.4 | (13) | 86.9 | (3) | 115.5 | (21) | 102.2 | (1) | 80.8 | (8) | 110.4 | (29) |
| G55A | 101.2 | (29) | 87.5 | (41) | 111.4 | (16) | 83.3 | (30) | 86.2 | (25) | 75.9 | (11) |
| I56A | 91.7 | (15) | 106.3 | (29) | 115.5 | (3) | 111.3 | (11) | 101.6 | (6) | 113.7 | (18) |
| G57A | 106.1 | (1) | 101.7 | (11) | 133.8 | (10) | 96.5 | (10) | 108 | (16) | 109.1 | (28) |
| T58A | 94.1 | (8) | 114.3 | (5) | 81.2 | (14) | 88.7 | (9) | 93.5 | (21) | 98.6 | (23) |
| D59A | 78.9 | (12) | 73.2 | (9) | 86.1 | (15) | 81.1 | (17) | 72.8 | (7) | 71.8 | (9) |
| D60A | 101.8 | (18) | 70.2 | (23) | 56.5 | (1) | 64.8 | (9) | 114 | (23) | 74 | (5) |
| S61A | 115.6 | (19) | 105.8 | (13) | 105 | (15) | 100.4 | (20) | 112.4 | (16) | 93.1 | (0) |
| H62A | 102.1 | (15) | 105.1 | (2) | 101.2 | (14) | 79.6 | (1) | 88.5 | (10) | 93.1 | (17) |
| D63A | 85 | (2) | 52.1 | (10) | 85 | (16) | 65.8 | (7) | 64.8 | (9) | 71.8 | (15) |
| W64A | 17.7 | (4) | 121.7 | (12) | 125.5 | (46) | 69.1 | (5) | 86.3 | (11) | 60.6 | (14) |
| T65A | 68.9 | (2) | 76.6 | (7) | 87.9 | (17) | 70.1 | (4) | 98.4 | (25) | 71.1 | (1) |
| K66A | 93.1 | (10) | 66.8 | (5) | 103 | (26) | 84.2 | (4) | 99.6 | (4) | 85.7 | (2) |
| L67A | 77.4 | (12) | 38.7 | (0) | 84.6 | (4) | 62.1 | (3) | 54.1 | (7) | 60.9 | (0) |
| R68A | 66.1 | (8) | 76.5 | (5) | 74.7 | (16) | 67.1 | (6) | 88.1 | (1) | 64.8 | (3) |
| Y69A | 105.8 | (4) | 4.2 | (1) | 95.4 | (0) | 60.7 | (3) | 46.3 | (1) | 55.3 | (12) |
| M70A | 109.5 | (20) | 60.1 | (8) | 93.7 | (2) | 81.7 | (0) | 103.9 | (6) | 81 | (6) |
| D71A | −0.3 | (0) | 0.2 | (1) | 108.4 | (14) | 98.9 | (6) | 102.1 | (9) | 91.8 | (14) |
| N72A | 127.6 | (31) | 4.6 | (1) | 108.8 | (8) | 114.3 | (29) | 102.7 | (15) | 111.8 | (19) |
| H73A | 101.9 | (7) | 72.8 | (5) | 91.8 | (4) | 84.2 | (26) | −0.9 | (3) | 89.6 | (7) |
| I74A | 44.8 | (9) | 131.6 | (8) | 106.2 | (27) | 106.1 | (17) | 0.3 | (0) | 108.3 | (25) |
| P75A | 86.7 | (8) | 85.5 | (11) | 114.9 | (26) | 96.4 | (12) | 84.9 | (6) | 98.5 | (0) |
| A76S | 85 | (6) | 128.8 | (8) | 122 | (13) | 106.6 | (14) | 125.3 | (2) | 107.3 | (3) |
| D77A | 83.7 | (11) | 73.4 | (6) | 94.7 | (41) | 92.2 | (1) | 61.2 | (16) | 93.3 | (5) |
| A78S | 105.6 | (17) | 63.1 | (5) | 126.4 | (14) | 99.1 | (6) | 107.3 | (3) | 84.1 | (11) |
| G79A | 94.6 | (19) | 116.2 | (21) | 121.2 | (11) | 86.9 | (5) | 98.5 | (6) | 81.4 | (6) |
| R80A | 130.9 | (28) | 100.9 | (25) | 127.9 | (2) | 86.3 | (16) | 125.2 | (16) | 68.8 | (8) |
| A81S | 85.2 | (13) | 119.8 | (3) | 82.3 | (1) | 93 | (13) | 96.6 | (12) | 86.6 | (8) |
| G82A | 122.6 | (12) | 80.9 | (4) | 91.5 | (2) | 91.1 | (7) | 103.5 | (18) | 90.2 | (10) |
| L83A | 17.3 | (2) | 5.2 | (0) | 38.8 | (4) | 18.7 | (2) | 6.2 | (2) | 18.7 | (2) |
| F84A | 77.6 | (8) | 38.9 | (6) | 83.9 | (7) | 61.7 | (1) | 51.6 | (5) | 76.2 | (15) |
| V85A | 84.6 | (3) | 59.7 | (6) | 75.9 | (2) | 74.5 | (2) | 88.1 | (2) | 77.8 | (11) |
| R86A | 91.5 | (3) | 84.7 | (22) | 109.6 | (18) | 127.3 | (1) | 102 | (6) | 120.8 | (15) |
| T87A | 23.5 | (7) | 11.8 | (1) | 52.9 | (7) | 18.8 | (3) | 7.6 | (3) | 20.7 | (3) |
| S88A | 94 | (22) | 101.2 | (2) | 110.8 | (30) | 89.5 | (13) | 100.9 | (2) | 122.1 | (25) |
| A89S | 102.4 | (23) | 93.2 | (8) | 117.7 | (4) | 84 | (15) | 111.4 | (16) | 108.6 | (11) |
| P90A | 113.5 | (10) | 103.2 | (19) | 119.4 | (22) | 95.3 | (9) | 108.5 | (7) | 100.2 | (18) |
| C91A | 1 | (0) | 1.1 | (2) | 4 | (5) | 1.5 | (2) | −2 | (0) | 0.6 | (1) |
| T92A | 98.6 | (11) | 99.3 | (14) | 109.4 | (21) | 84.5 | (24) | 93.2 | (12) | 96 | (2) |
| I93A | 14.6 | (6) | 1.7 | (0) | 29.1 | (5) | 8.4 | (1) | −0.9 | (0) | 10.4 | (3) |
| T94A | 99.7 | (19) | 83.1 | (6) | 109.1 | (12) | 95.5 | (5) | 101.5 | (5) | 115.6 | (18) |
| G95A | 94.6 | (32) | 81.8 | (24) | 89 | (7) | 88.9 | (25) | 92.6 | (23) | 86.2 | (2) |
| T96A | 95.6 | (6) | 110.6 | (3) | 102.8 | (13) | 111.2 | (10) | 113.2 | (12) | 99.2 | (10) |
| M97A | 95.1 | (20) | 85.3 | (9) | 85.1 | (10) | 69.9 | (9) | 65.7 | (3) | 76.3 | (3) |
| G98A | 102.5 | (1) | 68 | (15) | 106.1 | (27) | 99.9 | (42) | 111.6 | (3) | 101.5 | (13) |
| H99A | 58.1 | (0) | 55.3 | (5) | 85.6 | (23) | 63.8 | (14) | 27.5 | (1) | 57.4 | (5) |
| F100A | 26.2 | (0) | 26.6 | (0) | 69.1 | (3) | 33.6 | (0) | 15.1 | (3) | 34.2 | (3) |
| I101A | 23.7 | (2) | 9.9 | (1) | 44.5 | (4) | 19.8 | (2) | 6.3 | (3) | 26.6 | (9) |
| L102A | 22.8 | (1) | 33 | (1) | 61.4 | (9) | 24.1 | (1) | 4.7 | (0) | 31.2 | (5) |
| A103S | 91.3 | (4) | 65.4 | (6) | 85 | (6) | 65.8 | (5) | 46.8 | (7) | 69.7 | (7) |
| R104A | 49.9 | (15) | 70 | (16) | 76 | (8) | 75.7 | (5) | 66.9 | (7) | 91.6 | (0) |
| C105A | −0.1 | (0) | −1.6 | (0) | −0.1 | (0) | 0.4 | (1) | −2.9 | (0) | −0.2 | (1) |
| P106A | 91.2 | (24) | 66.7 | (8) | 105.1 | (1) | 94.3 | (7) | 98.1 | (4) | 88.9 | (9) |
| K107A | 84.6 | (13) | 97.2 | (13) | 99.2 | (12) | 86.8 | (19) | 95.2 | (13) | 95.6 | (32) |
| G108A | 76.7 | (15) | 35.9 | (10) | 71.1 | (9) | 59 | (2) | 34.8 | (2) | 71.9 | (3) |
| E109A | 97.4 | (3) | 40.9 | (9) | 123.8 | (29) | 76.6 | (1) | 82.6 | (10) | 108.9 | (22) |
| T110A | 99.6 | (8) | 89.4 | (3) | 107.3 | (11) | 88.1 | (17) | 72.1 | (2) | 88 | (1) |
| L111A | 25.7 | (6) | 13.9 | (2) | 50.9 | (4) | 20.3 | (0) | 6.6 | (1) | 23.2 | (5) |
| T112A | 44.2 | (6) | 22.2 | (9) | 71.6 | (3) | 43.1 | (1) | 16.2 | (3) | 50.5 | (5) |
| V113A | 78 | (18) | 61.8 | (2) | 112.4 | (2) | 79.8 | (0) | 70.4 | (11) | 81.8 | (11) |
| G114A | 79.1 | (6) | 31.8 | (5) | 65.3 | (10) | 58.4 | (6) | 35.2 | (8) | 67.5 | (5) |
| F115A | 3.3 | (1) | −0.2 | (0) | 25.1 | (1) | 3.6 | (1) | −0.2 | (0) | 4.3 | (0) |
| T116A | 31.2 | (0) | 84.1 | (4) | 110 | (9) | 104.2 | (16) | 103.3 | (29) | 98.2 | (2) |
| D117A | −0.3 | (0) | 6 | (0) | 102.8 | (16) | 74.2 | (25) | 70.9 | (4) | 74.4 | (27) |
| S118A | 89.6 | (8) | 107.7 | (19) | 117.2 | (17) | 93.1 | (7) | 86 | (13) | 97.4 | (4) |
| R119A | 0 | (1) | 20 | (2) | 99.5 | (14) | 72 | (4) | 119.5 | (5) | 88 | (11) |
| K120A | 1.8 | (1) | 171.7 | (16) | 82.3 | (8) | 13.1 | (2) | 0.4 | (2) | 15.4 | (1) |
| I121A | 12.1 | (1) | 96.3 | (4) | 110.7 | (22) | 108.6 | (42) | 111.4 | (5) | 96.7 | (22) |
| S122A | 101.4 | (8) | 99 | (3) | 84.3 | (3) | 82.8 | (4) | 91.1 | (31) | 98.7 | (7) |
| H123A | 33.6 | (1) | 25.6 | (3) | 61.5 | (0) | 44.2 | (3) | 30.8 | (1) | 53.6 | (2) |
| S124A | 92.8 | (1) | 80.4 | (3) | 111.6 | (35) | 97.8 | (31) | 97.3 | (23) | 115.9 | (22) |
| C125A | −0.2 | (0) | −1 | (0) | 3.7 | (2) | 0.5 | (1) | −2.9 | (0) | 0 | (0) |
| T126A | 76 | (5) | 51.2 | (16) | 95.1 | (11) | 83.4 | (3) | 67.2 | (11) | 106.1 | (12) |
| H127A | 78 | (7) | 92.5 | (8) | 91 | (0) | 96.1 | (12) | 81.9 | (4) | 92.4 | (23) |
| P128A | 122.5 | (23) | 103.3 | (6) | 101.8 | (6) | 86 | (2) | 80.3 | (0) | 112.2 | (6) |
| F129A | 4.1 | (2) | 2.6 | (1) | 25.1 | (1) | 3.9 | (0) | −1.1 | (1) | 6.7 | (2) |
| H130A | 101 | (24) | 56.3 | (13) | 109.9 | (5) | 91 | (13) | 103.8 | (7) | 105.6 | (13) |
| H131A | 15.7 | (1) | 8.4 | (0) | 56.3 | (5) | 16.5 | (1) | 3.2 | (3) | 24.4 | (5) |
| D132A | 104.6 | (27) | 93.5 | (18) | 120.1 | (15) | 116.4 | (5) | 125.8 | (16) | 94.4 | (23) |
| P133A | 81.3 | (5) | 52.5 | (9) | 92.3 | (5) | 73.9 | (7) | 66.6 | (5) | 87.2 | (11) |

DOMAIN B

| L180A | 57.0 | (18) | 86.7 | (3) | 85.9 | (1) | 114.1 | (35) | 226.7 | (36) | 125.5 | (8) |
|---|---|---|---|---|---|---|---|---|---|---|---|---|
| L181A | 53.2 | (11) | 96.3 | (8) | 92.2 | (7) | 87.9 | (15) | 57.9 | (14) | 110.3 | (33) |
| S182A | 70.9 | (5) | 91.7 | (20) | 123.3 | (27) | 120.8 | (33) | 75.8 | (11) | 131.9 | (3) |
| Q183A | 96.1 | (29) | 100.2 | (36) | 118.9 | (23) | 101.3 | (4) | 60.6 | (11) | 176.3 | (27) |
| Q184A | 130.5 | (3) | 76.5 | (13) | 100.3 | (6) | 120.7 | (11) | 123.8 | (15) | 134.6 | (45) |
| S185A | 88.1 | (8) | 95.7 | (18) | 1.5 | (0.6) | 108.2 | (18) | 114.0 | (0.7) | 104.8 | (8) |
| G186A | 108.2 | (11) | 85.8 | (3) | 113.0 | (27) | 95.4 | (3) | 69.7 | (1) | 101.0 | (3) |
| N187A | 90.5 | (8) | 78.1 | (17) | 93.4 | (4) | 113.8 | (18) | 135.6 | (1) | 123.3 | (11) |
| —, V188A | 55.2 | (5) | 163.6 | (4) | 67.2 | (4) | 114.3 | (27) | 124.8 | (51) | 140.7 | (11) |
| K189A | 156.5 | (19) | 97.2 | (18) | 60.2 | (7) | 109.1 | (0.1) | 96.9 | (0.5) | 128.7 | (19)_{—} |
| I190A | 9.4 | (0.8) | 109.2 | (9) | 24.4 | (0.4) | 56.1 | (17) | 25.0 | (2) | 81.8 | (9) |
| T191A | 87.4 | (25) | 88.6 | (2) | 121.5 | (31) | 94.8 | (5) | 72.1 | (17) | 117.7 | (7) |
| V192A | 56.9 | (19) | 71.8 | (8) | 85.6 | (5) | 98.3 | (34) | 34.9 | (9) | 114.3 | (0.5) |
| N193A | 120.2 | (9) | 66.6 | (12) | 76.0 | (4) | 85.7 | (23) | 128.4 | (9) | 112.8 | (14) |
| S194A | 87.0 | (15) | 65.9 | (25) | 92.3 | (2) | 94.0 | (18) | 105.0 | (9) | 118.6 | (26) |
| Q195A | 82.4 | (30) | 106.2 | (7) | 102.4 | (2) | 78.6 | (9) | 0.9 | (0.7) | 128.8 | (12) |
| T196A | 86.6 | (26) | 77.8 | (7) | 95.3 | (10) | 83.0 | (17) | 0.3 | (0.07) | 99.8 | (11) |
| V197A | 33.9 | (10) | 85.9 | (14) | 48.8 | (7) | 67.4 | (8) | 78.6 | (8) | 83.8 | (27) |
| R198A | 74.0 | (17) | 80.4 | (45) | 83.5 | (13) | 98.2 | (12) | 10.2 | (0.9) | 121.6 | (49) |
| Y199A | 15.9 | (8) | 73.3 | (57) | 42.1 | (5) | 79.8 | (14) | 20.8 | (4) | 122.4 | (12) |
| K200A | 75.5 | (5) | 101.9 | (17) | 136.1 | (48) | 119.31 | (14) | 56.8 | (27) | 143.6 | (17) |
| C201A | 9.6 | (0.9) | 66.2 | (0.3) | 28.2 | (7) | 47.7 | (16) | 12.3 | (0.7) | 65.0 | (6) |
| N202A | 80.6 | (16) | 65.4 | (4) | 83.8 | (7) | 92.8 | (4) | 42.7 | (5) | 96.2 | (3) |
| C203A | 10.4 | (1) | 72.0 | (18) | 30.3 | (3) | 45.1 | (3) | 24.5 | (1) | 58.3 | (12) |
| G204A | 76.7 | (17) | 87.2 | (16) | 102.6 | (47) | 91.5 | (9) | 29.1 | (9) | 105.6 | (29) |
| G205A | 78.9 | (3) | 85.8 | (9) | 73.7 | (11) | 62.0 | (22) | 41.7 | | 110.8 | (17) |
| S206A | 52.2 | (57) | 87.3 | (20) | 113.1 | (31) | 119.3 | (30) | 65.9 | (3) | 96.0 | (84) |
| G209A | 77.9 | (22) | 71.3 | (7) | 117.3 | (4) | 99.8 | (6) | 34.8 | (9) | 108.35 | (30) |
| L210A | 70.1 | (3) | 92.56 | (6) | 76.9 | (2) | 86.3 | (9) | 36.2 | (4) | 104.9 | (15) |
| I211A | 81.1 | (64) | 68.0 | (72) | 87.7 | (33) | 77.2 | (82) | 21.9 | (4) | 125.9 | (7) |
| T212A | 94.6 | (27) | 86.4 | (4) | 95.4 | (4) | 107.81 | (26) | 3.05 | (1) | 125.4 | (0.4) |
| K215A | 106.4 | (14) | 86.9 | (34) | 137.1 | (14) | 102.3 | (41) | 44.2 | (9) | 142.8 | (79) |
| V216A | 82.9 | (36) | 100.4 | (38) | 72.9 | (1) | 97.7 | (7) | 66.5 | (8) | 112.5 | (29) |
| I217A | 12.3 | (9) | 97.5 | (23) | 48.5 | (4) | 69.7 | (21) | 32.0 | (7) | 110.5 | (13) |
| N218A | 56.5 | (14) | 71.6 | (15) | 86.5 | (28) | 97.6 | (26) | 53.5 | (27) | 72.0 | (41) |
| N219A | 66.6 | (0.3) | 83.2 | (24) | 94.7 | (8) | 98.0 | (9) | 47.5 | (5) | 117.4 | (14) |
| C220A | 60.2 | (8) | 48.6 | (2) | 73.0 | (6) | 95.2 | (16) | 32.5 | (0.4) | 103.6 | (6) |

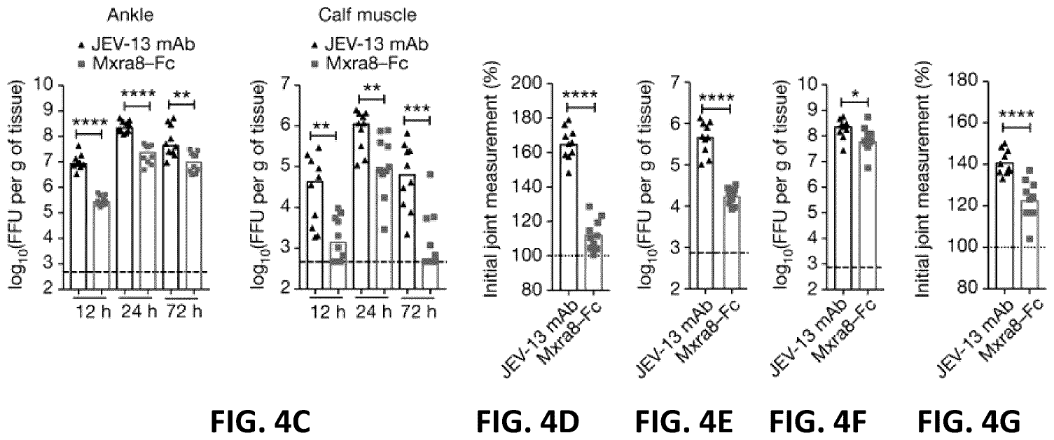

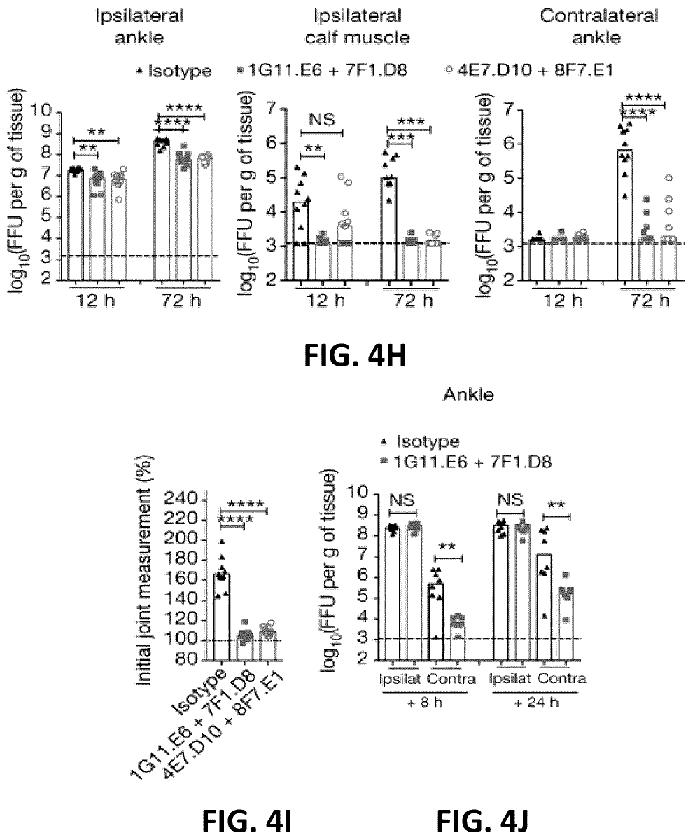

To begin to assess the physiological importance of MXRA8 interaction with CHIKV, surface expression of MXRA8 on primary human keratinocytes, dermal fibroblasts, synovial fibroblasts, osteoblasts, chondrocytes, and skeletal muscle cells were tested (see e.g., FIG. 4A), all of which are targets of infection by alphaviruses²⁴. Pretreatment with anti-MXRA8 blocking mAbs inhibited infection of CHIKV-AF15561 in all cells but keratinocytes, which lack MXRA8 expression (see e.g., FIG. 4A, FIG. 4B). It was next evaluated whether co-injection of Mxra8-Fc with CHIKV-AF15561 would diminish infection in C57BL/6 mice. The addition of Mxra8-Fc diminished CHIKV infection in the ipsilateral ankle and muscle (see e.g., FIG. 4C) and reduced foot swelling (see e.g., FIG. 4D) compared to a control mAb. Treatment with Mxra8-Fc also inhibited O'nyong nyong virus infection in the ipsilateral ankle of mice (see e.g., FIG. 4E). Mxra8-Fc was next administered via an intraperitoneal route, and 6 h later CHIKV was inoculated in the footpad. Mxra8-Fc treatment reduced foot swelling and viral burden in the ipsilateral ankle (see e.g., FIG. 4F, FIG. 4G), although the phenotype was less pronounced than in co-injection experiments. To extend these findings, hamster anti-Mxra8 blocking or control mAbs were transferred to mice via an intraperitoneal route 12 h before CHIKV inoculation. Reduced CHIKV titers were observed in the ipsilateral ankle and calf muscle, and contralateral ankle at 12 and 72 h after infection in anti-Mxra8 compared to control mAb-treated mice (see e.g., FIG. 4H). Treatment with anti-Mxra8 mAbs also reduced foot swelling (see e.g., FIG. 4I). In post-exposure therapeutic experiments, reduced CHIKV infection in the contralateral ankle and muscle when anti-Mxra8 mAbs were administered at 8 or 24 h after virus inoculation was observed (see e.g., FIG. 4J and FIG. 12E). These in vivo experiments establish a function for Mxra8 in the pathogenesis of infection of arthritogenic alphaviruses.

These studies establish that mouse Mxra8 contributes to CHIKV entry and is required for infection and disease. Human MXRA8 also bound CHIKV and supported infection, and MXRA8 expression in primary human cells overlapped with the tropism of CHIKV in vivo. Infection of several arthritogenic alphaviruses—including CHIKV, O'nyong nyong virus, Mayaro virus and Ross River virus—was reduced in ΔMxra8 cells, which suggests that Mxra8 may serve as a shared receptor. The disclosed data contrasts with those relating to natural resistance-associated macrophage protein (NRAMP2), which is an entry receptor for Sindbis virus but not for CHIKV or Ross River virus²⁵. Nonetheless, residual CHIKV infection in the absence of Mxra8 in cells and in mice, and the absence of an apparent mosquito orthologue, suggests that additional unidentified host factors contribute to cell binding and entry.

The mutagenesis mapping studies suggest that amino acids in the E2 Å and B domains contribute to the interaction of CHIKV with Mxra8. Higher-resolution structural experiments are needed to define the complete footprint of binding between Mxra8 and CHIKV E2 protein. Such studies could facilitate the development of small molecules or biological agents that disrupt Mxra8 interaction with E2 protein, which could form the basis of therapeutic strategies for the amelioration of diseases caused by multiple emerging alphaviruses.

Methods

Cells and Viruses.

Vero, NIH-3T3, MEF, HEK 293T, A549, HeLa (ATCC #CCL-2), MRC-5 (provided by D. Wang, Washington University), HFF-1 (ATCC #SCRC-1041), Hs 633T (Sigma-Aldrich #89050201), Huh7, RPE (provided by M. Mahjoub, Washington University), JEG3 (provided by I. Mysorekar, Washington University), U2OS (provided by S. Cherry, University of Pennsylvania), HT1080 (provided by J. Cooper, Washington University), Raji and K562 cells all were cultured at 37° C. in Dulbecco's Modified Eagle medium supplemented with 10% fetal bovine serum (FBS), 10 mM HEPES, 1 mM sodium pyruvate, 1×non-essential amino acids, and 100 U/m 1 of penicillin-streptomycin. HTR8/SV.neo cells (provided by I. Mysorekar, Washington University) were cultured in RPMI 1640 supplemented with 5% FBS and 1% penicillin and streptomycin. Jurkat cells were cultured at 37° C. in RPMI 1640 supplemented with 10% FBS and 10 mM HEPES. hCMEC/D3 cells (provided by R. Klein, Washington University) were cultured in EBM-2 medium (Lonza, 00190860) supplemented with 5% FBS, 5 μg/ml ascorbic acid, 10 mM HEPES, 1% lipid concentrate (Gibco, 11905-031) in plates pre-coated with collagen. All cell lines were tested and found to be free of mycoplasma contamination using a commercial kit. Cell lines were not authenticated.

Primary human keratinocytes (#102-05n), synovial fibroblasts (#408-05a), osteoblasts (#406-05f), chondrocytes (#402-05f) and skeletal muscle cells (#S150-050 were purchased from Cell Applications. Primary human dermal fibroblasts (#CC-2509) were obtained from Lonza. Cells were thawed and cultured in specified medium according to the instructions of the manufacturers, and used within one week.

The following alphaviruses were used: CHIKV (strains 2006 La Reunion OPY1, 37997, AF15561, 181/25, and 181/25-mKate2 (rescued from pJM6 CHIKV-181/25 mKate2 cDNA clone, provided by T. Morrison and M. Heise), RRV (T48), MAYV (BeH407), ONNV (MP30), SFV (Kumba), SINV (Toto1101, Girdwood), Bebaru virus (BEBV, MM 2354), Middleburg virus (MIDV, 30037), Getah virus (GETV, AMM-2021), Una virus (UNAV, CoAr2380) and Barmah Forest virus (BRV, K10521). Additional viruses tested included chimaeric encephalitic alphaviruses (SINV-EEEV and SINV-WEEV), VEEV-GFP (TC-83)), a flavivirus (WNV, New York 2000), a bunyavirus (RVFV-GFP, MP-12), a rhabdovirus (VSV-GFP, Indiana) and a picornavirus (encephalomyocarditis virus, EMCV). Replication-competent SINV chimaeric viruses were constructed by replacing the SINV TR339 structural protein genes with EEEV FL93-939 or WEEV Fleming structural protein genes under control of the SINV subgenomic promoter in the TR339 cDNA clone²⁷. All viruses were propagated in Vero cells and titrated by standard plaque or focus-forming assays²⁸.

Pooled sgRNA Screen and Data Analysis.

A GeCKOv2 CRISPR knockout pooled library encompassing 130,209 different sgRNAs against 20,611 mouse genes²⁹ was made available by F. Zhang (Addgene #1000000053), and amplified in Endura cells (Lucigen #60242) as previously described^{29,30}. The sgRNA library was divided in half (A+B), packaged into lentiviruses, and used for independent screening. The sgRNA plasmid library was packaged in 293FT cells after co-transfection with psPAX2 (Addgene #12260) and pMD2.G (Addgene #12259) at a ratio of 2:2:1 using FugeneHD (Promega). Approximately 48 h after transfection, supernatants were collected, clarified by centrifugation (3,500 r.p.m.×20 min) and aliquotted for storage at −80° C.

For the CRISPR screen, a clonal 3T3-Cas9 cell line was generated by transduction with a packaged lentivirus (lentiCas9-Blast, Addgene #52962), blasticidin selection and limiting dilution. 3T3-Cas9 cells were expanded and transduced with CRISPR sgRNA lentivirus library at a multiplicity of infection (MOI) of 0.3 by spinoculation (1,000 g) at 32° C. for 30 min in 12-well plates. After selection with puromycin for 7 days, ˜1×10⁸ cells were inoculated with CHIKV-181/25-mKate2 (MOI of 1) and then incubated for 24 h to allow nearly all cells to become infected. Cells were sorted for an absence of mKate2 expression using a Sony Biotechnology Synergy SY3200 Cell sorter (Siteman Flow Cytometry Core, Washington University). To enrich for the cell population that was resistant to CHIKV infection and increase the signal-to-noise ratio, the mKate2-negative cells were expanded in culture. Given the rapid replication rate and cytopathic effect of CHIKV, two humanized neutralizing mAbs⁵, CHK-152 and CHK-166 (2 μg/ml of each), were added to block infection by any residual virus. The expanded cells were re-infected with CHIKV-181/25-mKate2 in the absence of mAbs, sorted for mKate2 negative cells and the procedure was repeated for one additional round.

Genomic DNA was extracted from the uninfected cells (5×10⁷) or the mKate2-negative sorted cells (1×10⁷), and sgRNA sequences were amplified³¹ and subjected to next generation sequencing using an Illumina HiSeq 2500 platform (Genome Technology Access Center, Washington University). The sgRNA sequences against specific genes were determined after removal of the tag sequences using the FASTX-Toolkit (http://hannonlab.cshl.edu/fastx_toolkit/) and cutadapt 1.8.1. sgRNA sequences were analyzed using a published computational tool (MAGeCK)⁶ (see e.g., TABLES 1 Å, 1B, 2 Å, and 2B in provisional application Nos. 62/671,680 and 62/672,080, incorporated herein by reference).

Gene Validation.

Mxra8 was validated by using three independent sgRNAs as follows: Mxra8 sgRNA1, 5′-CTTGTGGATATGTATTCGGC-3′ (SEQ ID NO: 34); Mxra8 sgRNA2, 5′-TGTGCGCCTCGAGGTTACAG-3′ (SEQ ID NO: 35); Mxra8 sgRNA3, 5′-GCTGCATGATCGCCAGCGCG-3′ (SEQ ID NO: 36). The sgRNAs were cloned into the plasmid lentiCRISPR v.2 (Addgene #52961) and packaged with a lentivirus express system. 3T3 or MEF cells were transduced with lentiviruses expressing individual sg RNA and selected with puromycin for seven days before infection with different viruses. For some validation experiments, clonal cells edited by sgRNA1 were isolated by limiting dilution. To validate the human orthologue MXRA8, two different sgRNAs targeting all four isoforms were used: MXRA8 sgRNA1, 5′-GGCGCGGATGCCTTTGAGCG-3′ (SEQ ID NO: 37); MXRA8 sgRNA2, 5′-GTCCGCCTGGAGGTCACCGA-3′ (SEQ ID NO: 38). The sgRNAs were cloned similarly as above, and the gene-edited bulk cells were used for validation studies.

For flow cytometric analyses, gene-edited 3T3 cells were inoculated with different viruses as follows: CHIKV-181/25 (MOI of 3, 9.5 h), CHIKV-AF15561 (MOI of 10, 24 h), CHIKV-37997 (MOI of 3, 10 h), CHIKV-LR 2006 (MOI of 1, 9.5 h), ONNV (MOI of 3, 12 h), RRV (MOI of 3, 32 h), MAW (MOI of 3, 24 h), SFV (MOI of 1, 9 h), SINV (MOI of 10, 6 h), SIN-WEEV (MOI of 10, 10 h), SIN-EEEV (MOI of 10, 10 h), VEEV-GFP (MOI of 3, 6.5 h), RVFV-GFP (MOI of 10, 8 h), VSV-GFP (MOI of 3, 6 h), WNV (MOI of 10, 25 h) and EMCV (MOI of 3, 6 h). Gene-edited MEF cells were inoculated with CHIKV-181/25 (MOI of 3, 8 h), CHIKV-AF15561 (MOI of 10, 10 h), CHIKV-37997 (MOI of 1, 10 h) and CHIKV-LR 2006 (MOI of 1, 8 h). Gene-edited MRC-5, RPE and HFF-1 cells were inoculated with CHIKV-181/25 (MOI of 10, 10 h), CHIKV-AF15561 (MOI of 10, 10 h) and CHIKV-LR 2006 (MOI of 1, 10 h). Gene-edited Hs 633 T cells were inoculated with CHIKV-181/25 (MOI of 15), CHIKV-AF15561 (MOI of 15) and CHIKV-LR 2006 (MOI of 3) for 11.5 h. At the indicated times, cells were collected with trypsin, and fixed with 1% paraformaldehyde (PFA) diluted in PBS for 15 min at room temperature and permeabilized with Perm buffer (Hank's Balanced Salt Solution (HBSS) supplemented with 10 mM HEPES, 0.1% (w/v) saponin, and 2% FBS) for 10 min at room temperature. Cells were then incubated for 30 min at room temperature with 1 μg/ml of the following virus-specific antibodies: CH IKV (mouse CHK-11⁵), ONNV and MAYV (mouse CHK-48), RRV (human 1I9); SINV (ascites fluid, ATCC VR-1248AF), SFV (mouse CHK-124), VEEV (mouse 1 Å4 Å), WEEV (mouse 11 Å1), EEEV (mouse EEEV-10), WNV (human E16³²) and EMCV (mouse serum). After washing, cells were incubated with 2 μg/ml of Alexa Fluor 647-conjugated goat anti-mouse or anti-human IgG (Invitrogen) for 30 min at room temperature. Cells were processed on a MACSQuant Analyzer 10 (Miltenyi Biotec), and analyzed using FlowJo software (Tree Star).

Validation also was performed by an infectious virus yield assay. Gene-edited 3T3 cells were plated 12 h before infection. Cells were inoculated with CHIKV-181/25, CHIKV AF15561, CHIKV-LR 2006 at an MOI of 0.01 or other alphaviruses (BEBV, MOI 0.001; BFV, GETV, UNAV, MIDV, and SFV, all at MOI of 0.01) for 1 h, then washed once and maintained in reduced 2% FBS culture medium. Supernatants were collected at specific times after infection for titration on Vero cells by focus-forming assay (CHIKV) or standard plaque assay (all other viruses).

Genomic RNA Transfection and Analysis.

To assess for effects of Mxra8 on CHIKV replication, capped viral genomic RNA was transfected into MEF cells. Capped genomic RNA was generated using an mMESSAGE mMACHINE SP6 Transcription Kit according to the manufacturer's instructions (Thermo Fisher #AM1340) from the NotI-linearized CHIKV-181/25 cDNA clone. One microgram of purified RNA was transfected into control or ΔMxra8 cells using the Neon transfection system according to the manufacturer's instructions (Thermo Fisher Scientific). Cells were then incubated in 15 mM NH4Cl to prevent subsequent rounds of infection. At specified times, cells were collected with trypsin and processed for E2 expression levels by flow cytometry.

Pseudotyped Virus Experiments.

MLV-GFP pseudoviruses were made as described^{33,34} except plasmids encoding structural proteins of CHIKV (strain 37997), VEEV (strain TrD) and EEEV (strain FL91-4697) were used. Pseudovirus entry in 3T3 cells expressing or lacking Mxra8 was assessed 36 h later by measuring GFP expression by flow cytometry.

Plasmid Construction.

The C-terminal Flag-tagged mouse Mxra8 corresponding to the transcript (NM_024263) was synthesized (Integrated DNA Technologies) and cloned into the lentivirus vector pCSII-EF1-IRES-Venus with restriction sties NotI/BamHI. The Mxra8 sgRNA target sequences were mutated (cttgtggatatgtattcggcg (SEQ ID NO: 39) to ctGgtCgaCatgtaCAGCgcg (SEQ ID NO: 40)) to avoid re-cutting by Cas9 protein for the trans-complementation. Based on this plasmid, a truncation lacking the cytoplasmic domain was constructed by PCR-mediated mutagenesis, the AC-tail (378-442). To express the GPI-anchored Mxra8, the N-terminal 336 amino acids missing the transmembrane and cytoplasmic domains were fused with PLAP (ctggcgccccccgccggcaccaccgacgccgcgcacccggggcggtccgtggtccccgcgttgcttcctctgctggccg ggaccctgctgctgctggagacggccactgctccc) (SEQ ID NO: 41) or the rodent herpesvirus Peru (RHVP) open reading frame R1 gene (tacccatacgatgttccagattacgctacgtcctcaccatccattggcggcccaaacatgactttactattggccatgatcat gtttgcgttaaagatagggtcg, HA tag is underlined) (SEQ ID NO: 42) GPI anchor sequences. To assess the function of different human MXRA8 isoforms, the cDNA of isoform 2 (NM_032348.3; SEQ ID NO: 43) containing C-terminal Myc and Flag tags was purchased from OriGene (Cat. No. RC200955), and cloned into the lentivirus vector pCSII-EF1-IRES-Venus. Isoform 1 (NM_001282585.1; SEQ ID NO: 44), isoform 3 (NM_001282584.1; SEQ ID NO: 45) and isoform 4 (NM_001282583.1; SEQ ID NO: 46) were created by either mutagenesis of isoform 2 or gene synthesis (Integrated DNA Technologies), and cloned into the lentivirus vector pCSII-EF1-IRES-Venus containing C terminus Myc and Flag tags. Trans-complementation and ectopic expression experiments.

The plasmids constructed above were packaged using the lentivirus expression system. Cells transduced with these lentiviruses were sorted for Venus-positive cells by flow cytometry. 3T3 cells were inoculated with CHIKV-181/25 (MOI 3) for 9.5 h or CHIKV-AF15561 (MOI 10) for 20 h. HeLa and A549 cells were inoculated with CHIKV-181/25 (MOI 3), CHIKV-AF15561 (MOI 10) or CHIKV-LR 2006 (MOI 1) for 14 h. 293T cells were inoculated with CHIKV-181/25 (MOI 1), CHIKV-AF15561 (MOI 3) or CHIKV-LR 2006 (MOI 0.5) for 11.5 h. CHO cells (K1 and 745) were inoculated with CHIKV-181/25 (MOI 0.3), CHIKV-AF15561 (MOI 10) or CHIKV-LR 2006 (MOI 0.3) for 12 h. Cells were then collected and processed for E2 expression by flow cytometry.

Generation and Production of Mxra8-Fc and MXRA8-2-Fc.

A cDNA fragment encoding the mouse Mxra8 extracellular domain (residues 22-336, GenBank accession number NM_024263 (SEQ ID NO: 32)) or the human MXRA8-2 extracellular domain (residues 20-337, GenBank accession number NM_032348.3 (SEQ ID NO: 33)) and the mouse IgG2b-Fc were synthesized (Integrated DNA Technologies) and inserted into the pCDNA3.4 vector (Thermo Fisher) downstream of an IL-2 signal peptide sequence. After confirmation by Sanger sequencing, Mxra8-Fc and MXRA8-2-Fc were expressed into Expi293 cells (Thermo Fisher). Cells were seeded at 5×10⁶ cells per ml one day before transfection. Two hundred micrograms of plasmid were diluted in Opti-MEM (Thermo Fisher) and complexed with HYPE-5 transfection reagent before addition to cells. Transfected cells were supplemented daily with Expi293 medium and 2% (w/v) Hyclone Cell Boost. Four days after transfection, supernatant was collected, centrifuged at 3,000 g for 15 min, and purified by protein A sepharose 4B (Thermo Fisher) chromatography. After elution and buffer neutralization, the purified protein was dialysed into 20 mM HEPES, 150 mM NaCl (pH 7.5), filtered through a 0.20-μm filter, and stored at −80° C. Mxra8 that was cleaved from the IgG backbone was generated by inserting an HRV cleavage sequence (LEVLFQGP) (SEQ ID NO: 52) into Mxra8-Fc downstream of the Mxra8 coding sequence and before the mouse IgG sequence. Mxra8-HRV-Fc was expressed in Expi293 cells as described above. After purification, Mxra8-HRV-Fc was cleaved using HRV 3C protease (Thermo Fisher) at a 1:10 ratio overnight at 4° C. Cleaved Fc fragments were depleted using protein A sepharose chromatography, and the purity of Mxra8 was confirmed using SDS-PAGE analysis.

Expression of CHIKV Virus-Like Particles.

The CHIKV virus-like particles plasmid (strain 37997) was provided by G. Nabel (via the Vaccine Research Center, NIH) and expressed in Expi293 cells as previously described¹⁸. The supernatant was collected four days after transfection, 0.2-μm filtered and stored at 4° C.

ELISA-Based Mxra8-Fc Binding Assays.

Anti-CHIKV human mAb 4N12¹⁹ or anti-EEEV human mAb 53 (J.E.C., unpublished data) were immobilized (50 μl, 2 μg/ml) onto Maxisorp ELISA plates (Thermo Fisher) overnight in sodium bicarbonate buffer, pH 9.3. Plates were washed four times with PBS and blocked with PBS supplemented with 4% BSA for 1 h at room temperature. CHIKV-181/25 or SINV-EEEV was diluted to 1.5×10⁷ FFU per ml in PBS and 50 μl per well was added for 1 h at room temperature. Mxra8-Fc and respective positive (CHK-11⁵ and EEEV-10 (A.S.K. and M.S.D., unpublished results) and negative OPC-Fc (D.H.F., unpublished results) controls were diluted in PBS supplemented with 2% BSA and incubated for 1 h at room temperature. After serial washes with PBS, plates were incubated with horseradish peroxide conjugated goat anti-mouse IgG (H+L) (1:2000 dilution, Jackson ImmunoResearch) for 1 h at room temperature. After washing with PBS, plates were developed with 3,3′-5,5′ tetramethylbenzidine substrate (Dako) and 2N H2504. Absorbance was read at 450 nm with a TriStar Microplate Reader (Berthold). The mAb competition binding assay was performed by incubating 10 μg/ml of indicated anti-CHIKV human mAbs 19 for 30 min before the addition of mMxra8-Fc, as described above; the anti-CHIKV human mAbs were mapped previously to different epitopes by alanine scanning mutagenesis and evaluated for neutralizing activity¹⁹. Humanized anti-WNV mAb E16³² was included as a negative control in competition binding assays.

Surface Plasmon Resonance Based Mxra8 Binding Assay.

Surface plasmon resonance binding experiments were performed on a Biacore T200 system (GE Healthcare) to measure the kinetics and affinity of Mxra8 binding to CHIKV virus-like particles. Experiments were performed at 30 μl/min and 25° C. using 0.01 M HEPES pH 7.4, 0.15 M NaCl, 3 mM EDTA, 0.005% v/v surfactant P20 as running buffer. CHK-265 mAb 5 was immobilized onto a CM5 sensor chip (GE Healthcare) using standard amine coupling chemistry, and CHIKV virus-like particles were captured. Recombinant Mxra8 was injected over a range of concentrations (1 μM to 20 nM) for 5 min followed by a 10 min dissociation period. As a negative control, murine norovirus was captured with mAb A6.2, as previously described³⁵. Real-time data were analyzed using BIAevaluation 3.1 (GE Healthcare). Kinetic profiles and steady-state equilibrium concentration curves were fitted using a global 1:1 binding algorithm with drifting baseline.

Cell-Based Mxra8-Fc Binding Assay.

3T3 cells were inoculated with different viruses at the following MOI: CHIKV-181/25 (MOI of 3, 9.5 h), ONNV (MOI of 5, 12 h), MAW (MOI of 3, 24 h), RRV (MOI of 3, 32 h), SINV (MOI of 10, 9 h) and VEEV (MOI of 3, 6.5 h). Cells were detached using TrypLE (Thermo Fisher #12605010), washed twice with cold HBSS supplemented with 15 mM HEPES and 2% FBS. Cells were incubated with 1 μg/ml of Mxra8-Fc, OPG-Fc or viral E2-specific antibodies at 4° C. for 25 min. After washing, cells were stained with goat anti-mouse IgG (H+L) conjugated with Alexa Fluor 647 for 25 min at 4° C. After washing twice, cells were fixed with 2% PFA for 10 min at room temperature. After two additional washes, cells were subjected to flow cytometry analysis.

Virus Binding and Internalization Assays.

The assays were conducted in suspension. MEF cells were collected using TrypLE and washed twice with ice-cold medium supplemented with 2% FBS. CHIKV-AF15561 virions were purified through a 25% glycerol cushion at 25,000 r.p.m. for 2 h. For the binding assay, cells (5×10⁵) and virions (MOI of 20) were mixed in a 1.5-ml microcentrifuge tube and incubated on ice for 45 min. After five cycles of centrifugation and washing, cells were lysed in RLT buffer for RNA extraction using an RNeasy Mini Kit (QIAGEN #74104). For the internalization assay, after 5 cycles of centrifugation and washing, cells were resuspended into medium supplemented with 2% FBS and 15 mM NH₄Cl and then incubated at 37° C. for 1 h. Cells were chilled on ice and treated with 500 ng/ml proteinase K on ice for 1 h. After three additional washes, cells were lysed in RLT buffer for RNA extraction. qRT-PCR was conducted with Gapdh as an internal control using a TaqMan RNA-to-CT 1-Step Kit (Thermo Fisher #4392938). Primers and probes used are as follows: Fwd CHK181/AF: 5′-TCGACGCGCCATCTTTAA-3′ (SEQ ID NO: 47); rev CHK181/AF: 5′-ATCGAATGCACCGCACACT-3′ (SEQ ID NO: 48); probe CHK181/AF: 5′ 6-FAM/ACCAGCCTG/ZEN/CACCCACTCCTCAGAC/3′ IABkFQ (SEQ ID NO: 49); fwd Gapdh: 5′-GTGGAGTCATACTGGAACATGTAG-3′ (SEQ ID NO: 50); rev Gapdh: 5′-AATGGTGAAGGTCGGTGTG-3′ (SEQ ID NO: 51); and probe Gapdh: 5′ 6-FAM/TGCAAATGG/ZEN/CAGCCCTGGTG/3′ IABkFQ (SEQ ID NO: 52).

For the flow cytometry-based binding assay, experiments were conducted as above but with 5×10⁴ cells and an MOI of 200. After binding and washing, cells were fixed and stained with a mixture of mAbs (CHK-11, CHK-84, CHK-124 and CHK-166⁵) (1 μg/ml) at room temperature for 25 min. Cells were washed once and stained with 2 μg/ml of goat anti-mouse IgG (H+L) conjugated with Alexa Fluor 647 (Thermo Fisher #A21235) for 25 min. After two additional washes, cells were subjected to flow cytometry analysis.

Surface Staining of Mouse Mxra8 and Human MXRA8.

Mouse or human cells were collected with TrypLE and washed twice with cold HBSS supplemented with 15 mM HEPES and 2% FBS. Cells were incubated with anti-Mxra8 (mouse) Armenian hamster serum (1:300), anti-Mxra8 (mouse) hamster mAbs (1 μg/ml) or anti-MXRA8 (human) mAb (1 μg/ml) (MBL International #W040-3) at 4° C. for 25 min. After washing, cells were stained with 2 μg/ml goat anti-Armenian hamster IgG (H+L) conjugated with Alexa Fluor 647 (Abcam #ab173004) or goat anti-mouse IgG (H+L) conjugated with Alexa Fluor 647 (Thermo Fisher #A21235) for 25 min at 4° C. After two additional washes, cells were fixed with 2% PFA for 10 min at room temperature. Cells were then washed twice and subjected to flow cytometry analysis.

Cell Viability Assay.

A CellTiter-Glo Luminescent Cell Viability Assay (Promega) was performed according to the manufacturer's instructions. In brief, 2×10⁴ 3T3 or MEF cells in 100 μl culture medium were seeded into opaque-walled 96-well plates. 24 h later, 100 μl of CellTiter-Glo reagent was added to each well and allowed to shake for 2 min. After a 10-min incubation at room temperature, luminescence was recorded by using a Synergy H1 Hybrid Plate Reader (Biotek) with an integration time of 0.5 s per well.

Western Blotting.

Cells seeded in 6-well plates were washed once with PBS, chilled on ice in PBS and detached with a cell scraper. After spinning at 300 g for 5 min, cell pellets were lysed in 45 μl RIPA buffer (Cell Signaling #9806S) with a cocktail of protease inhibitors (Sigma-Aldrich #S8830). Samples were prepared in LDS buffer (Life Technologies) under reducing (+dithiothreitol) conditions. After heating (70° C., 10 min), samples were electrophoresed using 10% Bis-Tris gels (Life Technologies) and proteins were transferred to PVDF membranes using an iBlot2 Dry Blotting System (Life Technologies). Membranes were blocked with 5% non-fat dry powdered milk and probed with hamster mAb 3G2.F5 (0.5 μg/ml) against mouse Mxra8. Western blots were developed using SuperSignal West Pico Chemiluminescent Substrate or SuperSignal West Femto Maximum Sensitivity Substrate (Life Technologies).

Alanine Scanning Mutagenesis for Mapping.

A CHIKV E2, 6K and E1 envelope protein expression construct (strain S27, Uniprot Reference #Q8JUX5) with a C-terminal V5 tag was subjected to alanine scanning mutagenesis to generate a comprehensive mutation library³⁶. Each residue of the envelope proteins was mutated to alanine, with alanine codons mutated to serine. One hundred and forty-one mutations within the E2 Å and B domains were screened for binding by Mxra8-Fc. Binding of Mxra8-Fc to each mutant expressed in HEK-293T cells was determined by immunofluorescence detected with a high-throughput flow cytometer (Intellicyt), as previously described 36. Residues of domains A or B were identified as contributing to the binding site if their mutation eliminated Mxra8-Fc binding, but supported binding of CHIKV mAbs that bind to the appropriate domain (control mAbs were CHKV-84, CHKV-88, IM-CKV063, IM-CKV065 and C9^{5,36,37}).

Mapping of Mutations onto the CHIKV p62-E1 Crystal Structure.

Figures were prepared using the atomic coordinates of CHIKV p62-E1 (RCSB accession number 3N41) using the program PyMOL (The PyMOL Molecular Graphics System, Version 1.7.4 Schrödinger, LLC).

mAb Generation.

Four-week-old male Armenian hamsters were immunized intravenously with 100 μg of purified Mxra8-Fc. After two boosts (˜7 months of age), spleens were collected for hybridoma fusion and mAb production. Hybridoma supernatants were initially screened by ELISA using Mxra8-human Fc (mouse Fc was replaced by human Fc). As a second assay, the binding of hybridoma supernatants to Mxra8 on the surface of 3T3 cells was examined by flow cytometry. Finally, a tertiary screen evaluating blockade of CHIKV-181/25 infection by hybridoma supernatants was performed in 3T3 cells. After limiting dilution subcloning, the seven clones with the strongest blocking activities were selected and expanded. Antibodies were purified using protein A sepharose 4B (Invitrogen #101042), dialyzed in PBS, concentrated, and filtered for in vitro and in vivo experiments.

Blocking Assays with Mxra8-Fc, MXRA8-2-Fc or Anti-Mxra8 mAbs.

Twenty-five thousand 3T3 or MRC-5 cells were seeded into 96-well plates 12 h before treatment. CHIKV-181/25 virions were purified through a 25% glycerol cushion at 25,000 r.p.m. for 2 h. Serially diluted Mxra8-Fc or MXRA8-2-Fc protein was incubated with purified virions (MOI of 3) for 1 h at 37° C. in a volume of 100 μl. The mixture was added to 3T3 or MRC-5 cells for 9.5 h or 11.5 h, respectively. Cells were then collected for intracellular E2 expression as measured by flow cytometry. For hamster mAb blocking experiments, 3T3 or MRC-5 cells were pre-incubated with serially diluted mAbs for 1 h at 37° C. in a volume of 50 μl, and then purified virions (MOI of 3) in 50 μl were added and incubated for 9.5 or 11.5 h, respectively. Cells were collected and intracellular E2 expression was analyzed by flow cytometry. For hamster mAb blocking experiments on primary human cells (keratinocytes, dermal fibroblasts, synovial fibroblasts, osteoblasts, chondrocytes, and skeletal muscle cells), 2×10⁴ cells were seeded into 96-well plates 12 h before treatment. Cells were pre-incubated with Armenian hamster isotype control (Bio X Cell #6E0260), 1H1.F5 or 9G2.D6 mAb for 1 h at 37° C. in a volume of 50 μl (50 μg/ml) and, subsequently, purified CHIKV-AF15561 virions (MOI of 15) in 50 μl were added and incubated for 10.5 h. Cells were collected and intracellular E2 expression was analyzed by flow cytometry.

Phosphatidylinositol-Specific Phospholipase C Treatment.

3T3 cells (10⁵) expressing GPI-anchored Mxra8 were collected using TrypLE and washed twice with PBS. Cells were then treated with 1 U/ml of phosphatidylinositol-specific phospholipase C (PI-PLC) (Sigma-Aldrich #P8804) in 50 μl PBS at 37° C. for 1 h. After two more cycles of washing, cells were stained for Mxra8 expression and processed by flow cytometry analysis as described above.

Mouse Experiments.

Experiments were carried out in accordance with the recommendations in the Guide for the Care and Use of Laboratory Animals of the National Institutes of Health after approval by the Institutional Animal Care and Use Committee at the Washington University School of Medicine (Assurance number A3381-01). Mxra8-Fc or an IgG control (JEV-13 mAb) (250 μg per mouse in PBS) was administered via an intraperitoneal route to four week-old wild-type male C57BL/6J mice 6 h before subcutaneous inoculation in the footpad with 10³ FFU of CHIKV-AF15561. Alternatively, in co-injection experiments, CH IKV or ONNV was mixed directly with Mxra8-Fc or JEV-13 (25 μg per mouse in PBS), and incubated at 37° C. for 30 min before inoculation. At 12 h, 24 h and 72 h post-infection, animals were euthanized, and after perfusion with PBS, indicated tissues were collected. For antibody pre- or post-treatment experiments, 300 μg of purified hamster mAbs 1G11.E6+7F1. D8 or 4E7.D10+8F7.E1, and isotype control PIP (Bio X cell #6E0260) in PBS were administered via an intraperitoneal route to four week-old wild-type male C57BL/6 mice 12 h prior to, or 8 or 24 h after, subcutaneous inoculation in the footpad with 10³ FFU of CHIKV-AF15561. Virus was titrated by focus forming assay as described 5 using mouse CHK-11⁵ for CHIKV or mouse CHK-48 for ONNV as the detection antibody. Joint swelling was monitored at 72 h post-infection via left foot measurements (width×height) using digital calipers as previously described³⁸. Samples sizes were estimated to determine a difference of three- to fivefold, depending on data distribution. Blinding and randomization were not performed.

Statistical Analysis.

Statistical significance was assigned when P values were <0.05 using Prism Version 7 (GraphPad). Cell culture experiments were analyzed by multiple t-tests with a Holm-Sidak correction or ANOVA with a multiple comparison correction. Analysis of levels of joint swelling or viral burden in vivo was determined by a Mann-Whitney, ANOVA, Kruskal-Wallis or unpaired t-test depending on data distribution and the number of comparison groups.

### REFERENCES

### Example 2: Using Mxra8 to Assess Alphavirus Vaccine or Diagnostic Antigen Integrity

The following example shows that Mxra8 can be used to assess an alphavirus vaccine or a diagnostic antigen integrity.

Because Mxra8 is a receptor for multiple arthritogenic alphaviruses, it can be used to physically assess the integrity of alphavirus vaccine or diagnostic antigens, which is important to define lot-to-lot integrity or variation in the context of commercial production. Alphavirus vaccine or diagnostic antigens can include, but are not limited to, live-attenuated viruses, virus-like particles, viral structural proteins, or nucleic acids or vectors producing these viral proteins or particles.

The format of the integrity assay can include capturing Mxra8 or Mxra8 fusion proteins (e.g., Mxra8-Fc) on a substrate (e.g., microtiter well plate, chip, or pin) and then incubation of the alphavirus vaccine or diagnostic antigen and detection with a monoclonal or polyclonal anti-vaccine/diagnostic antigen antibody or even Mxra8-Fc itself. Alternatively, the alphavirus vaccine or diagnostic antigen can be captured in the solid phase (directly or via a bridging antibody) and then incubated with Mxra8 or Mxra8-Fc fusion proteins for binding. The assay could be performed by ELISA, biolayer interferometry, surface plasmon resonance, or any other detection based system for binding.

### Example 3: A 15-Amino Acid Insertion in Domain 1 of Mxra8 Abolishes Infection by Alphaviruses

The following example describes a small insertion in Mxra8 renders it resistant to infection by alphaviruses. Thus, it is possible to make Mxra8 resistant to infection.

CRISPR-Cas9 based gene editing of Mxra8 resulted in markedly diminished alphavirus infection in vitro and in vivo, and viral infectivity was restored after trans-complementation with either mouse Mxra8 or human MXRA8. Because some alphaviruses can infect a range of vertebrate hosts in epizootic cycles, whether Mxra8 orthologs from different mammalian species could support infection by arthritogenic alphaviruses was explored. 3T3 cells lacking Mxra8 expression (ΔMxra8) were trans-complemented via lentivirus transduction with Mxra8 genes from mouse (Mus musculus, positive control), rat (Rattus norvegicusdog (Canis lupus familiaris), cow (Bos taurus), goat (Capra hircus), sheep (Ovis aries), horse (Equus caballus), chimpanzee (Pan troglodytes). Surface expression of the different Mxra8 orthologs was confirmed with an oligoclonal pool of species cross-reactive monoclonal antibodies (mAbs) against Mxra8 (see e.g., FIG. 15A). Trans-complemented cells were inoculated with CHIKV and evaluated for infection 9.5 h later by assessing intracellular viral E2 protein expression by flow cytometry. Consistent with published results, CHIKV E2 antigen was absent in ΔMxra8 cells but present at high levels in ΔMxra8 cells trans-complemented with mouse Mxra8 (see e.g., FIG. 15B). Analogously, trans-complementation of ΔMxra8 cells with rat, dog, goat, sheep, horse, and chimpanzee Mxra8 orthologs restored CHIKV infectivity. Notably, Mxra8 from cow (Bos taurus) failed to restore infectivity even though it was expressed on the cell surface. Similar results were observed with related arthritogenic alphaviruses including MAYV, RRV, and ONNV.

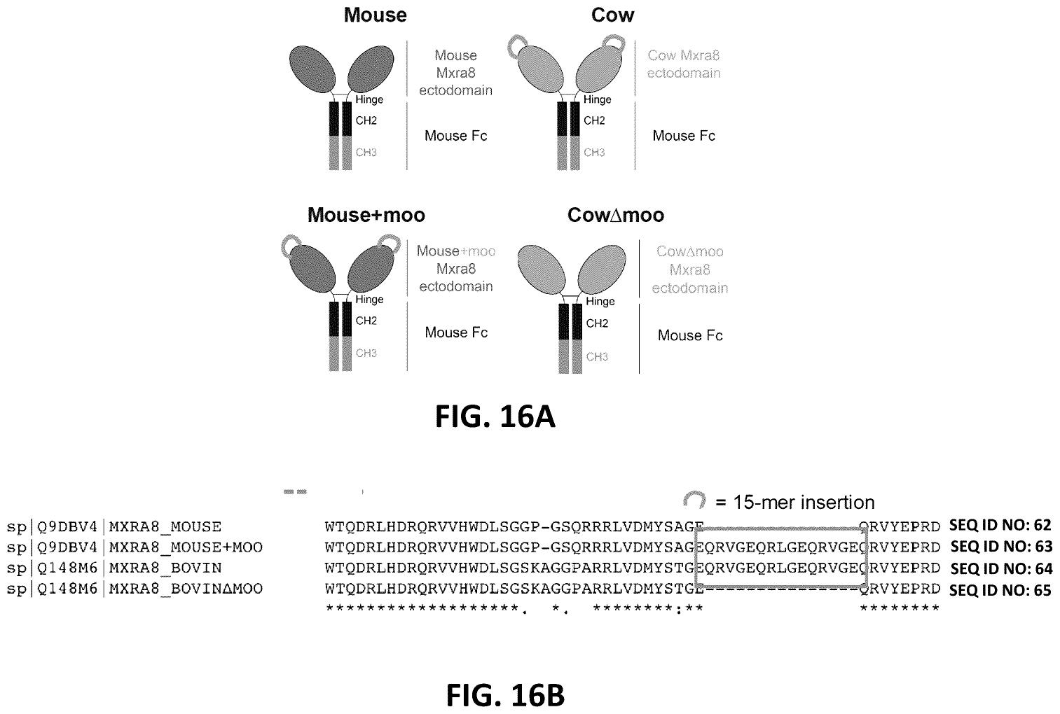

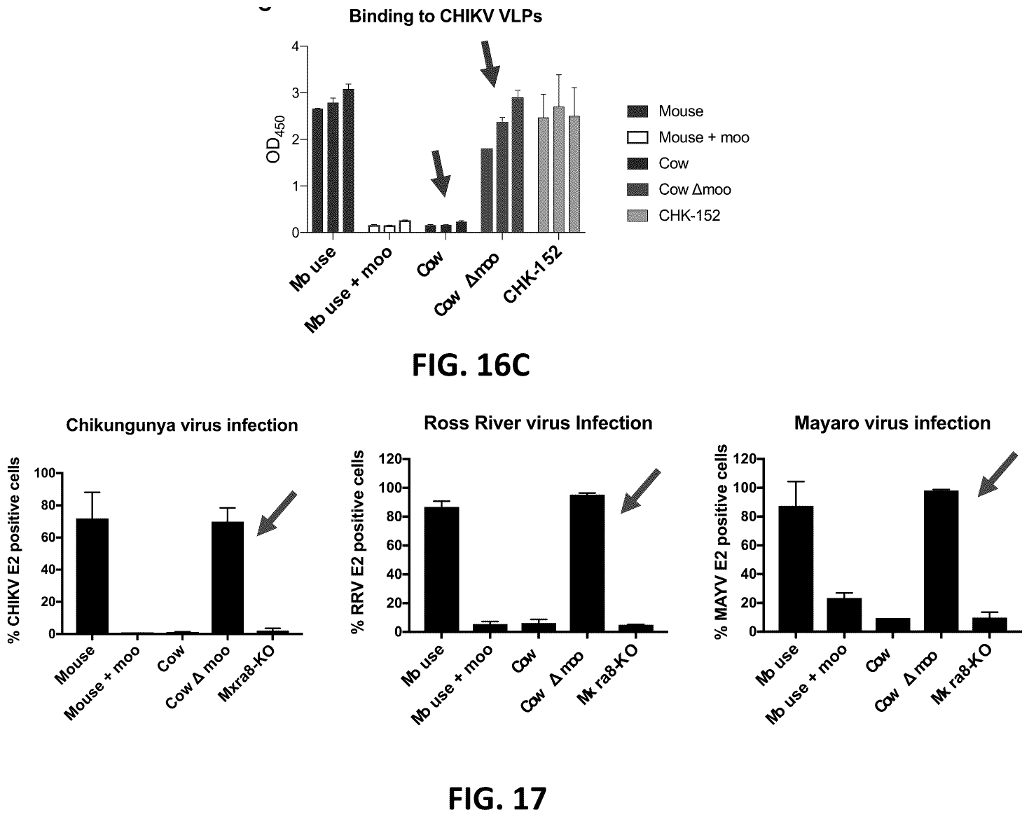

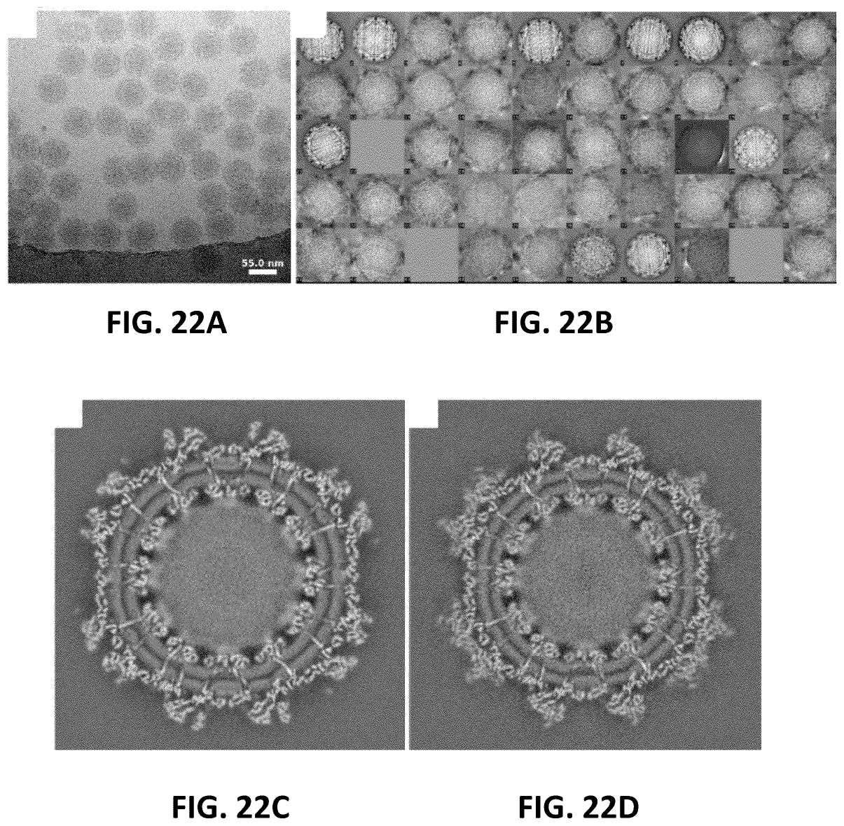

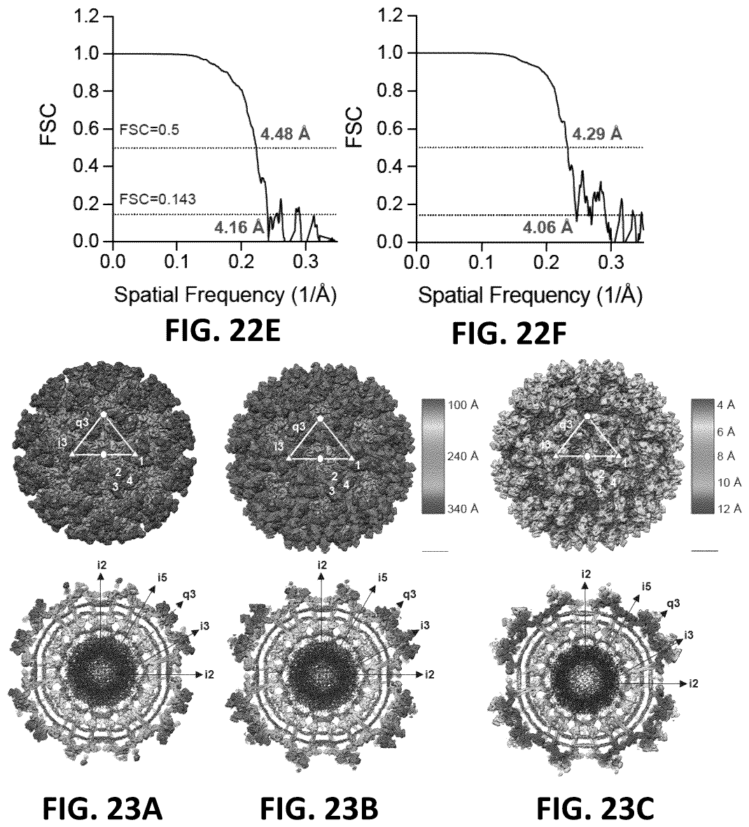

To begin to investigate why cow Mxra8 in particular failed to restore alphavirus infectivity, its protein sequence was aligned with other Mxra8 gene sequences (see e.g., FIG. 22A-FIG. 22F and FIG. 16B). Remarkably, cow Mxra8, but not any of the other species tested, contained a unique 15-amino acid insertion (⁷⁸GEQRLGEQRVGEQRV, SEQ ID NO: 91⁹²) composed of three quasi-identical (GEQRL/V, SEQ ID NO: 92) 5-residue repeats that likely were derived from the immediate upstream GEQRV gene sequence; the 45 nucleotides of the insertion correspond highly to upstream sequence. To gain insight into the significance of the 15-amino acid insertion, comparative structural analysis was performed. The 15-amino acid insertion of cow Mxra8 was located as a disordered extension of the C′-C″ loop in Domain 1.

The structural analysis suggested that the 15-amino acid insertion in Mxra8 of cows might disrupt interactions with the CHIKV virion. To begin to evaluate this hypothesis, mouse Mxra8-Fc fusion proteins were generated that lacked or contained the additional residues from cow (mouse Mxra8 and mouse Mxra8+moo) and reciprocally cow Mxra8-Fc fusion protein that lacked or contained the 15 residues (cow Mxra8 and cow Mxra8 Δmoo) (see e.g., FIG. 16A). The wild-type and chimeric Mxra8 proteins were tested for their ability to bind CHIKV VLPs in a capture ELISA. Mouse Mxra8 and cow Mxra8 Δmoo bound avidly in a dose-dependent manner to CHIKV VLPs whereas mouse Mxra8+moo and cow Mxra8 did not (see e.g., FIG. 16C). These data suggest strongly that the 15-amino acid insertion in Mxra8 directly inhibits binding to CHIKV particles.

To further evaluate the significance of the 15-amino acid insertion to alphavirus infectivity, ΔMxra8 3T3 cells were transduced with lentiviruses encoding mouse Mxra8, mouse Mxra8+moo, cow Mxra8, and cow Mxra8 Δmoo. The addition of the 15 residues to mouse Mxra8 abolished infectivity by CHIKV, MAYV, and RRV, whereas deletion of the residues from cow Mxra8 facilitated infection by these viruses (see e.g., FIG. 17). Thus, a 15-amino acid insertion in Domain 1 of Mxra8 can determine the infectivity of multiple alphaviruses.

### Example 4: Mxra8 Knockout Demonstrates Diminished Alpha Virus Infection and Swelling

The following example describes the generation of Mxra8 knockout mice and reduction of alphavirus infection in the KO mice.

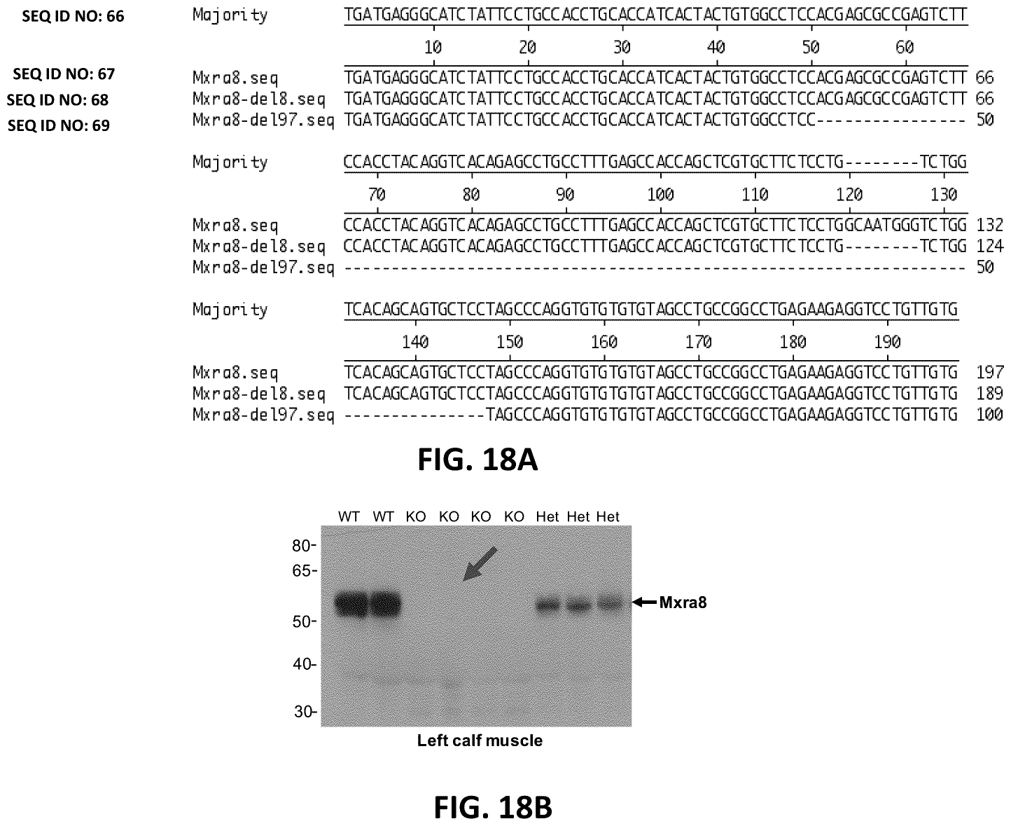

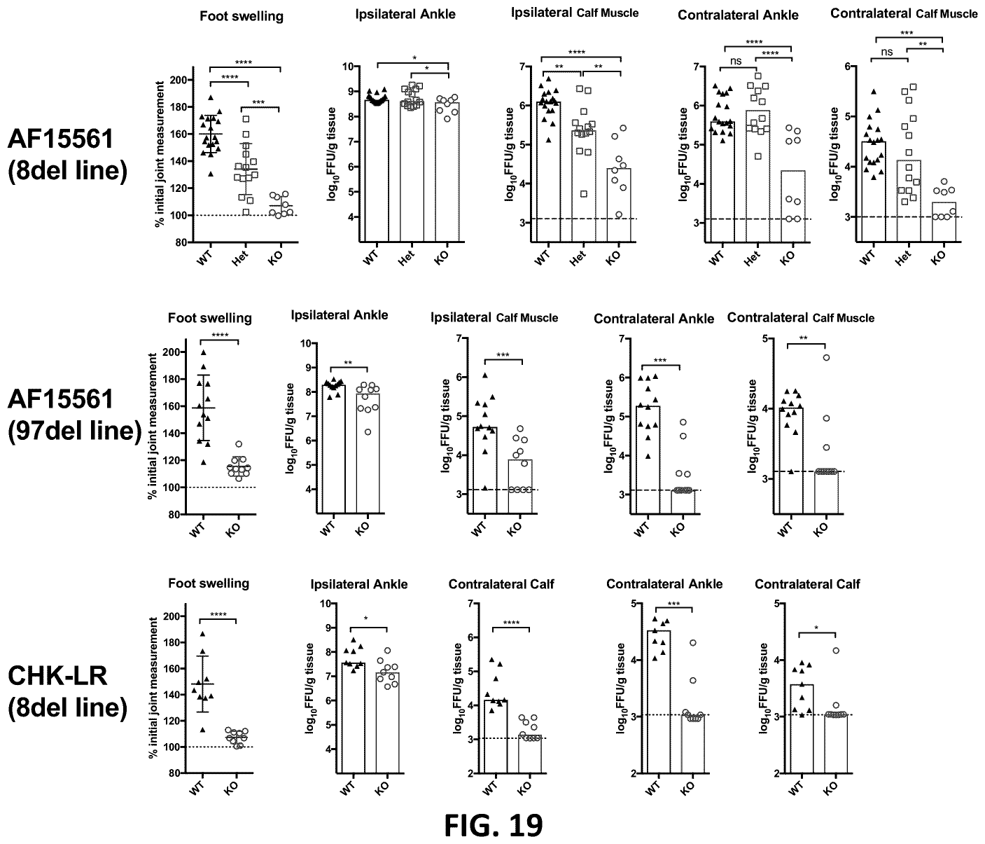

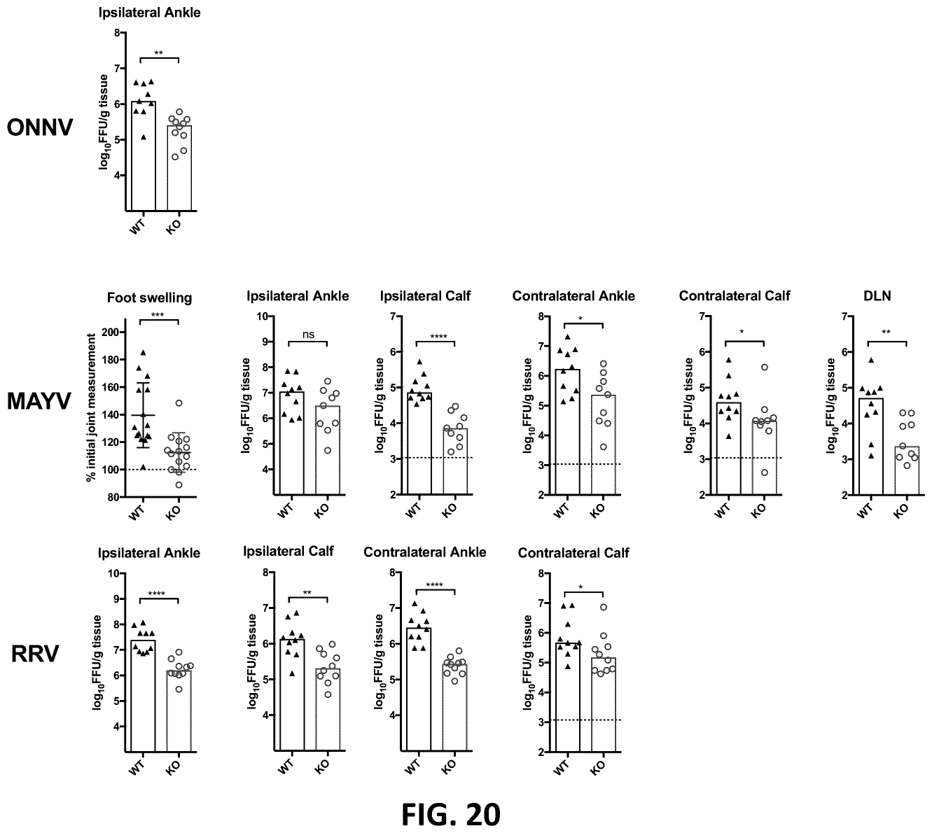

Generation of Mxra8^{mut/mut} mice by CRISPR-Cas9 gene targeting has been demonstrated (see e.g., FIG. 18A-FIG. 18B). It was shown that CHIKV infection and swelling was diminished in Mxra8-deficient mice during the acute phase (see e.g., FIG. 19). Furthermore, it was demonstrated that infection of other arthritogenic alphaviruses (ONNV, MAYV, and RRV) was also diminished in Mxra8 Å 808 mice (see e.g., FIG. 20).

### Example 5: Cryo-Em Structure of Chikungunya Virus in Complex with the Mxra8 Receptor

Mxra8 is a receptor for multiple arthritogenic alphaviruses that cause debilitating acute and chronic musculoskeletal disease in humans. Herein is described a 2 Å X-ray crystallographic structure of Mxra8 and 4 to 5 Å resolution cryo-electron microscopy reconstructions of Mxra8 bound to chikungunya (CHIKV) virus-like particles (VLPs) and infectious virus. Mxra8 wedges into a cleft created by adjacent CHIKV E2 proteins in one trimeric spike and engages E1 protein on an adjacent spike. Two binding modes are observed with the fully mature VLP, with one of the Mxra8 binding sites having unique E1 contacts. Only this high-affinity binding mode was observed in the complex with infectious CHIKV, as viral maturation and E3 occupancy appear to influence receptor binding site usage. Our studies provide structural insight into how Mxra8 binds CHIKV and creates a path for developing alphavirus entry inhibitors.

### INTRODUCTION

Alphaviruses are positive-sense, single-stranded RNA, enveloped viruses and are among the most important arthropod-borne viruses causing disease in humans (Powers et al., 2001). This genus includes chikungunya (CHIKV), Mayaro (MAW), O'nyong'nyong (ONNV), and Ross River (RRV) viruses, which are emerging beyond their historical boundaries and now cause debilitating acute and chronic polyarthritis affecting millions of people in Africa, Asia, Europe, and the Americas. Despite their epidemic potential, there are no specific therapies or licensed vaccines for any alphavirus infection.

Alphavirus genomes encode four non-structural and five structural proteins. The non-structural proteins are required for virus replication, protein modification, and immune evasion. The structural proteins (capsid (C)-envelope (E)3-E2-6K-E1) are synthesized from a subgenomic promoter and cleaved co- and post-translationally. The E1 envelope glycoprotein participates in cell fusion (Lescar et al., 2001), whereas the E2 envelope glycoprotein binds to entry factors (Smith et al., 1995; Zhang et al., 2005) and initiates clathrin-dependent endocytosis (DeTulleo and Kirchhausen, 1998; Lee et al., 2013; Ooi et al., 2013). The E3 protein is essential for the proper folding of p62 (precursor to E2) and the formation of the p62-E1 heterodimer (Carleton et al., 1997; Mulvey and Brown, 1995) but is cleaved by furin-like proteases during the maturation process in the trans-Golgi network (Heidner et al., 1996). Mature alphaviruses are ˜700 Å icosahedral particles that assemble at the plasma membrane and contain a lipid bilayer with 240 embedded E2-E1 heterodimers assembled into 80 trimeric spikes with T=4 icosahedral symmetry (Cheng et al., 1995; Kostyuchenko et al., 2011; Paredes et al., 1993), and a nucleocapsid containing a single copy of genomic RNA.

Crystallographic studies of the precursor p62-E1, the mature E2-E1 glycoprotein complex, and the E1 protein (Lescar et al., 2001; Li et al., 2010; Roussel et al., 2006; Voss et al., 2010) have elucidated the glycoprotein structures. Several alphavirus virions structures also have been elucidated by cryo-electron microscopy (cryo-EM) (Cheng et al., 1995; Kostyuchenko et al., 2011; Lee et al., 1998; Li et al., 2010; Pletnev et al., 2001; Zhang et al., 2011; Zhang et al., 2002). The E1 ectodomain consists of three β-barrel domains termed Domain I (DI), DII, and DIII. The fusion peptide is located at the distal end of DII. E1 monomers lie at the base of the surface spikes and form trimers surrounding the icosahedral axes. E2 localizes to a long, thin, leaf-like structure on the top of the spike. The mature E2 protein contains three domains with immunoglobulin (Ig)-like folds: the N-terminal domain A, located at the center; domain B at the lateral lip; and the C-terminal domain C, located close to E1 and the viral membrane.

A genome-wide CRISPR/Cas9-based screen was recently used to identify the two immunoglobulin (Ig)-like domain containing cell adhesion molecule, Mxra8 as a receptor for multiple arthritogenic alphaviruses including CHIKV, RRV, MAYV, and ONNV (Zhang et al., 2018). Importantly, the human gene ortholog, MXRA8, also bound to CHIKV and other alphaviruses, and its cell surface expression facilitated infection of primary human target cells including fibroblasts, skeletal muscle cells, and chondrocytes. Mxra8 bound directly to CHIKV particles and enhanced attachment and internalization into cells, and Mxra8-Fc fusion protein or anti-Mxra8 monoclonal antibodies (mAbs) blocked CHIKV infection of several cell types. Administration of Mxr8a-Fc protein or anti-Mxra8 blocking mAbs to mice reduced CHIKV or ONNV infection and associated joint swelling. Despite defining several biological characteristics of CHIKV interaction with this receptor, structural insight as to how Mxra8 engages the spike proteins on the virion is lacking.

Herein is described the X-ray crystallographic structure of Mxra8 and cryo-EM reconstructions of CHIKV virus-like particles (VLPs) (produced as capsid-E3-E2-6K-E1 but lacking viral RNA) and fully infectious CHIKV in complex with Mxra8. These are the first structures of alphavirus virions with their cognate host cell binding partner. Mxra8 has an unusual architecture, as its two Ig-like domains are oriented in a disulfide-linked head-to-head arrangement, with domain 2 emanating from the loops of domain 1 and the N- and C-terminal residues proximal to each other. Mxra8 binds into a cleft formed between two E2-E1 heterodimers within a trimeric spike, making dominant contacts with E2 domain A residues. Mxra8 also engages a determinant in E1 domain II on an adjacent glycoprotein spike. Two binding modes for Mxra8 were observed with mature CHIKV VLPs, which was consistent with a high and low affinity binding site model supported by surface plasmon resonance measurements. The low affinity binding sites, however, were sterically obscured by the retention of E3 on partially mature infectious CHIKV. Overall, this structural analysis defines how CHIKV engages its receptor Mxra8 to facilitate attachment and infection of cells. This information may inform the basis of therapies and improved vaccine designs that mitigate disease of multiple emerging alphaviruses.

Results

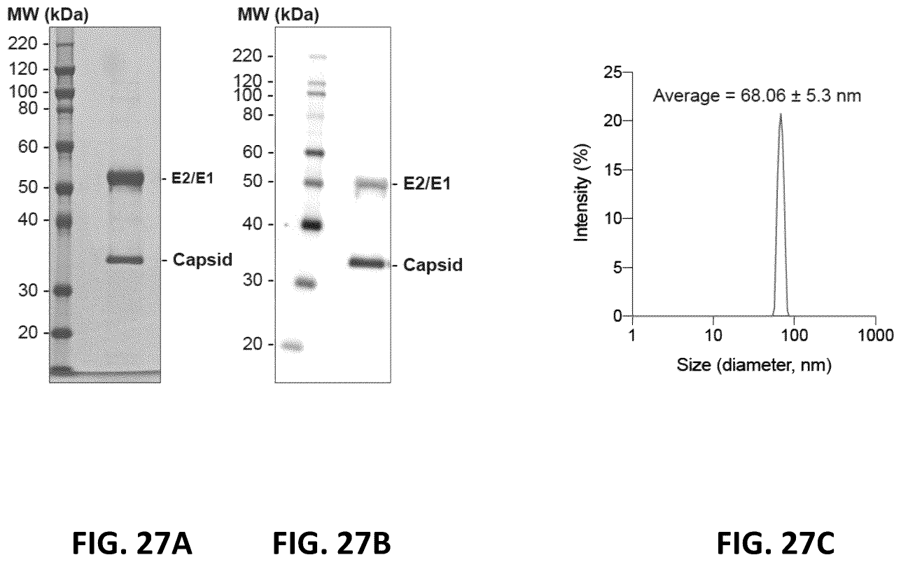

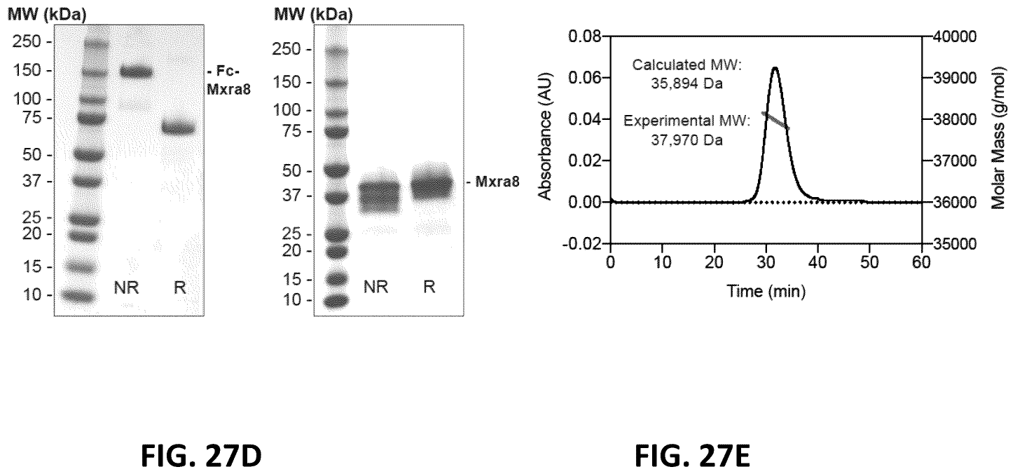

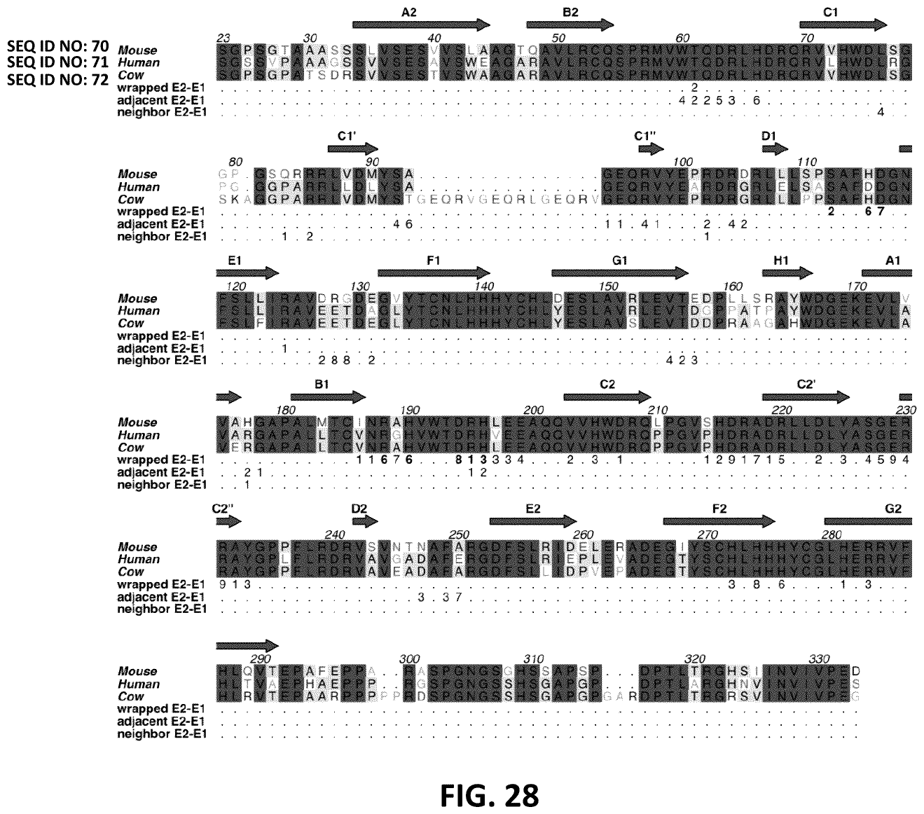

CHIKV VLPs and Mxra8 protein. Previous studies have obtained high-resolution structural information that elucidated how neutralizing antibodies engage CHIKV using VLPs, which contain the structural proteins but lack infectious viral RNA and can be imaged under lower biosafety conditions (Long et al., 2015; Sun et al., 2013). To begin to define the structural basis for interaction between Mxra8 and arthritogenic alphaviruses (Zhang et al., 2018), CHIKV VLPs (strain 37997) were produced after transient transfection of HEK-293 cells with a plasmid encoding C-E3-E2-6K-E1 (Akahata et al., 2010); equivalent preparation of VLPs were used in a phase 1 or are being used in phase 2 human clinical trials for vaccine protection against CHIKV disease ((Chang et al., 2014), NCT02562482 and NCT03483961). Soluble VLPs were collected, buffer exchanged, and monitored for purity by SDS-PAGE and antigenicity by Western blotting with anti-capsid and anti-E2/E1 antibodies (see e.g., FIG. 27A and FIG. 27B). Dynamic light scattering experiments revealed a particle size of ˜680 Å (see e.g., FIG. 27C), which is close to the expected external diameter of 700 Å for a CHIKV virion (Cheng et al., 1995). To produce soluble Mxra8, Mxra8-Fc fusion proteins were engineered with a human rhinovirus 3C (HRV) protease site between the C-terminus of Mxra8 and the Fc domain. After production of Mxra8-Fc fusion protein in HEK-293F cells and purification by protein A affinity chromatography, the Fc domain was cleaved with HRV, and isolated Mxra8 in the flow-through of a second round of protein A agarose affinity chromatography (see e.g., FIG. 27D). Size exclusion chromatography coupled with multi-angle light scattering analysis revealed that cleaved mammalian cell-generated Mxra8 was monomeric with a molecular weight of 38 kDa (see e.g., FIG. 27E).

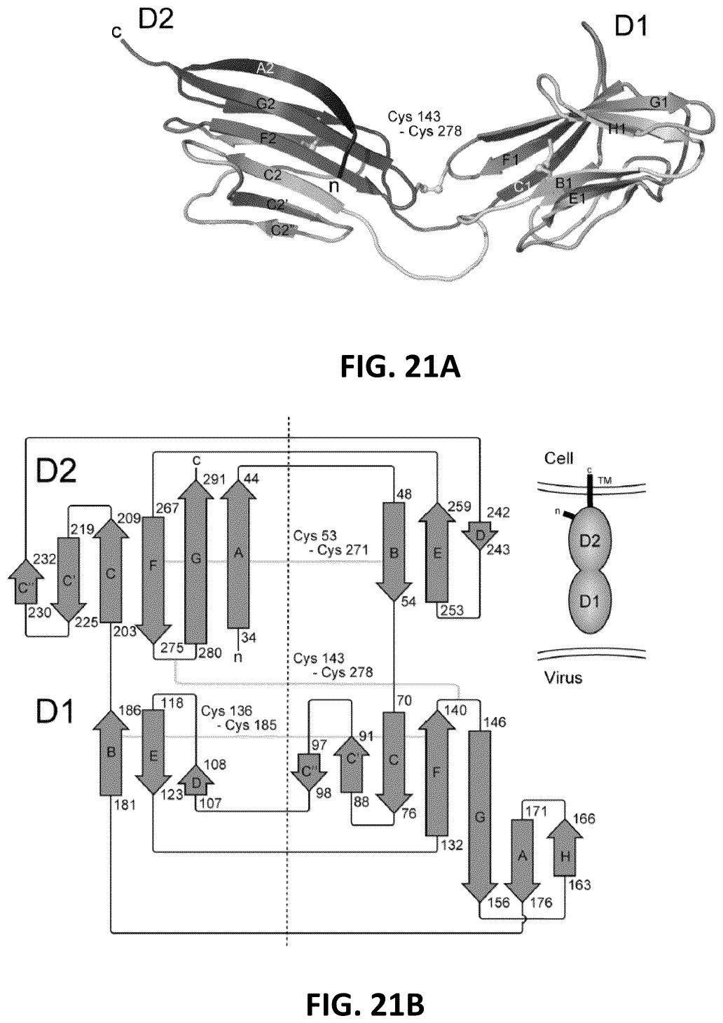

X-ray crystallographic structure of Mxra8. The ectodomain residues of Mxra8, corresponding to positions 23 through 295 of NCBI reference sequence: NP_077225, were crystallized and the structure solved by molecular replacement. The resulting protein consists of two Ig-like domains arranged in a head to head β-strand-swapped dimer that is connected by an interchain disulfide bond (see e.g., FIG. 21A-FIG. 21B).

Cryo-EM reconstruction of Mxra8 bound to CHIKV VLPs. Electron micrographs of CHIKV VLPs with or without bound Mxra8 were recorded using a 300 kV Titan Krios cryo-electron microscope with Gatan K2 detector at the Washington University Center for Cellular Imaging (WUCCI) (see e.g., FIG. 22A and TABLE 3). The images were corrected for beam-induced motion using MotionCor2 (Zheng et al., 2017). Particles were auto-picked from the micrographs and subjected to two-dimensional classification to remove ice contamination and debris (see e.g., FIG. 22B). The remaining particles underwent ab initio three-dimensional reconstruction and refinement in cisTEM (Grant et al., 2018). The reconstructions of CHIKV VLP alone and with Mxra8 were determined at overall resolutions of 4.16 Å and 4.06 Å (see e.g., FIGS. 22C and D), respectively, based on Fourier Shell Correlation (FCS) analysis (see e.g., FIG. 22E and FIG. 22F).

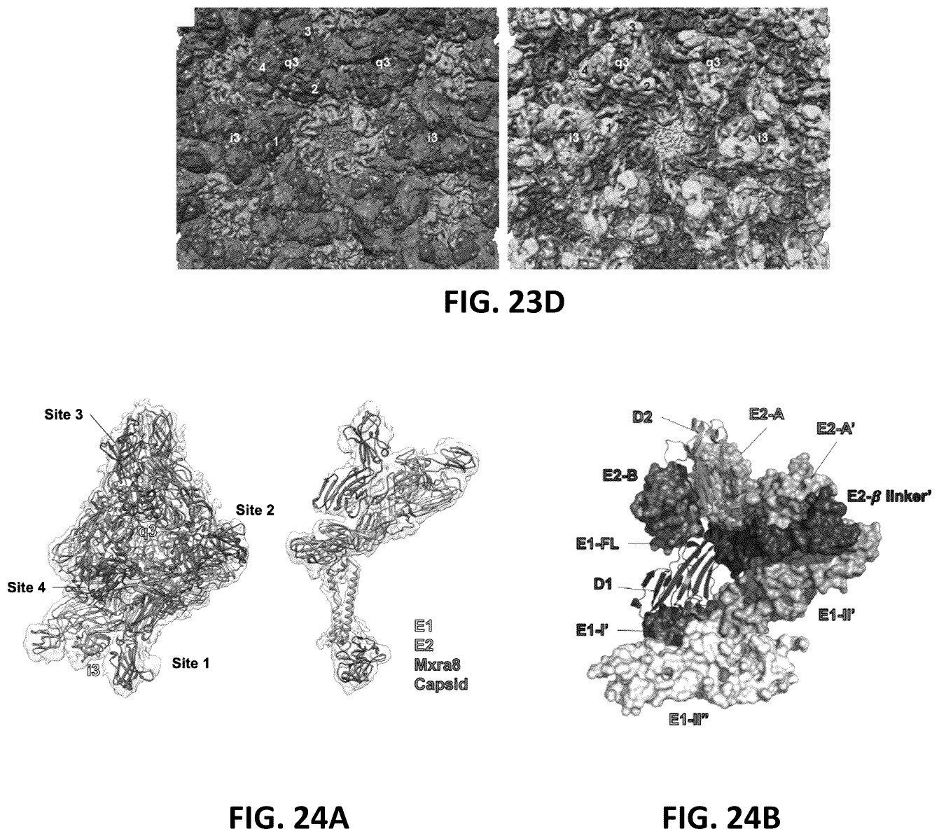

The three-dimensional reconstructions showed no large conformational changes in the structure of the CHIKV VLP when Mxra8 is bound (see e.g., FIG. 23A and FIG. 23B). Despite the different chemical environments of the structural glycoproteins within the asymmetric unit of the particle, Mxra8 bound E2-E1 heterodimers in a 1:1 ratio, with 240 Mxra8 molecules per virion. The local resolution of the reconstruction of CHIKV VLP-Mxra8 complex revealed that the viral E2-E1 proteins were the best resolved (see e.g., FIG. 23C and FIG. 23D). The Mxra8 regions distal to the viral envelope were the least resolved, suggesting a greater degree of flexibility. The local resolution does not vary between the copies of Mxra8 and envelope proteins within the asymmetric unit.

Refinement of the model. An atomic model of the CHIKV VLP structural proteins and Mxra8 was iteratively built using a combination of published CHIKV crystal structures and de novo modelling. As a starting point, the structures of the CHIKV capsid (PDB: 5H23), CHIKV p62-E1 (PDB: 3N42), Venezuelan equine encephalitis virus (VEEV) transmembrane domains of E1 and E2 (PDB: 3J0C), and the structure of Mxra8 (see e.g., FIG. 21A-FIG. 21B) were used to build one subunit. One asymmetric unit surrounded by the adjacent subunits in neighboring asymmetric units was then built. This process was essential to ensure accurate modeling of all interaction interfaces and prevent steric clashes. This model underwent manual and computational real-space refinement using the 4.06 Å cryo-EM map of CHIKV VLP bound to Mxra8 and employing programs COOT (Emsley et al., 2010) and PHENIX.REFINE (Afonine et al., 2012) (see e.g., FIG. 24A and FIG. 24B, TABLE 4).

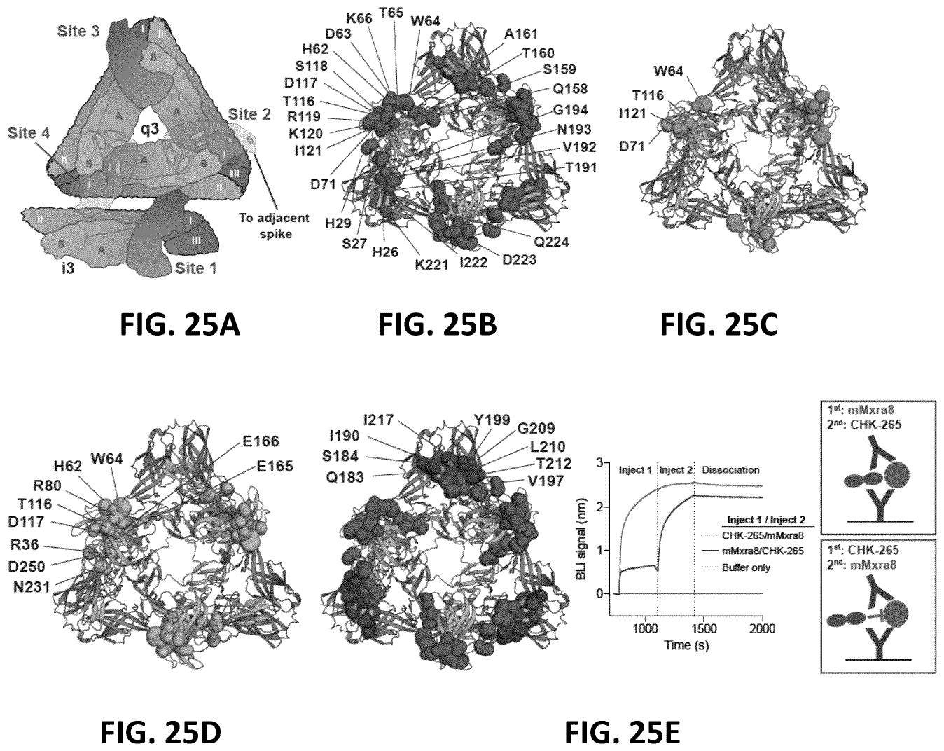

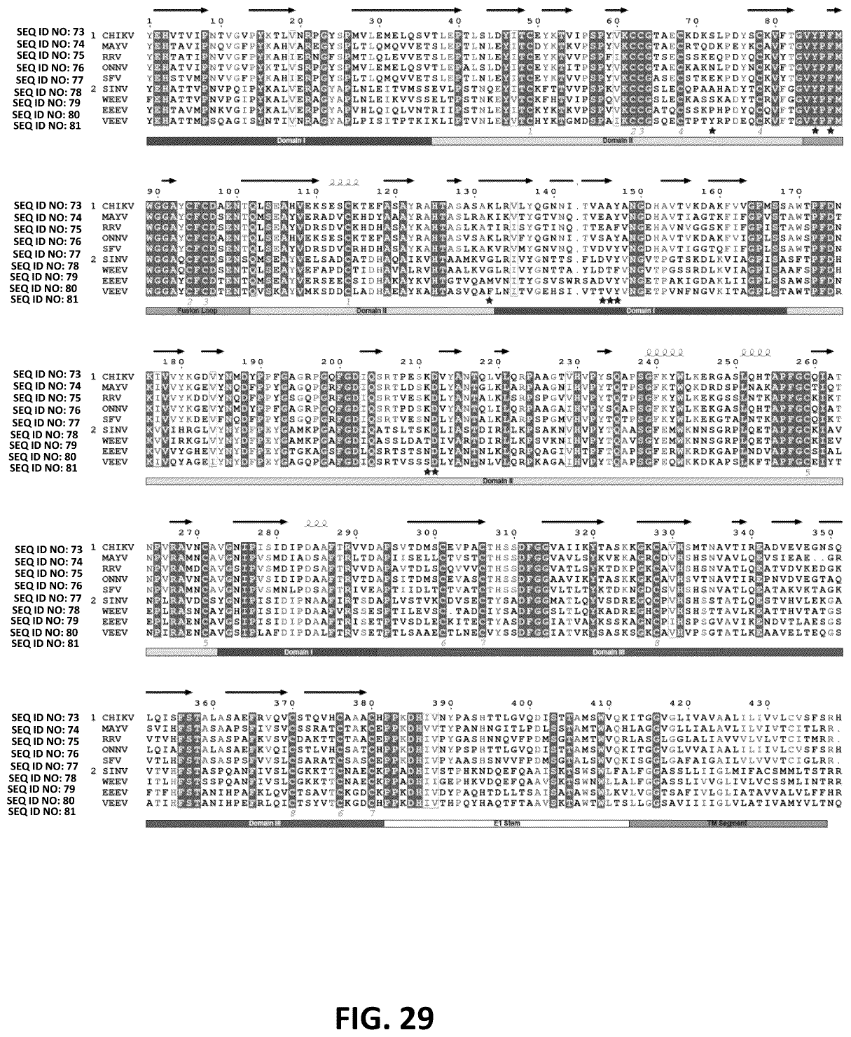

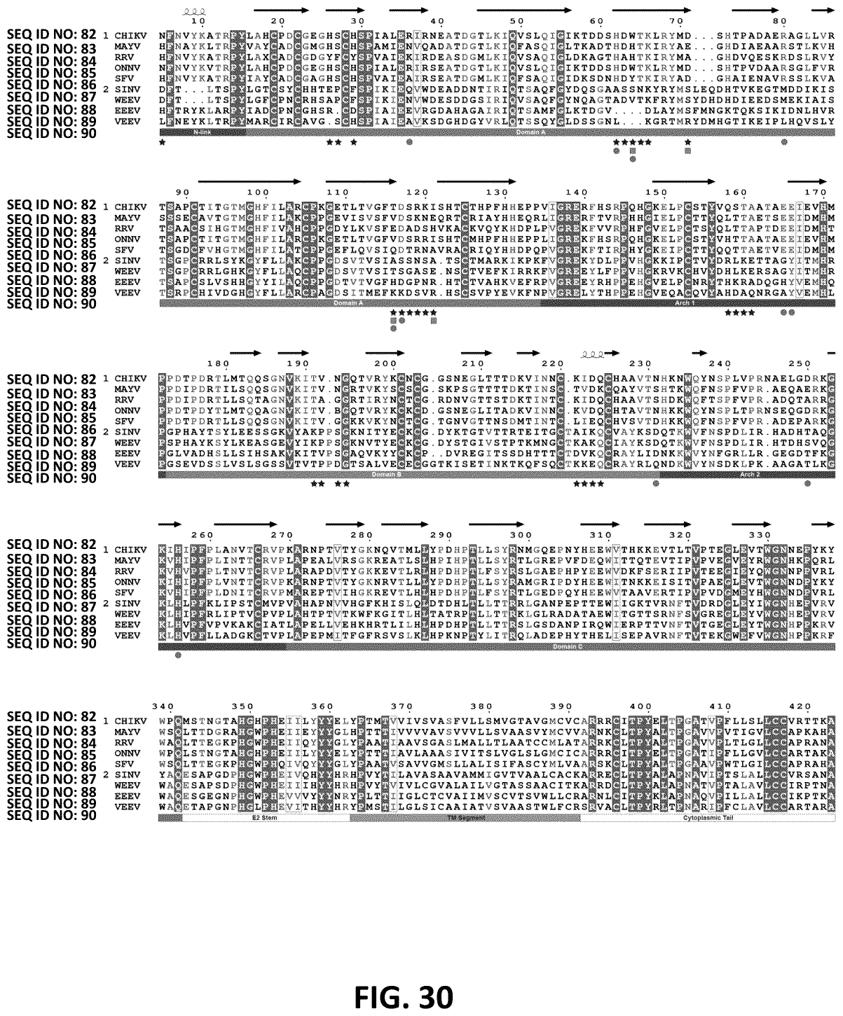

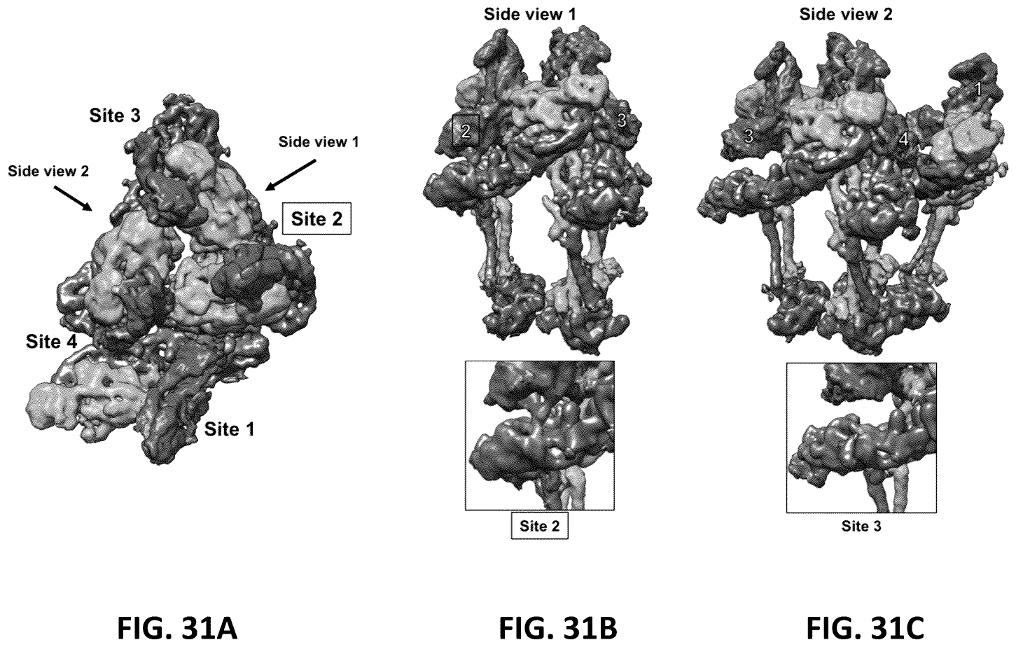

Our model details the residues of the CHIKV E2-E1 heterodimers at the Mxra8 binding interface. At all four sites of the icosahedral asymmetric unit, Mxra8 engages the A and B domains of the E2 protein (residues 26-29, 116-121, 191-194, 221-224), as well as the A domain and β-linker of the counter-clockwise, adjacent E2 neighbor within the same trimeric spike (residues 5, 62-66, 158-161). Mxra8 is also positioned near the fusion loop of E1 (residues 85 and 87) as well as E1 domain II of an adjacent spike (residues 72, 211, 212) (see e.g., FIG. 25A and FIG. 25B, FIG. 29 and FIG. 30, TABLE 5). Mxra8 binding at site 2 is associated with unique E1 contacts (residues 132 and 145-147) in the primary engaged spike; these interactions were not observed at sites 1, 3, and 4 (see e.g., FIG. 29 and FIG. 31A-FIG. 31C).

Many of the structurally identified contact residues are coincident with previous alanine scanning mutagenesis mapping data, which identified W64, D71, T116, and I121 in the A domain and Y199 and 1217 in the B domain of E2 as important for optimal Mxra8 binding (Zhang et al., 2018) (see e.g., FIG. 25C). Furthermore, several neutralizing human mAbs against CHIKV that disrupt Mxra8 binding (Zhang et al. 2018) map to epitopes in the A and B domains of E2 (Long et al., 2015; Smith et al., 2015) that are shared with the Mxra8 binding site (see e.g., FIG. 25D).

This finding prompted us to evaluate the effect on CHIKV-Mxra8 interactions of CHK-265, a murine mAb that cross-neutralizes infection of CHIKV, MAYV, RRV, and ONNV (Fox et al., 2015), all of which can use Mxra8 as an entry receptor (Zhang et al., 2018). CHK-265 cross-links trimeric spikes via bridging residues in the A and B domains of adjacent E2 proteins (Fox et al., 2015). Although the epitope of CHK-265 does not overlap directly the Mxra8 binding site, it was hypothesized that it might still hinder interaction since the Mxra8 binding groove lies beneath the antibody epitope. Order-of-addition binding experiments showed that CHK-265 can bind CHIKV VLPs when Mxra8 is pre-bound, but that Mxra8 cannot bind when CHK-265 is bound (see e.g., FIG. 25E).

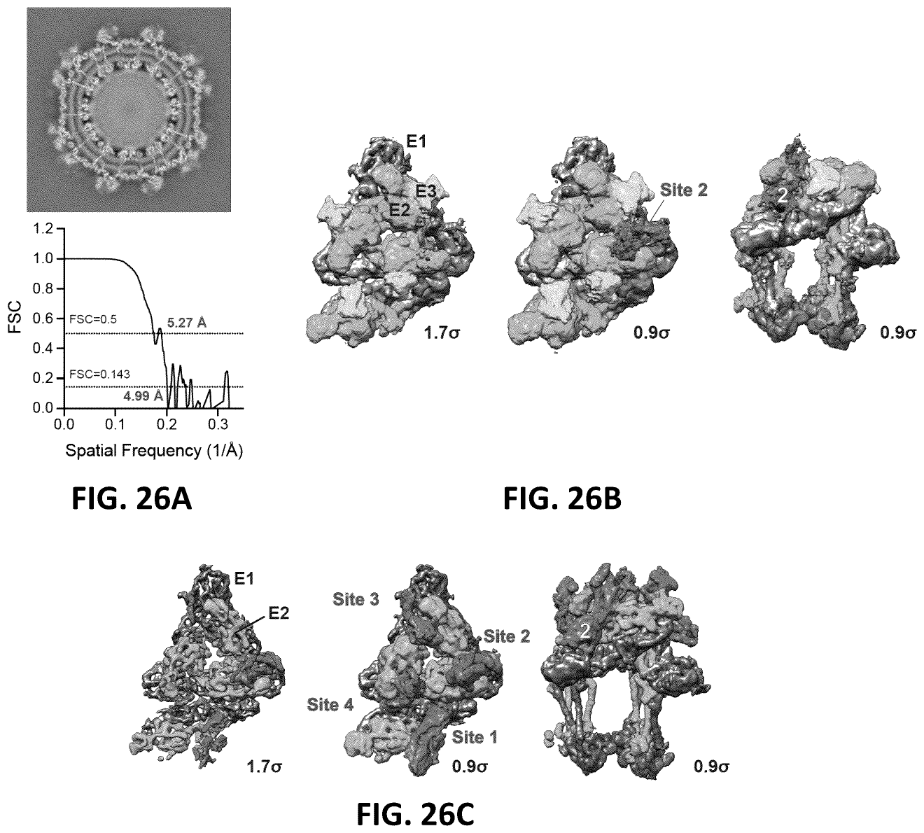

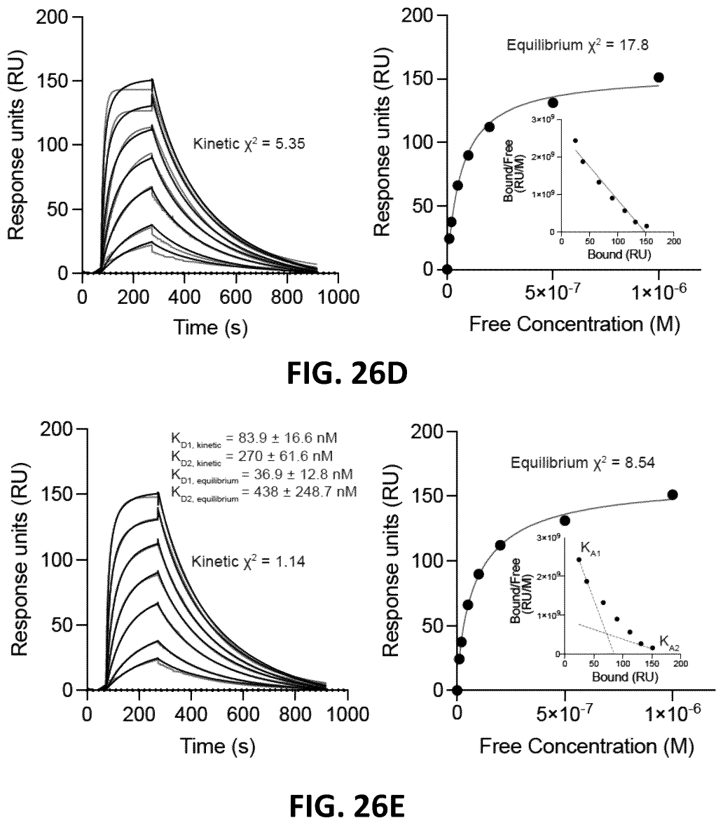

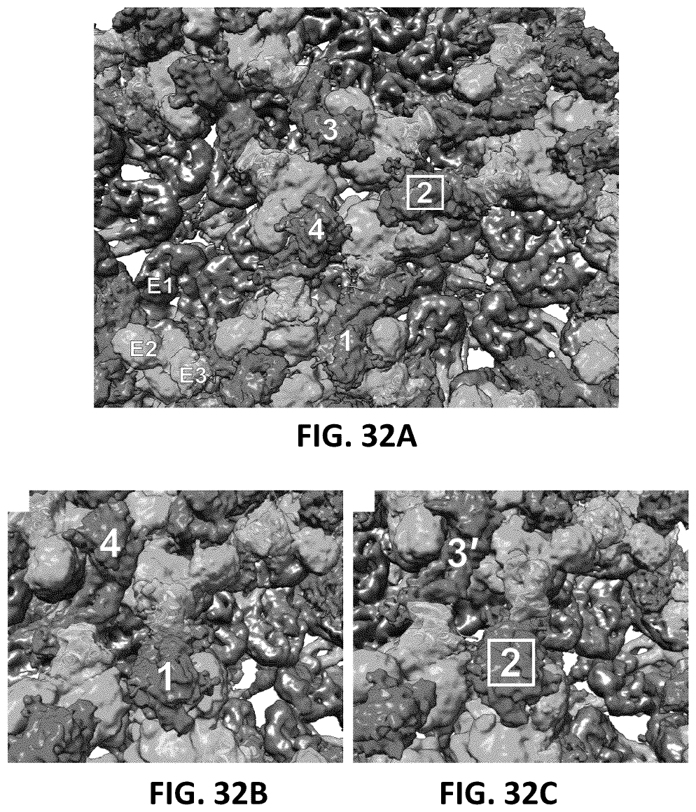

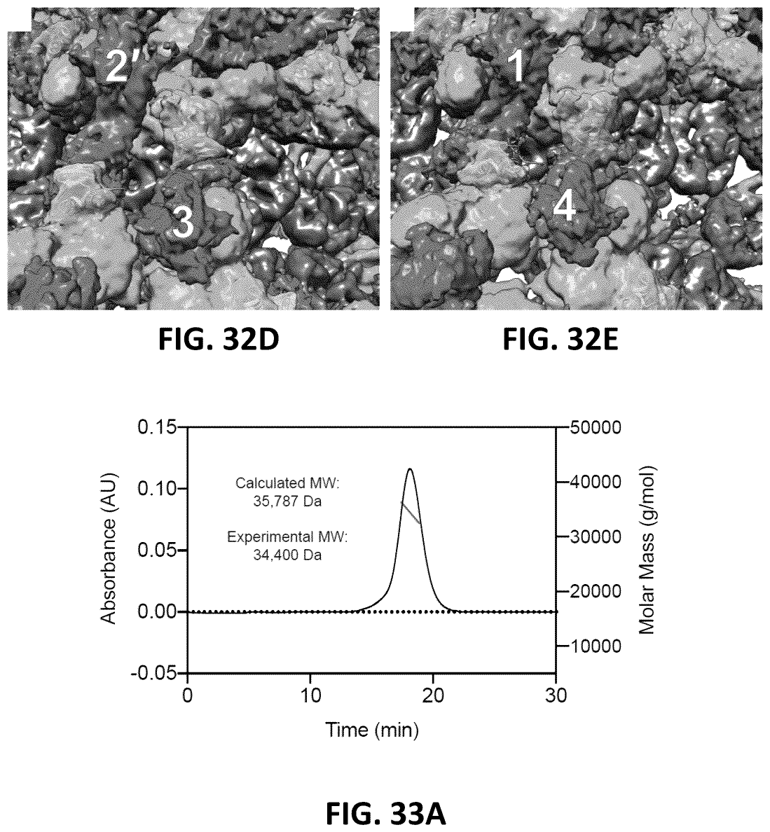

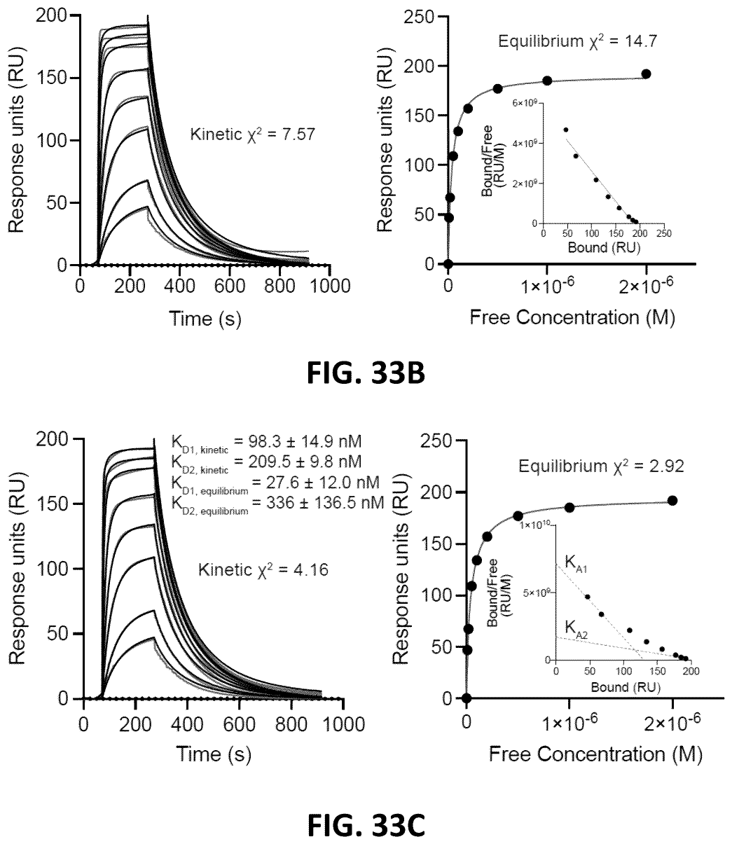

Modes of Mxra8 binding to CHIKV VLPs and infectious virus. A 4.99 Å cryo-EM reconstruction of CHIKV infectious particles with Mxra8 was also generated and an atomic model built (see e.g., FIG. 26A, TABLE 4). In contrast to the CHIKV VLP, strong electron density was seen for the E3 protein in all four chemical environments of the infectious CHIKV (see e.g., FIG. 26B-FIG. 26C), which similarly was observed in other infectious and non-infections alphavirus particle preparations (Yap et al., 2017; Zhang et al., 2011). In the CHIKV infectious particles, weaker electron density was seen for Mxra8 compared to the CHIKV VLP reconstruction. Only one of the four potential binding sites in the asymmetric unit showed interpretable Mxra8 density (see e.g., FIG. 26B). This suggested there might be a lower occupancy of Mxra8 binding on the infectious particle compared to the fully mature VLP, potentially due to steric hindrance by the residual E3 protein in three of four chemical environments. To test this hypothesis, the difference map of Mxra8 from the mature Mxra8-VLP complex was docked onto the Mxra8-bound CHIKV infectious virus reconstruction and assessed for clashes. E3 appears to sterically obstruct Mxra8 binding at the i3 (site 1) and two of three of the q3 binding sites (sites 3 and 4), allowing for 60 binding sites per virion at site 2 (see e.g., FIG. 32A-FIG. 32E). Electron density for Mxra8 on the CHIKV VLP is strongest at site 2, which corresponds to the Mxra8 binding site on the infectious virion (see e.g., FIG. 26C). This finding is consistent with our binding analysis of Mxra8 to CHIKV VLPs by surface plasmon resonance, where the raw traces did not fit well to kinetic and equilibrium 1:1 binding models (see e.g., FIG. 26D). One explanation for the higher occupancy of Mxra8 at one site in an asymmetric unit is a site-specific higher binding affinity, due to the slightly different chemical environments of each of the four E2-E1 heterodimers. A model assuming one high affinity site and three lower but equal affinity sites was generated (see Methods); this model produced a substantially better fit to our binding data (see e.g., FIG. 26E). Mxra8 binds the high affinity site with a kinetically derived K_{D} of ˜84 nM and the lower affinity sites with a K_{D} of ˜270 nM. Similar SPR measurements and two-site binding analysis were observed for human monomeric MXRA8 (as determined by SEC-MALS), which is 78% identical to the murine protein (see e.g., FIG. 33A-FIG. 33C).

### DISCUSSION

Cell culture infection experiments with mouse and human cells and in vivo pathogenesis studies in mice defined Mxra8 as a cell surface receptor required for optimal infectivity and induction of musculoskeletal disease by multiple arthritogenic alphaviruses (Zhang et al., 2018). Here, our single particle cryo-EM analysis of CHIKV VLPs and infectious virus provides structural insight into how CHIKV engages Mxra8 to facilitate interactions with target cells. Our study adds to the limited structural knowledge of how receptors bind to enveloped icosahedral virions. Only one other structure of an enveloped, icosahedral virus complexed with its receptor exists. In a 25 Å resolution cryo-EM structure, Dengue virus (DENV) was complexed with the carbohydrate recognition domain of DC-SIGN; the only contacts were with protruding N-linked glycans in domain II of the E protein (Pokidysheva et al., 2006). In contrast, a complex network of quaternary protein-protein interactions involving Mxra8 engaging two E2-E1 heterodimers within one trimeric spike and E1 on an adjacent spike was observed. These sites are coincident with alanine scanning mutagenesis analysis of E2 protein interactions with Mxra8 and epitope maps of neutralizing human mAbs against CHIKV that directly block engagement of Mxra8. Our structures indicate that Mxra8 can bind at four distinct sites in the icosahedral asymmetric unit of the CHIKV VLP but only one site in the infectious virus, which retains E3.

The quaternary interactions formed between Mxra8 and multiple envelope proteins would effectively cross-link CHIKV spikes in a manner analogous to a previously defined broadly neutralizing mAb (CHK-265) that binds domain B on one trimer and domain A on an adjacent spike (Fox et al., 2015). The cross-linking of the viral structural proteins by Mxra8, while facilitating attachment and entry, might create a conundrum for viral fusion in the endosome, which requires domain B on E2 to undergo a substantive conformational shift to expose the underlying hydrophobic fusion loop in domain II of E1 (Li et al., 2010; Voss et al., 2010). After clathrin-mediated endocytosis, fusion of CHIKV occurs within the acidic environment of early endosomes (Hoornweg et al., 2016). Although further studies are required, some of the Mxra8 binding interactions with CHIKV E1 and E2 may be sensitive to acidic pH, such that upon transiting to the early endosome the cross-linked trimers can separate and allow the structural transitions required for fusion to occur. In preliminary experiments, deceased affinity of binding of Mxra8 to recombinant pE2-E1 was observed under conditions of mildly acidic pH (K. Basore, unpublished results). It is plausible that the strength of Mxra8 interactions with viral proteins may regulate the stage of endocytosis and pH of fusion of some arthritogenic alphaviruses.

The alphavirus structural polyprotein is processed In the endoplasmic reticulum to yield E3-E2 (p62) and E1, which form heterodimers and oligomerize as trimers to generate the immature spike (Uchime et al., 2013). In the Golgi network, furin-like proteases cleave E3 from E2 (yielding E2-E1) to render the spikes optimally fusogenic. However, this cleavage event can be variable in a cell-type and virus type-specific manner, as E3 remains associated with the mature virus in some alphaviruses, including Sindbis virus, Semliki Forest virus, and VEEV (Heidner et al., 1996; Zhang et al., 2011; Ziemiecki and Garoff, 1978). The comparison of our cryo-EM structures of Mxra8 bound to CHIKV VLPs and infectious virus revealed a difference in stoichiometry of binding, with 240 Mxra8 proteins bound to CHIKV VLP and only 60 bound to our partially mature, infectious CHIKV particles. One reason for the decreased occupancy on the infectious CHIKV was the retention of the E3 protein, which appears to occlude Mxra8 binding at three of four chemical environments in the asymmetric unit. Biochemical and structural analysis confirmed that E3 was absent from our CHIKV VLP preparation, possibly because of the mildly alkaline buffers used in the chromatography purification steps. Our binding studies with soluble mouse or human Mxra8 and CHIKV VLPs defined two classes of sites, one of high affinity (˜60 nM) and a second of lower affinity (˜300 nM). In comparison, infectious CHIKV had only the high-affinity binding site. These data suggest that while Mxra8 can bind mature CHIKV virions in two binding modes with full occupancy, regions of partial maturity which retain E3 will bind in only a single mode. At present, the contribution of the low affinity site for Mxra8 to infectivity remains unclear although these same infectious virus preparations still showed a strong Mxra8-dependence (Zhang et al., 2018). It is plausible that E3 retention on some arthritogenic (e.g., SINV) alphaviruses could result in a lack of contribution of Mxra8 to entry and infection. Future studies with fully mature, infectious alphaviruses propagated in Vero cells over-expressing furin (Mukherjee et al., 2016) and lack E3 might address this hypothesis directly.

Our cryo-EM map of Mxra8 bound to CHIKV was corroborated by mutational analysis and coincidence mapping of neutralizing antibodies that blocked Mxra8 binding. Residues W64, D71, T116, and I121 in the A domain and Y199 and I217 in the B domain of E2 were previously identified as essential for optimal Mxra8 engagement using an E2 alanine scanning mutagenesis library (Fox et al., 2015; Smith et al., 2015; Zhang et al., 2018). Our cryo-EM analysis identified all of these A domain amino acids as contact residues and several B domain residues in close proximity. In the original study, mutations in E1 were not evaluated for binding to Mxra8 because it was expected that E2 and not E1 contributed to receptor engagement (Strauss et al., 1994; Tucker and Griffin, 1991). In further support of the structurally determined Mxra8 binding site on CHIKV, neutralizing human mAbs recognizing epitopes in the A domain or shared epitopes in the A and B domains inhibited Mxra8 binding, whereas others localizing to distinct sites had less effect (Zhang et al., 2018). The epitopes of the mAbs that blocked Mxra8 binding, as determined by alanine scanning mutagenesis (Smith et al., 2015), directly overlap the structural binding sites of Mxra8 and CHIKV E2.

Mxra8 is a receptor for multiple arthritogenic but not encephalitic alphaviruses (Zhang et al., 2018). An alignment of amino acid sequences corresponding to residues comprising the Mxra8 binding site revealed that contact residues generally are conserved among arthritogenic alphaviruses but are located in regions containing or proximal to insertions in encephalitic alphaviruses, which likely explains the negligible interaction or binding requirement for infectivity of Mxra8 with VEEV or related encephalitic alphaviruses (Zhang et al., 2018). Cryo-EM structures with other alphaviruses including ONNV, MAYV, and RRV should provide further insight into the critical contacts on E2, E1, and Mxra8 that facilitate attachment, entry, and infection. Such analysis could identity targets on either the viral structural proteins or Mxra8 to facilitate the development of agents capable of disrupting these interactions. This approach could form the basis of a therapy that mitigates infection caused by multiple arthritogenic alphaviruses.

Experimental Models

Viruses. CHIKV strain 181/25 was obtained from the World Reference Center for Emerging Viruses and Arboviruses (S. Weaver and K. Plante, Galveston, TX). The virus was propagated in Vero cells (ATCC) in DMEM supplemented with 10% FBS, concentrated by sucrose gradient ultracentrifugation, and titrated by standard focus-forming assays (Brien et al., 2013; Pal et al., 2013).

VLP production and purification. CHIKV VLPs were produced via transient transfection of C-E3-E2-6K-E1 (CHIKV strain 37997) plasmid DNA (Akahata et al., 2010) into HEK293 cells (obtained from Vaccine Research Center, NIH), and purified via Q Sepharose XL (GE Healthcare, GE17-5072-01) anion chromatography. The peak of interest was diafiltered into a buffer containing 218 mM sucrose, 10 mM potassium phosphate, and 25 mM citrate, pH 7.2. The material was sterile-filtered using a 0.2 μM filter, 500 μL aliquots were made and stored at −80° C. The final material was analyzed by BCA assay for protein concentration, Coomassie-stained SDS-PAGE gel with densitometry, and Western blotting with rabbit polyclonal anti-CHIKV 181/25 (04-0008, IBT Bioservices) and a secondary horseradish peroxidase conjugated goat anti-rabbit antibody (65-6120, ThermoFisher Scientific).

SPR-based Mxra8 binding assay. SPR binding experiments were performed on a Biacore T200 system (GE Healthcare) to measure the kinetics and affinity of Mxra8 binding to CHIKV VLPs. Experiments were performed at 30 μl/min and 25° C. using HBS-EP (0.01 M HEPES pH 7.4, 0.15 M NaCl, 3 mM EDTA, 0.005% v/v Surfactant P20) as running buffer. CHK-265 mAb (Pal et al., 2013) was immobilized onto a CM5 sensor chip (GE Healthcare) using standard amine coupling chemistry, and CHIKV VLPs were captured. Recombinant Mxra8 was injected over a range of concentrations (2 μM to 20 nM) for 200 sec, followed by a 600 sec dissociation period. As a negative control, murine norovirus was captured with mAb A6.2, as described previously (Taube et al., 2010). Real-time data was analyzed using BIAevaluation 3.1 (GE Healthcare). For the equal affinity binding sites model, kinetic profiles and steady-state equilibrium concentration curves were fitted using a global 1:1 binding algorithm with drifting baseline.

For the model of Mxra8 binding to 1 of 4 sites with higher affinity, the following fits were used:

Equilibrium Analysis:

$$R U = \frac{{k}_{1} [ A ]}{1 + {k}_{1} [ A ]} + \frac{3 {k}_{2} [ A ]}{1 + {k}_{2} [ A ]}$$

where k 1 corresponds to the high affinity site and k 2 denotes the low affinity sites.

Kinetic Analysis:

Reaction Equations:

$$A + B 1 \qquad {k}_{a 1} \\ \to \\ ← {k}_{d 1} \qquad AB 1$$

$$\begin{matrix}A + B 12 & 3 {k}_{a 2} \\ \to \\ ← 3 {k}_{d 2} & AB 2\end{matrix} \\$$

where A=analyte (Mxra8), B1=high affinity sites of ligand (CHIKV VLP), and B2=low affinity sites of ligand (CHIKV VLP)

Differential Equations:

$$\frac{d [ B 1 ]}{d t} = - ( {k}_{a 1} [ A ] [ B 1 ] - {k}_{d 1} [ A B 1 ] )$$

$$\frac{d [ B 2 ]}{d t} = - 3 ( {k}_{a 2} [ A ] [ B 2 ] - {k}_{d 2} [ A B 2 ] )$$

$$\frac{d [ A B 1 ]}{d t} = ( {k}_{a 1} [ A ] [ B 1 ] - {k}_{d 1} [ A B 1 ] )$$

$$\frac{d [ A B 2 ]}{d t} = 3 ( {k}_{a 2} [ A ] [ B 2 ] - {k}_{d 2} [ A B 2 ] )$$

BLI-based binding Assay. Binding of CHK-265 and Mxra8 to mAb-captured CHIKV VLP was monitored in real-time 25° C. using an Octet-Red96 device (Pall ForteBio). CHK-265 (100 μg) was mixed with biotin (EZ-Link-NHS-PEG4-Biotin, Thermo Fisher) at a molar ratio of 20:1 biotin:mAb, incubated at room temperature for 30 min, then unreacted biotin was removed by passage through a desalting column (5 mL Zeba Spin 7K MWCO, Thermo Fisher). Biotinylated-CHK-265 was loaded onto streptavidin biosensors (ForteBio) until saturation, typically 10 μg/ml for 2 min, in 10 mM HEPES (pH 7.4), 150 mM NaCl, 3 mM EDTA, and 0.005% P20 surfactant with 1% BSA. CHIKV VLP was then added for 5 min. The biosensors were dipped sequentially into saturating concentrations of Mxra8 and CHK-265 (2 μM and 100 nM, respectively) for 5 min or vice-versa, with 30 sec baseline measurements in between associations, followed by a final 10 min dissociation.

Cryo-EM sample preparation, data collection, and single particle reconstruction. CHIKV VLP with and without cleaved Mxra8 and CHIKV infectious virus (181/25 strain) with cleaved Mxra8 in molar excess were flash frozen on holey carbon EM grids in liquid ethane under BSL-2 containment conditions using an FEI Vitrobot (ThermoFisher). Movies of the samples were recorded with the software EPU (Thermo Fisher) using a K2 Summit electron detector (Gatan) mounted on a Bioquantum 968 GIF Energy Filter (Gatan) attached to a Titan Krios microscope operating at 300 keV with an electron dose of 50 e⁻/Å² and a magnification of 18,000×. The movies (30 frames, 300 msec exposure per frame) were corrected for beam-induced motion using MotionCor2 (Zheng et al., 2017). Contrast transfer function parameters of the electron micrographs were estimated using CTFFIND4 (Rohou and Grigorieff, 2015) in cisTEM (Grant et al., 2018). Particles were auto-picked, and underwent reference-free 2D classification, ab initio 3D reconstruction, and 3D refinement in cisTEM. Local resolution was estimated using RELION-3 (Zivanov et al., 2018). Additional information regarding the number of images and particles is listed in TABLE 2. All structural analyses of the 3D reconstructions were performed using the programs Chimera and PyMOL (Bramucci et al., 2012).

Model building. The initial models of the CHIKV structural proteins (E1, E2, TM regions, and Capsid) with or without Mxra8 were built into the density of a subunit by docking the components from the crystal structures of CHIKV p62-E1 (PDB: 3N42), the CHIKV capsid (PDB: 5J23), and the modeled TM regions of VEEV (PDB: 3J0C) using the fitmap command in Chimera. Amino acid substitutions and connections were added manually in COOT to reflect the strain of CHIKV used for VLP production (CHIKV-37997) and infectious virus (CHIKV-181/25) for cryo-EM studies. The subunit models then underwent real-space refinement using PHENIX with default parameters plus rigid body refinement. Rigid bodies for E1 were divided into domains I and II (residues 1-292), domain III (293-381), stem (382-412), and transmembrane region (413-442). E2 was divided into the N-linker region (residues 5-15), domain A (residues 16-134), domain B (residues 173-231), domain C (residues 269-341), β-linker (residues 135-172, 232-268), and the stem, transmembrane region, and cytoplasmic tail (residues 342-423). The capsid protein was divided into two rigid bodies (residues 111-176 and residues 177-261), and Mxra8 was divided into D1 (residues 44-174) and D2 (10-43; 175-269). The refined subunits were used to build the asymmetric unit with adjacent subunits to prevent clashes and optimize interactions. These models underwent further real-space refinement. COOT was used to fix regions manually with poor geometry. After optimization, coordinates of the asymmetric units were checked by MolProbity (TABLE 4). Contact residues were identified, and buried surface areas were calculated using PDBePISA.

Mapping of antibody or Mxra8 contact residues onto the CHIKV p62-E1 crystal structure. Figures were prepared using the atomic coordinates of CHIKV pE2-E1 (RCSB accession number 3N42) using the program PyMOL (The PyMOL Molecular Graphics System, Version 1.7.4 Schrödinger, LLC). Amino acid contact residues for neutralizing anti-human or anti-mouse mAbs that block Mxra8 binding were derived from published studies (Fox et al., 2015; Smith et al., 2015).

Data Availability. All structures described below are deposited in the PDB and EMDB databases and are incorporated by reference in their entireties: Crystal structure of murine Mxra8 ectodomain (PDB code for this deposition is 6NK3); electron Cryo-Microscopy Of Chikungunya VLP alone (PDB code for this deposition is 6NK5; EMDB code for this deposition is EMD-9393); Electron Cryo-Microscopy of Chikungunya VLP in complex with mouse Mxra8 receptor (PDB code for this deposition is 6NK6; EMDB code for this deposition is EMD-9394); Electron Cryo-Microscopy of Chikungunya in complex with mouse Mxra8 receptor (PDB code for this deposition is 6NK7; EMDB code for this deposition is EMD-9395).

TABLE 2
Data collection and refinement statistics - mouse Mxra8.

| Mouse Mxra8 | | |
|---|---|---|
| Resolution range | 36.88-2.0 | (2.072-2.0) |
| Space group | I 2 2 2 | |
| Unit cell | 72.588 142.847 195.585 90 90 90 | |
| Unique reflections | 64594 | (4630) |
| Completeness (%) | 93.65 | (67.72) |
| Wilson B-factor | 37.68 | |
| Reflections used in refinement | 64595 | (4631) |
| Reflections used for R-free | 1989 | (142) |
| R-work | 0.2238 | (0.5262) |
| R-free | 0.2414 | (0.5724) |

| | |
|---|---|
| macromolecules | 4258 |
| solvent | 193 |
| Protein residues | 528 |
| RMS(bonds) | 0.004 |
| RMS(angles) | 1.00 |
| Ramachandran favored (%) | 95.42 |
| Ramachandran allowed (%) | 4.20 |
| Ramachandran outliers (%) | 0.38 |
| Rotamer outliers (%) | 3.74 |
| Clashscore | 6.25 |
| Average B-factor | 63.01 |
| macromolecules | 63.22 |
| solvent | 58.19 |

Statistics for the highest-resolution shell are shown in parentheses.

TABLE 3
Summary of Cryo-EM data collection.

| Sample | VLP alone | VLP + Mxra8 | CHIKV + Mxra8 |
|---|---|---|---|
| # of Micrographs | 1332 | 7294 | 5318 |
| # of particles picked | 8888 | 26790 | 9547 |
| # of particles after 2D | 8113 | 22486 | 8357 |
| classification | | | |
| Resolution_{FSC=0.5} (Å) | 4.48 | 4.32 | 5.27 |
| Resolution_{FCS=0.143} (Å) | 4.16 | 4.06 | 4.99 |

The microscope settings for image collection were: Dose: 50 e⁻/Å²; Magnification: 18,000×; Pixel size: 1.4; and Voltage: 300 keV. Micrographs were corrected for the contrast transfer function using CTFFIND4 (Rohou and Grigorieff, 2015) in the program cisTEM (Grant et al., 2018).

TABLE 4
Refinement and model statistics.

| | | VLP | VLP + | CHIKV + |
|---|---|---|---|---|
| Asymmetric Unit Model | | alone | Mxra8 | Mxra8 |
| CHIKV strain | | CHIKV- | CHIKV- | CHIKV- |
| | | 37997 | 37997 | 181/25 |
| Number of chains | | 12 | 16 | 17 |
| Resolution (Å) | | 4.5 | 4.4 | 5.5 |
| MolProbity score | | 2.26 | 2.30 | 2.26 |
| all-atom clashscore | | 13.77 | 15.20 | 12.86 |
| Rotamer outliers (%) | | 0.53 | 0.63 | 0.54 |
| Ramachandran | Favored (%) | 87.19 | 87.36 | 85.9 |
| Plot values | Allowed (%) | 12.66 | 12.38 | 13.95 |
| | Outliers (%) | 0.15 | 0.26 | 0.16 |
| R.M.S. | Bond | 0.007 | 0.006 | 0.005 |
| Deviations | lengths (Å) | | | |
| | Bond | 1.128 | 1.097 | 1.151 |
| | angles | | | |

TABLE 5
List of contact residues of CHIKV E2-E1 at Mxra8 binding interface.

| | | | | Mxra8 |
|---|---|---|---|---|
| E2-E1 heterodimer | E2-E1 domain | E2-E1 residues | Mxra8 residues | domain |
| wrapped | Domain A of | E24, H26, S27, | R64, H196, L197, | D2 |
| | E2 | C28, H29, D71, | E198, E199, D223, | |
| | | S72 | Y225, S227, G228, | |
| | | | E229, R230, R231, | |
| | | | A232, Y233 | |
| | Domain B of | T179, K189, I190, | N187, R188, A189, | D1 |
| | E2 | T191, N193, G194, | H190 | |
| | | D214, K215, K221, | V203, H205, D207, | D2 |
| | | I222, D223 | S214, H215, D216, | |
| | | | R217, A218, D219, | |
| | | | R220, H272, H274, | |
| | | | H276, H281, R283 | |
| | Fusion loop of | Y85, F87, W89, | S112, H115, D116, | D1 |
| | E1 | G90, D97, A226 | I186, R188, H190, | |
| | | | D194, R195, H196 | |
| Adjacent intraspike | Domain A of | N5, H62, D63, | W60, T61, Q62, | D2 |
| | E2 | V64, T65 | D63, N247, F249, | |
| | | | A250 | |
| | β-linker of E2 | E150, V157, Q158, | Q62, D63, R64, | D1 |
| | | S159, N263, V264, | H66, S92, A93, | |
| | | T265, R267 | G94, R195, H196 | |
| | Domain I of E1 | T143, A145, A146, | R102, R104, D105, | D1 |
| | | K157 | R124, H177, G178 | |
| | Domain II of | K132, T263, N264, | E95, R97, V98, | D1 |
| | E1 | P265, R267 | R124 | |
| Neighboring | Domain II of | D70, K71, S72, | L76, Q83, R85, | D1 |
| interspike | E1 | L73, P74, E209, | R102, D127, R128, | |
| | | K211, D212 | G129, E131, V155, | |
| | | | T156, E157, H177 | |

Contact residues were identified using PDBePISA. Mxra8 is numbered according to NCBI reference sequence NP_077225.4. In bold are contacts unique to Mxra8 at binding site 2, and in italics are contacts at each Mxra8 position except binding site 2.

### Example 6: Vertebrate Class-Specific Binding Modes of the Alpha Virus Receptor Mxra8

MXRA8 is a receptor for chikungunya (CHIKV) and other arthritogenic alphaviruses with mammalian hosts. However, mammalian MXRA8 does not bind to alphaviruses that infect humans and have avian reservoirs. Herein is shown that avian, but not mammalian, MXRA8 can act as a receptor for Sindbis, western equine encephalitis (WEEV), and related alphaviruses with avian reservoirs. Structural analysis of duck MXRA8 complexed with WEEV reveals an inverted binding mode compared to mammalian MXRA8 bound to CHIKV. Whereas both domains of mammalian MXRA8 bind CHIKV E1 and E2, only domain 1 of avian MXRA8 engages WEEV E1, and no appreciable contacts are made with WEEV E2. Using these results, a chimeric avian-mammalian MXRA8 decoy-receptor was generated that neutralizes infection of multiple alphaviruses from distinct antigenic groups in vitro and in vivo. Thus, different alphaviruses can bind MXRA8 encoded by different vertebrate classes with distinct engagement modes, which enables development of broad-spectrum inhibitors.

### INTRODUCTION

Alphaviruses are a group of globally important, mosquito-transmitted, enveloped, positive-sense RNA viruses in the Togaviridae family, and those infecting humans have several reservoirs including non-human primates, macropods, reptiles, rodents, and birds. Two clinical syndromes occur in humans after alphavirus infection: acute encephalitis and neurological disease are caused by Venezuelan equine encephalitis (VEEV), eastern equine encephalitis (EEEV), and western equine encephalitis (WEEV) viruses; and acute and chronic musculoskeletal disease and arthritis are caused by chikungunya (CHIKV), Ross River (RRV), O'nyong'nyong (ONNV), Mayaro (MAYV) and Sindbis (SINV) viruses.

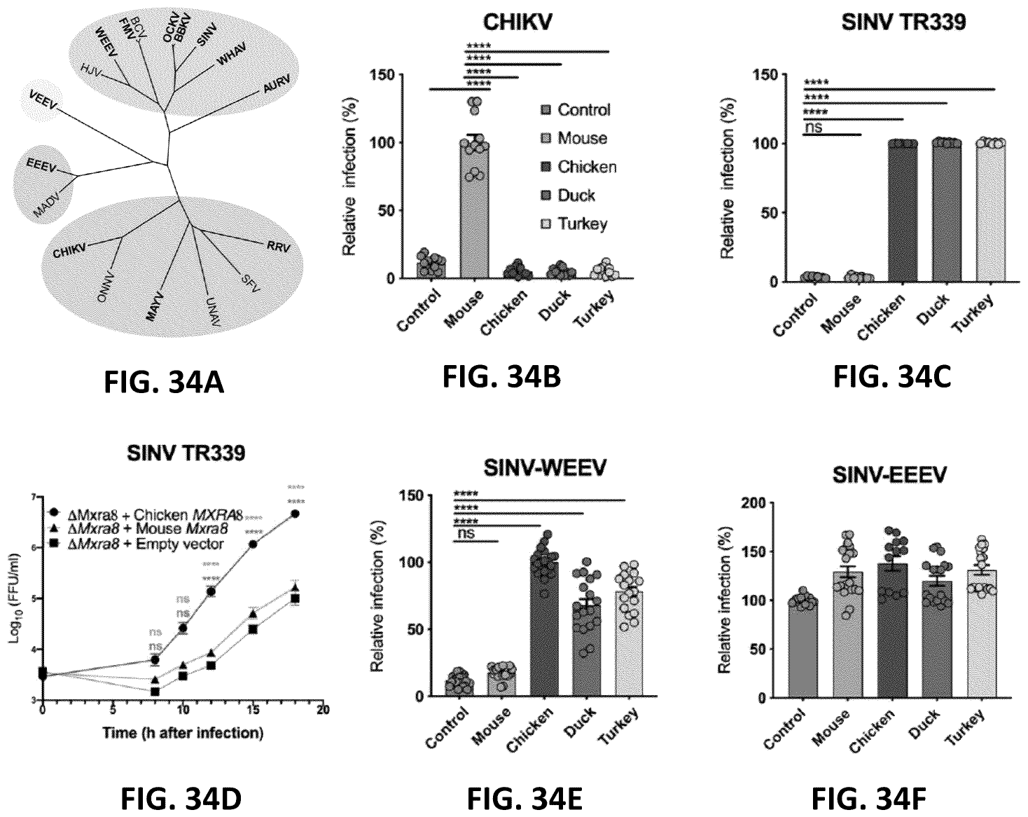

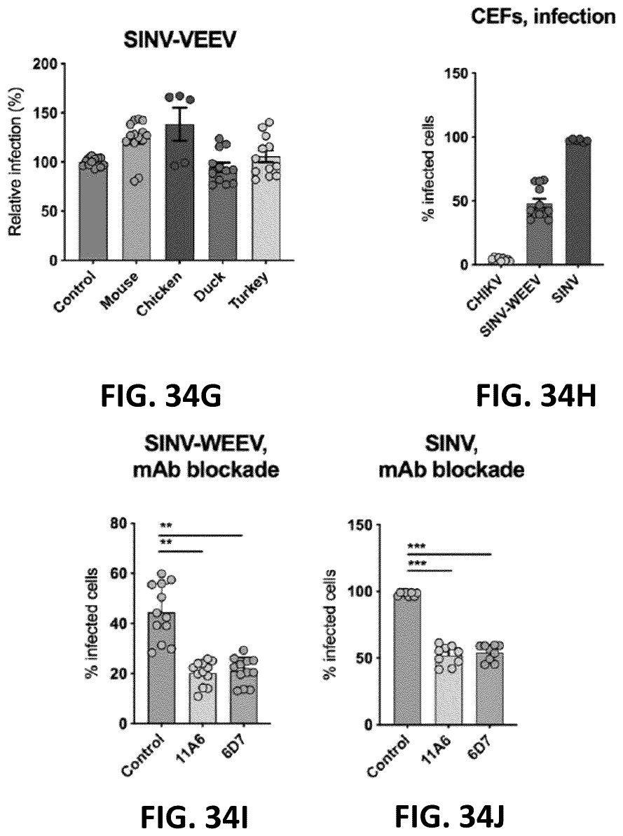

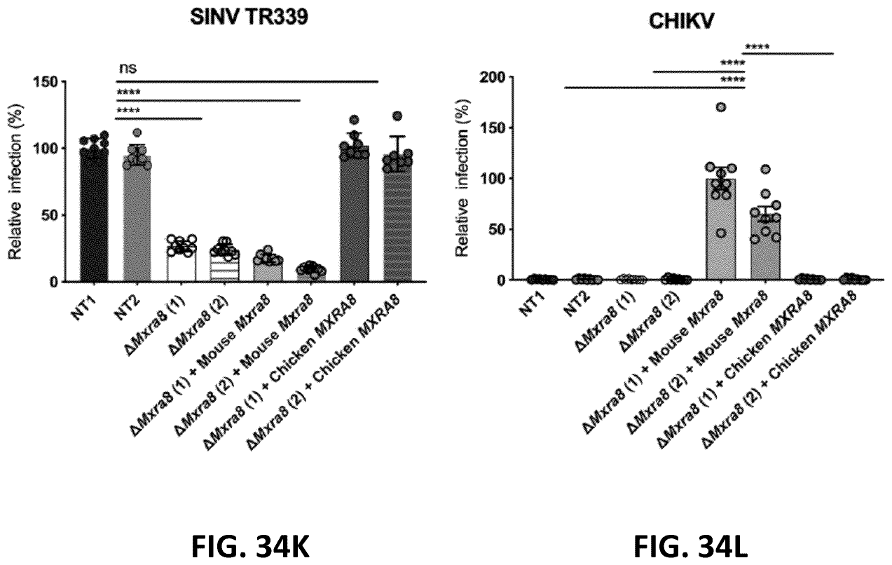

Over the last several thousand years, New World alphaviruses (e.g., VEEV and EEEV) evolved and separated from Old World alphaviruses (e.g., SINV and Semliki Forest (SFV) virus) (see e.g., FIG. 34A). The western equine encephalomyelitis (WEE) complex is an antigenically related group that includes four New World (Aura (AURAV), Fort Morgan (FMV), Highlands J (HJV), and WEEV) and several Old World (e.g., SINV and Whataroa (WHAV)) viruses. WEEV, HJV, and FMV are considered descendants of a recombination between a SINV-like virus and EEEV that is believed to have occurred in South America. The sequences of non-structural proteins, the capsid protein, and the (untranslated) 3′-terminal 80 nucleotides of WEEV, HJV, and FMV are related most closely to EEEV, whereas those of the E2 and E1 structural glycoproteins are related more to SINV. WEEV has the encephalitic properties of New World alphaviruses but the structural proteins and presumably receptor specificity of Old World alphaviruses. SINV, which causes periodic outbreaks in Africa, Europe, Asia, and Australia, has three antigenic subtypes: Babanki (BBKV; Africa), Ockelbo (OCKV; Sweden), and Kyzylagach (KYZV: Azerbaijan and China), all of which share clinical syndromes of fever, rash, and arthralgia in humans.

Matrix Remodeling Associated 8 (MXRA8) is a conserved cell-surface molecule in mammals, birds, reptiles, and fish, and comprised of two Ig-like domains arranged in a unique head-to-head orientation. MXRA8 serves as an entry receptor for CHIKV and several other arthritogenic alphaviruses that are members of the Semliki Forest (SF) antigenic group (see e.g., FIG. 34A), including MAYV, RRV, and ONNV, but not for encephalitic alphaviruses or WEE complex members. Whereas ectopic expression of many mammalian species of MXRA8 proteins (e.g., mouse, rat, chimpanzee, dog, horse, goat, and sheep) facilitates CHIKV infection in 3T3 cells, MXRA8 from several avian species tested (e.g., chicken, turkey, and duck) do not promote CHIKV infection.

Given the evolutionary relationships between alphaviruses, their differential utilization of mammalian MXRA8, and distinct dependence on bird reservoirs, explored herein is whether avian MXRA8 could act as a receptor for alphaviruses that use birds in their enzootic cycle. Herein is demonstrated that avian MXRA8 is an entry receptor for WEE complex alphaviruses. Cryo-electron microscopy (cryo-EM) structural analysis unexpectedly shows that WEEV binds to duck MXRA8 in a flipped orientation relative to mammalian MXRA8 binding to CHIKV. This discovery was applied to generate a chimeric duck-mouse MXRA8 receptor that supports infection of both WEE and SF complex viruses and a soluble decoy receptor inhibitor that blocks infection of viruses from both families in cell culture and in vivo. Cryo-EM analysis of a duck-mouse chimeric MXRA8 bound to CHIKV or WEEV confirmed the flipped binding orientation and revealed that D1 of duck MXRA8 is critical for binding to both WEE and SF complex alphaviruses despite engaging unique locations through different approaches. Overall, these findings demonstrate how zoonotic viruses within the same alphavirus family use distinct binding modes to allow for receptor usage and species tropism, and how this knowledge can be harnessed for the development of possible broad-spectrum therapies against a range of alphaviruses.

Results

WEEV complex alphaviruses with an avian reservoir use avian MXRA8 for infection of mammalian cells.

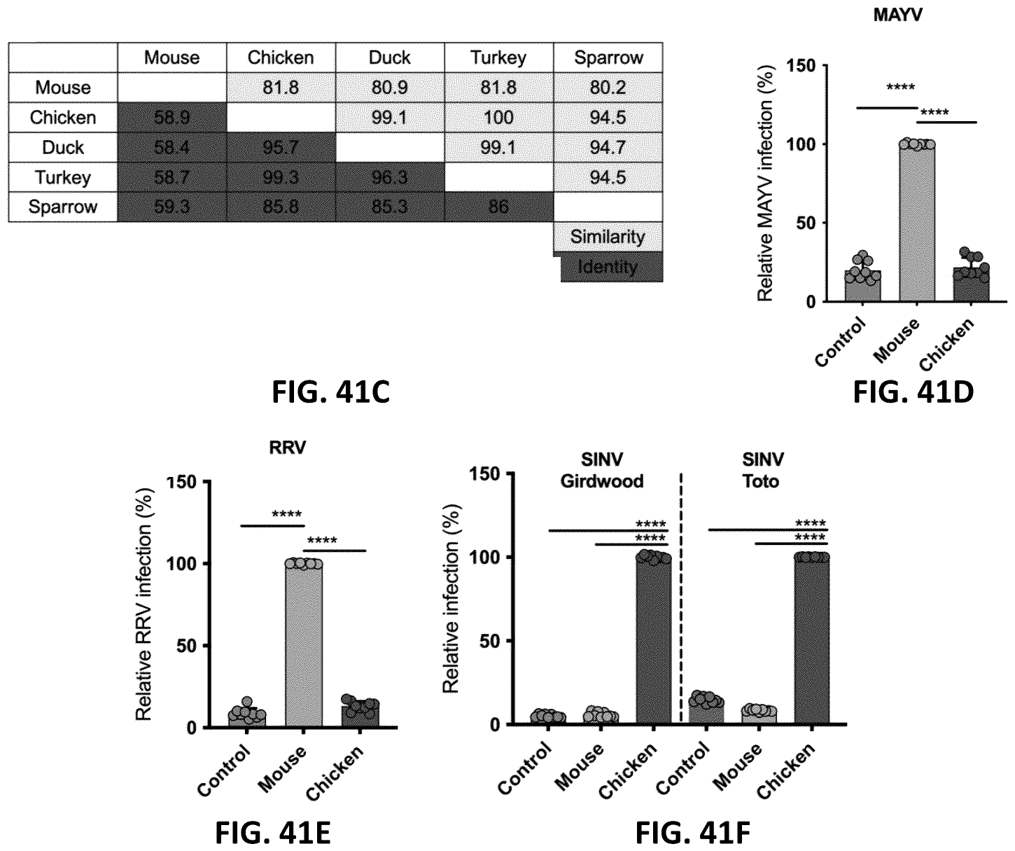

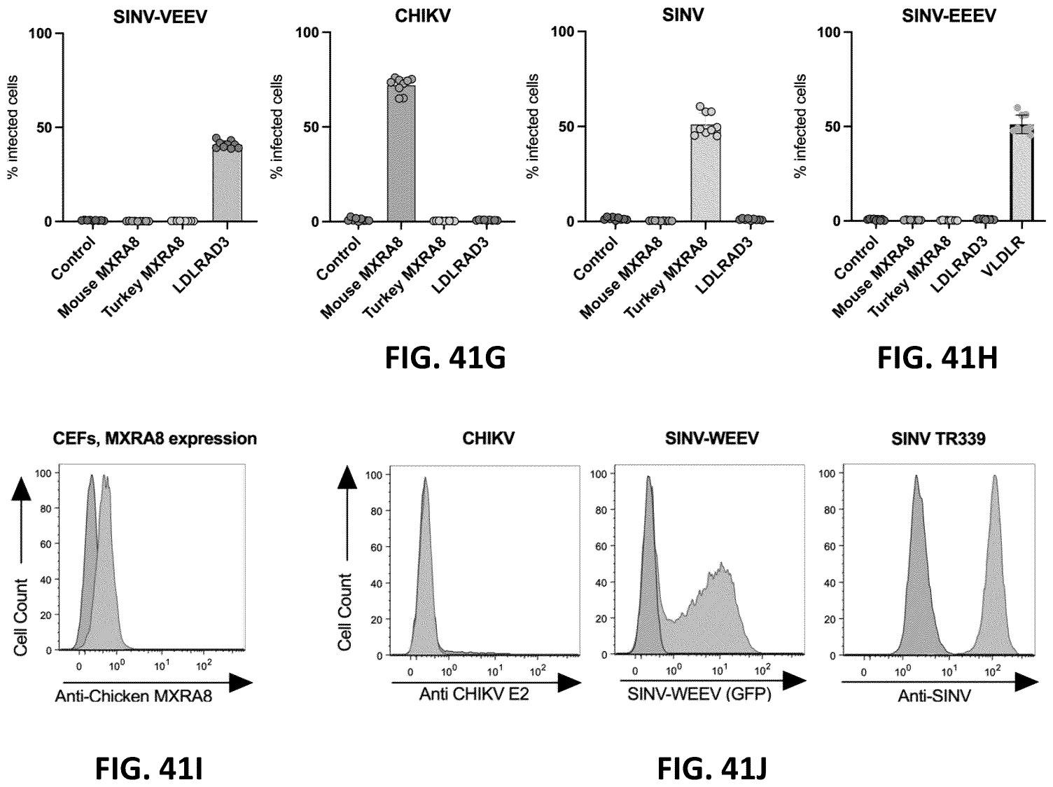

Mouse 3T3 fibroblasts lacking MXRA8 expression (ΔMxra8) were complemented with MXRA8 from chicken (Gallus gallus), duck (Anas platyrhynchos) or turkey (Meleagris gallopavo) (see e.g., FIG. 41A-FIG. 41B), which share approximately 59% amino acid identity with murine MXRA8 (see e.g., FIG. 41C). As expected, murine MXRA8 facilitated infection of the SF complex viruses CHIKV, RRV, and MAYV, whereas expression of chicken, duck, or turkey MXRA8 did not (see e.g., FIG. 34B and FIG. 41D-FIG. 41E). Next the impact of avian MXRA8 expression on other alphaviruses with bird reservoirs was evaluated. Whereas SINV (strain TR339) infection was low in ΔMxra8 3T3 cells or ΔMxra8 3T3 cells complemented with mouse Mxra8, high levels of SINV infection were detected in ΔMxra8 cells complemented with chicken, duck, or turkey MXRA8 (see e.g., FIG. 34C). Other SINV strains (SINV-Girdwood and SINV-Toto) had similar phenotypes (see e.g., FIG. 41F). Multi-step growth curve analysis corroborated a dependence of SINV infection on avian but not mammalian MXRA8 for optimal virus production (see e.g., FIG. 34D). Infection levels of SINV-WEEV, a chimeric alphavirus encoding the non-structural genes of SINV and structural genes of WEEV, also were higher in ΔMxra8 3T3 cells complemented with avian MXRA8 than ΔMxra8 cells or ΔMxra8 cells complemented with mouse Mxra8 (see e.g., FIG. 34E). In contrast, expression of avian MXRA8 proteins did not enhance infection of SINV-VEEV or SINV-EEEV chimeric viruses in ΔMxra8 3T3 cells (see e.g., FIG. 34F-FIG. 34G). These results were confirmed and extended with other cell types. SINV-VEEV infection was not enhanced in Jurkat cells expressing mouse or turkey MXRA8, whereas greater infection was observed in cells expressing LDLRAD3, a receptor for VEEV (see e.g., FIG. 41G). In comparison, expression of mouse and turkey MXRA8 respectively promoted CHIKV and SINV infection in Jurkat cells (see e.g., FIG. 41G). In K562 cells, mouse and turkey MXRA8 also did not promote infection of SINV-EEEV, whereas expression of VLDLR, a recently described EEEV receptor, did (see e.g., FIG. 41G). These results are consistent with alphavirus evolutionary relationships, as the structural genes of WEEV are more closely related to SINV than to EEEV or VEEV.

Avian MXRA8 Enhances Infection of WEEV Complex Alphaviruses with an Avian Reservoir in Avian Cells.

The findings were confirmed in a species-relevant cell system using chicken embryonic fibroblasts (CEFs), which express chicken MXRA8 on their surface (see e.g., FIG. 41I). First, it was tested whether CEFs could support infection of different mammalian and avian alphaviruses including CHIKV, SINV-WEEV, or SINV. Whereas CHIKV antigen was absent in CEFs at 24 h, WEEV and SINV antigens were detected at high levels at 9 h post-infection (see e.g., FIG. 34H and FIG. 41J). To determine whether CEFs infection by SINV-WEEV and SINV depended on chicken MXRA8, CEFs were pre-incubated with blocking anti-chicken MXRA8 mAbs (see Methods); this treatment reduced infection of SINV-WEEV (see e.g., FIG. 34I) or SINV (see e.g., FIG. 34J) compared to an isotype control mAb.

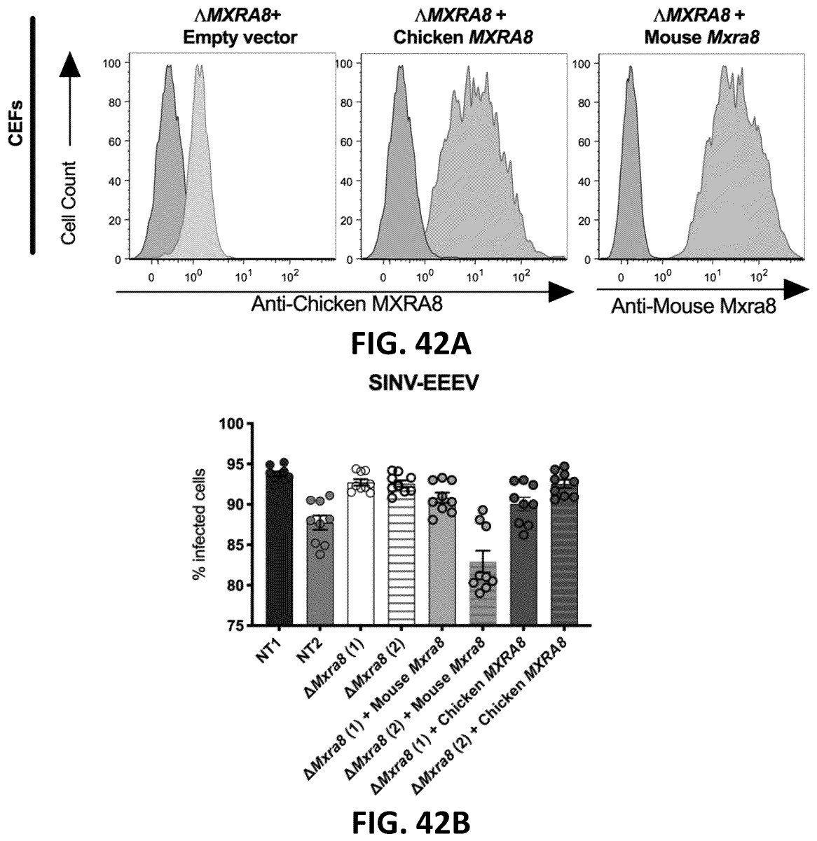

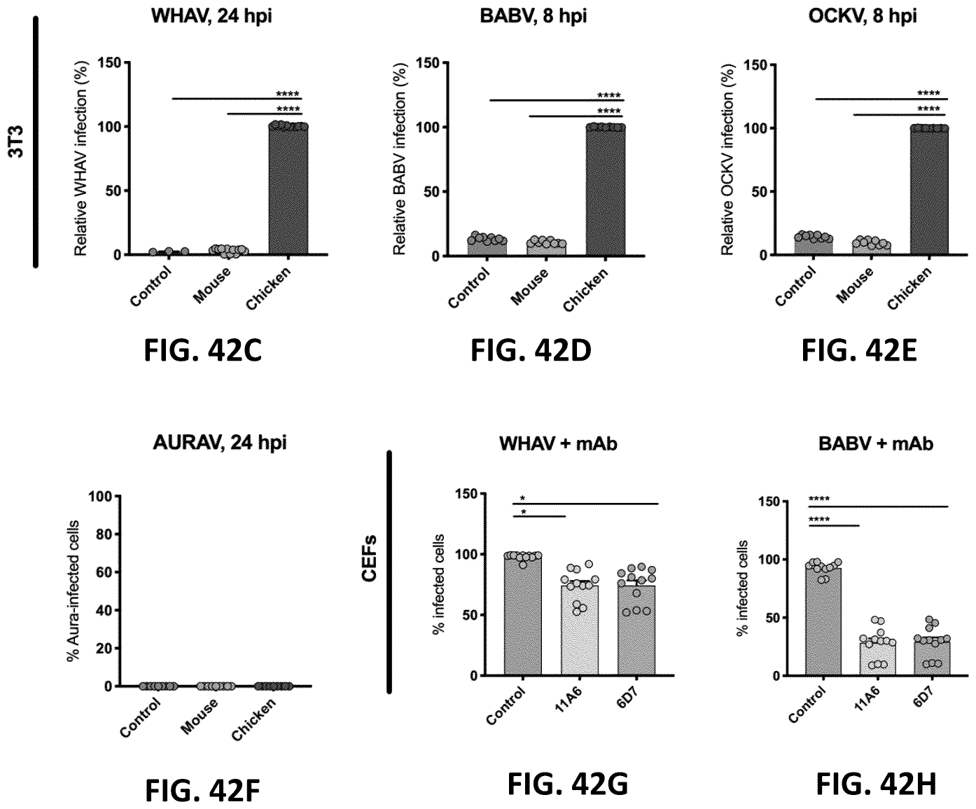

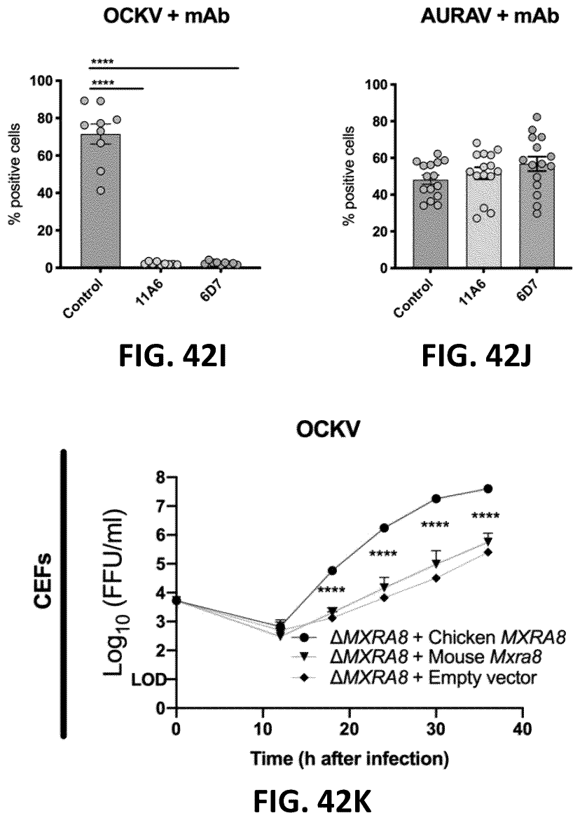

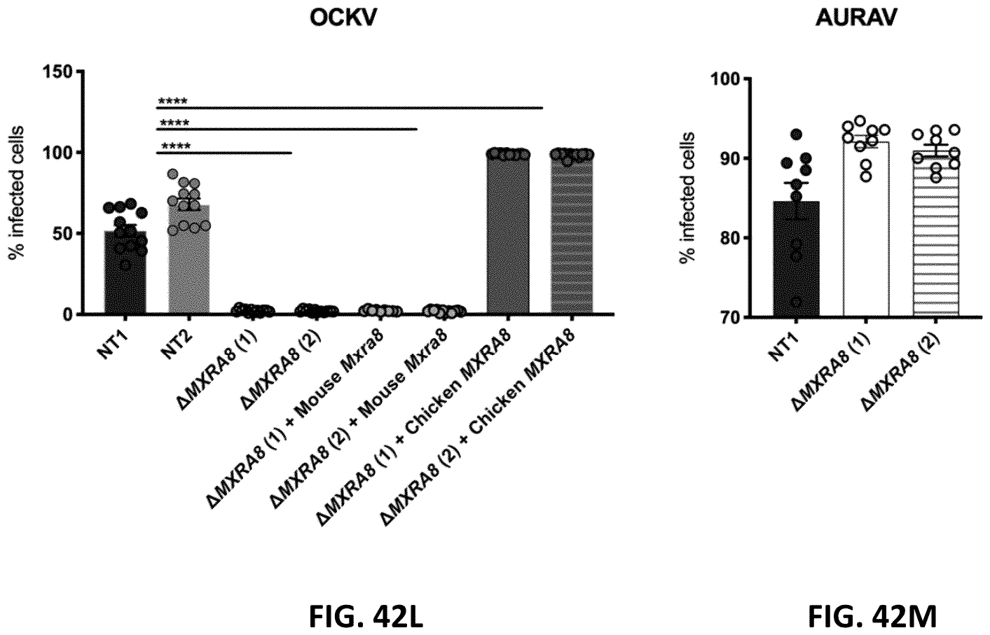

To corroborate these findings, ΔMXRA8 CEFs were generated using gene editing and then complemented with chicken MXRA8 or mouse Mxra8 (see e.g., FIG. 42A). This panel of cells, along with control CEFs edited with non-targeted single-guide RNA, were inoculated with SINV, CHIKV, or SINV-EEEV. ΔMXRA8 CEFs or ΔMXRA8 CEFs complemented with mouse Mxra8 showed less SINV antigen expression than non-targeted CEFs or ΔMXRA8 CEFs complemented with chicken MXRA8 (see e.g., FIG. 34K). At 10 h after CHIKV inoculation, ˜50% of ΔMXRA8 CEFs complemented with mouse Mxra8 stained positively for CHIKV antigen, whereas virtually none of the non-targeted CEFs or ΔMXRA8 CEFs complemented with chicken MXRA8 supported CHIKV infection (see e.g., FIG. 34L). SINV-EEEV infection levels were similar in all tested CEFs (see e.g., FIG. 42B). These experiments suggest that avian MXRA8 promotes infection of SINV and WEEV, CHIKV is non-permissive for CEFs unless mouse MXRA8 is expressed ectopically, and EEEV infects CEFs in a MXRA8-independent manner.

Avian MXRA8 Facilitates SINV Attachment and Entry into Cells and Binds Directly to WEEV Virus-Like Particles (VLPs).

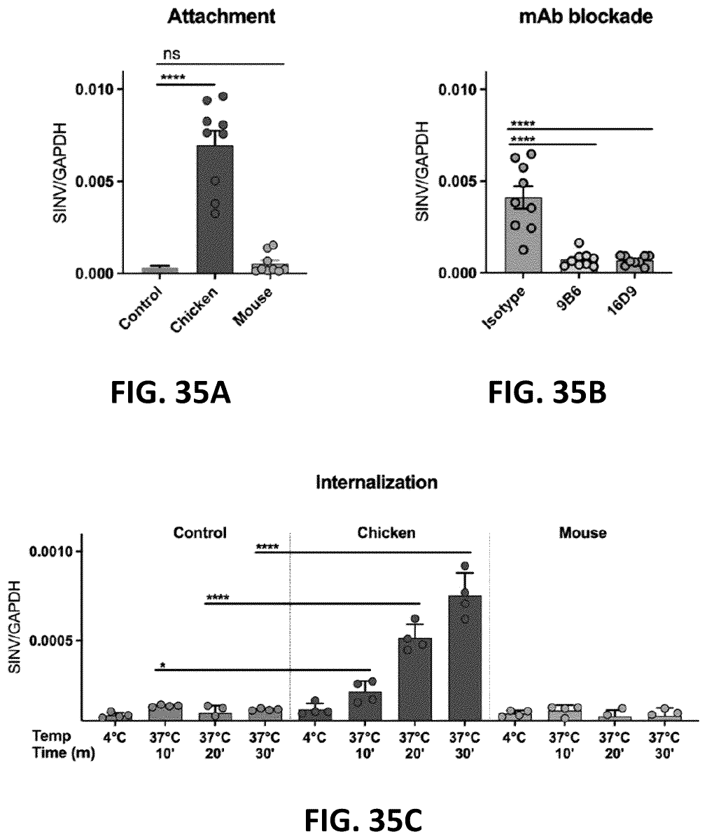

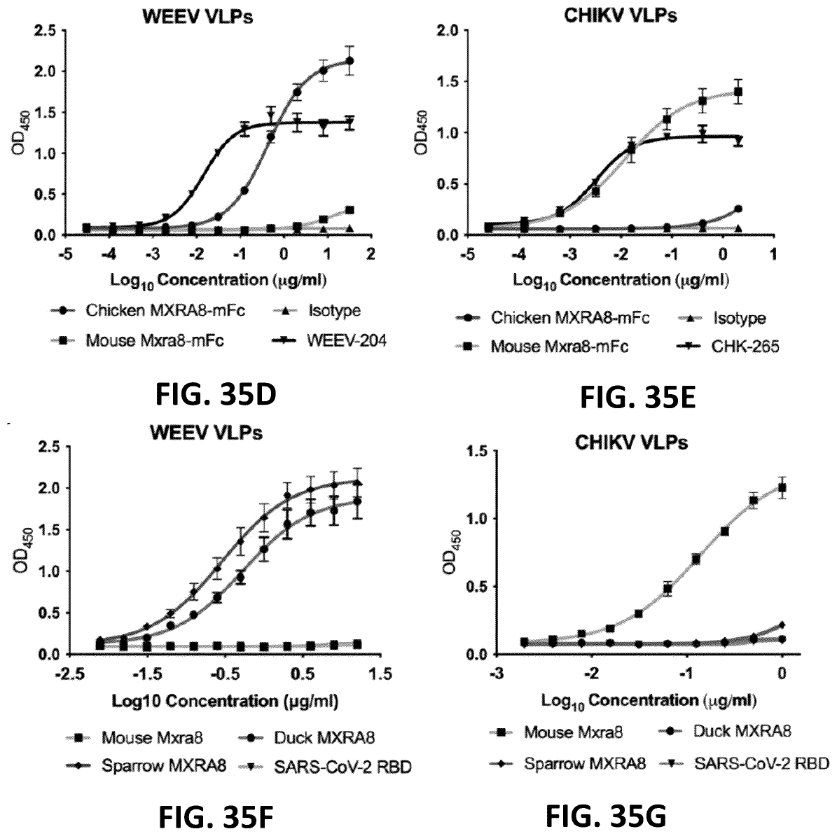

It was evaluated whether avian MXRA8 has a role in viral attachment and entry as demonstrated for mouse MXRA8 and CHIKV. Indeed, SINV showed increased binding at 4° C. to ΔMxra8 mouse 3T3 cells complemented with chicken MXRA8 compared to ΔMxra8 cells or ΔMxra8 cells complemented with mouse Mxra8 (see e.g., FIG. 35A). Consistent with an effect of avian MXRA8 on virus attachment, pre-incubation of cells with anti-chicken MXRA8 mAbs inhibited binding of SINV (see e.g., FIG. 35B). When virus internalization was allowed to occur after incubation at 37° C., higher levels of SINV RNA were detected within ΔMxra8 cells complemented with chicken MXRA8 than within ΔMxra8 cells or ΔMxra8 cells complemented with mouse Mxra8 (see e.g., FIG. 35C).

It was tested whether avian MXRA8 could bind directly to virus-like particles (VLPs) containing WEEV or CHIKV structural proteins. A chicken MXRA8-Fc fusion protein was generated, which bound directly to WEEV but not to CHIKV VLPs, whereas mouse MXRA8-Fc fusion protein bound to CHIKV but not to WEEV VLPs (see e.g., FIG. 35D-FIG. 35E). Since sparrows are reservoirs for some alphaviruses, and sparrow MXRA8 sequence varies from chicken MXRA8 by 4% at the amino acid level, this protein was also generated. WEEV VLPs bound to both duck and sparrow MXRA8, but did not bind to mouse MXRA8 (see e.g., FIG. 35F). Reciprocally, CHIKV VLPs bound to mouse MXRA8 but not to duck or sparrow MXRA8 (see e.g., FIG. 35G).

Multiple WEE Complex Members Use Avian but not Mammalian MXRA8.

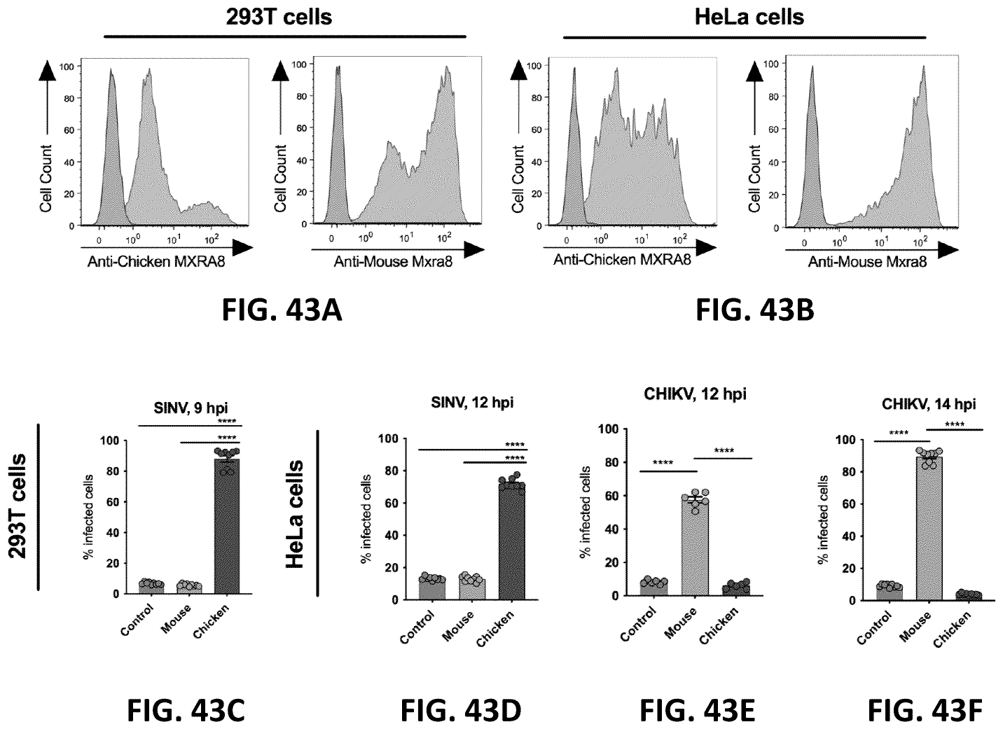

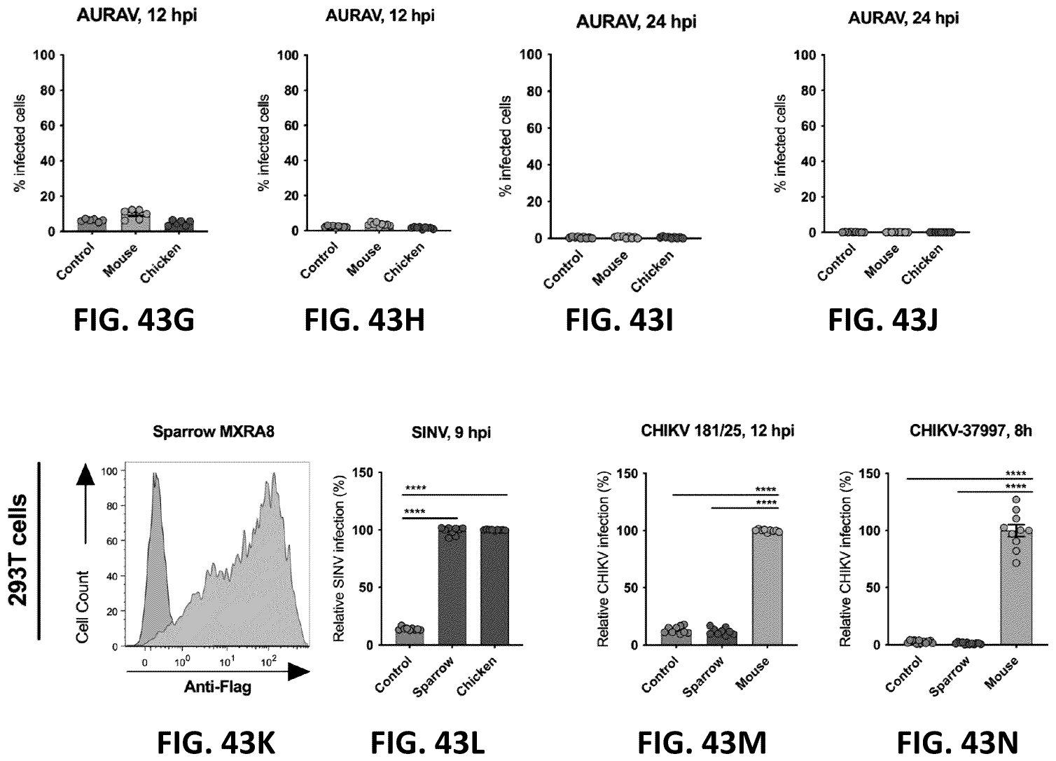

It was assessed whether other WEE complex members could use avian MXRA8 as an entry receptor. Based on phylogenetic relationships (see e.g., FIG. 34A), experiments were performed in control and MXRA8-expressing chicken (CEFs) and human (293T and HeLa) cells with three additional WEE antigenic complex members (WHAV, BBKV, and OCKV) with avian reservoirs that periodically cause infections in humans. AURAV, a WEE complex member restricted to South America that lacks an established animal reservoir, was also tested. Avian MXRA8 promoted infection of SINV, BBKV, OCKV, and WHAV, whereas mouse MXRA8 did not (see e.g., TABLE 6, FIG. 42A-FIG. 42M and FIG. 43A-FIG. 43N).

TABLE 6
Properties of avian and mammalian MXRA8 receptor activity for alphaviruses.

| | | | | Infection | |
|---|---|---|---|---|---|
| | | Infection | | inhibited | |
| | | enhanced | Infection | by | |
| | Infection | by | inhibited | knockout | |
| | enhanced by | mammalian | by chicken | of chicken | Cell line |
| Virus | avian MXRA8 | MXRA8 | MXRA8-Fc | MXRA8 | used |
| SINV | ✓ | x | ✓ | ✓ | 3T3, CEFs, |
| | | | | | 293T, HeLa, |
| | | | | | Jurkat |
| WEEV | ✓ | x | ✓ | — | 3T3, CEFs |
| WHAV | ✓ | x | ✓ | — | 3T3, CEFs |
| BABV | ✓ | x | ✓ | — | 3T3, CEFs |
| OCKV | ✓ | x | ✓ | ✓ | 3T3, CEFs, |
| AURA | x | x | x | x | 3T3, CEFs, |
| | | | | | 293T, HeLa |
| CHIKV | x | ✓ | — | — | 3T3, CEFs, |
| 181/25 | | | | | 293T, HeLa, |
| | | | | | Jurkat |
| CHIKV | x | ✓ | — | — | 293T |
| 37997 | | | | | |
| SINV- | x | x | — | x | 3T3, K562 |
| EEEV | | | | | |
| SINV- | x | x | — | — | 3T3, Jurkat |
| VEEV | | | | | |

✓, yes; x, no; — , not tested.

For AURAV, neither mouse nor chicken MXRA8 enabled infection in mammalian 3T3, 293T or HeLa cells or in CEFs (see e.g., TABLE 6, FIG. 42A-FIG. 42M, and FIG. 43A-FIG. 43N).

Chicken MXRA8 Expression in Mice Enhances SINV Infection In Vivo.

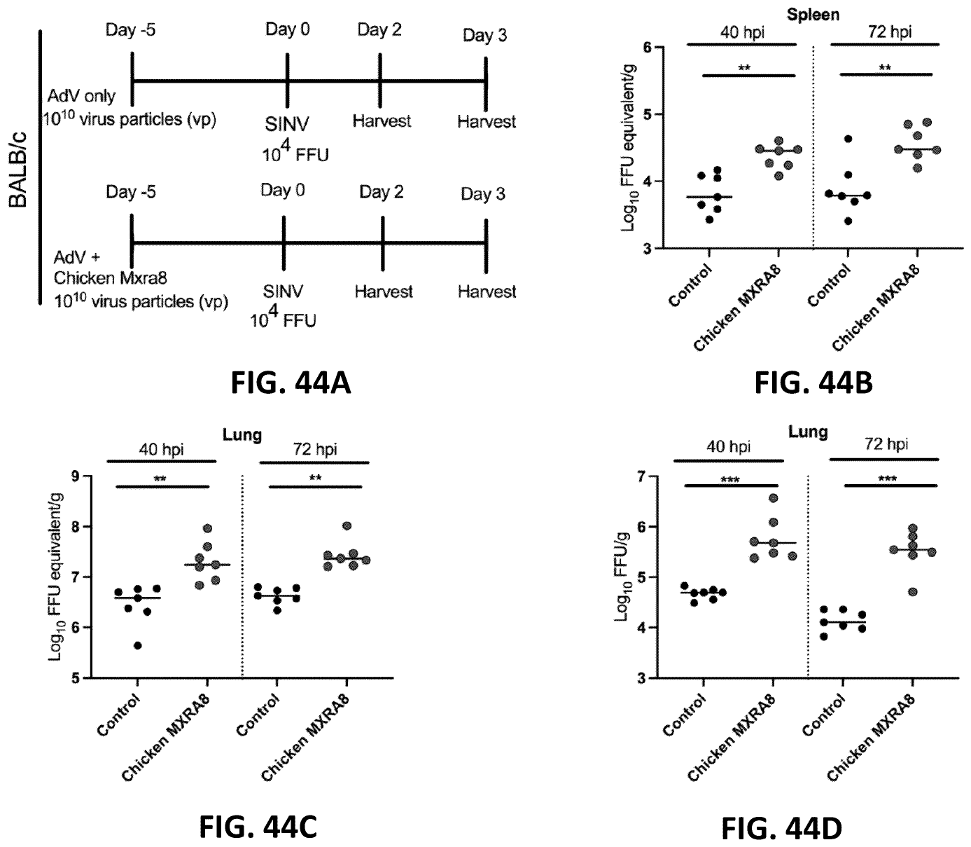

Since genetic studies are not easily performed in avian hosts, the impact of chicken MXRA8 on SINV infection was evaluated in vivo by transiently expressing it in mice using an adenoviral vector. Five-week-old BALB/c mice were inoculated via an intranasal route with 10¹⁰ viral particles of a replication-defective human adenovirus encoding chicken MXRA8 or an empty vector (see e.g., FIG. 44A). Five days later, mice were inoculated via an intranasal route with 10⁴ focus-forming units (FFU) of SINV that was preceded by administration of anti-IFNAR1 mAb one day earlier to facilitate virus dissemination. At 40 and 72 h post-inoculation, SINV RNA and infectious virus levels were higher in the spleen and lungs of mice transduced with chicken MXRA8 than animals receiving the empty adenoviral vector (see e.g., FIG. 44B-FIG. 44D). Thus, avian MXRA8 can functionally enhance SINV infection in vivo.

Duck MXRA8 Binds WEEV in an Inverted Orientation Compared to Mammalian MXRA8 and CHIKV.

To determine how WEE and SF complex members respectively bind avian and mammalian MXRA8, cryo-EM was used to obtain a reconstruction of duck MXRA8 bound to WEEV-VLPs (see e.g., TABLE 7) and these data were compared to structures of mouse and human MXRA8 bound to CHIKV-VLPs.

TABLE 7
Cryo-EM model building and refinement statistics.

| | | | CHIKV-VLP | WEEV-VLP + |
|---|---|---|---|---|
| | WEEV-VLP alone | WEEV-VLP + duck | Du-D1-Mo-D2 | Du-D1-Mo-D2 |
| | (EMDB-27272 | Mxra8 (EMDB-27271 | (EMDB-28644 | (EMDB-40711 |
| Asymmetric Unit Model | PDB: 8DAQ) | PDB: 8DAN) | PDB: 8EWF) | PDB: 8SQN) |
| # of chains | 8 | 12 | 9 | 9 |
| # of residues | 3412 | 4472 | 3697 | 3677 |
| # of carbohydrates | 8 | 12 | 9 | 9 |
| Resolution (Å) | 4.35 | 4.74 | 3.96 | 3.89 |
| MolProbity Score | 1.98 | 1.84 | 2.25 | 1.91 |
| All-atom clash score | 13.52 | 10.23 | 21.25 | 12.07 |
| Rotamer outliers (%) | 0.00 | 0.00 | 0.00 | 0.00 |
| Cβ outliers | 0.00 | 0.00 | 0.00 | 0.00 |
| Ramachandran Plot | | | | |
| Values | | | | |
| Favored (%) | 95.05 | 95.50 | 93.42 | 95.52 |
| Allowed (%) | 4.95 | 4.50 | 6.58 | 4.48 |
| Outlier (%) | 0.00 | 0.00 | 0.00 | 0.00 |
| R.M.S. Deviations | | | | |
| Bond lengths (Å) | 0.003 | 0.002 | 0.004 | 0.003 |
| Bond angles (°) | 0.554 | 0.517 | 0.679 | 0.598 |
| Correlation Coefficient | 0.75 | 0.68 | 0.71 | 0.72 |

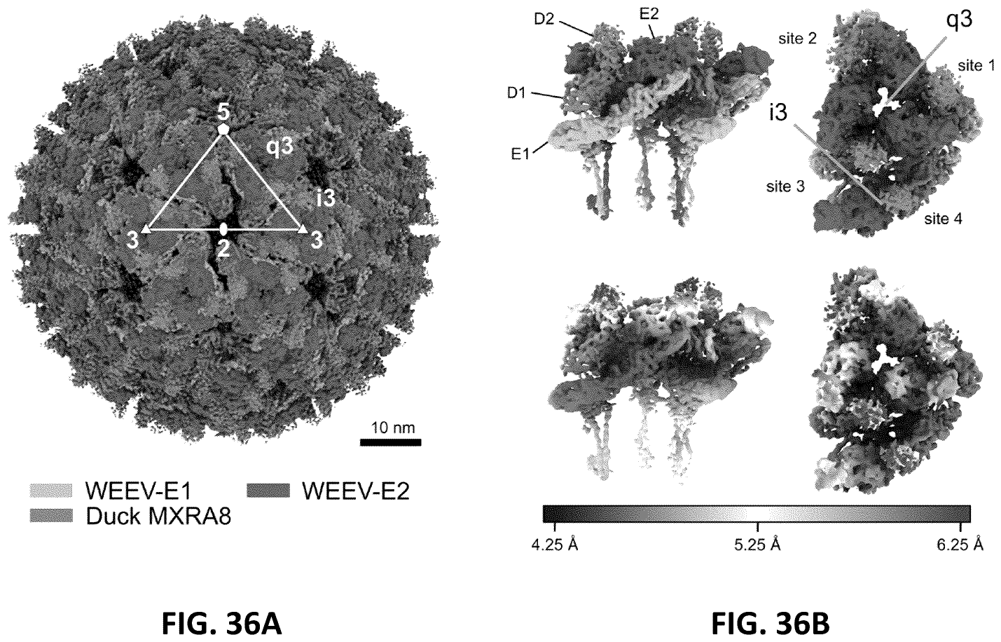

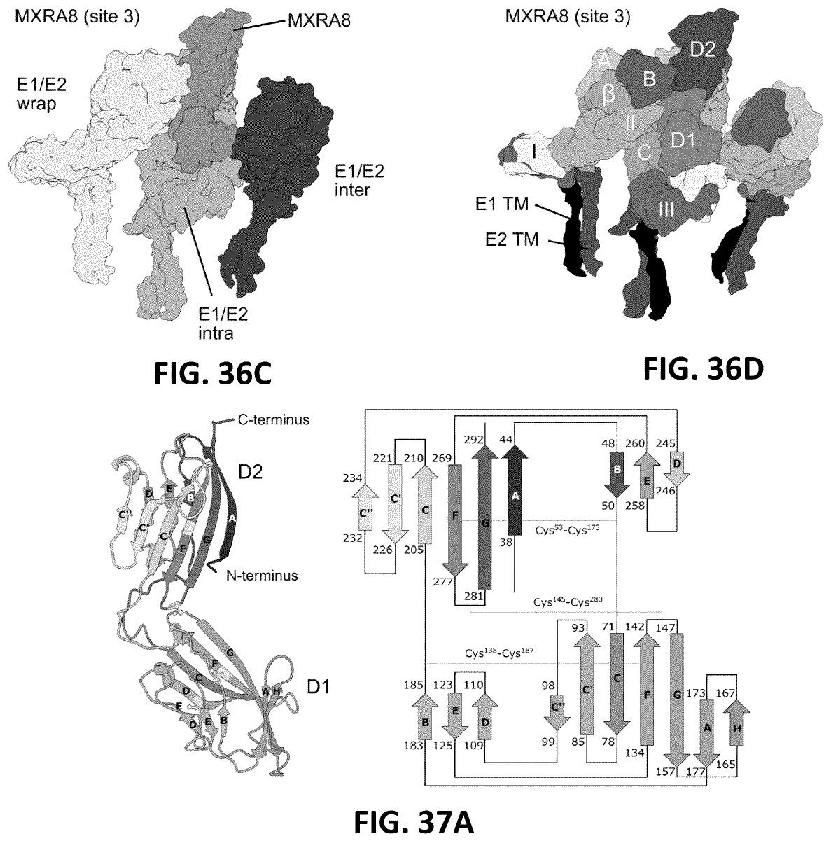

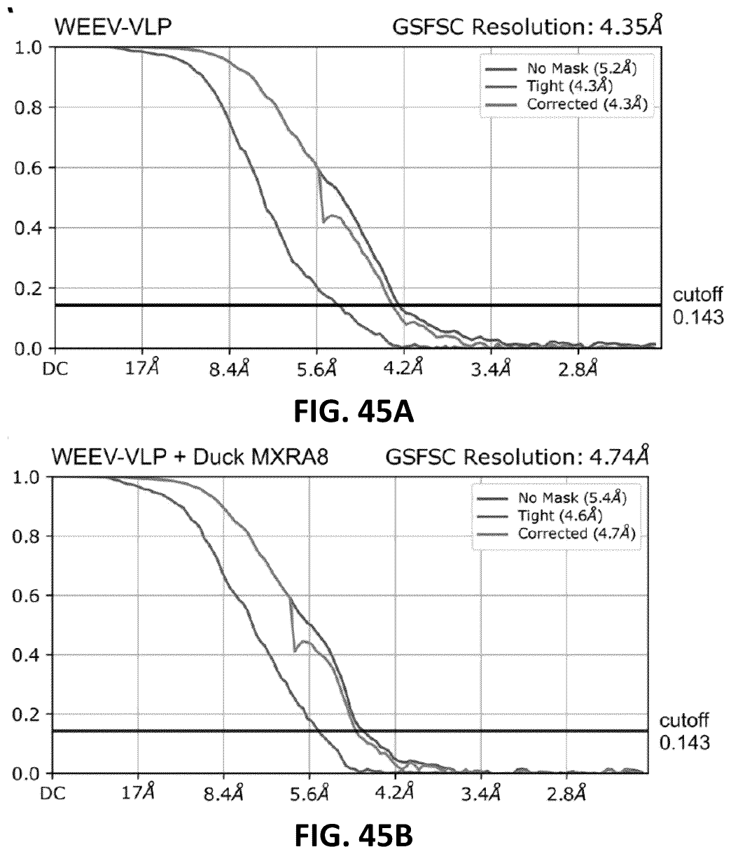

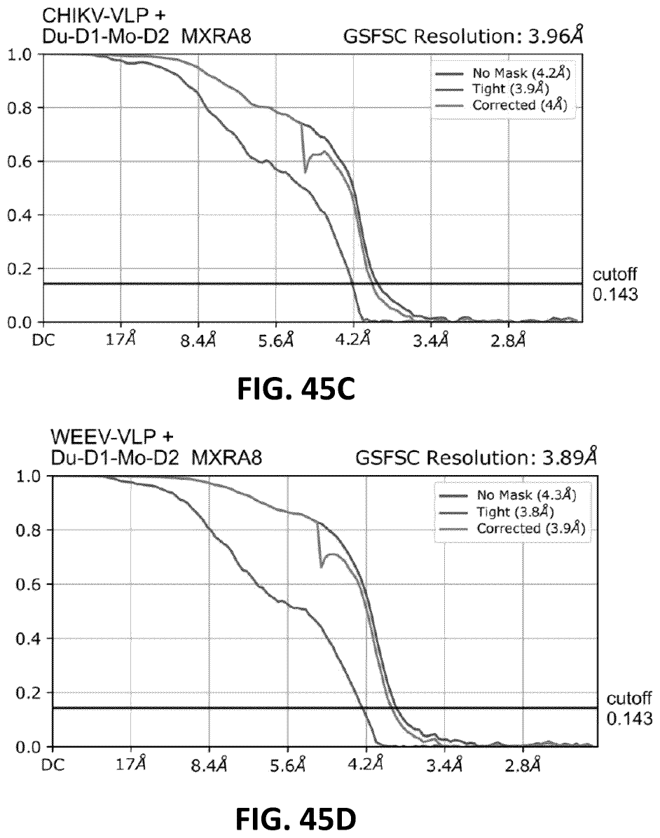

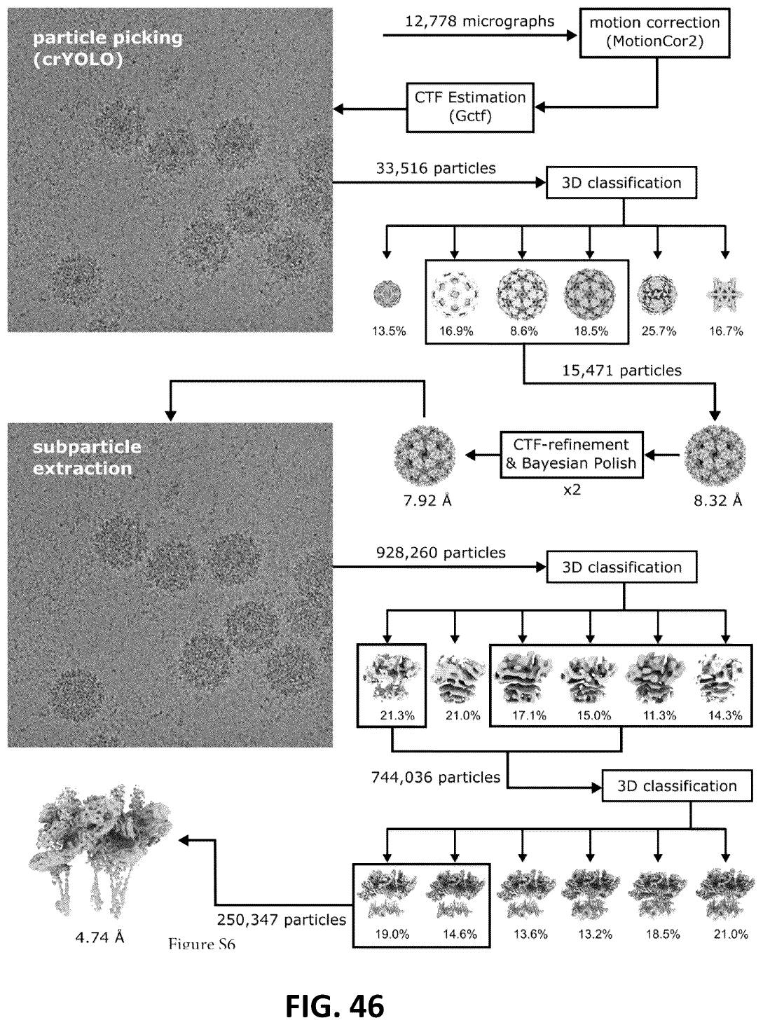

As expected, the WEEV-VLP structural proteins exhibited T=4 icosahedral symmetry with 60 asymmetric units (see e.g., FIG. 36A). Each virion has 240 E1-E2 heterodimers arranged into 80 trimeric spikes, located around icosahedral threefold (“i3”, n=20) and quasi-threefold (“q3”, n=60) axes. Each asymmetric unit contains a q3 spike and a single i3 E1-E2 heterodimer, with MXRA8 molecules occupying four unique binding sites in each unit, conventionally labeled as sites 1 through 4 (see e.g., FIG. 36B). To account for deviations from ideal icosahedral symmetry, these asymmetric units were extracted for focused refinement, achieving a final resolution of 4.74 Å (see e.g., FIG. 45A-FIG. 45D and FIG. 46). Local resolution ranged from ˜5.0 to 6.0 Å for the transmembrane helices and the MXRA8 domain most distal from the virus to ˜4.0 to 4.5 Å for the remainder of the structure. Similar to the binding interface of mouse MXRA8 and CHIKV, each duck MXRA8 molecule has a complex quaternary binding mode, with three different heterodimer interactions: (1) wrapped; interacting laterally against one E1-E2 heterodimer, near the E1 fusion loop, (2) intraspike; engaging the neighboring E1-E2 heterodimer within a trimeric spike, and (3) interspike, binding across different E1-E2 trimeric spikes (see e.g., FIG. 36C-FIG. 36D).

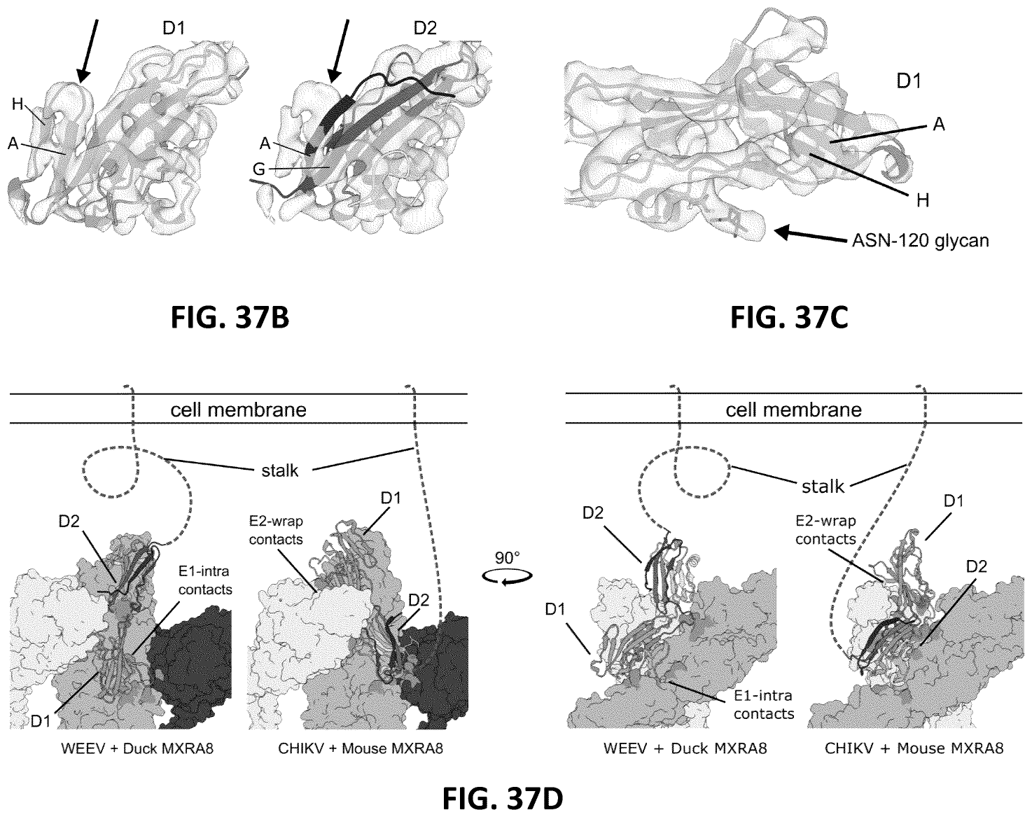

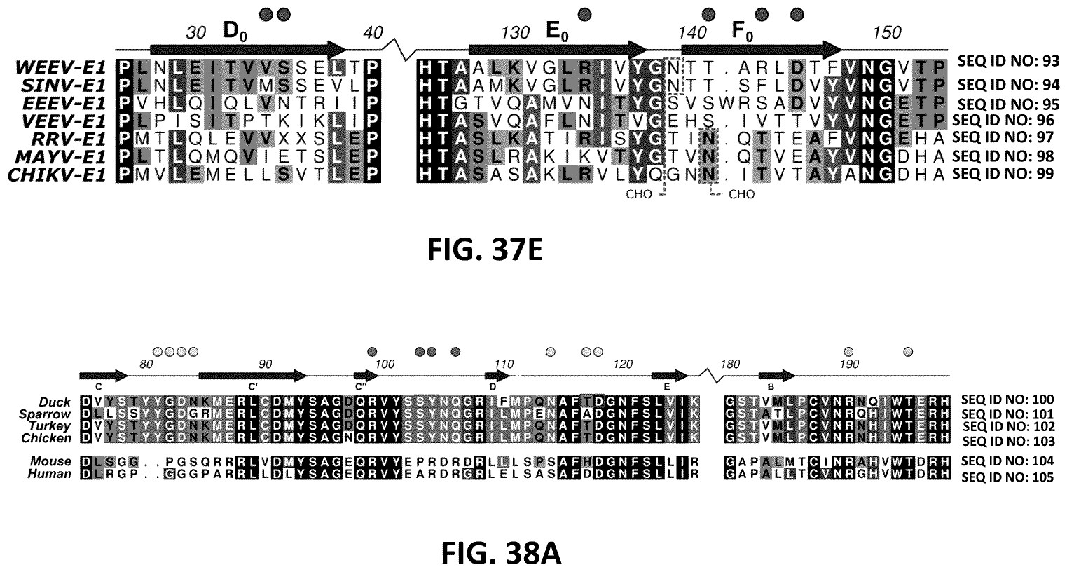

It was hypothesized that duck MXRA8 would bind WEEV in an orientation similar to that of mammalian MXRA8 and CHIKV. Unexpectedly, the binding mode of duck MXRA8 to WEEV is inverted with respect to how mammalian MXRA8 binds to CHIKV (see e.g., FIG. 37A-FIG. 37D and TABLE 8).

TABLE 8
List of contact residues of duck MXRA8 (bound to WEEV-VLP),
Du-D1-Mo-D2 (bound to WEEV-VLP), mouse MXRA8 (bound to
CHIKV-VLP), or Du-D1-Mo-D2 MXRA8 (bound to CHIKV-VLP).

| | | Duck Mxra8 | | Mouse Mxra8 | Du-D1-Mo-D2 |
|---|---|---|---|---|---|
| | | (+WEEV) | Du-D1-Mo-D2 | (+CHIKV) | (+CHIKV) |
| E1-E2 | MXRA8 | residues | (+WEEV) | residues | residues |
| heterodimer | domain | PDB: 8DAN | residues | PDB: 6NK6 | PDB: 8EWF |
| Wrap | Domain-I | N114(1), | T117(1), | Q83(1), | M86(5), |
| | | T117(8), | R190(8), | R84(16), | R88(2), |
| | | D118(20), | R197(4) | R86(2), | Y93(4), |
| | | R190(12), | | Y91(1), | A95(1), |
| | | T195(2) | | A93(1), | G96(2), |
| | | | | G94(1), | D97(17), |
| | | | | E95(7), | R99(1), |
| | | | | R97(14), | H141(2), |
| | | | | H139(3), | Y148(12) |
| | | | | H141(2), | |
| | | | | H141(1), | |
| | | | | D146(4) | |
| | Domain-II | None | None | P56(3), | P56(5), |
| | | | | R57(8), | V59(1), |
| | | | | V59(2), | D63(7), |
| | | | | T61(1), | R64(9), |
| | | | | D63(14), | L65(27), |
| | | | | R64(5), | N66(1), |
| | | | | L65(5), | A252(2), |
| | | | | H66(5), | R253(15), |
| | | | | N247(1), | D255(3) |
| | | | | A250(4), | |
| | | | | R251(9), | |
| intra | Domain-I | Y81(5), | A95(9), | H115(2), | E196(1) |
| | | A95(1), | G96(2), | D194(5), | |
| | | R99(7), | S103(5), | H196(21), | |
| | | S103(6), | Y104(18), | E198(7) | |
| | | Y104(16), | Q106(2), | | |
| | | Q106(4), | R108(1), | | |
| | | T117(1), | T117(2), | | |
| | | A129(2), | D127(2), | | |
| | Domain-II | R64(30), | D63(1), | Q62(1), | E201(3), |
| | | L65(3), | R64(4), | P236(2), | S229(7), |
| | | E201(3) | L65(9), | D240(1), | F239(11) |
| | | | N66(11), | E260(1) | |
| | | | E201(4) | | |
| inter | Domain-I | Y81(2), | D83(6) | None | None |
| | | G82(13), | | | |
| | | D83(7), | | | |
| | | N84(2) | | | |
| | Domain-II | None | None | L210(3), | None |
| | | | | P211(5), | |
| | | | | G212(5), | |
| | | | | S214(1), | |
| | | | | F237(2), | |
| | | | | R263(12), | |
| | | | | A264(4), | |
| | | | | E266(3) | |

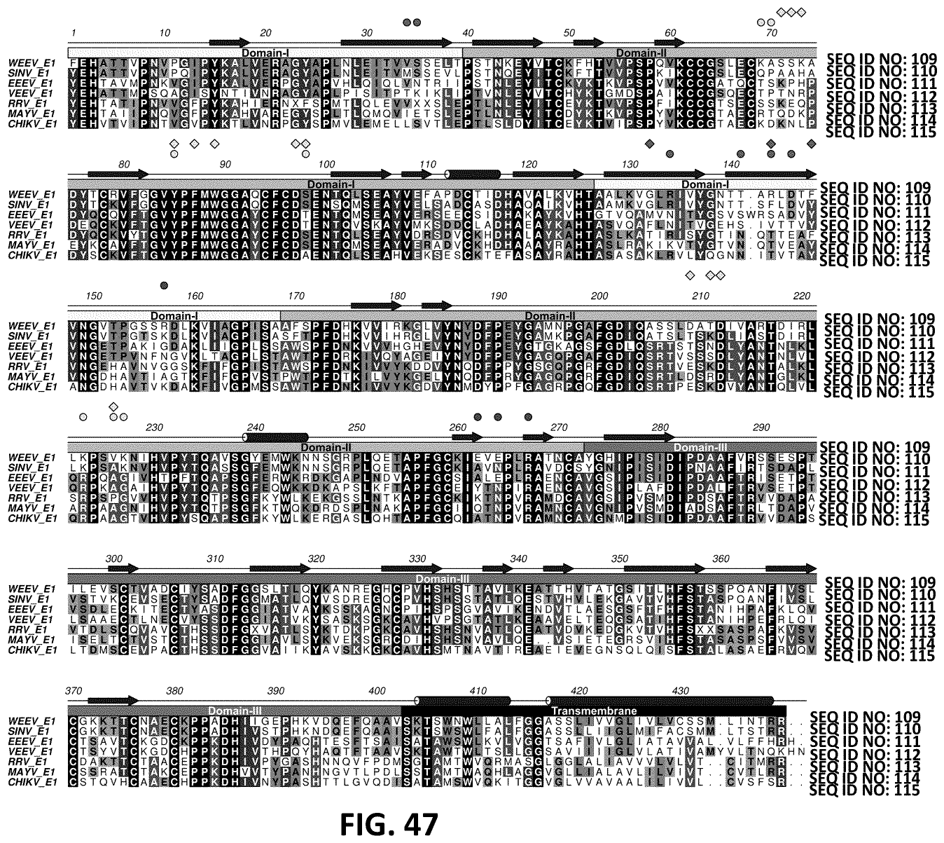

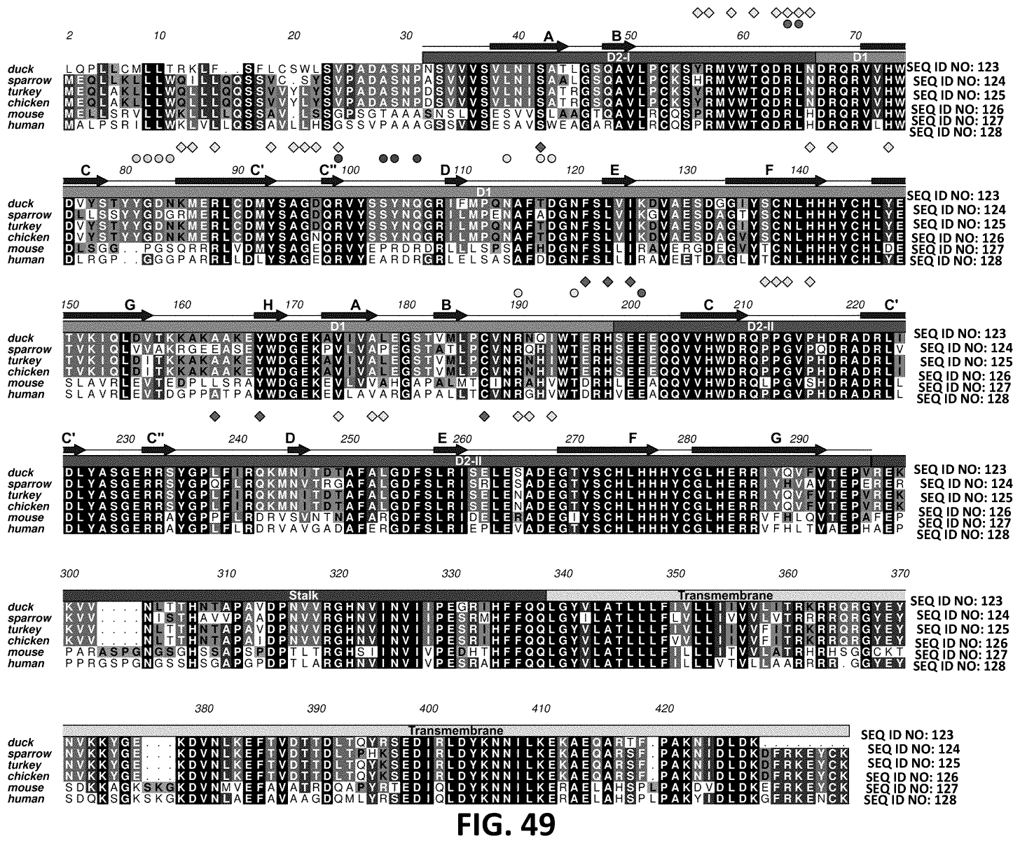

Close-contacts between atomic coordinates are defined as the heavy atoms within 0.39 nm. Number of contacts per residue are indicated within parentheses.

Whereas domain 1 (D1) of mammalian MXRA8 predominantly contacts CHIKV E2, D1 of duck MXRA8 contacts E1 of WEEV, forming interactions with the wrapped (E1 domain-II), intraspike (E1 domain-I), and interspike (E1 domain-II) heterodimers (see e.g., FIG. 47-FIG. 49 and TABLE 9).

TABLE 9
List of contact residues of WEEV-VLP bound to duck MXRA8 (PDB:
8DAN) or Du-D1-Mo-D2 MXRA8 (PDB: 8SQN) and CHIKV-VLP bound
to mouse MXRA8 (PDB: 6NK6) or Du-D1-Mo-D2 (PDB: 8EWF).

| E1-E2 | E1-E2 | WEEV residues | WEEV residues | CHIKV residues | CHIKV residues |
|---|---|---|---|---|---|
| heterodimer | domain | (duck MXRA8) | (Du-D1-Mo-D2) | (mouse MXRA8) | (Du-D1-Mo-D2) |
| Wrap | E1 | Y85(27), | Y85(8), | Y85(8), | Y85(2), |
| | Domain-II | S98(3), | S225(1), | F87(5), | F87(3), |
| | | K223(1), | K227(4) | W89(1), | D97(16) |
| | | V226(10), | | D97(2), | |
| | | K227(2) | | A98(1), | |
| | | | | A226(1) | |
| | E2 | None | None | H18(1), | C22(2), |
| | Domain- | | | E24(1), | G23(1), |
| | A | | | H26(6), | E24(7), |
| | | | | S27(14), | H26(33), |
| | | | | C28(4), | C28(1), |
| | | | | H29(3), | D71(3), |
| | | | | S72(1), | R119(15), |
| | | | | T74(7), | K120(6), |
| | | | | R119(12), | I121(1) |
| | | | | K120(2), | |
| | | | | I121(2) | |
| | E2 | None | None | R178(2), | T179(2), |
| | Domain- | | | T179(5), | T182(6), |
| | B | | | M181(1), | Q183(4), |
| | | | | T182(3), | Q184(2), |
| | | | | T191(5), | N193(5), |
| | | | | V192(1), | D214(2), |
| | | | | N193(5), | D223(5) |
| | | | | T213(1), | |
| | | | | D214(7), | |
| | | | | I222(1), | |
| | | | | D223(8) | |
| intra | E1 | V34(1), | V34(4), | K132(1), | S130(1), |
| | Domain-I | S35(4), | S35(1), | T143(1), | K132(2), |
| | | R134(1), | K130(2), | Y147(2) | A145(6) |
| | | T141(1), | R134(3), | | |
| | | R143(16), | R143(12), | | |
| | | D145(5), | D145(6) | | |
| | | R157(5) | | | |
| | E1 | E262(5), | K69(4), | None | None |
| | Domain-II | E264(1), | A70(2) | | |
| | | R267(1) | | | |
| | E2 | D7(3) | None | W64(7) | W64(1) |
| | Domain- | | | | |
| | A | | | | |
| | E2 β- | L152(1), | L152(2), | V157(2), | V157(2), |
| | linker | L161(3), | K154(8), | Q158(15), | S159(1), |
| | | E163(16), | D159(1), | S159(11), | V264(2), |
| | | T164(4), | L161(7), | T265(1) | T265(5) |
| | | S165(2), | S165(1), | | |
| | | H259(7), | R263(6), | | |
| | | R263(1), | L264(1), | | |
| | | V268(1) | T265(6), | | |
| | | | P266(3), | | |
| | | | T267(4), | | |
| | | | V268(3) | | |
| inter | E1 | K69(8), | K69(4), | K71(2), | None |
| | Domain-II | A70(16) | A70(2) | S72(5), | |
| | | | | L73(2), | |
| | | | | E209(1), | |
| | | | | K211(14), | |
| | | | | D212(5) | |
| | E2 β- | None | None | A164(4), | None |
| | linker | | | E165(1), | |
| | | | | E166(1) | |

Close-contacts between atomic coordinates are defined as the heavy atoms within 0.39 nm. Number of contacts per residue are indicated within parentheses.

Additionally, although D2 of mammalian MXRA8 makes substantial contacts with E1 of CHIKV, D2 of duck MXRA8 contributes minimally to WEEV binding. Thus, duck and mouse MXRA8 appear to engage with a domain-inverted binding mode using both distinct MXRA8 domains and different alphavirus structural proteins.

Given this unanticipated finding, the accuracy of the atomic model and the inverted binding modes of avian and mammalian MXRA8 was assessed using multiple methods. Although the substantial pseudo-symmetry between D1 and D2 of duck MXRA8 makes determination of domain orientation challenging, particular domain-specific features support the binding model. First, there is an extra β-strand (strand-H) in D1 of duck MXRA8, antiparallel to strand-A. Second, duck MXRA8 domains have different N-linked glycosylation patterns: D2 has two N-linked glycans (Asn-40 and Asn-245) and D1 has one (Asn-120), each located within different topological regions. Because of these differences, the duck

MXRA8 density in the binding-groove of WEEV is fit better by D1, featuring clear density for strand-H and a single N-linked glycan near Asn-120 (see e.g., FIG. 37B-FIG. 37C).

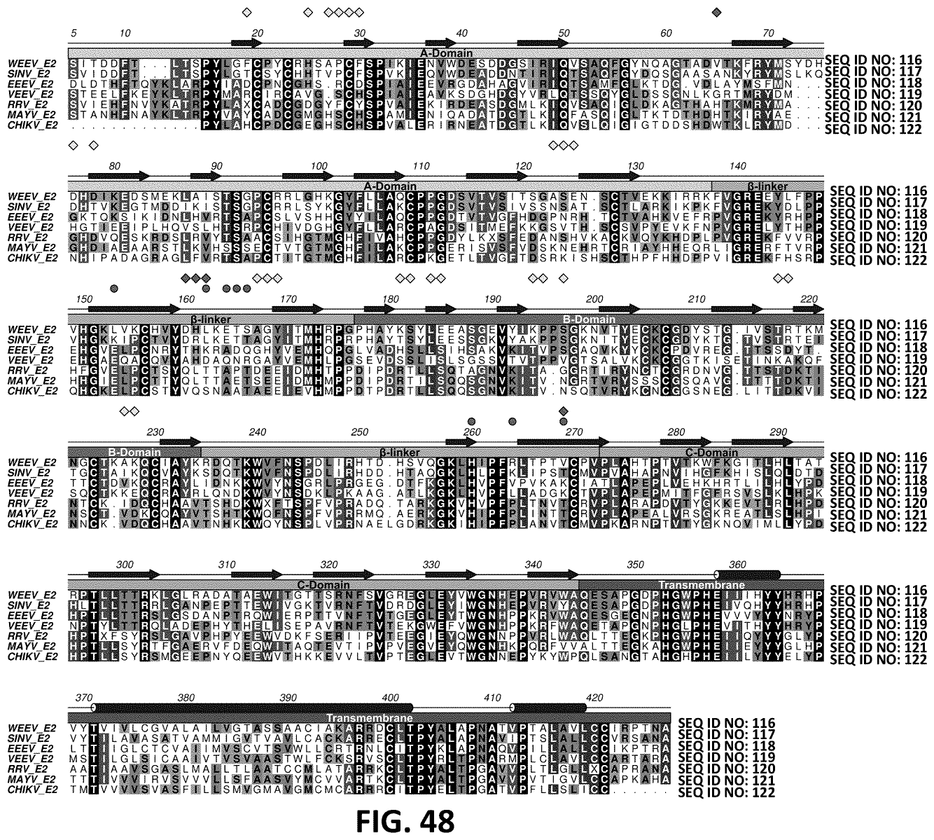

Sequence alignments of the alphavirus contact residues were performed to elucidate why WEE and SF complex members show unique MXRA8 species-specific binding. At regions predicted to be sites of contact with duck MXRA8, E1 amino acid sequences of WEE complex members (WEEV and SINV) vary from SF complex members (CHIKV, MAYV, and RRV), and VEEV/EEEV (see e.g., FIG. 37E and FIG. 47) with an average sequence identity of only 27.9% for segments within 3 residues of the contact sites, compared to 52.5% for the entire E1 sequence. Analogously, at predicted sites of contact with mouse MXRA8, there is substantial sequence variation in regions of E2 of WEE and SF complex members (see e.g., FIG. 48) with an average sequence identity of 30.9% for segments within 3 residues of the contact sites. These sequence features might explain why mammalian MXRA8 does not bind to the analogous region on WEEV E1-intraspike heterodimer.

Genetic Assessment of the Atomic Model.

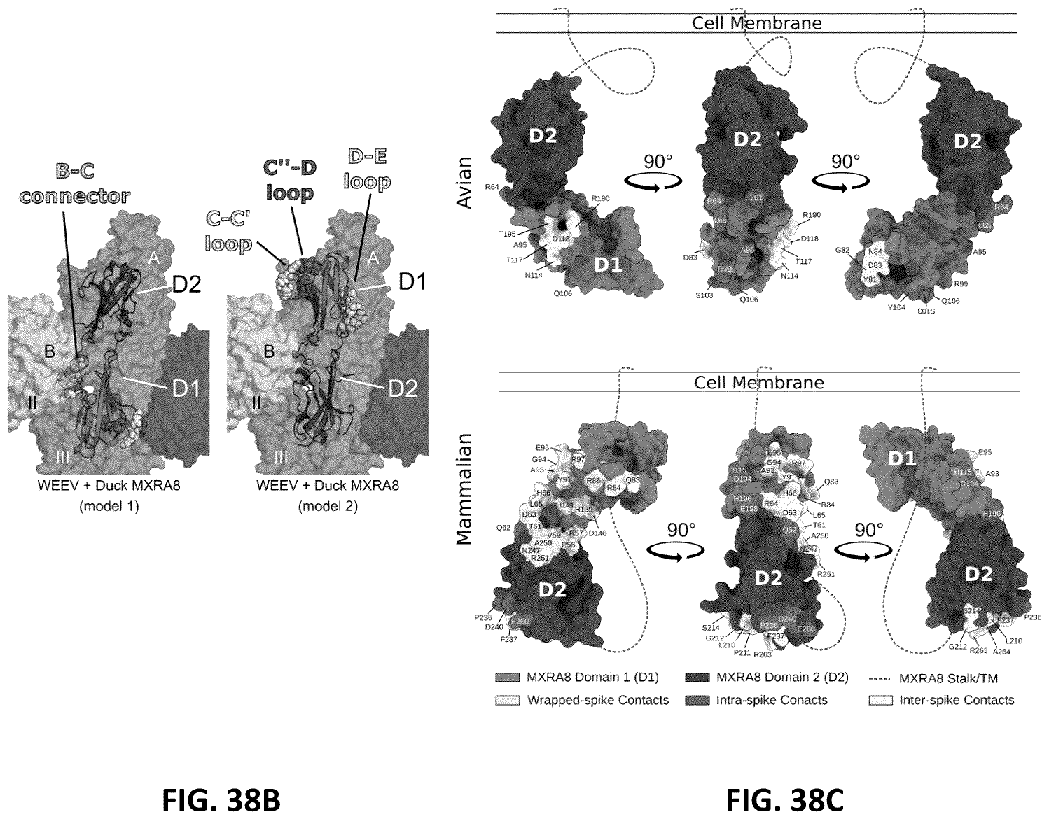

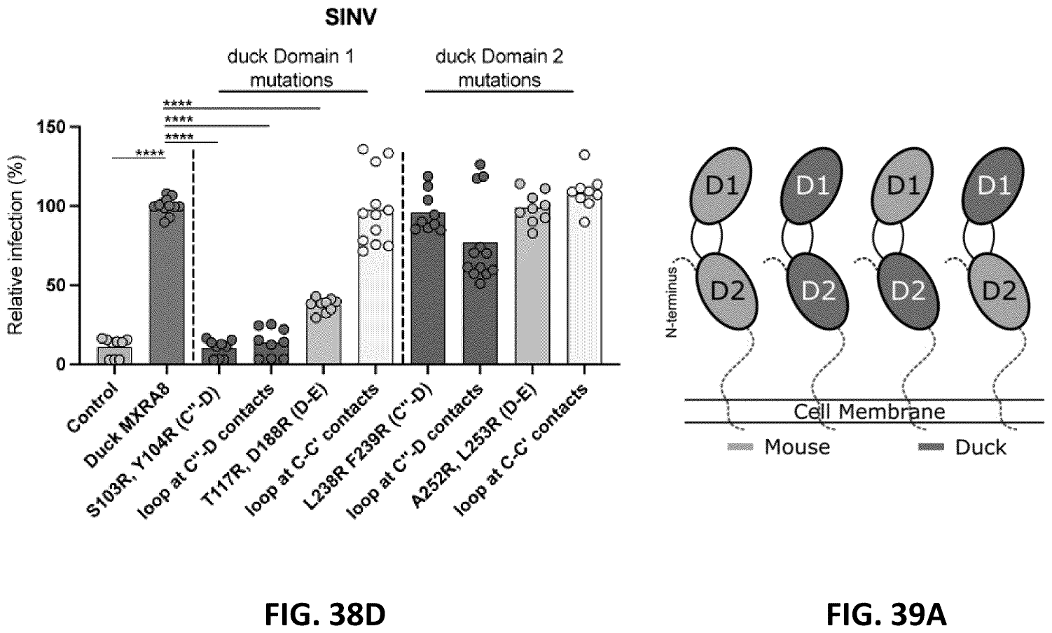

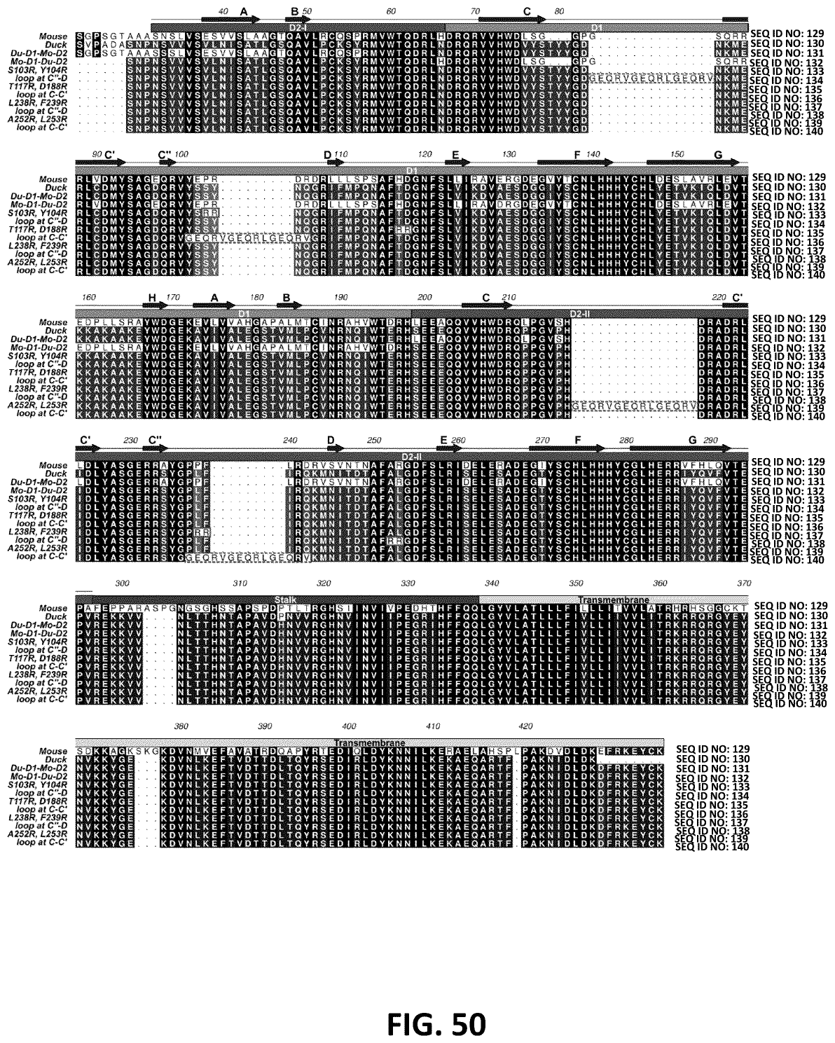

Based on the cryo-EM data, duck MXRA8 D1 makes primary contact with WEEV E1 at four distinct sites: C-C′ loop (interspike), C″-D loop (intraspike), and D-E loop and B-C connector (wrapped) (see e.g., FIG. 38A-FIG. 38C). To evaluate the structural model, MXRA8 mutations and insertions in these regions were designed that should disrupt binding to WEEV and attenuate the ability of duck MXRA8 to support WEE complex virus infection. ΔMxra8 3T3 cells were complemented with duck MXRA8 encoding a set of double arginine mutations or 15 amino-acid insertions at the interface of predicted contact sites at the D-E loop, C″-D loop, and C-C′ loop in either D1 or D2 (see e.g., FIG. 38A-FIG. 38B and FIG. 50). ΔMxra8 3T3 cells complemented with the different duck MXRA8 mutants were inoculated with SINV and analyzed for infection by flow cytometry. As predicted by the model, mutations or insertions in duck D1 (C″-D loop insertion, C″-D loop mutations [S103R/T1041R], and D-E loop mutation [T117R/D188R]) but not duck D2 resulted in reduced SINV infection (see e.g., FIG. 38B-FIG. 38D). However, the C-C′ loop insertion in duck MXRA8 D1 or D2, which corresponds to a loop insertion in bovine MXRA8 D1 that blocks CHIKV infection, had no effect on SINV infection, indicating that the E1 interspike contacts, which are critical for MXRA8-dependent infections by SF complex members, are less consequential for MXRA8-dependent infection by WEE complex members (see e.g., FIG. 38D).

Chimeric Duck-Mouse MXRA8 Supports Infection of Alpha Viruses from Both SFV and WEE Complexes.

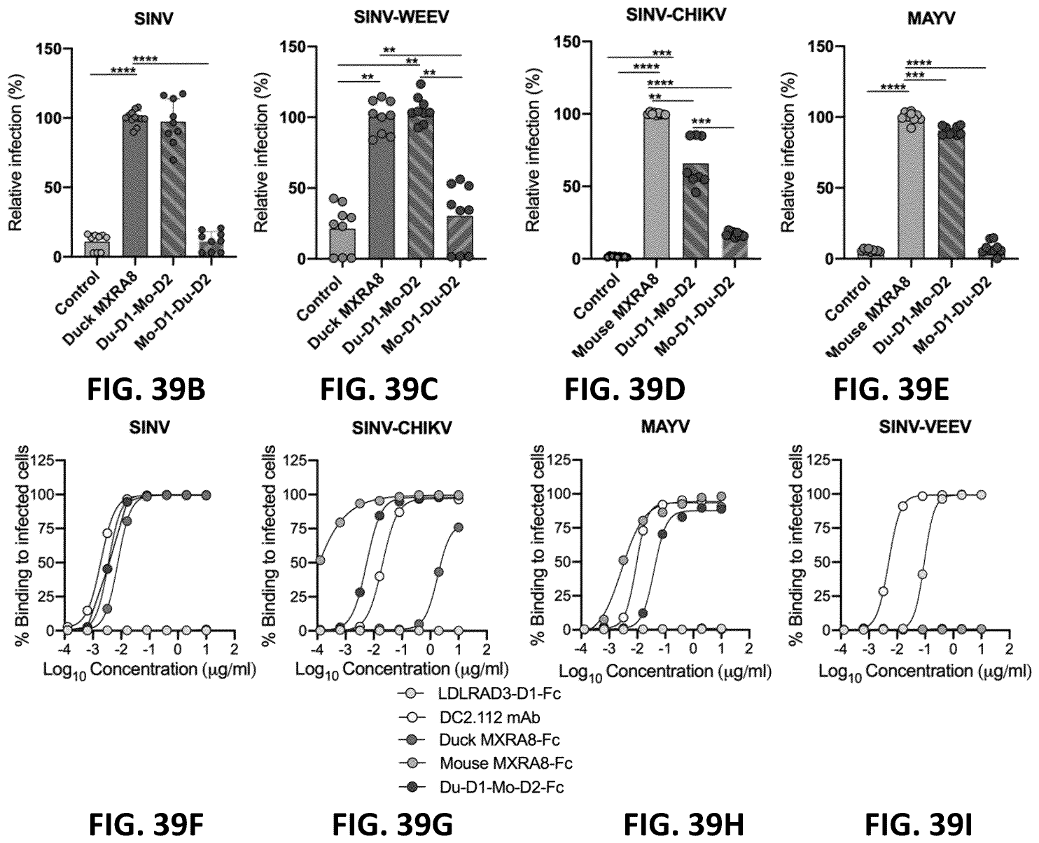

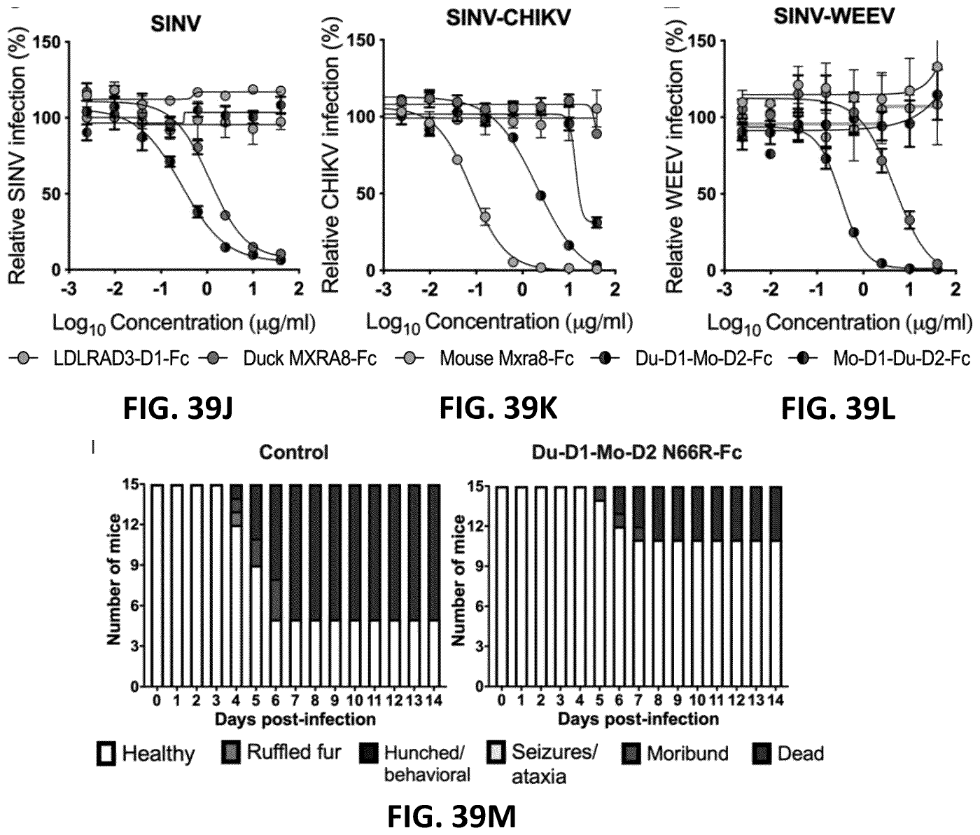

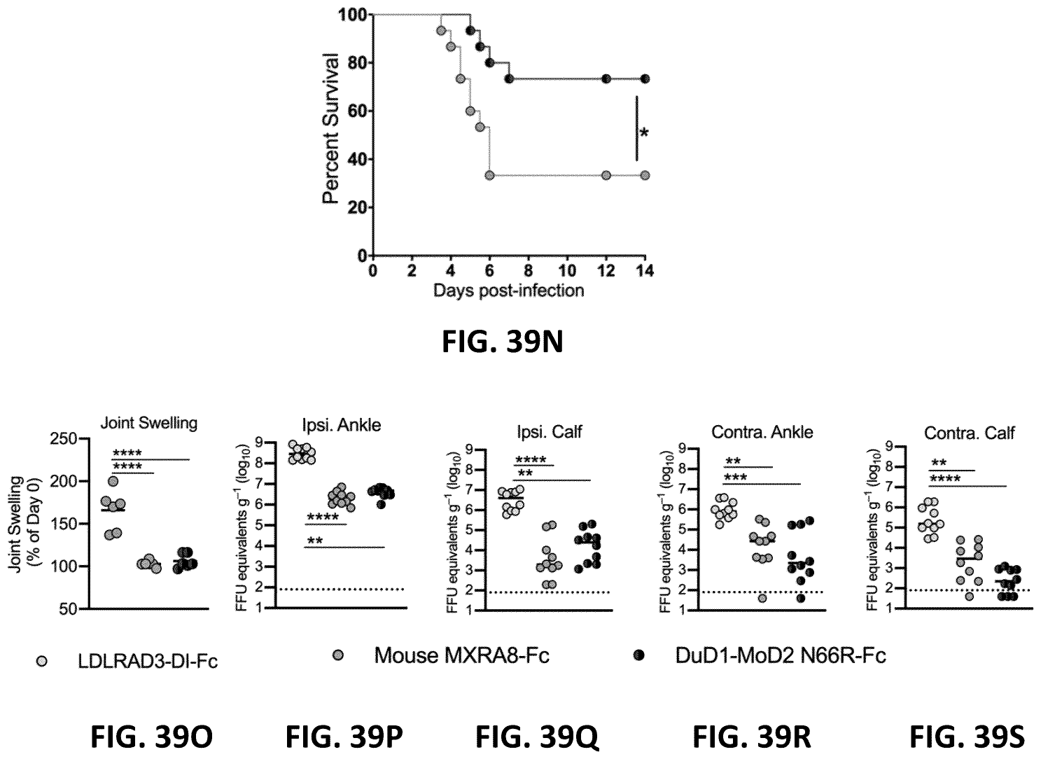

As a complementary experiment, chimeric domain-swapped MXRA8 proteins were engineered (see e.g., FIG. 39A and TABLE 10).

TABLE 10
Properties of chimeric protein Du-D1-Mo-D2 and Mo-
D1-Du-D2 MXRA8 receptor activity for alphaviruses.

| | | | Binding of | | |
|---|---|---|---|---|---|
| | Infection enhanced | Infection enhanced | Du-D1-Mo-D2 | Inhibition | In vivo |
| | by cell surface | by cell surface | MXRA8-Fc | of infection | protection by |
| | expression of | expression of | to surface | by Du-D1- | Du-D1-Mo-D2 |
| | Du-D1-Mo-D2 | Mo-D1-Du-D2 | of infected | Mo-D2 | N66R |
| Virus | MXRA8 | MXRA8 | cells | MXRA8-Fc | MXRA8-Fc |
| SINV | ✓ | x | ✓ | ✓ | — |
| SINV- | ✓ | x | ✓ | ✓ | ✓ |
| WEEV | | | | | |
| SINV- | — | — | x | — | — |
| VEEV | | | | | |
| SINV- | ✓ | x | ✓ | ✓ | ✓ |
| CHIKV | | | | | |
| MAYV | ✓ | x | ✓ | — | — |

✓, yes; x, no; —, not tested.

This approach was used because expressing isolated MXRA8 single domains (D1 or D2) was unsuccessful, possibly because of the unique strand-swapped domain topology. Because mouse MXRA8 does not bind WEEV, the model predicts that when D1 of duck is replaced with D1 of mouse MXRA8, binding to WEEV should be abrogated. ΔMxra8 3T3 cells complemented with the duck-mouse (Du-D1-Mo-D2) and mouse-duck (Mo-D1-Du-D2) MXRA8 chimera, both with duck MXRA8 stalk sequences, were inoculated with SINV, SINV-WEEV, SINV-CHIKV, and MAYV. While similar levels of SINV and SINV-WEEV infection were observed in cells complemented with Du-D1-Mo-D2 and wild-type duck MXRA8, substantially less infection was detected in cells expressing Mo-D1-Du-D2 MXRA8 (see e.g., FIG. 39B-FIG. 39C). Unexpectedly, when cells were inoculated with SINV-CHIKV and MAYV, two viruses that use mammalian but not avian MXRA8, high levels of infection were detected in cells expressing Du-D1-Mo-D2 but not Mo-D1-Du-D2 MXRA8 (see e.g., FIG. 39D-FIG. 39E). These experiments with Du-D1-Mo-D2 establish that D1 of duck MXRA8 is compatible with SF complex virus binding and infection when expressed with D2 of mouse MXRA8. Indeed, 8 of the 14 residues in D1 of mouse MXRA8 that contact CHIKV are conserved in D1 of duck MXRA8. The lack of infection with Mo-D1-Du-D2 MXRA8 and CHIKV suggest either a requisite role of D2 of mouse MXRA8 in facilitating binding to CHIKV or an inhibitory role of D2 of duck MXRA8 in preventing engagement of CHIKV.

To corroborate the infection patterns of chimeric MXRA8 using an orthogonal assay, Vero cells were inoculated with SINV, CHIKV, MAYV, or SINV-VEEV and stained the surface of infected cells, which display viral E1-E2 proteins prior to virion morphogenesis and budding, with mouse MXRA8-Fc, duck MXRA8-Fc, Du-D1-Mo-D2 MXRA8-Fc, or LDLRAD3 D1-Fc fusion proteins (see e.g., FIG. 39F-FIG. 39I and TABLE 10). Whereas mouse MXRA8-Fc showed dose-dependent binding to cells infected with CHIKV and MAYV (see e.g., FIG. 39G-FIG. 39H), duck MXRA8-Fc bound avidly only to SINV-infected cells (see e.g., FIG. 39F), and neither bound to cells expressing VEEV structural proteins (see e.g., FIG. 39I). In contrast to data showing that duck MXRA8 does not support CHIKV infection (see e.g., FIG. 34B), higher (>1 mg/ml) concentrations of duck-MXRA8-Fc bound to the surface of CHIKV-infected cells (see e.g., FIG. 39G); this binding could reflect differential interaction with cell surface-associated (unencapsidated) and virion-associated CHIKV E1-E2 heterodimers, which have different structural features. In comparison, and consistent with infection experiments, Du-D1-Mo-D2 MXRA8-Fc bound to the surface of cells infected with SF and WEE complex viruses but not to those expressing VEEV structural proteins (see e.g., FIG. 39F-391); SINV-VEEV infected cells bound LDLRAD3-D1-Fc (see e.g., FIG. 39I), as expected.

Chimeric Duck-Mouse MXRA8 Inhibits Infection of Alphaviruses from Both SFV and WEE Complexes In Vitro and In Vivo.

Given the broader cell-surface reactivity of Du-D1-Mo-D2 for alphavirus E1-E2 proteins, the inhibitory activity of Du-D1-Mo-D2 MXRA8-Fc and Mo-D1-Du-D2-Fc was assessed against viruses displaying SF and WEE complex structural proteins. SINV TR399, SINV-WEEV, and SINV-CHIKV were pre-incubated with LDLRAD3-D1-Fc, duck MXRA8-Fc, mouse MXRA8-Fc, Du-D1-Mo-D2 MXRA8-Fc, or Mo-D1-Du-D2 MXRA8-Fc and then ΔMxra8 3T3 cells expressing either duck or mouse MXRA8 were inoculated (see e.g., FIG. 39J-FIG. 39L and TABLE 10). Duck and Du-D1-Mo-D2 MXRA8-Fc dose-dependently inhibited infection of SINV TR399 or SINV-WEEV in ΔMxra8 3T3 cells expressing duck MXRA8, whereas mouse MXRA8-Fc, Mo-D1-Du-D2 MXRA8-Fc, and LDLRAD3 D1-Fc did not (see e.g., FIG. 39J-FIG. 39K). However, Du-D1-Mo-D2 MXRA8-Fc showed more potent inhibition (EC50 values) of SINV TR399 (3-fold, P=0.05) and SINV-WEEV (20-fold, P<0.05) infection than duck MXRA8-Fc. Both mouse and Du-D1-Mo-D2 MXRA8-Fc proteins inhibited SINV-CHIKV infection in ΔMxra8 3T3 cells expressing mouse MXRA8, although the potency of mouse-Mxra8-Fc was greater (25-fold, P<0.0001) than Du-D1-Mo-D2 MXRA8-Fc. In comparison, duck MXRA8-Fc and LDLRAD3-D1-Fc did not inhibit SINV-CHIKV infection. Together, these experiments support the cryo-EM-derived flipped binding orientation model (see e.g., FIG. 38B) and show this information can be used to develop decoy receptors that more broadly inhibit infection of alphaviruses.

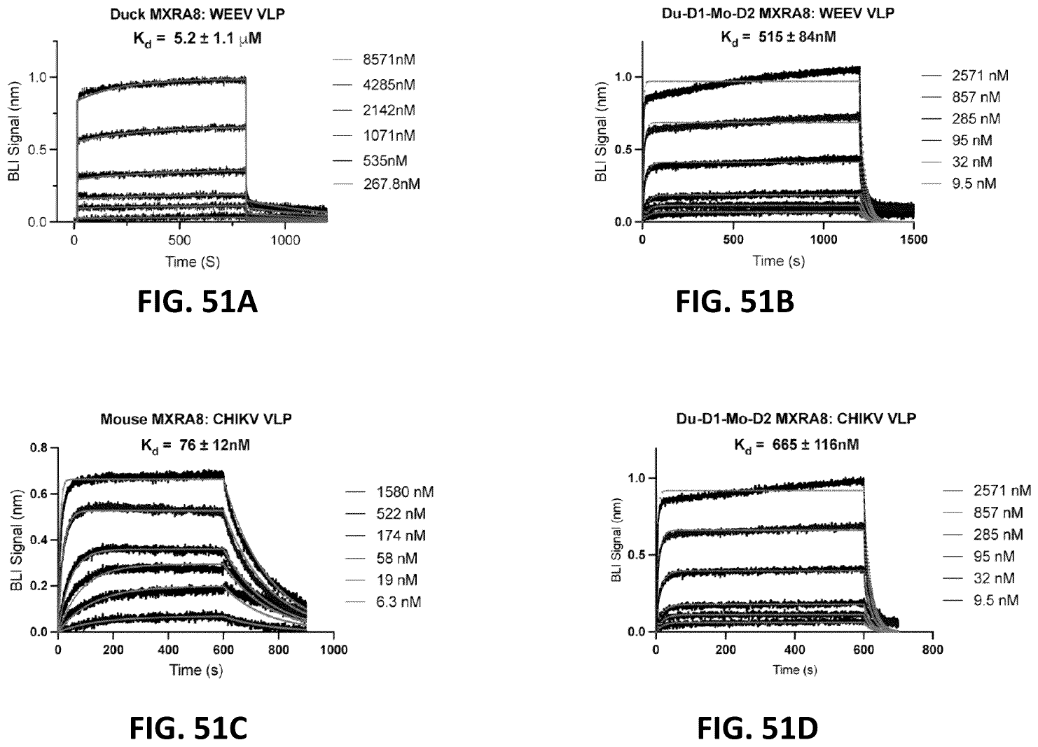

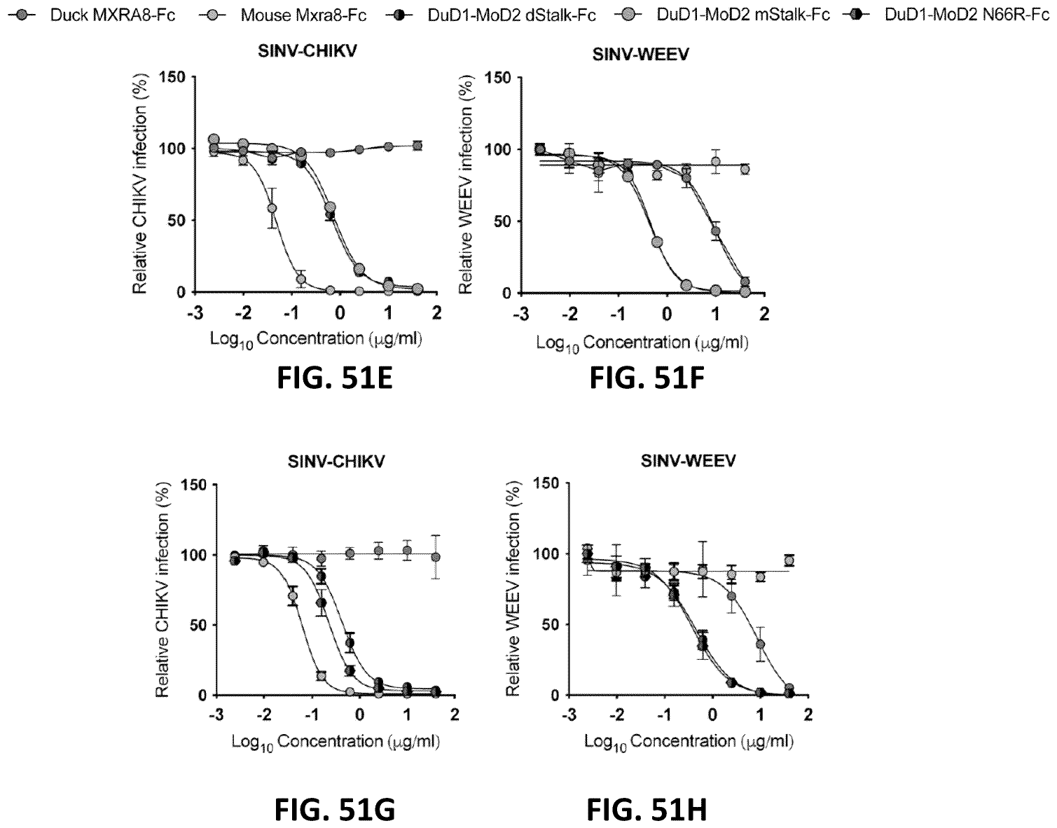

To determine the basis for the different neutralization potencies of the MXRA8 decoy molecules, the affinity (K_{D}) of monomeric MXRA8 for VLPs was measured. Consistent with the neutralization data, chimeric Du-D1-Mo-D2 MXRA8 showed higher affinity binding than duck MXRA8 to WEEV VLPs (K_{D} of 493±6 nM and 5.2±1.1 μM, respectively; FIG. 51A-FIG. 51B), and mouse MXRA8 bound more strongly than Du-D1-Mo-D2 MXRA8 chimera to CHIKV VLPs (K_{D} of 68±17 nM and 545±71 nM, respectively; FIG. 51C-51D). The influence of the stalk region of chimeric MXRA8 on binding was also evaluated since its length modulated binding of human MXRA8 to CHIKV in a prior study. Du-D1-Mo-D2 MXRA8-Fc molecules were generated with a mouse (moStalk, 43 amino acids) or duck (duStalk, 38 amino acids) stalk, which are 35% identical at the amino acid level. Notably, Du-D1-Mo-D2 duStalk MXRA8-Fc and Du-D1-Mo-D2 moStalk MXRA8-Fc showed similar inhibitory activity against SINV-CHIKV and SINV-WEEV infection (see e.g., FIG. 51E-FIG. 51F).

To test the ability of chimeric Du-D1-Mo-D2 MXRA8-Fc to protect against alphaviruses in vivo, Du-D1-Mo-D2 MXRA8-Fc was generated with an N66R point mutation that facilitated greater expression in cell culture. Du-D1-Mo-D2 N66R MXRA8-Fc had similar neutralizing activity against CHIKV and WEEV (see e.g., FIG. 51G-FIG. 51H). It was evaluated whether co-injection of Du-D1-Mo-D2 N66R MXRA8-Fc with WEEV (McMillan strain) would protect CD-1 mice from infection. Less clinical disease and higher survival rates after WEEV infection were observed in mice that received Du-D1-Mo-D2 N66R MXRA8-Fc than the LDLRAD3-D1-Fc control (see e.g., FIG. 39M-FIG. 39N; and TABLE 10). In an analogous experimental set-up, it was tested whether co-injection of Du-D1-Mo-D2 N66R MXRA8-Fc with CHIKV (La Reunion 2006 strain) would diminish infection in C57BL/6 mice, as reported previously for mouse MXRA8-Fc. Seventy-two hours after inoculation, joint swelling in the ipsilateral foot and CHIKV RNA levels in the ipsilateral and contralateral ankles and calf muscles were lower in mice treated with Du-D1-Mo-D2 N66R MXRA8-Fc or mouse MXRA8-Fc compared to the LDLRAD3-D1-Fc control (see e.g., FIG. 39O-FIG. 39S).

Reptile MXRA8 Supports SINV Infection.

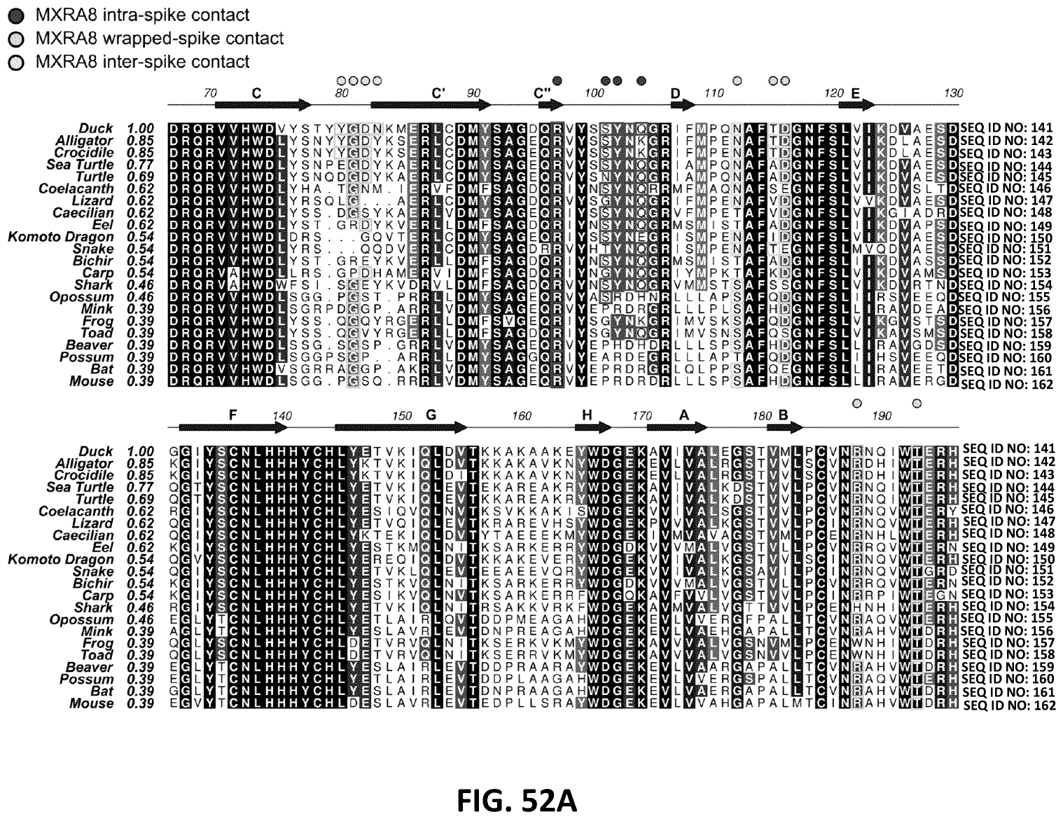

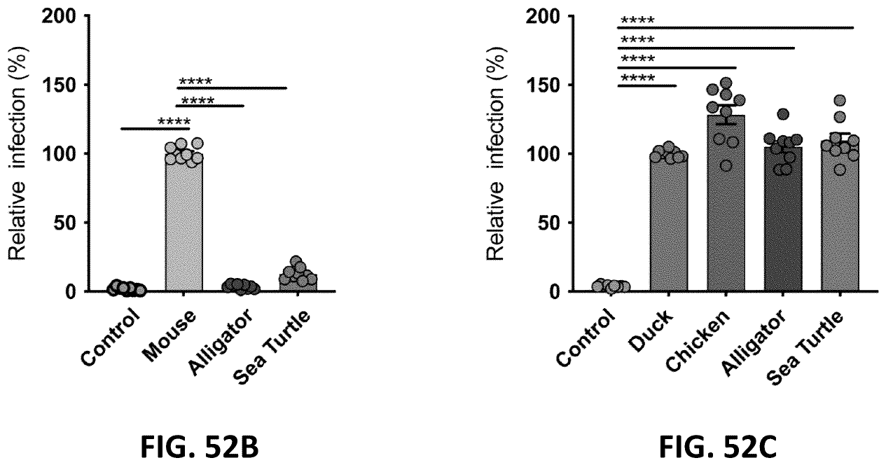

To gain further insight into vertebrate class-specific MXRA8 binding, it was assessed whether other non-avian MXRA8 species could support WEE complex virus infection. Based on the infection results with Du-D1-Mo-D2 MXRA8, species with D1 of MXRA8 were searched for that were similar to D1 of avian MXRA8, focusing in particular on the C″-D loop, which is a dominant contact area of duck MXRA8 and WEEV E1 (see e.g., FIG. 52A-FIG. 52C). The top ‘hits’ included alligator, crocodile, and sea turtle (96%, 96%, and 93% identical to duck C″-D loop, respectively) MXRA8; for these proteins, no more than a single residue varied from duck MXRA8 in the C″-D loop binding pocket of D1 (see e.g., FIG. 52A). Of note, several species of reptiles can be infected by encephalitic alphaviruses, including WEE complex members. Indeed, ΔMxra8 3T3 cells complemented with alligator or sea turtle MXRA8 (see e.g., FIG. 52B) supported SINV but not CHIKV infection (see e.g., FIG. 52C). These findings support the domain binding model for WEEV and D1 of duck MXRA8 and suggest that alphaviruses that infect reptiles might use MXRA8 to enter cells.

Du-D1-Mo-D2 Binds to CHIKV and WEEV in Opposing Orientations.

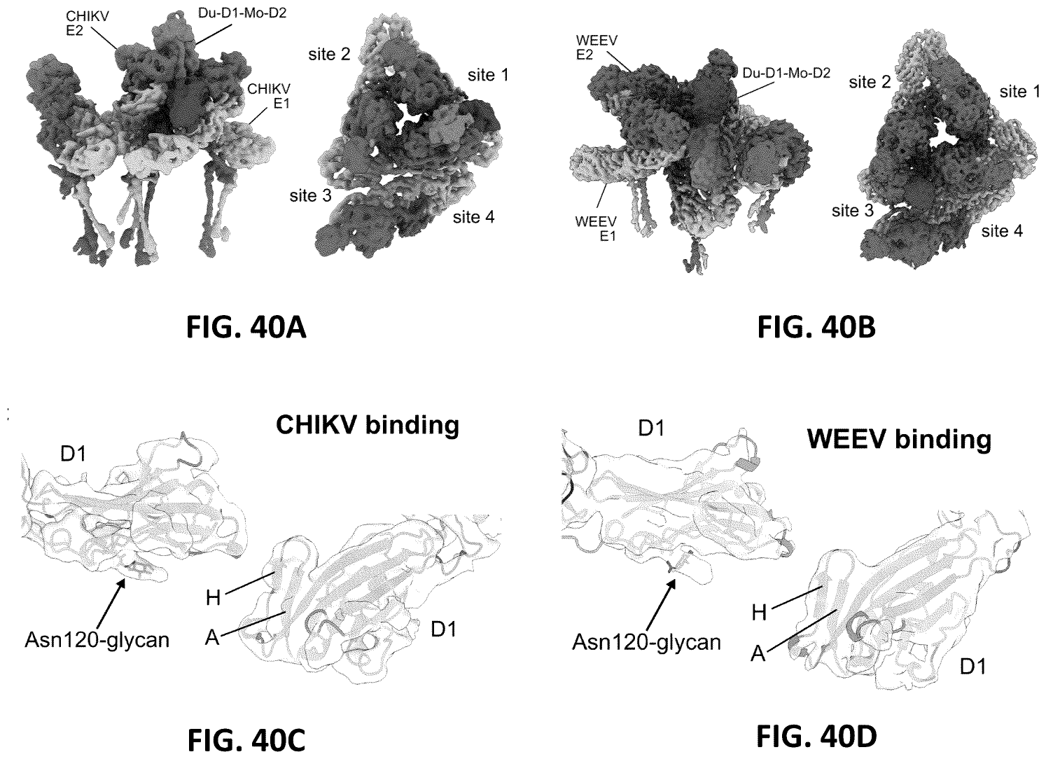

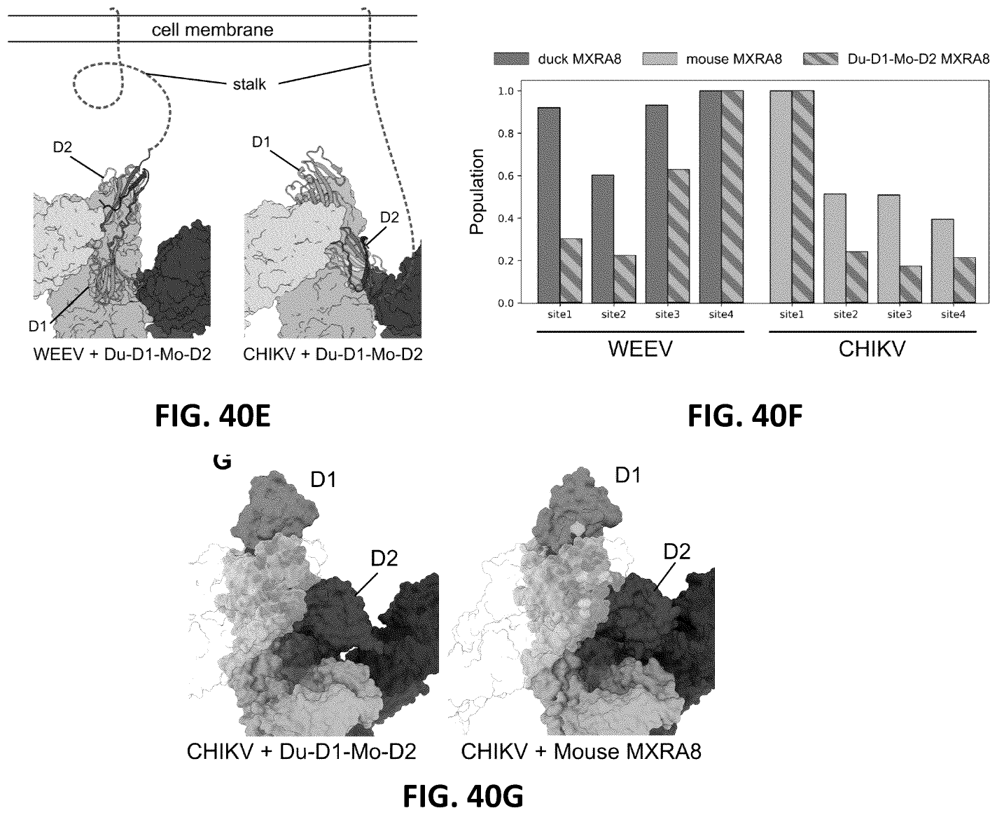

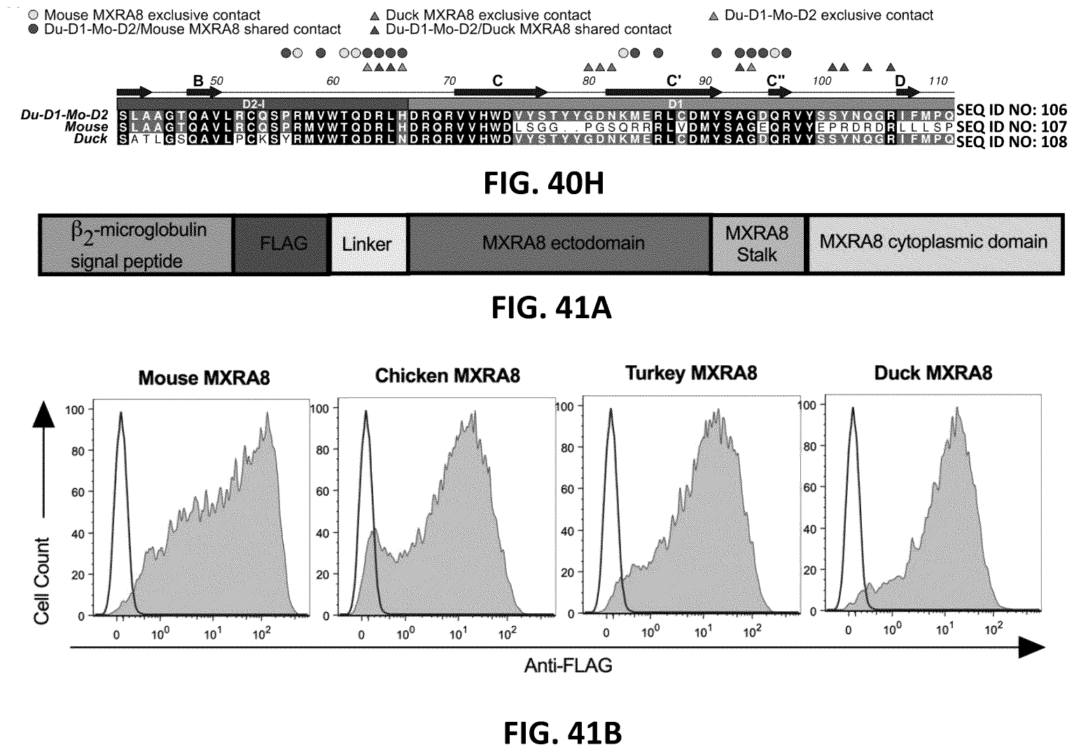

To understand better how chimeric MXRA8 inhibits infection, cryo-EM structures of Du-D1-Mo-D2 in complex with CHIKV or WEEV at 3.9 Å were generated for both maps (see e.g., FIG. 40A-FIG. 40E, TABLE 8, and TABLE 9). Du-D1-Mo-D2 preferentially binds to CHIKV at site 1, similar to mouse MXRA8, and WEEV at sites 4 and 3, similar to duck MXRA8 (see e.g., FIG. 40F). Du-D1-Mo-D2 binds to CHIKV and WEEV in flipped orientations with respect to each other (see e.g., FIG. 40E), consistent with how mouse and duck MXRA8 bind to CHIKV and WEEV, respectively. Du-D1-Mo-D2 can use duck D1 to bind either CHIKV E2-wrapped or WEEV E1-intraspike heterodimers. This binding topology model is supported by the location of the A and H b-strands in D1 and glycan density at Asn-120 in each model (see e.g., FIG. 40B and FIG. 40D).

D1 of duck MXRA8 is a suitable replacement for D1 of mouse MXRA8 when contacting CHIKV. Contacts on D1 of duck MXRA8 with CHIKV E2, when expressed as the Du-D1-Mo-D2 chimera, are congruent with D1 of mouse MXRA8 and CHIKV E2; for every residue of Du-D1 that contacts CHIKV E2, there is an equivalently positioned contact residue in D1 of mouse MXRA8 (see e.g., FIG. 40G and FIG. 40H). There are only 5 contact residues in D1 of mouse MXRA8 for which equivalently positioned residues in Du-D1-Mo-D2 do not contact CHIKV. In comparison, when mouse MXRA8 D2 is expressed in the Du-D1-Mo-D2 chimeric protein, it has no or few observed interspike and intraspike contacts with CHIKV E1, both of which are present in the structure of mouse MXRA8 bound to CHIKV. In the Du-D1-Mo-D2 chimera, mouse MXRA8 D2 does not alter the binding of duck D1 to WEEV E1 when compared to duck MXRA8. Primary contact residues between β-strands C″ and D are identical between D1 of duck MXRA8 and the Du-D1-Mo-D2 chimera. Overall, these studies show that chimeric Du-D1-Mo-D2 MXRA8 can bind to both CHIKV and WEEV in a similar topology as mouse MXRA8 and duck MXRA8, respectively, and that D1 of duck MXRA8 can functionally replace D1 of mouse MXRA8 to bind CHIKV when it is paired with D2 of mouse MXRA8.

### DISCUSSION

In this study, it was discovered that avian MXRA8 can function as a receptor for members of the WEE complex even though it does not act as a receptor for SF complex members that have mammalian reservoirs and can bind mammalian MXRA8. Members of the SF complex use mammalian but not avian MXRA8, and members of the WEE complex use avian but not mammalian MXRA8. VEEV, EEEV, and at least one virus (AURAV) in the WEE complex do not appear to utilize either avian or mammalian MXRA8.

Although it was initially hypothesized that members of the SF and WEE complexes would engage mammalian or avian MXRA8 similarly, the cryo-EM analysis of avian MXRA8 bound to WEEV VLPs revealed a domain-inverted binding mode compared to mammalian MXRA8 and CHIKV. Whereas both D1 and D2 of mammalian MXRA8 contact CHIKV, the binding of avian MXRA8 to WEEV is principally through D1 with minimal contact of D2 with WEEV. CHIKV E2 binds D1 of mammalian MXRA8 distal to the viral membrane, whereas CHIKV E1 binds D2 proximal to the viral membrane. In contrast, WEEV E1 engages a different set of residues in D1 of avian MXRA8 in a reverse orientation proximal to the viral membrane, with WEEV E2 making minimal or no contacts with D1 or D2 of avian MXRA8. A unique feature of MXRA8 is the head-to-head orientation of its two Ig-like domains. This architecture generates a “pseudo-symmetric” molecule, such that a flip results in topological similarity and a broader range of virus engagement opportunities. This could offer a potential explanation as to how different MXRA8-alphavirus binding modes evolved across species.

The results herein establish a critical interaction role of D1 of MXRA8, irrespective of the species, binding mode, or alphavirus. D1 of duck MXRA8 forms intraspike contacts with WEEV E1, and D1 of mouse MXRA8 forms wrapped contacts with CHIKV E2, with distinct residues at each interface. This result was unexpected because MXRA8 domains D1 and D2 have pseudo-symmetric folds and sequence relatedness across species: duck and mouse D1 are 43.7% identical, and duck and mouse D2 are 62.2% identical at the amino acid level. Despite the pseudo-symmetry relationships of MXRA8, shown herein is that each virus utilizes independent binding schemes for initiation of infection. The chimera and mutagenesis experiments, and the cryo-EM co-structures of Du-D1-Mo-D2, supported the domain-inverted binding model, as expression of Du-D1-Mo-D2 MXRA8 promoted infection of both WEE and SF complex members, whereas the Mo-D1-Du-D2 MXRA8 did not. The ability of Du-D1-Mo-D2 MXRA8-Fc protein to neutralize SINV and SINV-WEEV infection suggests a dominant role of D1 of avian MXRA8 in binding WEE complex viruses. The structure of Du-D1-Mo-D2 in complex with CHIKV also revealed that corresponding residues on D1 of duck MXRA8 can serve as replacements for interaction with D1 of mouse MXRA8, which likely contributes to the ability of membrane-associated and soluble Du-D1-Mo-D2 to support or inhibit infection, respectively, of SF-complex members. Overall, the structural data with Du-D1-Mo-D2 MXRA8 and CHIKV and the functional results with Du-D1-Mo-D2 MXRA8 and Mo-D1-Du-D2 MXRA8 and CHIKV are consistent with a required role of D2 of mammalian MXRA8 in facilitating binding and/or an inhibitory role of D2 of duck MXRA8 in preventing binding.

In support of the flipped-domain orientation model, introduction of mutations or loop insertions into D1 but not D2 of duck MXRA8 abrogated or reduced infection of WEE complex members. Associated with the swap in MXRA8 domains was a differential reliance on the viral structural proteins; WEEV principally uses E1 of the intraspike heterodimer to bind duck MXRA8, and based on the number of residue contacts at the binding interface, CHIKV and MAYV predominantly use E2 of the wrapped spike heterodimer to engage murine or human MXRA8. Structural features of the complexes also offer potential explanations for the distinct binding specificities including shifted N-linked glycosylation of E1 at the intraspike contact site, which in the case of CHIKV might clash with avian MXRA8 and prevent engagement. Of note, SFV binds VLDLR through interactions in domain III of E1, which differ from the binding site on E1 that CHIKV and WEEV use to engage mammalian and avian MXRA8, respectively.

Based on deletion experiments, a prior study established that the 48-amino acid stalk length of human MXRA8 was critical for supporting CHIKV entry into cells. The swap experiments herein showed that in the context of the chimeric Du-D1-Mo-D2-Fc decoy receptor, duck (38 amino acids) and mouse (43 amino acids) stalk regions did not alter neutralization potency against SINV-CHIKV or SINV-WEEV. Thus, the stalk sequence does not appear to dictate avian or mammalian MXRA8 binding preferences to different alphaviruses, and the flip of duck-mouse chimeric MXRA8 orientation between its binding modes with WEEV and CHIKV is not sensitive to the small 5-amio acid difference in duck and mouse MXRA8 stalk lengths. However, because the cryo-EM images did not directly visualize the stalk region, conformations required for binding the different alphaviruses may be speculated on.

During the evolution of alphaviruses between birds, other vertebrates, and mammals, adaptation to MXRA8 as a receptor likely occurred resulting in changes in binding modes of the viral structural proteins in a vertebrate-class specific-manner. Although speculative, one of three evolutionary scenarios might explain the MXRA8 receptor usage patterns observed: (a) there was a common viral ancestor of SF and WEE complexes that could use mammalian, avian, and reptile MXRA8. During evolution, the SF branch specialized to infect mammals and lost its capacity to engage avian or reptile MXRA8, whereas the WEE complex lost its ability to use mammalian MXRA8; (b) the ancestor of WEE complex viruses bound only avian or reptile MXRA8. During evolution, the virus adapted to mammalian MXRA8, which enabled the SF complex to branch and infect mammalian hosts. However, during the process of adaptation to mammalian MXRA8, SF complex viruses lost their ability to bind avian MXRA8 and use avian hosts; or (c) WEE and SF complexes alphaviruses adapted independently to use reptile, avian, and mammalian MXRA8.

Avian MXRA8 is not the only receptor reported for SINV in chicken cells, as a 67 kDa protein was identified previously. Chicken MXRA8 is 50 kDa, which makes it unlikely that it is the previously recognized SINV receptor. Several other attachment or entry factors have been described for SINV in mammalian cells including a high-affinity laminin receptor, VLDLR, C-type lectins (DC-SIGN and L-SIGN), NRAMP2, and heparan sulfate, although their significance in vivo remains uncertain and role in avian cell infection is unknown. The findings of residual yet diminished infection by WEE complex alphaviruses in chicken cells after antibody blockade or gene editing of chicken MXRA8 suggests the existence of additional avian receptors.

The finding of an inverted binding mode enabled the engineering of a Du-D1-Mo-D2 chimera that can support infection of both WEE complex and SF complex viruses. Produced as a bivalent soluble decoy receptor, this chimera has neutralizing activity in cell culture and in vivo, and has potential as a broad-spectrum countermeasure against alphaviruses. Shown herein using cryo-EM is that the Du-D1-Mo-D2 chimeric MXRA8 can bind to CHIKV and WEEV using the respective flipped binding modes observed with mouse and duck MXRA8. Structural and evolutionary analyses were also used to identify other vertebrate MXRA8 proteins from reptiles that support WEE complex virus infection. These results could help to identify potential reservoirs of WEE complex viruses and the sources of future zoonotic outbreaks.

Although the usage of avian MXRA8 by WEE complex alphaviruses in mammalian and chicken cells and in a mouse ectopically expressing chicken MXRA8 was established herein, the role of avian MXRA8 in the pathogenesis of these viruses in their enzootic hosts, birds, may be a subject of future work. At what point in alphavirus evolution the flip in MXRA8 binding orientation and class specificity occurred is yet to be identified. While an avian-mammalian chimeric MXRA8 decoy with broad-spectrum inhibitory activity against alphaviruses was generated, its post-exposure activity has not yet been tested. The moderate resolutions of the cryo-EM reconstructions limit ability to make mechanistic claims based on specific atomic interactions.

In summary, this study reveals how different domains of the E1 and E2 proteins of zoonotic alphaviruses engage divergent vertebrate receptors in distinct orientations to allow for unique tropisms. These findings enhance understanding of how viruses evolve to infect new hosts and reveal insight into viral structure, receptor utilization, and class adaptation. Finally, the ability of chimeric Du-D1-Mo-D2 MXRA8-Fc to inhibit infection of both SF and WEE complex members in vitro and in vivo suggests the possible development of decoy proteins that broadly neutralize infection of alphaviruses from different antigenic groups.

Experimental Model and Study Participant Details

Cells.

NIH-3T3 (CRL-1658), HEK-293 (CRL-1573), BHK-21 (CCL-10), HeLa (ATCC CCL-1), K562 (CRL-3344), Jurkat (ATCC TIB-152), and Vero (CCL-81) cells, and CEFs (CRL-12203) were obtained from ATCC and cultured at 37° C. in DMEM supplemented with 10% heat inactivated FBS (Hyclone), 100 U/ml penicillin, 100 mg/ml streptomycin, and 10 mM HEPES. Expi293 cells were obtained from Thermo Fisher and cultured shaking (100 RPM) at 37° C. and 8% CO2 in Expi293 Expression Medium.

Viruses.

The following alphavirus strains were obtained from the World Reference Center for Emerging Viruses and Arboviruses (University of Texas Medical Branch, Galveston, TX; generous gifts of S. Weaver and K. Plante), propagated in BHK-21 or Vero cells, and titered by focus forming assay (FFA) as described (see e.g., Fox et al. (2015) Cell 163, 1095-1107): BBKV (DAK AR Y 251), CHIKV (181/25), CHIKV Senegal (37997), OCKV (EDS 14), SINV (TR339, Toto, and Girdwood), WHAV (M 78), and WEEV (McMillan). Chimeric viruses including SINV-EEEV (FL93-939)-GFP, SINV-VEEV (TrD-GFP), SINV-WEEV (CBA87)-GFP, SINV-CHIKV (LR-2006)-GFP were constructed by replacing the structural protein genes of SINV TR339 with those from EEEV, VEEV, WEEV, or CHIKV. eGFP expressing versions of these viruses and SINV TR339 were generated by inserting an eGFP sequence followed by the Thosea Asigna virus 2 Å self-cleaving peptide sequence after the capsid gene in the SINV TR339 molecular clone. The chimeric alphaviruses and SINV TR339 were propagated in Vero cells and titered by focus forming assay (FFA) as described (see e.g., Ma et al. (2020) Nature. 10.1038/s41586-020-2915-3). Viral RNA was generated by in vitro transcription (mMessage mMachine, Ambion) of linearized cDNA after digestion with Xhol. Viral RNA was introduced to BHK-21 cells by electroporation, supernatants were harvested 24 to 36 hours later, and stock titers were determined by focus-forming assay on Vero CCL81 cells. AURAV (BE AR 10315) was propagated in CEFs and tittered by FFA. AURAV, BBKV, OCKV, SINV, and WHAV antigens were detected using mouse SINV immune ascites fluid or mAb DC2.112.

Mouse Studies.

Animal studies were carried out in accordance with the recommendations in the Guide for the Care and Use of Laboratory Animals of the National Institutes of Health. The protocols were approved by the Institutional Animal Care and Use Committee at the Washington University School of Medicine (assurance number A3381-01) or University of Pittsburgh (assurance number D16-00118). Virus inoculations were performed under anesthesia that was induced and maintained with ketamine hydrochloride and xylazine, and all efforts were made to minimize animal suffering. Wild-type C57BL/6J and BALB/c mice were purchased commercially from the Jackson Laboratory and CD-1 mice were obtained from Charles River Laboratories. Female BALB/c and CD-1 mice and male C57BL/6 mice were used in these studies.

House Sparrow Sample.

Tissues were collected from a captured adult house sparrow (Passer domesticus) from a ground trap. All experiments were performed after approval by the Institutional Animal Care and Use Committee at Colorado State University.

Method Details

Recombinant Adenoviral Vector.

The human Ad5-chicken Mxra8 construct was designed using a codon optimized version of chicken Mxra8 (Gallus; NP_989967) and synthesized commercially (Integrated DNA Technologies). The control Ad5 was reported previously (see e.g., Jia et al. (2005) J Virol 79, 14614-14621). Ad5-chicken Mxra8 and Ad5-empty vector control were propagated in 293T cells and purified using cesium chloride density-gradient ultracentrifugation. The virus particle number was determined by spectrophotometry using optical density (260 nm) measurement and plaque assays, as described (see e.g., Mittereder et al. (1996) J Virol 70, 7498-7509). The ability of the Ad5-chicken Mxra8 to induce surface expression of the transgene was established in HEK293 cells 20 h after transduction with Ad5-chicken Mxra8 (MOI, 20). Cells were detached using TrypLE (Thermo Fisher), washed with PBS and stained with a mixture of mouse anti-chicken mAb Mxra8 antibody (6D7, and 11 Å6 at 2 mg/mL) in PBS, 1% BSA, for 1 h at 4° C. followed by APC-conjugated goat anti-mouse secondary antibody (Thermo Fisher, 2 mg/mL) for 30 min at 4° C. After washing and fixation with medium A (Thermo Fisher), data were collected on a MACSQuant X Flow cytometer and analyzed using FlowJo V9.

Mouse Experiments.

BALB/c mice were purchased commercially (Jackson Laboratories). Animals were housed in groups and fed standard chow diets. Five-week-old female BALB/c mice were administered 10¹⁰ virus particles of hAdV5-chicken Mxra8 or hAdV5-control via intranasal administration. In some experiments, 0.5 mg of anti-IFNAR1 mAb (MAR1-5 Å3 (Leinco) was administered via intraperitoneal route four days after hAdV5 treatment. Five days later mice were inoculated with 10⁴ PFU of SINV. Weights were monitored daily, animals were sacrificed at 40 hpi or 3 dpi, and tissues were harvested.

10³ FFU of CHIKV (La Reunion 2006) was mixed with mouse MXRA8-human Fc, Du-D1-Mo-D2-N66R MXRA8-human Fc or LDLRAD3-D1-Fc (50 μg per mouse in PBS), and incubated at 37° C. for 30 min before subcutaneous inoculation of 4-week-old male C57BL/6 mice in the footpad. At 72 h post-inoculation, animals were euthanized, and after perfusion with PBS, indicated tissues were collected and processed for viral RNA levels. Joint swelling in the ipsilateral foot was monitored at 72 h post-infection by measuring width×height using digital calipers as previously described (see e.g., Hawman et al. (2013) J Virol 87, 13878-13888). 10³ PFU of WEEV was mixed with 50 mg of Du-D1-Mo-D2-N66R MXRA8 or control (LDLRAD3-DI-human Fc) for 30 min at room temperature before subcutaneous inoculation of 4-week-old CD-1 female mice in the footpad. Mice were monitored daily for survival and clinical signs of disease. Clinical signs were assigned by the following criteria: 0—healthy; 1—ruffled fur, mild behavioral changes; 2—hunched posture, significant behavioral changes; 3—seizures, ataxia, catatonia; 4-recumbent moribundity; 5—death. Mice scoring 3 or higher were immediately euthanized.

Plasmid Construction for Complementation Studies.

MXRA8 cDNA fragments were generated. The MXRA8 signal peptide was replaced with a b2-microglobulin signal peptide (MARSVTLVFLVLVSLTGLYA, SEQ ID NO: 163), FLAG tag (DYKDDDDK, SEQ ID NO: 164) and a short linker (GGS). Nucleotide sequences were codon-optimized, synthesized, and inserted into the lentivirus vector pLV-EF1a using an In-Fusion HD Cloning Kit (Takara) for the following species: mouse Mus musculus (Genbank accession no. NM 024263); turkey, Meleagris gallopavo (XP_010721105.1); duck, Anas platyrhynchos (XM_027443263); chicken, Gallus (NP_989967); house sparrow, Passer domesticus; green sea turtle, Chelonia mydas (XP_007070253.1); and alligator, Alligator mississippiensis (XP_006263071.1). Chimeric Mo-D1-Du-D2 MXRA8, Du-D1-Mo-D2 MXRA8 along with duck C-C′, C″-D, and D-E loop MXRA8 mutants were designed (see e.g., FIG. 50), codon-optimized, synthesized, and inserted into the lentivirus vector pLV-EF1a as described above. Plasmids were transformed into One Shot Stbl3 Chemically Competent E. coli (Thermo Fisher), and bacteria were grown at 30° C. on LB Agar plates with carbenicillin (100 μg/ml). Colonies were picked and grown overnight at 30° C. in LB supplemented with carbenicillin (100 μg/ml). Plasmids were extracted (Qiagen) and sequenced using the following primers: GCACTTGATGTAATTCTCCTTGGAATTTGC (SEQ ID NO: 165), CTCAAGCCTCAGACAGTGGTTCAAAGT (SEQ ID NO: 166) and GGTGGAAAATAACATATAGACAAACGCAC (SEQ ID NO: 167). Primers were used to sequence the following species: Meleagris gallopavo: ATGCTTTTACAGATGGTAACTTCAG (SEQ ID NO: 185) and TGAACATCACTGATACTGCCTTTG (SEQ ID NO: 168); Anas platyrhynchos: TCAGGGGAGAATATTTATGCCACAA (SEQ ID NO: 169) and TGAATATAACCGATACTGCTTTCGC (SEQ ID NO: 170); Gallus: CAGGGGAGGATACTGATGCC (SEQ ID NO: 171) and TGGGCCCCTCTTTATCCGA (SEQ ID NO: 172).

Expression and Purification of MXRA8 Proteins.

A cDNA fragment encoding residues 22-327 of the chicken MXRA8 extracellular domain (Gallus; NP_989967) was codon-optimized, synthesized, and inserted into the pET21a vector using the NdeI/NotI sites. After sequence confirmation, the plasmid was transformed into BL21(DE3) chemically competent cells (Thermo Fisher). Cells were grown at 37° C. to an optimal density (600 nm) of 0.8 and induced with 0.1 mM IPTG for 4 h. Cells were harvested and resuspended in 50 mM Tris-HCl, 1 mM EDTA, 0.01% NaN₃, 1 mM DTT, 25% sucrose (TENDS) buffer, and lysed in 50 mM Tris-HCl, 1 mM EDTA, 0.01% NaN₃, 1 mM DTT, 200 mM sodium chloride, 1% sodium deoxycholate and 1% Triton X-100. Inclusion bodies were isolated from the cellular lysate after centrifugation at 6,000×g for 20 min and washed in TENDS buffer supplemented with 100 mM NaCl and 0.5% Triton X-100. A final wash was performed in the same buffer without 0.5% Triton X-100. Inclusion bodies were denatured in in 100 mM Tris-HCl, 6 M guanidinium chloride and 20 mM β-mercaptoethanol for 1 h. Denatured protein was oxidatively refolded overnight at 4° C. in 400 mM L-arginine, 100 mM Tris-HCl, 5 mM reduced glutathione, 0.5 mM oxidized glutathione, 10 mM EDTA and 200 mM phenylmethylsulphonyl fluoride. Refolded protein was concentrated using a 10,000-molecular weight cut-off stirred cell concentrator (EMD Millipore). Concentrated protein was purified by HiLoad 16/600 Superdex 75 size exclusion chromatography (GE Healthcare) and HiTrap Q HP anion exchange chromatography (GE Healthcare). Purity and oligomeric state were confirmed by SDS-PAGE analysis and size exclusion chromatography.

Chicken MXRA8-Fc, duck MXRA8-Fc, and chimeric Du-D1-Mo-D2 duStalk MXRA8-Fc, Du-D1-Mo-D2 moStalk MXRA8-Fc, Du-D1-Mo-D2-N66R MXRA8-Fc, or Mo-D1-Du-D2 MXRA8-Fc fusion proteins were generated based on a prior protocol used to generate mouse-MXRA8-Fc (see e.g., Zhang et al. (2018) Nature 557, 570-574). Briefly, cDNA fragments encoding chicken MXRA8 (Gallus; NP_989967; residues 22-328), duck MXRA8 (Anas platyrhynchos; XM_027443263; residues 23-334), Chimeric Du-D1-Mo-D2 duStalk MXRA8 (duck residues 66-196 and 296-334; mouse residues 23-65 and 195-293), chimeric Du-D1-Mo-D2 moStalk MXRA8 (duck residues 66-196; mouse residues 23-65 and 195-334) or chimeric Mo-D1-Du-D2 MXRA8 (mouse residues 65-196; duck residues 29-64 and 199-334) were appended with a GGGGSGGGGS linker and the mouse IgG2b Fc or human Fc region before synthesis (Integrated DNA Technologies) and inserted into the pCDNA3.4 vector. MXRA8-Fc plasmids were diluted in Opti-MEM, incubated with HYPE-5 reagent (OZ Biosciences), and the complex was transfected into 10⁶ cells/ml of Expi-293 cells (Thermo Fisher). Cells were supplemented daily with Expi293 medium and 2% (w/v) Hyclone Cell Boost. Four days post transfection, the supernatant was harvested by centrifuging at 3,000×g for 15 min and protein was purified using Protein A Sepharose 4B (Thermo Fisher). Chicken MXRA8-Fc fusion protein was eluted using Pierce™ Gentle Ag/Ab Binding and Elution Buffer Kit (Thermo Fisher). After elution, MXRA8-Fc proteins underwent buffer exchange with PBS and stored at 4° C. Purity was confirmed by SDS-PAGE analysis.

A histidine-tagged duck MXRA8 was generated in Expi293 mammalian cells. Briefly, a cDNA fragment encoding duck MXRA8 (Anas platyrhynchos; XM_027443263; Residues 23-328), GGS linker, and C terminal 8× His tag. The duck MXRA8-His tag was synthesized (Integrated DNA Technologies) and inserted into the pCDNA3.4 vector. The duck MXRA8-His plasmid was diluted in Opti-MEM, incubated with HYPE-5 reagent (OZ Biosciences), and the complex was transfected into 10⁶ cells/ml of Expi-293 cells (Thermo Fisher). Cells were supplemented daily with Expi293 medium and 2% (w/v) Hyclone Cell Boost. Four days post-transfection, the supernatant was harvested by centrifuging at 3,000×g for 15 min, and protein was purified using Cobalt resin (G-Biosciences). After elution with 200 mM imidazole, duck MXRA8-His tag protein underwent buffer exchange with PBS and stored at 4° C. Purity was confirmed by SDS-PAGE analysis.

Sparrow Sample Collection and Sequencing

Muscle and liver samples were obtained from a captured house sparrow. Total RNA was extracted using a MagMax Viral Isolation Kit (Thermo Fisher). First strand cDNA was generated using a SuperScript III First-Strand Synthesis System (Thermo Fisher). House sparrow MXRA8 specific primers were designed from deposited sequences of related species and assembled whole genome sequences. PCR master mixes were prepared in a nucleic acid-free PCR workstation. House sparrow MXRA8 was amplified using PCR with 1×Q5 Reaction Buffer, 200 mM dNTPs, 200 nM Forward primer, 200 nM Reverse primer, 2 mL of cDNA, lx Q5 High GC Enhancer, and 1 mL of Q5 High-Fidelity DNA Polymerase (NEB). The following amplification protocol was used for both first- and second-round amplifications: (1) 98° C. for 30 sec; (2) 98° C. for 20 sec; (3) 60-68° C. for 30 sec; (4) 72° C. for 60 sec; and (5) 72° C. for 2 min; with steps 2-4 repeated for 35 cycles. All primer sequences and annealing temperatures are listed in Table 51. PCR products were separated on a 1% agarose gel. PCR products were cloned using Zero Blunt™ TOPO™ PCR Cloning Kit and transformed into One Shot TOP10 Chemically Competent E. coli (Thermo Fisher). Ten selected colonies were sequenced using predefined primers (M13 forward and M13 reverse).

Anti-Chicken MXRA8 mAb Generation.

Four-week-old BALB/c mice were sequentially immunized and boosted via intravenous route with 20 μg of bacterially-generated, purified chicken MXRA8 at two-week intervals. Four and eight weeks later, mice were boosted with 20 μg of mammalian cell-derived chicken MXRA8-mouse-Fc. After boosting was completed, serum samples were collected and tested for binding to chicken MXRA8 on the surface of complemented ΔMxra8 3T3 cells. Three days after the final boost, the mouse with the highest serum titer (e.g., 1/27,000) for binding chicken MXRA8 underwent a terminal bleed and euthanasia. The spleen of this animal was collected for splenocyte-myeloma fusion and hybridoma production.

Hybridoma supernatants were screened by ELISA for binding to chicken MXRA8. As a second confirmatory assay, the binding of hybridoma supernatants to chicken MXRA8 on the surface of complemented ΔMxra8 3T3 cells was evaluated using flow cytometry. Finally, as a tertiary screen, hybridoma supernatants were tested for blockade of SINV TR339 infection in CEFs. After limiting dilution subcloning, the four clones with the strongest blocking activities (6D7, 9B6, 11 Å6 and 16D9) were expanded. Antibodies were purified using Protein A Sepharose 4B chromatography (Invitrogen #101042), dialyzed in PBS, concentrated, and sterile-filtered.

Complementation and Infection Experiments.

Lentiviruses encoding MXRA8 were packaged with psPAX2 (Addgene #12260) and pMD2.G (Addgene #12259) vectors in HEK293 cells using FugeneHD (Promega). ΔMxra8 3T3 cells, ΔMXRA8 CEFs (see below), 293T, HeLa, K562 and Jurkat cells, were transduced with lentiviruses and selected with blasticidin for 7 days. Surface expression of mouse or avian MXRA8 proteins was assessed using rabbit anti-FLAG mAb (Cell Signaling; 1 mL per 500 mL) and Alexa Flour 647 conjugated anti-rabbit IgG (Cell Signaling; 1 mL per 500 mL). Surface expression of mouse MXRA8 also was assessed by flow cytometry after staining with a pool of seven hamster anti-mouse MXRA8 mAbs 9 (2 mg/mL), and Alexa Fluor 647 conjugated goat anti-Armenian hamster IgG (2 mg/mL) at 4° C. Surface expression of chicken MXRA8 in some instances was assessed by flow cytometry after staining with a pool of four mouse anti-chicken MXRA8 mAbs (6D7, 9B6, 11 Å6 and 16D9; 2 mg/mL) and Alexa Flour 647 conjugated goat anti-mouse IgG (2 mg/mL). ΔMxra8 3T3, 293T, HeLa, K562, Jurkat, or CEF cells complemented with avian MXRA8 or murine Mxra8 with surface expression levels of MXRA8 less than 90% after blasticidin selection were enriched further by fluorescence activated cell sorting. Cells (2.5×10⁵) were incubated with a rabbit anti-Flag mAb (Cell Signaling) (2 mg/ml) in 1% BSA/PBS for 30 min at 4° C. After 30 min, cells were washed and incubated with Alexa Fluor 647 conjugated goat anti-rabbit IgG (2 mg/mL). After a 30-min incubation, cells were washed, resuspended in PBS supplemented with 2% FBS and 1 mM EDTA, and sorted using a BD FACSAria II. MXRA8+ cells were expanded in culture.

Human LDLRAD3 (NM_174902.4) and human VLDLR (NP_001018066.1) were codon-optimized and synthesized (GeneWiz) and inserted into the lentivirus vector pLV-EF1a-IRES-Hygro (Addgene no. 85134) between the BamHI and MluI restriction enzyme sites using In-Fusion HD Cloning (Takara). The signal peptide of the genes was replaced by human b2-microglobulin signal peptide/FLAG-tag/GGS linker. The plasmids were packaged in HEK-293 cells with psPAX2 (Addgene no. 12260) and pMD2.G (Addgene no. 12259) using Lipofectamine 3000 (Thermo Fisher) and then transduced into Jurkat cells or K562 grown in RPMI-1640 with 10% FBS, 100 U/ml penicillin, 100 U/ml streptomycin and 0.05 mM b-mercaptoethanol. One day later, Jurkat or K562 cells selection with 200 μg/ml of hygromycin (InvivoGen) was initiated for seven days. LDLRAD3 and VLDLR expression was verified with anti-FLAG antibody as described above.

Complemented ΔMxra8 3T3 cells were inoculated with CHIKV 181/25 (MOI 3, 9 h) and SINV-CHIKV-LR (MOI 1, 9.5 h) in DMEM supplemented with 2% FBS or with SINV (TR339, Toto, or Girdwood; MOI 1, 9 h; TR339-GFP MOI 1, 10 h), BBKV (MOI 1, 8 h), OCKV (MOI 1, 8 h), AURAV (MOI 1, 24 h), WHAV (MOI 1, 24 h), SINV-EEEV (MOI 0.1, 9 h), SINV-WEEV (MOI 0.1, 9 h) or SINV-VEEV (TrD) (MOI 0.1, 9 h) in DMEM supplemented with 10% FBS. At indicated time points, cells were harvested, incubated sequentially with Fixation medium A (Thermo Fisher) and Permeabilization medium B (Thermo Fisher), and stained for viral antigen after incubation with the following antibodies: CHIKV (mouse mAb CHK-11, mouse anti-SINV immune ascites fluid (ATCC) or human anti-E1 DC2.112 and DC2.315. Cells were washed, incubated with Alexa Fluor 647 conjugated goat anti-mouse IgG (Thermo Fisher) or goat anti-human IgG (Southern Biotech), and analyzed by flow cytometry using a MACSQuant Analyzer 10 (Miltenyi Biotec).

CEFs, ΔMxra8 CEFs, and complemented CEFs cells were inoculated with CHIKV 181/25 (MOI 1, 10 or 24 h), SINV-WEEV (MOI 0.1, 9 h), SINV (MOI 1, 8 h), BBKV (MOI 1, 8 h), OCKV (MOI 1, 22 h), AURAV (MOI 1, 24 h), WHAV (MOI 1, 17 h) and SINV-VEEV (TrD) (MOI 0.1, 9 h) in 10% FBS growth medium. After infection, cells were harvested, fixed, permeabilized, stained with virus-specific antibodies, and analyzed by flow cytometry as described above.

293T cells complemented with chicken, sparrow or murine MXRA8 were inoculated with CHIKV 181/25 (MOI 1, 12 h), CHIKV-37997 (MOI 1, 8 h), SINV (MOI 1, 9 h) and AURAV (MOI 1, 24 h) in 10% FBS growth medium. HeLa cells complemented with chicken or mouse MXRA8 were inoculated with CHIKV 181/25 (MOI 1, 14 h), SINV (MOI 1, 12 h), AURAV (MOI 1, 24 h), in 10% FBS growth medium. After infection, all cells were harvested, fixed, permeabilized, stained with virus-specific antibodies, and analyzed by flow cytometry as described above.

Jurkat cells complemented with mouse MXRA8, turkey MXRA8, or human LDLRAD3 were inoculated with CHIKV 181/25 (MOI 10, 16 h), SINV-TR339-GFP (MOI 10, 16 h), or SINV-VEEV-GFP (MOI 10, 16 h) in 2% FBS growth medium. After infection, all cells were harvested, fixed, permeabilized, stained with virus-specific antibodies (if not GFP-tagged), and analyzed by flow cytometry as described above.

K562 cells complemented with mouse MXRA8, turkey MXRA8, or human VLDLR were inoculated with SINV-EEEV-GFP (MOI 10, 16 h) in 2% FBS growth medium. After infection, all cells were harvested, fixed, and analyzed by flow cytometry as described above.

For multi-step growth curves, complemented ΔMxra8 3T3 and CEFs were inoculated (MOI 0.01) with SINV TR399 or OCKV for 2 h, washed 3 times with warmed media (37° C.), and maintained in 2% FBS growth medium. Viral supernatants were harvested at indicated time points, titered on Vero cells using a focus-forming assay as previously described (see e.g., Brien et al. (2013) Curr Protoc Microbiol 31, 15D1311-15D1318). After fixation, cells were permeabilized, and stained with anti-SINV ascites and horseradish peroxidase-conjugated goat anti-mouse IgG. Infected foci were visualized using TrueBlue peroxidase substrate (KPL) and quantitated on an ImmunoSpot 5.0.37 Macroanalyzer (Cellular Technologies).

Virus Binding and Internalization Assays.

For virus binding assays, ΔMxra8 3T3 cells complemented with mouse Mxra8 or chicken MXRA8 or an empty vector were incubated with SINV TR339 (MOI of 1) in DMEM 2% FBS for 1 h on ice. After six rinses with ice cold PBS, cells were lysed in RLT buffer (Qiagen). For attachment inhibition assays, 100 μg/mL of the indicated mAbs were incubated with 3T3 cells for 30 min at 4° C. SINV (MOI, 1) was added to the chilled cells and incubated at 4° C. for 1 h. Cells were rinsed six times with ice cold PBS and lysed in RLT buffer (Qiagen). For the internalization assay, virus binding was repeated but after rinsing, DMEM with 2% FBS at 37° C. was added for 10, 20, or 30 min. Cells were washed and treated with 100 μg/mL of proteinase K (Invitrogen) for 15 min at 37° C. Proteinase K was rinsed away, and cells were treated with 100 μg/mL of RNAse A for 30 min at 37° C. Cells were rinsed six times with ice cold PBS and lysed in RLT buffer (Qiagen). RNA was isolated using a MagMax Viral Isolation Kit. SINV RNA and GAPDH RNA was quantified using a Taqman RNA-to-Ct 1-step kit with either SINV primers/probe (see above) or murine Gapdh primers/probe: FOR Gapdh: 5′-GTGGAGTCATACTGGAACATGTAG-3′ (SEQ ID NO: 173); REV Gapdh: 5′-AATGGTGAAGGTCGGTGTG-3′ (SEQ ID NO: 174); and probe: 5′ 6-FAM/TGCAAATGG/ZEN/CAGCCCTGGTG/3′ IABkFQ (SEQ ID NO: 175).

Blocking Assays with Anti-Chicken MXRA8 mAbs.

CEFs (2.5×10⁴) were seeded into 96-well plates. Twelve hours later, cells were incubated with mAbs (10 mg/mL) for 1 h at 37° C. in a volume of 50 μL, and then viruses (SINV (MOI 1, 8 h), SINV-WEEV (MOI 0.1, 8 h), AURAV (MOI 1, 24 h), WHAV (MOI 1, 17 h), BBKV (MOI 1, 8 h), OCKV (MOI 1, 22 h) or SINV-VEEV (TrD) (MOI 1, 9 h) in 50 μL were added and incubated for 8 h. Cells were collected, fixed, permeabilized, and E1 protein expression (except for SINV-VEEV-GFP) was monitored by flow cytometry after incubation with anti-E1 DC2.112 and DC2.315.

Neutralization Assays with MXRA8-Fc Fusion Proteins.

ΔMxra8 3T3 cells (2.5×10 4) complemented with either mouse or duck MXRA8 were seeded into 96-well plates overnight. SINV-CHIKV-LR 2006 virus (MOI of 1), SINV-TR339-GFP (MOI of 1) or SINV-WEEV-GFP (MOI of 0.1) were pre-incubated with increasing doses of LDLR3-D1-Fc, duck MXRA8-Fc, mouse MXRA8-Fc, Du-D1-Mo-D2 MXRA8-Fc, Du-D1-Mo-D2-N66R MXRA8-Fc or Mo-D1-Du-D2 MXRA8-Fc proteins for 1 h at 37° C. in a volume of 100 μL. Subsequently, viruses-receptor complexes were added to cells for 9 (SINV-CHIKV) or 10 (SINV-TR339-GFP and SINV-WEEV-GFP) h. Cells then were collected, fixed, and viral antigen expression was measured by flow cytometry.

Surface Staining of Infected Cells with MXRA8-Fc Fusion Proteins.

Vero cells were inoculated (multiplicity of infection [MOI] of 5 to 10) with SINV-TR339, CHIKV 181/25, MAYV or SINV-VEEV in DMEM supplemented with 2% FBS. After allowing infection to proceed for 14 to 18 h, cells were detached using TrypLE (Thermo Fisher) and washed with PBS. Cells were incubated with LDLRAD3-D1-Fc, Mouse MXRA8-Fc, Duck MXRA8-Fc, Du-D1-Mo-D2-Fc fusion proteins, or human anti-E1 DC2.112 mAb for 30 min at 4° C. in PBS, 2% FBS. Cells were washed and incubated with Alexa Fluor 647-conjugated goat anti-human or anti-mouse IgG (1:2000 dilution; Thermo Fisher) for 30 min at 4° C. Cells were washed and resuspended in PBS, 2% FBS, buffer containing 4′,6-diamidino-2-phenylindole (DAPI, 1 mg/mL) to stain dead cells and subjected to flow cytometry analysis using an iQue3 flow cytometer (Sartorius).

BLI-Based Competition Binding Assay.

Binding of mouse, duck, or Du-D1-Mo-D2 MXRA8 to captured CHIKV and WEEV VLP was monitored in real-time at 25° C. using an GatorPlus device (GatorBio) and analyzed using on-board software (GatorBio) or BiaEvaluation Software (Biacore). All experiments were performed with 1×PBS supplemented with 1% BSA. Anti-mouse IgG Fc biosensors (GatorBio #160004) were incubated with 5 μg/mL of CHK-265 (Fox 2015) or WEEV-209 (unpublished) for 100 sec then, after washing in running buffer for 60 sec, CHIKV VLPs or WEEV VLPs were captured at a nominal concentration of ˜20 μg/mL for 300 sec. VLP-coated biosensors were then submerged into the indicated concentrations of monovalent mouse, duck and Du-D1-Mo-D2 MXRA8 proteins cleaved from the Fc-fusions MXRA8 proteins using GlySERIAS (Genovis #A0-GS6).

Gene Editing of CEFs.

Chicken Mxra8 was targeted for gene editing in CEFs using sgRNAs to chicken Mxra8 (Chicken Mxra8 sgRNA: ACAGCTCCTACAACCAAGGG, SEQ ID NO: 176). Two sg RNA (CTGAAAAAGGAAGGAGTTGAG, SEQ ID NO: 177, and AAGATGAAAGGAAAGGCGTT, SEQ ID NO: 178) that do not target the chicken genome were included as negative controls. The sgRNAs were cloned into lentiCRISPR v.2 (Addgene no. 52961) and packaged in HEK-293 cells with psPAX2 (Addgene no. 12260) and pMD2.G (Addgene no. 12259) using FuGENE® HD (Promega). CEFs were transduced with lentiviruses and selected for 7 days in the presence of 1 mg/mL of puromycin. Clonal cell lines were obtained by limiting dilution. Mxra8 gene editing was confirmed by next generation sequencing on an Illumina HiSeq 2500 platform (Genome Technology Access Center of Washington University) with 300-base-pair paired-end sequencing.

MXRA8 Direct Binding Assays.

Maxisorp ELISA (Thermo Fisher) plates were coated with 4N12 anti-CHIKV mAb (2 μg/mL) overnight in sodium bicarbonate buffer, pH 9.3. Plates were washed four times with PBS and 0.05% Tween-20, and blocked with 4% BSA for 1 h at 25° C. CHIKV VLPs (1 μg/mL) were added in 2% BSA, and incubated for 2 h at 25° C. Chicken MXRA8-Fc, murine MXRA8-Fc, H77 (anti-HCV) mAb, and CHK-265 mAb were added in serial dilutions in 2% BSA, for 1 h at 25° C. Plates were washed with PBS and 0.05% Tween-20, and incubated with horseradish peroxide conjugated goat anti-mouse IgG (H+L) (1:2,000 dilution, Jackson ImmunoResearch) for 0.5 h at 25° C. After washing, plates were developed with 3,3′-5,5′ tetramethylbenzidine substrate (Thermo Fisher) and stopped with 2 N H2504. Plates were read at 450 nM using a TriStar Microplate Reader (Berthold).

Maxisorp ELISA plates were coated directly with WEEV VLPs (10 μg/mL) overnight in sodium bicarbonate buffer, pH 9.3. Plates were washed four times with PBS and 0.05% Tween-20, and blocked with 4% BSA for 1 h at 25° C. Chicken MXRA8-Fc, mouse MXRA8-Fc, H77 mAb and CH IK-265 mAb were added in serial dilutions in 2% BSA, for 1 h at 25° C. Plates were washed with PBS and 0.05% Tween-20, and incubated with horseradish peroxide conjugated goat anti-mouse IgG (H+L) (1:2,000 dilution, Jackson ImmunoResearch) for 0.5 h at 25° C. After washing, plates were developed with 3,3′-5,5′ tetramethylbenzidine substrate (Thermo Fisher) and stopped with 2 N H2504. Plates were read at 450 nM using a TriStar Microplate Reader (Berthold).

Maxisorp ELISA plates were coated with anti-His tag antibody (2 μg/mL, GenScript) overnight in sodium bicarbonate buffer, pH 9.3. Plates were washed four times with PBS and 0.05% Tween-20, and blocked with 4% BSA for 1 h at 25° C. His-tagged mouse MXRA8, duck MXRA8, sparrow MXRA8, and SARS-CoV-2 receptor binding domain (RBD) were added at 10 μg/mL in 2% BSA, for 1 h at 25° C. Plates were washed with PBS and 0.05% Tween-20, and incubated with WEEV VLPs or CHIKV VLPs at 10 μg/mL in 2% BSA, for 1 h at 25° C. Plates were washed with PBS and 0.05% Tween-20, and incubated with anti-WEEV-204 mAb or anti-CHIKV-265 mAb after serial dilutions, in 2% BSA, for 1 h at 25° C. Plates were washed with PBS and 0.05% Tween-20, and incubated with horseradish peroxide conjugated goat anti-mouse IgG (H+L) (1:2,000 dilution, Jackson ImmunoResearch) for 30 min at 25° C. After washing, plates were developed with 3,3′-5,5′ tetramethylbenzidine substrate (Thermo Fisher) and stopped with 2 N H2504. Plates were read at 450 nM using a TriStar Microplate Reader (Berthold).

Viral Burden Analysis.

RNA was extracted from serum and tissues using the MagMax-96 Viral RNA Isolation Kit (Thermo Fisher). SINV viral RNA levels were quantified by qRT-PCR using a TaqMan RNA-to-Ct 1-Step Kit (Thermo Fisher), compared to a SINV and OCKV RNA standard curve, and expressed on a log₁₀ scale as viral focus-forming unit (FFU) equivalents per gram of tissue or milliliter of serum. Primers and probes used are as follows: SINV FOR: 5′-AAGATCATCGACGCAGTCATC-3′ (SEQ ID NO: 179); SINV REV: 5′-GCTGTGGAAGTAACCGAATCT-3′ (SEQ ID NO: 180); SINV Probe: 5′-/56 FAM/CCACCTTAC/ZEN/TTCTGCGGCGGATTTA/3IABkFQ/-3′ (SEQ ID NO: 181). OCKV FOR: 5′-AGTTGGCTGTTTGCCCTT-3′ (SEQ ID NO: 182); OCKV REV: 5′-CGTGTGCTAGTCAGCATCAT-3′ (SEQ ID NO: 183); SINV Probe: 5′-/56-FAM/TAATTAATA/ZEN/GCGACGAGGCGCCGC/31ABkFQ/-3′ (SEQ ID NO: 184).

SINV infectious virus burden was evaluated using focus-forming assays as described above. Vero cells were incubated with serial dilutions of homogenized lung tissue samples. After 16 h, cells were fixed, permeabilized and stained as described above. Infected foci were visualized using TrueBlue peroxidase substrate (KPL) and quantitated on an ImmunoSpot 5.0.37 Macroanalyzer (Cellular Technologies).

Cryo-EM Sample Preparation, Data Collection, and Single Particle Reconstruction.

WEEV-VLP with and without avian or Du-D1-Mo-D2 MXRA8, and CHIKV with Du-D1-Mo-D2 MXRA8, in molar excess were flash-cooled on holey carbon EM grids in liquid ethane using an FEI Vitrobot (ThermoFisher). Movies of the samples were recorded with EPU software (Thermo Fisher) using a Falcon 4 electron detector (Thermo Fisher) on a Glacios microscope operating at 200 keV. Movies were collected with 48 frames and an electron total dose of 48 e⁻/Å² (1 e⁻/Å² per frame). The movies were corrected for beam-induced motion using MotionCor2. Contrast transfer function parameters of the electron micrographs were estimated using Gctf 1.18. Particles were auto-picked from a trained model using crYOLO. Subsequent 3D-classifications and 3D refinements were performed using RELION-3. Following whole-virus reconstruction, sub-particles representing the icosahedral asymmetric unit were extracted and refined using cryoSPARC. Following refinements, local resolution was estimated using RELION-3. Additional information regarding the work-flow, number of images, and particles is found in FIG. 46. Structures were visualized using ChimeraX and PYMOL.

Model Building and Analysis.

The initial models of the WEEV and CHIKV structural proteins (E1, E2, and TM regions) and MXRA8 (duck and Du-D1-Mo-D2) were built as threaded models using SWISS-MODEL. Threaded models then were simulated using Namdinator using the Molecular Dynamics Flexible Fitting Method to generate a potential to relax the structures into the observed experimental density. All components were docked into the map of an asymmetric unit as individual rigid bodies. The subunit models then underwent real-space refinement using PHENIX, first as rigid-body docking, then density morphing, and an iterative procedure of minimization using secondary-structure/torsion-restraints and manual editing using COOT 0.9.6, with additional refinement being performed using Isolde. A summary of refinement statistics is shown in TABLE 7. Close-contacts are computed as heavy-atoms within 0.39 nm and were calculated using MDTraj.

Virus Sequence Alignments.

All multiple sequence alignments were performed using the MUSCLE algorithm and visualized with ALINE. Sequences were obtained from GenBank as follows: WEEV-E1 (ABD98014.1), SINV-E1 (NP_740677.1), EEEV-E1 (NP_740648.1), VEEV-E1 (NP_741967.1), RRV-E1 (NP_740686.1), MAYV-E1 (NP_740694.1), CHIKV-E1 (NP_690589.2), WEEV-E2 (ABD98014.1), SINV-E2 (NP_740675.1), EEEV-E2 (NP_740646.1), VEEV-E2 (NP_741966.1), RRV-E2 (NP_740684.1), MAYV-E2 (NP_740693.1), CHIKV-E2 (NP_690589.2), duck MXRA8 (NWZ22901.1), sparrow MXRA8 (XP_039583663.1), turkey MXRA8 (XP_042687609.1), chicken MXRA8 (NP_989967.1), mouse MXRA8 (AAH26438.1), and human MXRA8 (NP_115724.1).

Phylogenetic Inference.

Structural protein (E1 and E2) sequences were retrieved from the NCBI GenBank for each alphavirus, including CHIKV (QKY67868.1), MAYV (QED21311.1), Una (UNAV, YP_009665989.1), ONNV (AAC97205.1), SFV (NP_463458.1), RRV (AAA47404.1), EEEV (ANB41743.1), Madariaga (MADV, AXV43855.1), VEEV (AGE98294.2), SINV (AAM10630.1), AURAV (NP_632024.1), OCKV (P27285.1), WEEV (QEX51909.1), Buggy Creek virus (BCV, AEJ36227.1), BBKV (AVN98166.1), FMV (YP_003324588.1), HJV (YP_002802300.1), and WHAV (AEJ36239.1). Sequences were aligned via Clustal Omega, with simple phylogeny inferred via neighbor-joining. Results were visualized in R using the ggtree package.

Sequences of D1 of MXRA8 were obtained with a BLAST search on the NCBI GenBank. The top 5,000 non-redundant sequences were obtained with an expect threshold of 0.05 and scored with BLOSUM62 for alignments. These sequences were aligned using Biopython, where residues corresponding to the position of duck D1-WEEV E1 contacts were extracted. Extracted sequences were characterized by the fraction of similarity to duck D1 and sorted, which were then inspected and curated manually to include in FIG. 4I. Sequences shown include Duck (NWZ22901.1), Alligator (XP_006263071.1), Crocodile (XP_019397120.1), Sea Turtle (XP_007070253.1), Turtle (KAG6936352.1), Coelacanth (XP_005986077.1), Lizard (XP_042335573.1), Caecilian (XP_030041825.1), Eel (XP_028663187.1), Komoto Dragon (XP_044301186.1), Snake (XP_034290786.1), Birchir (XP_039612125.1), Carp (XP_042590098.1), Shark (XP_041062630.1), Opossum (XP_007481143.1), Mink (XP_044092840.1), Frog (KAG9482299.1), Toad (XP_040283849.1), Beaver (XP_020011302.1), Possum (XP_036598586.1), Bat (XP_015999954.1), and Mouse (AAH26438.1).

Quantification and Statistical Analysis

Statistical significance was assigned using Prism Version 8.0 (GraphPad) when P<0.05. Statistical analysis of viral infection levels was determined by one-way ANOVA with Dunnett's post-test. Statistical analysis of in vivo experiments was determined by either one-way or two-way ANOVA with a Kruskal-Wallis or Dunnett's post-test depending on the data distribution and the number of comparison groups. The statistical tests, number of independent experiments, and number of experimental replicates are indicated in the Figure legends.

What is claimed is:

1. A fusion protein comprising:
an Fc region;
a first Mxra8 region; and
a second Mxra8 region;
and wherein the fusion protein reduces inflammation and infection by an arthritogenic alphavirus.

2. The fusion protein of claim 1, wherein the Fc region is selected from human IgG1, human IgG1 with N297Q mutation, and mouse IgG2b.

3. The fusion protein of claim 1, wherein at least one of the first Mxra8 region and the second Mxra8 region is a Mxra8 ectodomain region.

4. The fusion protein of claim 1, wherein the first Mxra8 region comprises a mammalian Mxra8 region and the second Mxra8 region comprises a non-mammalian Mxra8 region.

5. The fusion protein of claim 4, wherein the mammalian Mxra8 region comprises a D2 mammalian Mxra8 region.

6. The fusion protein of claim 5, wherein the D2 mammalian Mxra8 region is a D2 mouse Mxra8 region.

7. The fusion protein of claim 4, wherein the non-mammalian Mxra8 region comprises a D1 avian Mxra8 region or a D1 reptile Mxra8 region.

8. The fusion protein of claim 7, wherein the D1 avian Mxra8 region is a D1 duck Mxra8 region.

9. A method of reducing Mxra8-associated alphavirus infection in a subject, the method comprising administering to the subject a therapeutically effective amount of a Mxra8 inhibiting agent comprising a fusion protein, wherein the fusion protein comprises:
an Fc region;
a first Mxra8 region; and
a second Mxra8 region.

10. The method of claim 9, wherein the Fc region is selected from human IgG1, human IgG1 with N297Q mutation, and mouse IgG2b.

11. The method of claim 9, wherein the first Mxra8 region comprises a mammalian Mxra8 region and the second Mxra8 region comprises a non-mammalian Mxra8 region.

12. The method of claim 9, wherein the Mxra8-associated alphavirus is an arthritogenic alphavirus selected from Chikungunya Virus (CHIKV), Mayaro Virus (MAYV), Ross River Virus (RRV), O'nyong nyong (ONNV), Barmah Forest Virus (BFV), Semliki Forest Virus (SFV), and Getah virus.

13. The method of claim 9, wherein the Mxra8-associated alphavirus is a WEE complex alphavirus selected from Aura Virus (AURAV), Fort Morgan Virus (FMV), Highlands J Virus (HJV), Western Equine Encephalitis Virus (WEEV), Sindbis Virus (SINV), and Whataroa Virus (WHAV).

14. The method of claim 9, wherein the subject is a mammalian subject selected from a human, a non-human primate, a horse, a dog, a cat, a sheep, a pig, a mouse, a rat, a monkey, a hamster, and a guinea pig.

15. The method of claim 9, wherein the subject is a non-mammalian subject selected from a reptile, a duck, a turkey, and a chicken.

16. A method of binding at least one of a surface-displayed alphavirus E1 protein and a surface-displayed alphavirus E2 protein in alphavirus-infected cells, the method comprising administering to an alphavirus-infected cell a therapeutically effective amount of a Mxra8 inhibiting agent comprising a fusion protein, wherein the fusion protein comprises:
an Fc region;
a first Mxra8 region; and
a second Mxra8 region.

17. The method of claim 16, wherein the Fc region is selected from human IgG1, human IgG1 with N297Q mutation, and mouse IgG2b.

18. The method of claim 16, wherein the first Mxra8 region comprises a mammalian Mxra8 region and the second Mxra8 region comprises a non-mammalian Mxra8 region.

19. The method of claim 16, wherein the alphavirus-infected cell is infected with an arthritogenic alphavirus selected from Chikungunya Virus (CHIKV), Mayaro Virus (MAYV), Ross River Virus (RRV), O'nyong nyong (ONNV), Barmah Forest Virus (BFV), Semliki Forest Virus (SFV), and Getah virus.

20. The method of claim 16, wherein the alphavirus-infected cell is infected with a WEE complex alphavirus selected from Aura Virus (AURAV), Fort Morgan Virus (FMV), Highlands J Virus (HJV), Western Equine Encephalitis Virus (WEEV), Sindbis Virus (SINV), and Whataroa Virus (WHAV).


SEQUENCE LISTING (185 sequences)

SEQ ID NO: 1 - DNA, 954 bp, Homo sapiens
  source 1..954: mol_type = genomic DNA
gtcctgttgcatagcggctcctctgtcccggccgcagcgggatcaagcgtagtgagcgagtcagcagtttcctgggaagcgggggcaagggctgttctgaggtgtcagagcccaaggatggtgtggacgcaagaccggctccacgacaggcaaagagtgcttcactgggacctcaggggacccggtggtggcccagcacgccggctcctggacctctatagcgcgggagaacaacgagtatacgaagctagggacagaggaagactggagctttcagccagtgcgttcgatgatggaaatttttcacttctgatccgggctgtggaagaaactgatgctggactgtatacttgtaatctccaccatcactactgccacctctatgagtcattggctgtcagactggaagttaccgatggcccgcccgccacaccagcttactgggatggcgaaaaagaagtacttgctgtcgcgcgaggtgcccctgcgctcctgacatgcgtcaacaggggtcacgtttggactgatcggcacgtcgaagaggctcagcaagtagttcactgggaccgccagccgcctggcgtacctcacgatagggccgaccgcctcttggacctctacgcctccggtgaaaggagggcctatgggcctctgttcctcagagatagggttgcggtgggcgcagatgcctttgaacgaggcgatttcagcctccgcattgagcccctggaggtagccgatgagggcacctactcctgtcatctgcatcatcattattgcggattgcacgagaggagggtcttccacttgactgtagctgaaccgcatgctgaaccgccacctcgcggatcacccggaaacggtagcagtcattccggggctcccgggccggatccgacattggcacgagggcataacgtaataaatgtcatcgttcccgagtcacgcgctcat

SEQ ID NO: 2 - protein, 318 aa, Homo sapiens
VLLHSGSSVPAAAGSSVVSESAVSWEAGARAVLRCQSPRMVWTQDRLHDRQRVLHWDLRGPGGGPARRLLDLYSAGEQRVYEARDRGRLELSASAFDDGNFSLLIRAVEETDAGLYTCNLHHHYCHLYESLAVRLEVTDGPPATPAYWDGEKEVLAVARGAPALLTCVNRGHVWTDRHVEEAQQVVHWDRQPPGVPHDRADRLLDLYASGERRAYGPLFLRDRVAVGADAFERGDFSLRIEPLEVADEGTYSCHLHHHYCGLHERRVFHLTVAEPHAEPPPRGSPGNGSSHSGAPGPDPTLARGHNVINVIVPESRAH

SEQ ID NO: 3 - DNA, 942 bp, Mus musculus
  source 1..942: mol_type = genomic DNA
tctggcccgagcggtactgctgccgcaagctcaagcctcgtctctgaatcagttgtgtctctcgcggccggaactcaggcggtcctgaggtgtcaaagtccaaggatggtttggacccaagatcgcttgcatgatcgacaaagagttgtgcactgggacctcagcggtggtcctggatcacaacgaagaaggctggtggacatgtactcagccggagaacagcgcgtttacgagccacgagaccgagatcgactccttttgtcaccttctgcgttccatgacggcaacttttctttgttgataagagccgtagatcgcggagatgaaggagtttatacgtgtaaccttcaccaccattactgccatctcgatgaaagtctcgccgtacggctggaagttacggaggacccacttctttcacgggcatattgggatggcgagaaggaggtcctggtcgttgctcacggcgcaccagccctgatgacatgcatcaacagagcccatgtttggaccgacaggcaccttgaagaagcacaacaagttgtccattgggaccgacagctcccaggtgttagccatgatcgggccgatcgcttgcttgacttgtatgccagcggggaacggagggcatacggcccgccttttcttcgggatcgagttagcgtaaacactaacgcctttgcacgcggtgacttcagccttcgcatagatgaactcgaacgagcagatgaaggaatatactcctgccatttgcatcatcattattgcggcctgcatgagcgaagggtatttcacctccaagtgactgaacctgcattcgagccaccagcgagggcctcaccgggaaacggctccggccacagcagcgcaccgtctccggacccgactttgacacggggccatagtataataaacgtaatagttcctgaggaccatacccac

SEQ ID NO: 4 - protein, 314 aa, Mus musculus
SGPSGTAAASSSLVSESVVSLAAGTQAVLRCQSPRMVWTQDRLHDRQRVVHWDLSGGPGSQRRRLVDMYSAGEQRVYEPRDRDRLLLSPSAFHDGNFSLLIRAVDRGDEGVYTCNLHHHYCHLDESLAVRLEVTEDPLLSRAYWDGEKEVLVVAHGAPALMTCINRAHVWTDRHLEEAQQVVHWDRQLPGVSHDRADRLLDLYASGERRAYGPPFLRDRVSVNTNAFARGDFSLRIDELERADEGIYSCHLHHHYCGLHERRVFHLQVTEPAFEPPARASPGNGSGHSSAPSPDPTLTRGHSIINVIVPEDHTH

SEQ ID NO: 5 - DNA, 933 bp, Mus musculus
  source 1..933: mol_type = genomic DNA
cccagcgggcccatttcaacaatcaacccctgtcctccatgcaaggagtgtcacaaatgcccaggtaagtcactaccagagctccactcccaggagaatggtaagtgctgtaaaaatccctgtaatggaggataagccatgtacaaatccatttccatctctcctcatcagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaaggtgggacctgcaggacaactgcatgggggctgggatgggcataagaataaatgtctatgtggacagccttccacttcagccatgacctctatgtatgtttctaaccccacagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaa

SEQ ID NO: 6 - protein, 238 aa, Mus musculus
PSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 7 - DNA, 681 bp, Homo sapiens
  source 1..681: mol_type = genomic DNA
gacaaaacccatacatgcccaccttgtccagcaccagagctgctgggaggaccaagcgtgttcctgtttccacccaagcccaaagataccctgatgatctcccggactcccgaagtcacctgcgtggtcgtggacgtgtctcacgaggaccccgaagtcaagttcaactggtacgtggacggggtcgaggtgcataatgccaagacaaaacccagagaggaacagtacaacagtacctatagagtcgtgtcagtcctgacagtgctgcatcaggattggctgaacgggaaagagtataagtgcaaagtgagtaataaggctctgcctgcaccaatcgagaaaactatttccaaggccaaaggacagcccagggaacctcaggtgtacaccctgcctccatcacgcgaggaaatgacaaagaaccaggtcagcctgacttgtctggtgaaaggcttctatccttctgacatcgctgtggagtgggaaagtaatgggcagccagagaacaattacaagacaactccccctgtcctggactctgatggaagtttctttctgtatagcaagctgaccgtggataaatcccggtggcagcagggcaatgtctttagctgttccgtgatgcacgaagccctgcacaaccactacacccagaagagcctgagcctgtcacccggcaaa

SEQ ID NO: 8 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 681 bp, Homo sapiens
  source 1..681: mol_type = genomic DNA
gacaaaacccatacatgcccaccttgtccagcaccagagctgctgggaggaccaagcgtgttcctgtttccacccaagcccaaagataccctgatgatctcccggactcccgaagtcacctgcgtggtcgtggacgtgtctcacgaggaccccgaagtcaagttcaactggtacgtggacggggtcgaggtgcataatgccaagacaaaacccagagaggaacagtaccagagtacctatagagtcgtgtcagtcctgacagtgctgcatcaggattggctgaacgggaaagagtataagtgcaaagtgagtaataaggctctgcctgcaccaatcgagaaaactatttccaaggccaaaggacagcccagggaacctcaggtgtacaccctgcctccatcacgcgaggaaatgacaaagaaccaggtcagcctgacttgtctggtgaaaggcttctatccttctgacatcgctgtggagtgggaaagtaatgggcagccagagaacaattacaagacaactccccctgtcctggactctgatggaagtttctttctgtatagcaagctgaccgtggataaatcccggtggcagcagggcaatgtctttagctgttccgtgatgcacgaagccctgcacaaccactacacccagaagagcctgagcctgtcacccggcaaa

SEQ ID NO: 10 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 60 bp, Synthetic construct
  source 1..60: mol_type = other DNA
atgtacaggatgcaactcctgtcttgcattgcactaagtcttgcacttgtcacgaattcg

SEQ ID NO: 12 - protein, 20 aa, Synthetic construct
MYRMQLLSCIALSLALVTNS

SEQ ID NO: 13 - DNA, 27 bp, Synthetic construct
  source 1..27: mol_type = other DNA
tcaagcggcagcagcggcagttcaggg

SEQ ID NO: 14 - protein, 9 aa, Synthetic construct
SSGSSGSSG

SEQ ID NO: 15 - DNA, 84 bp, Synthetic construct
  source 1..84: mol_type = other DNA
atggggatccttcccagccctgggatgcctgcgctgctctccctcgtgagccttctctccgtgctgctgatgggttgcgtagct

SEQ ID NO: 16 - protein, 28 aa, Synthetic construct
MGILPSPGMPALLSLVSLLSVLLMGCVA

SEQ ID NO: 17 - DNA, 1968 bp, Synthetic construct
  source 1..1968: mol_type = other DNA
atgtacaggatgcaactcctgtcttgcattgcactaagtcttgcacttgtcacgaattcgatatcggtcctgttgcatagcggctcctctgtcccggccgcagcgggatcaagcgtagtgagcgagtcagcagtttcctgggaagcgggggcaagggctgttctgaggtgtcagagcccaaggatggtgtggacgcaagaccggctccacgacaggcaaagagtgcttcactgggacctcaggggacccggtggtggcccagcacgccggctcctggacctctatagcgcgggagaacaacgagtatacgaagctagggacagaggaagactggagctttcagccagtgcgttcgatgatggaaatttttcacttctgatccgggctgtggaagaaactgatgctggactgtatacttgtaatctccaccatcactactgccacctctatgagtcattggctgtcagactggaagttaccgatggcccgcccgccacaccagcttactgggatggcgaaaaagaagtacttgctgtcgcgcgaggtgcccctgcgctcctgacatgcgtcaacaggggtcacgtttggactgatcggcacgtcgaagaggctcagcaagtagttcactgggaccgccagccgcctggcgtacctcacgatagggccgaccgcctcttggacctctacgcctccggtgaaaggagggcctatgggcctctgttcctcagagatagggttgcggtgggcgcagatgcctttgaacgaggcgatttcagcctccgcattgagcccctggaggtagccgatgagggcacctactcctgtcatctgcatcatcattattgcggattgcacgagaggagggtcttccacttgactgtagctgaaccgcatgctgaaccgccacctcgcggatcacccggaaacggtagcagtcattccggggctcccgggccggatccgacattggcacgagggcataacgtaataaatgtcatcgttcccgagtcacgcgctcatgctagcgttagatctcccagcgggcccatttcaacaatcaacccctgtcctccatgcaaggagtgtcacaaatgcccaggtaagtcactaccagagctccactcccaggagaatggtaagtgctgtaaaaatccctgtaatggaggataagccatgtacaaatccatttccatctctcctcatcagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaaggtgggacctgcaggacaactgcatgggggctgggatgggcataagaataaatgtctatgtggacagccttccacttcagccatgacctctatgtatgtttctaaccccacagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaa

SEQ ID NO: 18 - protein, 583 aa, Synthetic construct
MYRMQLLSCIALSLALVTNSISVLLHSGSSVPAAAGSSVVSESAVSWEAGARAVLRCQSPRMVWTQDRLHDRQRVLHWDLRGPGGGPARRLLDLYSAGEQRVYEARDRGRLELSASAFDDGNFSLLIRAVEETDAGLYTCNLHHHYCHLYESLAVRLEVTDGPPATPAYWDGEKEVLAVARGAPALLTCVNRGHVWTDRHVEEAQQVVHWDRQPPGVPHDRADRLLDLYASGERRAYGPLFLRDRVAVGADAFERGDFSLRIEPLEVADEGTYSCHLHHHYCGLHERRVFHLTVAEPHAEPPPRGSPGNGSSHSGAPGPDPTLARGHNVINVIVPESRAHASVRSPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 19 - DNA, 1956 bp, Synthetic construct
  source 1..1956: mol_type = other DNA
atgtacaggatgcaactcctgtcttgcattgcactaagtcttgcacttgtcacgaattcgatatcgtctggcccgagcggtactgctgccgcaagctcaagcctcgtctctgaatcagttgtgtctctcgcggccggaactcaggcggtcctgaggtgtcaaagtccaaggatggtttggacccaagatcgcttgcatgatcgacaaagagttgtgcactgggacctcagcggtggtcctggatcacaacgaagaaggctggtggacatgtactcagccggagaacagcgcgtttacgagccacgagaccgagatcgactccttttgtcaccttctgcgttccatgacggcaacttttctttgttgataagagccgtagatcgcggagatgaaggagtttatacgtgtaaccttcaccaccattactgccatctcgatgaaagtctcgccgtacggctggaagttacggaggacccacttctttcacgggcatattgggatggcgagaaggaggtcctggtcgttgctcacggcgcaccagccctgatgacatgcatcaacagagcccatgtttggaccgacaggcaccttgaagaagcacaacaagttgtccattgggaccgacagctcccaggtgttagccatgatcgggccgatcgcttgcttgacttgtatgccagcggggaacggagggcatacggcccgccttttcttcgggatcgagttagcgtaaacactaacgcctttgcacgcggtgacttcagccttcgcatagatgaactcgaacgagcagatgaaggaatatactcctgccatttgcatcatcattattgcggcctgcatgagcgaagggtatttcacctccaagtgactgaacctgcattcgagccaccagcgagggcctcaccgggaaacggctccggccacagcagcgcaccgtctccggacccgactttgacacggggccatagtataataaacgtaatagttcctgaggaccatacccacgctagcgttagatctcccagcgggcccatttcaacaatcaacccctgtcctccatgcaaggagtgtcacaaatgcccaggtaagtcactaccagagctccactcccaggagaatggtaagtgctgtaaaaatccctgtaatggaggataagccatgtacaaatccatttccatctctcctcatcagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaaggtgggacctgcaggacaactgcatgggggctgggatgggcataagaataaatgtctatgtggacagccttccacttcagccatgacctctatgtatgtttctaaccccacagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaa

SEQ ID NO: 20 - protein, 579 aa, Synthetic construct
MYRMQLLSCIALSLALVTNSISSGPSGTAAASSSLVSESVVSLAAGTQAVLRCQSPRMVWTQDRLHDRQRVVHWDLSGGPGSQRRRLVDMYSAGEQRVYEPRDRDRLLLSPSAFHDGNFSLLIRAVDRGDEGVYTCNLHHHYCHLDESLAVRLEVTEDPLLSRAYWDGEKEVLVVAHGAPALMTCINRAHVWTDRHLEEAQQVVHWDRQLPGVSHDRADRLLDLYASGERRAYGPPFLRDRVSVNTNAFARGDFSLRIDELERADEGIYSCHLHHHYCGLHERRVFHLQVTEPAFEPPARASPGNGSGHSSAPSPDPTLTRGHSIINVIVPEDHTHASVRSPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 21 - DNA, 1743 bp, Synthetic construct
  source 1..1743: mol_type = other DNA
atggggatccttcccagccctgggatgcctgcgctgctctccctcgtgagccttctctccgtgctgctgatgggttgcgtagctgaaaccggttctggcccgagcggtactgctgccgcaagctcaagcctcgtctctgaatcagttgtgtctctcgcggccggaactcaggcggtcctgaggtgtcaaagtccaaggatggtttggacccaagatcgcttgcatgatcgacaaagagttgtgcactgggacctcagcggtggtcctggatcacaacgaagaaggctggtggacatgtactcagccggagaacagcgcgtttacgagccacgagaccgagatcgactccttttgtcaccttctgcgttccatgacggcaacttttctttgttgataagagccgtagatcgcggagatgaaggagtttatacgtgtaaccttcaccaccattactgccatctcgatgaaagtctcgccgtacggctggaagttacggaggacccacttctttcacgggcatattgggatggcgagaaggaggtcctggtcgttgctcacggcgcaccagccctgatgacatgcatcaacagagcccatgtttggaccgacaggcaccttgaagaagcacaacaagttgtccattgggaccgacagctcccaggtgttagccatgatcgggccgatcgcttgcttgacttgtatgccagcggggaacggagggcatacggcccgccttttcttcgggatcgagttagcgtaaacactaacgcctttgcacgcggtgacttcagccttcgcatagatgaactcgaacgagcagatgaaggaatatactcctgccatttgcatcatcattattgcggcctgcatgagcgaagggtatttcacctccaagtgactgaacctgcattcgagccaccagcgagggcctcaccgggaaacggctccggccacagcagcgcaccgtctccggacccgactttgacacggggccatagtataataaacgtaatagttcctgaggaccatacccactcaagcggcagcagcggcagttcaggggacaaaacccatacatgcccaccttgtccagcaccagagctgctgggaggaccaagcgtgttcctgtttccacccaagcccaaagataccctgatgatctcccggactcccgaagtcacctgcgtggtcgtggacgtgtctcacgaggaccccgaagtcaagttcaactggtacgtggacggggtcgaggtgcataatgccaagacaaaacccagagaggaacagtacaacagtacctatagagtcgtgtcagtcctgacagtgctgcatcaggattggctgaacgggaaagagtataagtgcaaagtgagtaataaggctctgcctgcaccaatcgagaaaactatttccaaggccaaaggacagcccagggaacctcaggtgtacaccctgcctccatcacgcgaggaaatgacaaagaaccaggtcagcctgacttgtctggtgaaaggcttctatccttctgacatcgctgtggagtgggaaagtaatgggcagccagagaacaattacaagacaactccccctgtcctggactctgatggaagtttctttctgtatagcaagctgaccgtggataaatcccggtggcagcagggcaatgtctttagctgttccgtgatgcacgaagccctgcacaaccactacacccagaagagcctgagcctgtcacccggcaaa

SEQ ID NO: 22 - protein, 581 aa, Synthetic construct
MGILPSPGMPALLSLVSLLSVLLMGCVAETGSGPSGTAAASSSLVSESVVSLAAGTQAVLRCQSPRMVWTQDRLHDRQRVVHWDLSGGPGSQRRRLVDMYSAGEQRVYEPRDRDRLLLSPSAFHDGNFSLLIRAVDRGDEGVYTCNLHHHYCHLDESLAVRLEVTEDPLLSRAYWDGEKEVLVVAHGAPALMTCINRAHVWTDRHLEEAQQVVHWDRQLPGVSHDRADRLLDLYASGERRAYGPPFLRDRVSVNTNAFARGDFSLRIDELERADEGIYSCHLHHHYCGLHERRVFHLQVTEPAFEPPARASPGNGSGHSSAPSPDPTLTRGHSIINVIVPEDHTHSSGSSGSSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 1743 bp, Synthetic construct
  source 1..1743: mol_type = other DNA
atggggatccttcccagccctgggatgcctgcgctgctctccctcgtgagccttctctccgtgctgctgatgggttgcgtagctgaaaccggttctggcccgagcggtactgctgccgcaagctcaagcctcgtctctgaatcagttgtgtctctcgcggccggaactcaggcggtcctgaggtgtcaaagtccaaggatggtttggacccaagatcgcttgcatgatcgacaaagagttgtgcactgggacctcagcggtggtcctggatcacaacgaagaaggctggtggacatgtactcagccggagaacagcgcgtttacgagccacgagaccgagatcgactccttttgtcaccttctgcgttccatgacggcaacttttctttgttgataagagccgtagatcgcggagatgaaggagtttatacgtgtaaccttcaccaccattactgccatctcgatgaaagtctcgccgtacggctggaagttacggaggacccacttctttcacgggcatattgggatggcgagaaggaggtcctggtcgttgctcacggcgcaccagccctgatgacatgcatcaacagagcccatgtttggaccgacaggcaccttgaagaagcacaacaagttgtccattgggaccgacagctcccaggtgttagccatgatcgggccgatcgcttgcttgacttgtatgccagcggggaacggagggcatacggcccgccttttcttcgggatcgagttagcgtaaacactaacgcctttgcacgcggtgacttcagccttcgcatagatgaactcgaacgagcagatgaaggaatatactcctgccatttgcatcatcattattgcggcctgcatgagcgaagggtatttcacctccaagtgactgaacctgcattcgagccaccagcgagggcctcaccgggaaacggctccggccacagcagcgcaccgtctccggacccgactttgacacggggccatagtataataaacgtaatagttcctgaggaccatacccactcaagcggcagcagcggcagttcaggggacaaaacccatacatgcccaccttgtccagcaccagagctgctgggaggaccaagcgtgttcctgtttccacccaagcccaaagataccctgatgatctcccggactcccgaagtcacctgcgtggtcgtggacgtgtctcacgaggaccccgaagtcaagttcaactggtacgtggacggggtcgaggtgcataatgccaagacaaaacccagagaggaacagtaccagagtacctatagagtcgtgtcagtcctgacagtgctgcatcaggattggctgaacgggaaagagtataagtgcaaagtgagtaataaggctctgcctgcaccaatcgagaaaactatttccaaggccaaaggacagcccagggaacctcaggtgtacaccctgcctccatcacgcgaggaaatgacaaagaaccaggtcagcctgacttgtctggtgaaaggcttctatccttctgacatcgctgtggagtgggaaagtaatgggcagccagagaacaattacaagacaactccccctgtcctggactctgatggaagtttctttctgtatagcaagctgaccgtggataaatcccggtggcagcagggcaatgtctttagctgttccgtgatgcacgaagccctgcacaaccactacacccagaagagcctgagcctgtcacccggcaaa

SEQ ID NO: 24 - protein, 581 aa, Synthetic construct
MGILPSPGMPALLSLVSLLSVLLMGCVAETGSGPSGTAAASSSLVSESVVSLAAGTQAVLRCQSPRMVWTQDRLHDRQRVVHWDLSGGPGSQRRRLVDMYSAGEQRVYEPRDRDRLLLSPSAFHDGNFSLLIRAVDRGDEGVYTCNLHHHYCHLDESLAVRLEVTEDPLLSRAYWDGEKEVLVVAHGAPALMTCINRAHVWTDRHLEEAQQVVHWDRQLPGVSHDRADRLLDLYASGERRAYGPPFLRDRVSVNTNAFARGDFSLRIDELERADEGIYSCHLHHHYCGLHERRVFHLQVTEPAFEPPARASPGNGSGHSSAPSPDPTLTRGHSIINVIVPEDHTHSSGSSGSSGDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 343 aa, Homo sapiens
MALPSRILLWKLVLLQSSAVLLHSAVEETDAGLYTCNLHHHYCHLYESLAVRLEVTDGPPATPAYWDGEKEVLAVARGAPALLTCVNRGHVWTDRHVEEAQQVVHWDRQPPGVPHDRADRLLDLYASGERRAYGPLFLRDRVAVGADAFERGDFSLRIEPLEVADEGTYSCHLHHHYCGLHERRVFHLTVAEPHAEPPPRGSPGNGSSHSGAPGPDPTLARGHNVINVIVPESRAHFFQQLGYVLATLLLFILLLVTVLLAARRRRGGYEYSDQKSGKSKGKDVNLAEFAVAAGDQMLYRSEDIQLDYKNNILKERAELAHSPLPAKYIDLDKDPSGLCPLGA

SEQ ID NO: 26 - protein, 444 aa, Homo sapiens
MALPSRILLWKLVLLQSSAVLLHSGSSVPAAAGSSVVSESAVSWEAGARAVLRCQSPRMVWTQDRLHDRQRVLHWDLRGPGGGPARRLLDLYSAGEQRVYEARDRGRLELSASAFDDGNFSLLIRAVEETDAGLYTCNLHHHYCHLYESLAVRLEVTDGPPATPAYWDGEKEVLAVARGAPALLTCVNRGHVWTDRHVEEAQQVVHWDRQPPGVPHDRADRLLDLYASGERRAYGPLFLRDRVAVGADAFERGDFSLRIEPLEVADEGTYSCHLHHHYCGLHERRVFHLTVAEPHAEPPPRGSPGNGSSHSGAPGPDPTLARGHNVINVIVPESRAHFFQQLGYVLATLLLFILLLVTVLLAARRRRGGYEYSDQKSGKSKGKDVNLAEFAVAAGDQMLYRSEDIQLDYKNNILKERAELAHSPLPAKYIDLDKDPSGLCPLGA

SEQ ID NO: 27 - protein, 442 aa, Homo sapiens
MALPSRILLWKLVLLQSSAVLLHSGSSVPAAAGSSVVSESAVSWEAGARAVLRCQSPRMVWTQDRLHDRQRVLHWDLRGPGGGPARRLLDLYSAGEQRVYEARDRGRLELSASAFDDGNFSLLIRAVEETDAGLYTCNLHHHYCHLYESLAVRLEVTDGPPATPAYWDGEKEVLAVARGAPALLTCVNRGHVWTDRHVEEAQQVVHWDRQPPGVPHDRADRLLDLYASGERRAYGPLFLRDRVAVGADAFERGDFSLRIEPLEVADEGTYSCHLHHHYCGLHERRVFHLTVAEPHAEPPPRGSPGNGSSHSGAPGPDPTLARGHNVINVIVPESRAHFFQQLGYVLATLLLFILLLVTVLLAARRRRGGYEYSDQKSGKSKGKDVNLAEFAVAAGDQMLYRSEDIQLDYKNNILKERAELAHSPLPAKYIDLDKGFRKENCK

SEQ ID NO: 28 - protein, 433 aa, Homo sapiens
MIRCAATGSAVLLHSGSSVPAAAGSSVVSESAVSWEAGARAVLRCQSPRMVWTQDRLHDRQRVLHWDLRGPGGGPARRLLDLYSAGEQRVYEARDRGRLELSASAFDDGNFSLLIRAVEETDAGLYTCNLHHHYCHLYESLAVRLEVTDGPPATPAYWDGEKEVLAVARGAPALLTCVNRGHVWTDRHVEEAQQVVHWDRQPPGVPHDRADRLLDLYASGERRAYGPLFLRDRVAVGADAFERGDFSLRIEPLEVADEGTYSCHLHHHYCGLHERRVFHLTVAEPHAEPPPRGSPGNGSSHSGAPGPDPTLARGHNVINVIVPESRAHFFQQLGYVLATLLLFILLLVTVLLAARRRRGGYEYSDQKSGKSKGKDVNLAEFAVAAGDQMLYRSEDIQLDYKNNILKERAELAHSPLPAKYIDLDKGFRKENCK

SEQ ID NO: 29 - protein, 341 aa, Homo sapiens
MALPSRILLWKLVLLQSSAVLLHSAVEETDAGLYTCNLHHHYCHLYESLAVRLEVTDGPPATPAYWDGEKEVLAVARGAPALLTCVNRGHVWTDRHVEEAQQVVHWDRQPPGVPHDRADRLLDLYASGERRAYGPLFLRDRVAVGADAFERGDFSLRIEPLEVADEGTYSCHLHHHYCGLHERRVFHLTVAEPHAEPPPRGSPGNGSSHSGAPGPDPTLARGHNVINVIVPESRAHFFQQLGYVLATLLLFILLLVTVLLAARRRRGGYEYSDQKSGKSKGKDVNLAEFAVAAGDQMLYRSEDIQLDYKNNILKERAELAHSPLPAKYIDLDKGFRKENCK

SEQ ID NO: 30 - protein, 450 aa, Homo sapiens
MALPSRILLWKLVLLQSSAVLLHSGSSVPAAAGSSVVSESAVSWEAGARAVLRCQSPRMVWTQDRLHDRQRVLHWDLRGPGGGPARRLLDLYSAGEQRVYEARDRGRLELSASAFDDGNFSLLIRAVEETDAGLYTCNLHHHYCHLYESLAVRLEVTDGPPATPAYWDGEKEVLAVARGAPALLTCVNRGHVWTDRHVEEAQQVVHWDRQPPGVPHDRADRLLDLYASGERRAYGPLFLRDRVAVGADAFERGDFSLRIEPLEVADEGTYSCHLHHHYCGLHERRVFHLTVAEPHAEPPPRGSPGNGSSHSGAPGPDPTLARGHNVINVIVPESRAHFFQQLGYVLATLLLFILLLVTVLLAARRRRGGYEYSDQKSGKSKGKDVNLAEFAVAAGDQMLYRSEDIQLASSPPTDYKNNILKERAELAHSPLPAKYIDLDKDPSGLCPLGA

SEQ ID NO: 31 - protein, 442 aa, Homo sapiens; identical to SEQ ID NO: 27

SEQ ID NO: 32 - protein, 2280 aa, Mus musculus
AGTCAAGATGGGGGAGGCCAAGCAGTTGAGCAGCGCAGTGGGCGGGGCCCAGACCCGGCCCAGGGCTCCGTGATGTCAGCGGGTGCTGGCAGGGAGAGGGGGTCGGCTGTTTTTCTGGAGAGTTAGAGCTGAGTAAGACAAAGCACGTCCCCCGCAGGCGCCATGGAGCTGCTGTCCCGCGTCCTGCTGTGGAAACTGCTGCTTCTTCAGAGCTCTGCAGTCCTGTCCTCAGGGCCTTCAGGGACCGCAGCAGCCAGCAGCTCTCTGGTGTCTGAGTCTGTGGTGAGCTTGGCAGCCGGAACCCAGGCTGTGCTACGCTGCCAGAGCCCCCGCATGGTGTGGACCCAAGACCGGCTGCATGATCGCCAGCGCGTGGTCCACTGGGACCTCAGCGGGGGCCCGGGCAGCCAACGGCGCCGACTTGTGGATATGTATTCGGCGGGTGAACAGCGCGTGTACGAGCCGCGCGATCGCGACCGCCTCCTGCTGTCGCCTTCTGCTTTCCACGACGGCAACTTCTCGCTGCTCATTCGCGCTGTGGACAGAGGCGATGAAGGGGTGTACACCTGCAACCTGCACCATCACTACTGCCACCTCGATGAGAGCCTGGCTGTGCGCCTCGAGGTTACAGAGGATCCCCTATTAAGTCGCGCATACTGGGACGGTGAGAAGGAAGTGTTGGTGGTGGCCCATGGCGCGCCGGCACTGATGACCTGCATCAACCGTGCGCACGTGTGGACTGACCGCCATTTAGAGGAGGCGCAGCAGGTGGTCCATTGGGACCGACAGCTACCTGGGGTGTCACACGACCGCGCCGACCGCCTGCTTGACCTGTATGCATCTGGCGAGCGCCGCGCCTATGGGCCACCCTTCCTGCGTGATCGCGTGTCAGTGAACACCAACGCTTTTGCACGCGGTGACTTCTCCCTACGCATCGATGAGCTGGAGCGAGCTGATGAGGGCATCTATTCCTGCCACCTGCACCATCACTACTGTGGCCTCCACGAGCGCCGAGTCTTCCACCTACAGGTCACAGAGCCTGCCTTTGAGCCACCAGCTCGTGCTTCTCCTGGCAATGGGTCTGGTCACAGCAGTGCTCCTAGCCCAGATCCCACCCTGACCCGAGGCCACAGCATCATCAATGTCATTGTCCCAGAGGACCACACACATTTCTTCCAGCAACTGGGCTATGTGTTGGCCACGCTGCTGCTCTTCATCTTGCTGCTCATCACTGTAGTCCTGGCTACACGATATCGTCACAGCGGAGGATGCAAGACGTCGGACAAAAAAGCTGGGAAGTCAAAGGGGAAGGATGTGAACATGGTGGAGTTTGCTGTAGCCACAAGGGATCAGGCTCCATATAGGACTGAGGACATCCAGCTAGATTACAAAAACAACATCCTGAAGGAGAGGGCTGAGCTGGCCCATAGTCCTCTGCCTGCCAAGGATGTGGATCTGGATAAAGAGTTCAGGAAGGAGTACTGCAAATAAATGGACCCTGAGCTTCTGGCTGGGCCAGCAGCTCTGTATCAAAGGACATCTCCCTGACCCTCCTGCGGTATTCCTGGCTCTTCTCAGCGGCTGGTCCGACTTACCTAGAAACTTGGCAGAGCAGCTGCCTGTACTTTGCCCTTCCTAGAATCGCCACCCCTCATCTTGGTGAGCAACTGTGGGTTCCCTAGAGACTCTGGTATAGTACGATTGCTGCCCTTCACCTGTGCCCACTGATGGTTGTACCCCCAACTTAAACACAACAAAGATCCCTTGTTAATATCCACCAAATGCAAAGTCCCTCGTGGCCTCTTACTGCTAGGGTCAGGAAGACACTTAAAAATTCCAGTTAAGACTCCCTAGCCACCAGTTAAACACATTAGCCATTGTCCTGGGGGGTCTTCCTGAGCTGCATTGTGCCTGTGTACTGTTCAGAGCCCTGCTGTTATAGGTTCTGACTCATGGGCCCGCCTTGCTGCTTTGGGCAACTTGAGGCTAGCCCAGGGCCCTTTCTCTGCTTCTGATTCCTTTCTGCCGAATGCCTCCCAAGAGCTACACCAGCAGTTACTGGGTACCGTATGACTCTTGGCCTTGACATCCCTCCCTAGGCTGGAGTCTGGGGTTGGGGCCCCATTTGTCCTCTGTTTTGGCTGAAGATGGGGCGAAGATTTGGCTGAGTGGCCTATGCTGTCACATCAAACAGCTATCATTTACTCCTACTTGGGAAGTTTTCATGTGACAATAAAAGATACATCTGATTTTTAGTCTTGGAAAAAAAAAAAAAAAAAA

SEQ ID NO: 33 - DNA, 2534 bp, Homo sapiens
  source 1..2534: mol_type = genomic DNA
atgtcctcggaccttcttgtccctccaagggtgcggtcaccacccctccccaggcctgaccggtgaggggctgggcctctgctcccacactgcccctccccagcaggccagcaacgatgggggaggccaaggggcccggcaggagccagagggggcggtccccagaccgtagacaggcccaggcctccgtgatgtcaccgcgggtgctaaggagggggagggggtagggctgtttttctggagagagacttagagccgagtgggacaaagcctggggctgggcgggggccatggcgctgccatcccgaatcctgctttggaaacttgtgcttctgcagagctctgctgttctcctgcactcagggtcctcggtacccgccgctgctggcagctccgtggtgtccgagtccgcggtgagctgggaggcgggcgcccgggcggtgctgcgctgccagagcccgcgcatggtgtggacccaggaccggctgcacgaccgccagcgcgtgctccactgggacctgcgcggccccgggggtggccccgcgcggcgcctgctggacttgtactcggcgggcgagcagcgcgtgtacgaggcgcgggaccgcggccgcctggagctctcggcctcggccttcgacgacggcaacttctcgctgctcatccgcgcggtggaggagacggacgcggggctgtacacctgcaacctgcaccatcactactgccacctctacgagagcctggccgtccgcctggaggtcaccgacggccccccggccacccccgcctactgggacggcgagaaggaggtgctggcggtggcgcgcggcgcacccgcgcttctgacctgcgtgaaccgcgggcacgtgtggaccgaccggcacgtggaggaggctcaacaggtggtgcactgggaccggcagccgcccggggtcccgcacgaccgcgcggaccgcctgctggacctctacgcgtcgggcgagcgccgcgcctacgggcccctttttctgcgcgaccgcgtggctgtgggcgcggatgcctttgagcgcggtgacttctcactgcgtatcgagccgctggaggtcgccgacgagggcacctactcctgccacctgcaccaccattactgtggcctgcacgaacgccgcgtcttccacctgacggtcgccgaaccccacgcggagccgcccccccggggctctccgggcaacggctccagccacagcggcgccccaggcccagaccccacactggcgcgcggccacaacgtcatcaatgtcatcgtccccgagagccgagcccacttcttccagcagctgggctacgtgctggccacgctgctgctcttcatcctgctactggtcactgtcctcctggccgcccgcaggcgccgcggaggctacgaatactcggaccagaagtcgggaaagtcaaaggggaaggatgttaacttggcggagttcgctgtggctgcaggggaccagatgctttacaggagtgaggacatccagctagattacaaaaacaacatcctgaaggagagggcggagctggcccacagccccctgcctgccaagtacatcgacctagacaaagggttccggaaggagaactgcaaatagggaggccctgggctcctggctgggccagcagctgcacctctcctgtctgtgctcctcggggcatctcctgatgctccggggctcaccccccttccagcggctggtcccgctttcctggaatttggcctgggcgtatgcagaggccgcctccacacccctctcccaggggcttggtggcagcatagcccccacccctgcggcctttgctcacgggtggccctgcccacccctggcacaaccaaaatcccactgatgcccatcatgccctcagacccttctgggctctgcccgctgggggcctgaagacattcctggaggacactcccatcagaacctggcagccccaaaactggggtcagcctcagggcaggagtcccactcctccagggctctgctcgtccggggctgggagatgttcctggaggaggacactcccatcagaacttggcagccttgaagttggggtcagcctcggcaggagtcccactcctcctggggtgctgcctgccaccgagagctcccccacctgtaccaccatgtgggactccaggcaccatctgttctccccagggacctgctgacttgaatgccagcccttgctcctctgtgttgctttgggccacctggggctgcaccccctgccctttctctgccccatccctaccctagccttgctctcagccaccttgatagtcactgggctccctgtgacttctgaccctgacacccctcccttggactctgcctgggctggagtctagggctggggctacatttggcttctgtactggctgaggacaggggagggagtgaagttggtttggggtggcctgtgttgccactctcagcaccccacatttgcatctgctggtggacctgccaccatcacaataaagtccccatctgatttttaga

SEQ ID NO: 34 - DNA, 20 bp, Synthetic construct
  source 1..20: mol_type = other DNA
cttgtggatatgtattcggc

SEQ ID NO: 35 - DNA, 20 bp, Synthetic construct
  source 1..20: mol_type = other DNA
tgtgcgcctcgaggttacag

SEQ ID NO: 36 - DNA, 20 bp, Synthetic construct
  source 1..20: mol_type = other DNA
gctgcatgatcgccagcgcg

SEQ ID NO: 37 - DNA, 20 bp, Synthetic construct
  source 1..20: mol_type = other DNA
ggcgcggatgcctttgagcg

SEQ ID NO: 38 - DNA, 20 bp, Synthetic construct
  source 1..20: mol_type = other DNA
gtccgcctggaggtcaccga

SEQ ID NO: 39 - DNA, 21 bp, Synthetic construct
  source 1..21: mol_type = other DNA
cttgtggatatgtattcggcg

SEQ ID NO: 40 - DNA, 21 bp, Synthetic construct
  source 1..21: mol_type = other DNA
ctggtcgacatgtacagcgcg

SEQ ID NO: 41 - DNA, 114 bp, Synthetic construct
  source 1..114: mol_type = other DNA
ctggcgccccccgccggcaccaccgacgccgcgcacccggggcggtccgtggtccccgcgttgcttcctctgctggccgggaccctgctgctgctggagacggccactgctccc

SEQ ID NO: 42 - DNA, 105 bp, Synthetic construct
  source 1..105: mol_type = other DNA
tacccatacgatgttccagattacgctacgtcctcaccatccattggcggcccaaacatgactttactattggccatgatcatgtttgcgttaaagatagggtcg

SEQ ID NO: 43 - DNA, 2534 bp, Homo sapiens; identical to SEQ ID NO: 33
  source 1..2534: mol_type = genomic DNA

SEQ ID NO: 44 - DNA, 2008 bp, Homo sapiens
  source 1..2008: mol_type = genomic DNA
acttagagccgagtgggacaaagcctggggctgggcgggggccatggcgctgccatcccgaatcctgctttggaaacttgtgcttctgcagagctctgctgttctcctgcactcagggtcctcggtacccgccgctgctggcagctccgtggtgtccgagtccgcggtgagctgggaggcgggcgcccgggcggtgctgcgctgccagagcccgcgcatggtgtggacccaggaccggctgcacgaccgccagcgcgtgctccactgggacctgcgcggccccgggggtggccccgcgcggcgcctgctggacttgtactcggcgggcgagcagcgcgtgtacgaggcgcgggaccgcggccgcctggagctctcggcctcggccttcgacgacggcaacttctcgctgctcatccgcgcggtggaggagacggacgcggggctgtacacctgcaacctgcaccatcactactgccacctctacgagagcctggccgtccgcctggaggtcaccgacggccccccggccacccccgcctactgggacggcgagaaggaggtgctggcggtggcgcgcggcgcacccgcgcttctgacctgcgtgaaccgcgggcacgtgtggaccgaccggcacgtggaggaggctcaacaggtggtgcactgggaccggcagccgcccggggtcccgcacgaccgcgcggaccgcctgctggacctctacgcgtcgggcgagcgccgcgcctacgggcccctttttctgcgcgaccgcgtggctgtgggcgcggatgcctttgagcgcggtgacttctcactgcgtatcgagccgctggaggtcgccgacgagggcacctactcctgccacctgcaccaccattactgtggcctgcacgaacgccgcgtcttccacctgacggtcgccgaaccccacgcggagccgcccccccggggctctccgggcaacggctccagccacagcggcgccccaggcccagaccccacactggcgcgcggccacaacgtcatcaatgtcatcgtccccgagagccgagcccacttcttccagcagctgggctacgtgctggccacgctgctgctcttcatcctgctactggtcactgtcctcctggccgcccgcaggcgccgcggaggctacgaatactcggaccagaagtcgggaaagtcaaaggggaaggatgttaacttggcggagttcgctgtggctgcaggggaccagatgctttacaggagtgaggacatccagctagcctcctctcctcccacagattacaaaaacaacatcctgaaggagagggcggagctggcccacagccccctgcctgccaagtacatcgacctagacaaagacccttctgggctctgcccgctgggggcctgaagacattcctggaggacactcccatcagaacctggcagccccaaaactggggtcagcctcagggcaggagtcccactcctccagggctctgctcgtccggggctgggagatgttcctggaggaggacactcccatcagaacttggcagccttgaagttggggtcagcctcggcaggagtcccactcctcctggggtgctgcctgccaccgagagctcccccacctgtaccaccatgtgggactccaggcaccatctgttctccccagggacctgctgacttgaatgccagcccttgctcctctgtgttgctttgggccacctggggctgcaccccctgccctttctctgccccatccctaccctagccttgctctcagccaccttgatagtcactgggctccctgtgacttctgaccctgacacccctcccttggactctgcctgggctggagtctagggctggggctacatttggcttctgtactggctgaggacaggggagggagtgaagttggtttggggtggcctgtgttgccactctcagcaccccacatttgcatctgctggtggacctgccaccatcacaataaagtccccatctgatttttaga

SEQ ID NO: 45 - DNA, 2231 bp, Homo sapiens
  source 1..2231: mol_type = genomic DNA
atgtcctcggaccttcttgtccctccaagggtgcggtcaccacccctccccaggcctgaccggtgaggggctgggcctctgctcccacactgcccctccccagcaggccagcaacgatgggggaggccaaggggcccggcaggagccagagggggcggtccccagaccgtagacaggcccaggcctccgtgatgtcaccgcgggtgctaaggagggggagggggtagggctgtttttctggagagagacttagagccgagtgggacaaagcctggggctgggcgggggccatggcgctgccatcccgaatcctgctttggaaacttgtgcttctgcagagctctgctgttctcctgcactcagcggtggaggagacggacgcggggctgtacacctgcaacctgcaccatcactactgccacctctacgagagcctggccgtccgcctggaggtcaccgacggccccccggccacccccgcctactgggacggcgagaaggaggtgctggcggtggcgcgcggcgcacccgcgcttctgacctgcgtgaaccgcgggcacgtgtggaccgaccggcacgtggaggaggctcaacaggtggtgcactgggaccggcagccgcccggggtcccgcacgaccgcgcggaccgcctgctggacctctacgcgtcgggcgagcgccgcgcctacgggcccctttttctgcgcgaccgcgtggctgtgggcgcggatgcctttgagcgcggtgacttctcactgcgtatcgagccgctggaggtcgccgacgagggcacctactcctgccacctgcaccaccattactgtggcctgcacgaacgccgcgtcttccacctgacggtcgccgaaccccacgcggagccgcccccccggggctctccgggcaacggctccagccacagcggcgccccaggcccagaccccacactggcgcgcggccacaacgtcatcaatgtcatcgtccccgagagccgagcccacttcttccagcagctgggctacgtgctggccacgctgctgctcttcatcctgctactggtcactgtcctcctggccgcccgcaggcgccgcggaggctacgaatactcggaccagaagtcgggaaagtcaaaggggaaggatgttaacttggcggagttcgctgtggctgcaggggaccagatgctttacaggagtgaggacatccagctagattacaaaaacaacatcctgaaggagagggcggagctggcccacagccccctgcctgccaagtacatcgacctagacaaagggttccggaaggagaactgcaaatagggaggccctgggctcctggctgggccagcagctgcacctctcctgtctgtgctcctcggggcatctcctgatgctccggggctcaccccccttccagcggctggtcccgctttcctggaatttggcctgggcgtatgcagaggccgcctccacacccctctcccaggggcttggtggcagcatagcccccacccctgcggcctttgctcacgggtggccctgcccacccctggcacaaccaaaatcccactgatgcccatcatgccctcagacccttctgggctctgcccgctgggggcctgaagacattcctggaggacactcccatcagaacctggcagccccaaaactggggtcagcctcagggcaggagtcccactcctccagggctctgctcgtccggggctgggagatgttcctggaggaggacactcccatcagaacttggcagccttgaagttggggtcagcctcggcaggagtcccactcctcctggggtgctgcctgccaccgagagctcccccacctgtaccaccatgtgggactccaggcaccatctgttctccccagggacctgctgacttgaatgccagcccttgctcctctgtgttgctttgggccacctggggctgcaccccctgccctttctctgccccatccctaccctagccttgctctcagccaccttgatagtcactgggctccctgtgacttctgaccctgacacccctcccttggactctgcctgggctggagtctagggctggggctacatttggcttctgtactggctgaggacaggggagggagtgaagttggtttggggtggcctgtgttgccactctcagcaccccacatttgcatctgctggtggacctgccaccatcacaataaagtccccatctgatttttaga

SEQ ID NO: 46 - DNA, 2731 bp, Homo sapiens
  source 1..2731: mol_type = genomic DNA
aagaaggctggcggtgtttcctcttagaggggagaaactcagcctgggtaggagacccagccccacgcagggaaaactgtgctaacgcttccgatgtgcgtggcaggtgcggcggcggcgaatacggtttgtcctcgagcctaaccctgtctgtgttggtgtcagcagtggcccccctaccacacacacagggtccctggcgtcccaagaccactcctggcagccccgccactggctgcgcctggaagccgcgtcctcaggcctcgcctggcatttgctgtcacagaggttgcttccttgggtccgtccgtcctcgcccctccagcctgggcgccccccgacccctgtctcattccctccaccacatgcagcacagtccaggaggctggggtccaagagggccatggctggcacccggcttagtgctgagtccccagtgcccagcaagcctggcacccaggaggtggtcagcaaacgcttctgaacgacaggaagtgggactcttcagccatcgatgatccgctgtgcggccacaggctctgctgttctcctgcactcagggtcctcggtacccgccgctgctggcagctccgtggtgtccgagtccgcggtgagctgggaggcgggcgcccgggcggtgctgcgctgccagagcccgcgcatggtgtggacccaggaccggctgcacgaccgccagcgcgtgctccactgggacctgcgcggccccgggggtggccccgcgcggcgcctgctggacttgtactcggcgggcgagcagcgcgtgtacgaggcgcgggaccgcggccgcctggagctctcggcctcggccttcgacgacggcaacttctcgctgctcatccgcgcggtggaggagacggacgcggggctgtacacctgcaacctgcaccatcactactgccacctctacgagagcctggccgtccgcctggaggtcaccgacggccccccggccacccccgcctactgggacggcgagaaggaggtgctggcggtggcgcgcggcgcacccgcgcttctgacctgcgtgaaccgcgggcacgtgtggaccgaccggcacgtggaggaggctcaacaggtggtgcactgggaccggcagccgcccggggtcccgcacgaccgcgcggaccgcctgctggacctctacgcgtcgggcgagcgccgcgcctacgggcccctttttctgcgcgaccgcgtggctgtgggcgcggatgcctttgagcgcggtgacttctcactgcgtatcgagccgctggaggtcgccgacgagggcacctactcctgccacctgcaccaccattactgtggcctgcacgaacgccgcgtcttccacctgacggtcgccgaaccccacgcggagccgcccccccggggctctccgggcaacggctccagccacagcggcgccccaggcccagaccccacactggcgcgcggccacaacgtcatcaatgtcatcgtccccgagagccgagcccacttcttccagcagctgggctacgtgctggccacgctgctgctcttcatcctgctactggtcactgtcctcctggccgcccgcaggcgccgcggaggctacgaatactcggaccagaagtcgggaaagtcaaaggggaaggatgttaacttggcggagttcgctgtggctgcaggggaccagatgctttacaggagtgaggacatccagctagattacaaaaacaacatcctgaaggagagggcggagctggcccacagccccctgcctgccaagtacatcgacctagacaaagggttccggaaggagaactgcaaatagggaggccctgggctcctggctgggccagcagctgcacctctcctgtctgtgctcctcggggcatctcctgatgctccggggctcaccccccttccagcggctggtcccgctttcctggaatttggcctgggcgtatgcagaggccgcctccacacccctctcccaggggcttggtggcagcatagcccccacccctgcggcctttgctcacgggtggccctgcccacccctggcacaaccaaaatcccactgatgcccatcatgccctcagacccttctgggctctgcccgctgggggcctgaagacattcctggaggacactcccatcagaacctggcagccccaaaactggggtcagcctcagggcaggagtcccactcctccagggctctgctcgtccggggctgggagatgttcctggaggaggacactcccatcagaacttggcagccttgaagttggggtcagcctcggcaggagtcccactcctcctggggtgctgcctgccaccgagagctcccccacctgtaccaccatgtgggactccaggcaccatctgttctccccagggacctgctgacttgaatgccagcccttgctcctctgtgttgctttgggccacctggggctgcaccccctgccctttctctgccccatccctaccctagccttgctctcagccaccttgatagtcactgggctccctgtgacttctgaccctgacacccctcccttggactctgcctgggctggagtctagggctggggctacatttggcttctgtactggctgaggacaggggagggagtgaagttggtttggggtggcctgtgttgccactctcagcaccccacatttgcatctgctggtggacctgccaccatcacaataaagtccccatctgatttttaga

SEQ ID NO: 47 - DNA, 18 bp, Synthetic construct
  source 1..18: mol_type = other DNA
tcgacgcgccatctttaa

SEQ ID NO: 48 - DNA, 19 bp, Synthetic construct
  source 1..19: mol_type = other DNA
atcgaatgcaccgcacact

SEQ ID NO: 49 - DNA, 25 bp, Synthetic construct
  source 1..25: mol_type = other DNA
accagcctgcacccactcctcagac

SEQ ID NO: 50 - DNA, 24 bp, Synthetic construct
  source 1..24: mol_type = other DNA
gtggagtcatactggaacatgtag

SEQ ID NO: 51 - DNA, 20 bp, Synthetic construct
  source 1..20: mol_type = other DNA
tgcaaatggcagccctggtg

SEQ ID NO: 52 - protein, 8 aa, Synthetic construct
LEVLFQGP

SEQ ID NO: 53 - protein, 44 aa, Synthetic construct
WTQDRLHDRQRVVHWDLSGGPGSQRRRLVDMYSAGEQRVYEPRD

SEQ ID NO: 54 - protein, 60 aa, Synthetic construct
WTQDRLHDRQRVVHWDLSGSKAGGPARRLVDMYSTGEQRVGEQRLGEQRVGEQRVYEPRD

SEQ ID NO: 55 - DNA, 64 bp, Mus musculus
  source 1..64: mol_type = genomic DNA
gccaacggcgccgacttgtggatatgtattcggcgggtgaacagcgcgtgtacgagccgcgcga

SEQ ID NO: 56 - DNA, 53 bp, Synthetic construct
  source 1..53: mol_type = other DNA
gccaacggcgccgacttgtggatatgtgaacagcgcgtgtacgagccgcgcga

SEQ ID NO: 57 - DNA, 62 bp, Synthetic construct
  source 1..62: mol_type = other DNA
gccaacggcgccgacttgtggatatgtatggcgggtgaacagcgcgtgtacgagccgcgcga

SEQ ID NO: 58 - DNA, 64 bp, Mus musculus; identical to SEQ ID NO: 55
  source 1..64: mol_type = other DNA

SEQ ID NO: 59 - DNA, 58 bp, Synthetic construct
  source 1..58: mol_type = other DNA
gccaacggcgccgacttgtggatatggcgggtgaacagcgcgtgtacgagccgcgcga

SEQ ID NO: 60 - DNA, 60 bp, Synthetic construct
  source 1..60: mol_type = other DNA
gccaacggcgccgacttgtggatatgtggcgggtgaacagcgcgtgtacgagccgcgcga

SEQ ID NO: 61 - DNA, 65 bp, Synthetic construct
  source 1..65: mol_type = other DNA
gccaacggcgccgacttgtggatatgtattctggcgggtgaacagcgcgtgtacgagccgcgcga

SEQ ID NO: 62 - protein, 44 aa, Mus musculus; identical to SEQ ID NO: 53

SEQ ID NO: 63 - protein, 59 aa, Synthetic construct
WTQDRLHDRQRVVHWDLSGGPGSQRRRLVDMYSAGEQRVGEQRLGEQRVGEQRVYEPRD

SEQ ID NO: 64 - protein, 60 aa, Bos taurus; identical to SEQ ID NO: 54

SEQ ID NO: 65 - protein, 45 aa, Synthetic construct
WTQDRLHDRQRVVHWDLSGSKAGGPARRLVDMYSTGEQRVYEPRD

SEQ ID NO: 66 - DNA, 196 bp, Synthetic construct
  source 1..196: mol_type = other DNA
tgatgagggcatctattcctgccacctgccacctgcaccatcactactgtggcctccacgagcgccgagtcttccacctacaggtcacagagcctgcctttgagccaccagctcgtgcttctcctgtctggtcacagcagtgctcctagcccaggtgtgtgtgtagcctgccggcctgagaagaggtcctgttgtg

SEQ ID NO: 67 - DNA, 204 bp, Mus musculus
  source 1..204: mol_type = genomic DNA
tgatgagggcatctattcctgccacctgccacctgcaccatcactactgtggcctccacgagcgccgagtcttccacctacaggtcacagagcctgcctttgagccaccagctcgtgcttctcctggcaatgggtctggtcacagcagtgctcctagcccaggtgtgtgtgtagcctgccggcctgagaagaggtcctgttgtg

SEQ ID NO: 68 - DNA, 196 bp, Synthetic construct; identical to SEQ ID NO: 66
  source 1..196: mol_type = other DNA

SEQ ID NO: 69 - DNA, 107 bp, Synthetic construct
  source 1..107: mol_type = other DNA
tgatgagggcatctattcctgccacctgccacctgcaccatcactactgtggcctcctagcccaggtgtgtgtgtagcctgccggcctgagaagaggtcctgttgtg

SEQ ID NO: 70 - protein, 311 aa, Mus musculus
SGPSGTAAASSSLVSESVVSLAAGTQAVLRCQSPRMVWTQDRLHDRQRVVHWDLSGGPGSQRRRLVDMYSAGEQRVYEPRDRDRLLLSPSAFHDGNFSLLIRAVDRGDEGVYTCNLHHHYCHLDESLAVRLEVTEDPLLSRAYWDGEKEVLVVAHGAPALMTCINRAHVWTDRHLEEAQQVVHWDRQLPGVSHDRADRLLDLYASGERRAYGPPFLRDRVSVNTNAFARGDFSLRIDELERADEGIYSCHLHHHYCGLHERRVFHLQVTEPAFEPPARASPGNGSGHSSAPSPDPTLTRGHSIINVIVPED

SEQ ID NO: 71 - protein, 311 aa, Homo sapiens
SGSSVPAAAGSSVVSESAVSWEAGARAVLRCQSPRMVWTQDRLHDRQRVLHWDLRGPGGGPARRLLDLYSAGEQRVYEARDRGRLELSASAFDDGNFSLLIRAVEETDAGLYTCNLHHHYCHLYESLAVRLEVTDGPPATPAYWDGEKEVLAVARGAPALLTCVNRGHVWTDRHVEEAQQVVHWDRQPPGVPHDRADRLLDLYASGERRAYGPLFLRDRVAVGADAFERGDFSLRIEPLEVADEGTYSCHLHHHYCGLHERRVFHLTVAEPHAEPPPRGSPGNGSSHSGAPGPDPTLARGHNVINVIVPES

SEQ ID NO: 72 - protein, 332 aa, Bos taurus
SGPSGPATSDRSVVSESTVSWAAGARAVLRCQSPRMVWTQDRLHDRQRVVHWDLSGSKAGGPARRLVDMYSTGEQRVGEQRLGEQRVGEQRVYEPRDRGRLLLPPSAFHDGNFSLFIRAVEETDEGLYTCNLHHHYCHLYESLAVSLEVTDDPRAAGAHWDGEKEVLAVERGAPALLTCVNRAHVWTDRHLEEAQQVVHWDRQPPGVPHDRADRLLDLYASGERRAYGPPFLRDRVAVEADAFARGDFSLLIDPVEPADEGTYSCHLHHHYCGLHERRVFHLRVTEPAARPPPPPRDSPGNGSSHSGAPGPGARDPTLTRGRSVINVIVPEG

SEQ ID NO: 73 - protein, 439 aa, Chikungunya virus
YEHVTVIPNTVGVPYKTLVNRPGYSPMVLEMELQSVTLEPTLSLDYITCEYKTVIPSPYVKCCGTAECKDKSLPDYSCKVFTGVYPFMWGGAYCFCDAENTQLSEAHVEKSESCKTEFASAYRAHTASASAKLRVLYQGNNITVAAYANGDHAVTVKDAKFVVGPMSSAWTPFDNKIVVYKGDVYNMDYPPFGAGRPGQFGDIQSRTPESKDVYANTQLVLQRPAAGTVHVPYSQAPSGFKYWLKERGASLQHTAPFGCQIATNPVRAVNCAVGNIPISIDIPDAAFTRVVDAPSVTDMSCEVPACTHSSDFGGVAIIKYTASKKGKCAVHSMTNAVTIREADVEVEGNSQLQISFSTALASAEFRVQVCSTQVHCAAACHPPKDHIVNYPASHTTLGVQDISTTAMSWVQKITGGVGLIVAVAALILIVVLCVSFSRH

SEQ ID NO: 74 - protein, 436 aa, Mayaro virus
YEHTAVIPNQVGFPYKAHVAREGYSPLTLQMQVVETSLEPTLNLEYITCDYKTKVPSPYVKCCGTAECRTQDKPEYKCAVFTGVYPFMWGGAYCFCDSENTQMSEAYVERADVCKHDYAAAYRAHTASLRAKIKVTYGTVNQTVEAYVNGDHAVTIAGTKFIFGPVSTAWTPFDTKIVVYKGEVYNQDFPPYGAGQPGRFGDIQSRTLDSKDLYANTGLKLARPAAGNIHVPYTQTPSGFKTWQKDRDSPLNAKAPFGCTIQTNPVRAMNCAVGNIPVSMDIADSAFTRLTDAPIISELLCTVSTCTHSSDFGGVAVLSYKVEKAGRCDVHSHSNVAVLQEVSIEAEGRSVIHFSTASAAPSFIVSVCSSRATCTAKCEPPKDHVVTYPANHNGITLPDLSSTAMTWAQHLAGGVGLLIALAVLILVIVTCITLRR

SEQ ID NO: 75 - protein, 438 aa, Ross River virus
YEHTATIPNVVGFPYKAHIERNGFSPMTLQLEVVETSLEPTLNLEYITCEYKTVVPSPFIKCCGTSECSSKEQPDYQCKVYTGVYPFMWGGAYCFCDSENTQLSEAYVDRSDVCKHDHASAYKAHTASLKATIRISYGTINQTTEAFVNGEHAVNVGGSKFIFGPISTAWSPFDNKIVVYKDDVYNQDFPPYGSGQPGRFGDIQSRTVESKDLYANTALKLSRPSPGVVHVPYTQTPSGFKYWLKEKGSSLNTKAPFGCKIKTNPVRAMDCAVGSIPVSMDIPDSAFTRVVDAPAVTDLSCQVVVCTHSSDFGGVATLSYKTDKPGKCAVHSHSNVATLQEATVDVKEDGKVTVHFSTASASPAFKVSVCDAKTTCTAACEPPKDHIVPYGASHNNQVFPDMSGTAMTWVQRLASGLGGLALIAVVVLVLVTCITMRR

SEQ ID NO: 76 - protein, 439 aa, Onyong-nyong virus
YEHATVIPNTVGVPYKTLVSRPGYSPMVLEMELQSVTLEPALSLDYITCEYKTITPSPYVKCCGTAECKAKNLPDYNCKVFTGVYPFMWGGAYCFCDAENTQLSEAHVEKSESCKTEFASAYRAHTASVSAKLRVFYQGNNITVSAYANGDHAVTVKDAKFVIGPLSSAWSPFDNKIVVYKGEVYNMDYPPFGAGRPGQFGDIQSRTPDSKDVYANTQLILQRPAAGAIHVPYSQAPSGFKYWLKEKGASLQHTAPFGCQIATNPVRAVNCAVGNIPVSIDIPDAAFTRVTDAPSITDMSCEVASCTHSSDFGGAAVIKYTASKKGKCAVHSVTNAVTIREPNVDVEGTAQLQIAFSTALASAEFKVQICSTLVHCSATCHPPKDHIVNYPSPHTTLGVQDISTTAMSWVQKITGGVGLVVAIAALILIIVLCVSFSRH

SEQ ID NO: 77 - protein, 438 aa, Semliki Forest virus [sequence omitted]

SEQ ID NO: 78 - protein, 439 aa, Sindbis virus
YEHATTVPNVPQIPYKALVERAGYAPLNLEITVMSSEVLPSTNQEYITCKFTTVVPSPKVKCCGSLECQPAAHADYTCKVFGGVYPFMWGGAQCFCDSENSQMSEAYVELSADCATDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQAASGFEMWKNNSGRPLQETAPFGCKIAVNPLRAVDCSYGNIPISIDIPNAAFIRTSDAPLVSTVKCDVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 79 - protein, 438 aa, Western equine encephalitis virus
FEHATTVPNVPGIPYKALVERAGYAPLNLEIKVVSSELTPSTNKEYVTCKFHTVIPSPQVKCCGSLECKASSKADYTCRVFGGVYPFMWGGAQCFCDSENTQLSEAYVEFAPDCTIDHAVALRVHTAALKVGLRIVYGNTTAYLDTFVNGVTPGSSRDLKVIAGPISAAFSPFDHKVVIRKGLVYNYDFPEYGAMKPGAFGDIQASSLDATDIVARTDIRLLKPSVKNIHVPYTQAVSGYEMWKNNSGRPLQETAPFGCKIEVEPLRASNCAYGHIPISIDIPDAAFVRSSESPTILEVSCTADCIYSADFGGSLTLQYKADREGHCPVHSHSTTAVLKEATTHVTATGSITLHFSTSSPQANFIVSLCGKKTTCNAECKPPADHIIGEPHKVDQEFQAAVSKTSWNWLLALFGGASSLIVVGLIVLVCSSMLINTRR

SEQ ID NO: 80 - protein, 440 aa, Eastern equine encephalitis virus
YEHTAVMPNKVGIPYKALVERPGYAPVHLQIQLVNTRIIPSTNLEYITCKYKTKVPSPVVKCCGATQCSSKPHPDYQCQVFTGVYPFMWGGAYCFCDTENTQMSEAYVERSEECSIDHAKAYKVHTGTVQAMVNITYGSVSWRSADVYVNGETPAKIGDAKLIIGPLSSAWSPFDNKVVVYGHEVYNYDFPEYGTGKAGSFGDLQSRTSTSNDLYANTNLKLQRPQAGIVHTPFTQAPSGFERWKRDKGAPLNDVAPFGCSIALEPLRAENCAVGSIPISIDIPDAAFTRISETPTVSDLECKITECTYASDFGGIATVAYKSSKAGNCPIHSPSGVAVIKENDVTLAESGSFTFHFSTANIHPAFKLQVCTSAVTCKGDCKPPKDHIVDYPAQHTDLLTSAISATAWSWLKVLVGGTSAFIVLGLIATAVVALVLFFHR

SEQ ID NO: 81 - protein, 439 aa, Venezuelan equine encephalitis virus
YEHATTMPSQAGISYNTIVNRAGYAPLPISITPTKIKLIPTVNLEYVTCHYKTGMDSPAIKCCGSQECTPTYRPDEQCKVFTGVYPFMWGGAYCFCDTENTQVSKAYVMKSDDCLADHAEAYKAHTASVQAFLNITVGEHSIVTTVYVNGETPVNFNGVKITAGPLSTAWTPFDRKIVQYAGEIYNYDFPEYGAGQPGAFGDIQSRTVSSSDLYANTNLVLQRPKAGAIHVPYTQAPSGFEQWKKDKAPSLKFTAPFGCEIYTNPIRAENCAVGSIPLAFDIPDALFTRVSETPTLSAAECTLNECVYSSDFGGIATVKYSASKSGKCAVHVPSGTATLKEAAVELTEQGSATIHFSTANIHPEFRLQICTSYVTCKGDCHPPKDHIVTHPQYHAQTFTAAVSKTAWTWLTSLLGGSAVIIIIGLVLATIVAMYVLTNQ

SEQ ID NO: 82 - protein, 419 aa, Chikungunya virus
NFNVYKATRPYLAHCPDCGEGHSCHSPIALERIRNEATDGTLKIQVSLQIGIKTDDSHDWTKLRYMDSHTPADAERAGLLVRTSAPCTITGTMGHFILARCPKGETLTVGFTDSRKISHTCTHPFHHEPPVIGRERFHSRPQHGKELPCSTYVQSTAATAEEIEVHMPPDTPDRTLMTQQSGNVKITVNGQTVRYKCNCGGSNEGLTTTDKVINNCKIDQCHAAVTNHKNWQYNSPLVPRNAELGDRKGKIHIPFPLANVTCRVPKARNPTVTYGKNQVTMLLYPDHPTLLSYRNMGQEPNYHEEWVTHKKEVTLTVPTEGLEVTWGNNEPYKYWPQMSTNGTAHGHPHEIILYYYELYPTMTVVIVSVASFVLLSMVGTAVGMCVCARRRCITPYELTPGATVPFLLSLLCCVRTTKA

SEQ ID NO: 83 - protein, 418 aa, Mayaro virus
HFNAYKLTRPYVAYCADCGMGHSCHSPAMIENVQADATDGTLKIQFASQIGLTKADTHDHTKIRYAEGHDIAEAARSTLKVHSSSECAVTGTMGHFILAKCPPGEVISVSFVDSKNEQRTCRIAYHHEQRLIGRERFTVRPHHGIELPCTTYQLTTAETSEEIDMHMPPDIPDRTILSQQSGNVKITVNGRTVKYSCSCGSKPSGTTTTDKTINSCTVDKCQAYVTSHTKWQFNSPFVPRAEQAERKGKVHIPFPLINTTCRVPLAPEALVRSGKREATLSLHPIHPTLLSYRTLGREPVFDEQWITTQTEVTIPVPVEGVEYRWGNHKPQRLWSQLTTDGRAHGWPHEIIEYYYGLHPTTTIVVVVAVSVVVLLSVAASVYMCVVARNKCLTPYALTPGAVVPVTIGVLCCAPKAHA

SEQ ID NO: 84 - protein, 418 aa, Ross River virus
HFNVYKATRPYLAXCADCGDGYFCYSPVAIEKIRDEASDGMLKIQVSAQIGLDKAGTHAHTKIRYMAGHDVQESKRDSLRVYTSAACSIHGTMGHFIVAHCPPGDYLKVSFEDADSHVKACKVQYKHDPLPVGREKFVVRPHFGVELPCTSYQLTTAPTDEEIDMHTPPDIPDRTLLSQTAGNVKITAGGRTIRYNCTCGRDNVGTTSTDKTINTCKIDQCHAAVTSHDKWQFTSPFVPRADQTARRGKVHVPFPLTNVTCRVPLARAPDVTYGKKEVTLRLHPDHPTLFSYRSLGAEPHPYEEWVDKFSERIIPVTEEGIEYQWGNNPPVRLWAQLTTEGKPHGWPHEIIQYYYGLYPAATIAAVSGASLMALLTLAATCCMLATARRKCLTPYALTPGAVVPLTLGLLCCAPRANA

SEQ ID NO: 85 - protein, 419 aa, Onyong-nyong virus
NFNVYKVTRPYLAHCPDCGEGHSCHSPIALERIRSEATDGTLKIQVSLQIGIKTDDSHDWTKLRYMDSHTPVDAARSGLFVRTSAPCTITGTMGHFILARCPKGETLTVGFVDSRRISHTCMHPFHHEPPLIGREKFHSRPQHGKELPCSTYVHTTAATAEEIEVHMPPDTPDYTLMTQQAGNVKITVBGQTVRYKCKCDGSNEGLITADKVINNCKVDQCHTAVTNHKKWQYNSPLTPRNSEQGDRKGKIHIPFPLVNTTCRVPKARNPTVTYGKNRVTLLLYPDHPTLLSYRAMGRIPDYHEEWITNKKEISITVPAEGLEVTWGNNDPYKYWPQLSTNGTAHGHPHEIILYYYELYPTTTIAVLAAASIVITSLVGLSLGMCICARRRCITPYELTPGATIPFLLGVLCCARTAKA

SEQ ID NO: 86 - protein, 418 aa, Semliki Forest virus
HFNVYKATRPYIAYCADCGAGHSCHSPVAIEAIRSEATDGMLKIQFSAQIGIDKSDNHDYTKIRYADGHAIENAVRSSLKVATSGDCFVHGTMGHFILATCPPGEFLQVSIQDTRNAVRACRIQYHHDPQPVGREKFTIRPHYGKEIPCTTYQQTTAETVEEIDMHMPPDTPDRTLLSQQSGNVKITVGGKKVKYNCTCGTGNVGTTNSDMTINTCLIEQCHVSVTDHKKWQFNSPFVPRADEPARKGKVHIPFPLDNITCRVPMAREPTVIHGKREVTLHLHPDHPTLFSYRTLGEDPQYHEEWVTAAVERTIPVPVDGMEYHWGNNDPVRLWSQLTTEGKPHGWPHQIVQYYYGLYPAATVSAVVGMSLLALISIFASCYMLVAARSKCLTPYALTPGAAVPWTLGILCCAPRAHA

SEQ ID NO: 87 - protein, 419 aa, Sindbis virus
DFTLTSPYLGTCSYCHHTEPCFSPIKIEQVWDEADDNTIRIQTSAQFGYDQSGAASSNKYRYMSLEQDHTVKEGTMDDIKISTSGPCRRLSYKGYFLLAKCPPGDSVTVSIASSNSATSCTMARKIKPKFVGREKYDLPPVHGKKIPCTVYDRLKETTAGYITMHRPGPHAYTSYLEESSGKVYAKPPSGKNITYECKCGDYKTGTVTTRTEITGCTAIKQCVAYKSDQTKWVFNSPDLIRHADHTAQGKLHLPFKLIPSTCMVPVAHAPNVVHGFKHISLQLDTDHLTLLTTRRLGANPEPTTEWIIGKTVRNFTVDRDGLEYIWGNHEPVRVYAQESAPGDPHGWPHEIVQHYYHRHPVYTILAVASAAVAMMIGVTVAALCACKARRECLTPYALAPNAVIPTSLALLCCVRSANA

SEQ ID NO: 88 - protein, 419 aa, Western equine encephalitis virus
DFTLTSPYLGFCPNCRHSAPCFSPIKIENVWDESDDGSIRIQVSAQFGYNQAGTADVTKFRYMSYDHDHDIEEDSMEKIAISTSGPCRRLGHKGYFLLAQCPPGDSVTVSITSGASENSCTVEFKIRRKFVGREEYLFPPVHGKRVKCHVYDHLKERSAGYITMHRPSPHAYKSYLKEASGEVYIKPPSGKNVTYECKCGDYSTGIVSTPTKMNGCTKAKQCIAYKSDQTKWVFNSPDLIRHTDHSVQGKLHIPFRLIPTVCPVPLAHTPTVTKWFKGITLHLTATRPTLLTTRKLGLRADATAEWITGTTSRNFSVGREGLEYVWGNHEPVRVWAQESAPSDPHGWPHEIIIHYYHRHPVYTVIVLCGVALAILVGTASSAACITKARRDCLTPYALAPNATVPTALAVLCCIRPTNA

SEQ ID NO: 89 - protein, 416 aa, Eastern equine encephalitis virus
HFTRYKLARPYIADCPNCGHSRCDSPIAIEEVRGDAHAGAIRIQTSAMFGLKTDGVDLAYMSFMNGKTQKSIKIDNLHVRTSAPCSLVSHHGYYILAQCPPGDTVTVGFHDGPNRHTCTVAHKVEFRPVGREEYRHPPEHGVELPCNRYTHKRADQGHYVEMHQPGLVADHSLLSIHSAKVKITVPSGAQVKYYCKCPDVREGITSSDHTTTCTDVKQCRAYLIDNKKWVYNFGRLLRGEGDTFKGKLHVPFVPVKAKCIATLAPELLVEHKHRTLILHLHPDHPTLLTTRSLGSDANPIRQWIERPTTVNFTVTGEGLEYTWGNHPPKRVWAQESGEGNPHGWPHEVVVYYYNRYPLTTIIGLCTCVAIIMVSCVTSVWLLCRARNLCITPYKLAPNAQVPILLALLCCIKPTKA

SEQ ID NO: 90 - protein, 419 aa, Venezuelan equine encephalitis virus
LFNEYKLTRPYMARCIRCAVGSCHSPIAIEAVKSDGHDGYVRLQTSSQYGLDSSGNLKGRTMRYDMHGTIKEIPLHQVSLYTSRPCHIVDGHGYFLLARCPAGDSITMEFKKDSVRHSCSVPYEVKFNPVGRELYTHPPEHGVEQACQVYAHDAQNRGAYVEMHLPGSEVDSSLVSLSGSSVTVTPPDGTSALVECECGGTKISETINKTKQFSQCTKKEQCRAYRLQNDKWVYNSDKLPKAAGATLKGKLHVPFLLADGKCTVPLAPEPMITFGFRSVSLKLHPKNPTYLITRQLADEPHYTHELISEPAVRNFTVTEKGWEFVWGNHPPKRFWAQETAPGNPHGLPHEVITHYYHRYPMSTILGLSICAAIATVSVAASTWLFCRSRVACLTPYRLTPNARIPFCLAVLCCARTARA

SEQ ID NO: 91 - protein, 15 aa, Synthetic construct
GEQRLGEQRVGEQRV

SEQ ID NO: 92 - protein, 5 aa, Synthetic construct
  VARIANT 5: note = L or V
GEQRX

SEQ ID NO: 93 - protein, 44 aa, Western equine encephalitis virus
PLNLEITVVSSELTPHTAALKVGLRIVYGNTTARLDTFVNGVTP

SEQ ID NO: 94 - protein, 44 aa, Sindbis virus
PLNLEITVMSSEVLPHTAAMKVGLRIVYGNTTSFLDVYVNGVTP

SEQ ID NO: 95 - protein, 45 aa, Eastern equine encephalitis virus
PVHLQIQLVNTRIIPHTGTVQAMVNITYGSVSWRSADVYVNGETP

SEQ ID NO: 96 - protein, 44 aa, Venezuelan equine encephalitis virus
PLPISITPTKIKLIPHTASVQAFLNITVGEHSIVTTVYVNGETP

SEQ ID NO: 97 - protein, 44 aa, Ross River virus
PMTLQLEVVXXSLEPHTASLKATIRISYGTINQTTEAFVNGEHA

SEQ ID NO: 98 - protein, 44 aa, Mayaro virus
PLTLQMQVIETSLEPHTASLRAKIKVTYGTVNQTVEAYVNGDHA

SEQ ID NO: 99 - protein, 44 aa, Chikungunya virus; identical to SEQ ID NO: 98

SEQ ID NO: 100 - protein, 71 aa, Asarcornis scutulata
DVYSTYYGDNKMERLCDMYSAGDQRVYSSYNQGRIFMPQNAFTDGNFSLVIKGSTVMLPCVNRNQIWTERH

SEQ ID NO: 101 - protein, 71 aa, Passer montanus
DLLSSYYGDGRMERLCDMYSAGDQRVYSSYNQGRILMPENAFADGNFSLVIKGSTATLPCVNRQHIQTERH

SEQ ID NO: 102 - protein, 70 aa, Centrocercus urophasianus
DVYSTYYGDNKMERLCDMYSAGDQRVYSSYNQGRILMPQNAFTDGNSLVIKGSTVMLPCVNRNHIQTERH

SEQ ID NO: 103 - protein, 71 aa, Gallus gallus
DVYSTYYGDNKMERLCDMYSAGNQRVYSSYNQGRILMPQNAFTDGNFSLVIKGSTVMLPCVNRNHIWTERH

SEQ ID NO: 104 - protein, 69 aa, Mus musculus
DLSGGPGSQRRRLVDMYSAGEQRVYEPRDRDRLLLSPSAFHDGNFSLLIRGAPALMTCINRAHVWTDRH

SEQ ID NO: 105 - protein, 69 aa, Homo sapiens
DLRGPGGGPARRLLDLYSAGEQRVYEARDRGRLELSASAFDDGNFSLLIRGAPALLTCVNRGHVWTDRH

SEQ ID NO: 106 - protein, 72 aa, Synthetic construct
SLAAGTQAVLRCQSPRMVWTQDRLHDRQRVVHWDVYSTYYGDNKMERLCDMYSAGDQRVYSSYNQGRIFMPQ

SEQ ID NO: 107 - protein, 69 aa, Mus musculus
SLAAGTQAVLRCQSPRMVWTQDRLHDRQRVVHWDLSGGPGSQRRRLVDMYSAGEQRVYEPRDRDRLLLS

SEQ ID NO: 108 - protein, 72 aa, Asarcornis scutulata
SATLGSQAVLPCKSYRMVWTQDRLNDRQRVVHWDVYSTYYGDNKMERLCDMYSAGDQRVYSSYNQGRIFMPQ

SEQ ID NO: 109 - protein, 439 aa, Western equine encephalitis virus
FEHATTVPNVPGIPYKALVERAGYAPLNLEITVVSSELTPSTNKEYVTCKFHTVVPSPQVKCCGSLECKASSKADYTCRVFGGVYPFMWGGAQCFCDSENTQLSEAYVEFAPDCTIDHAVALKVHTAALKVGLRIVYGNTTARLDTFVNGVTPGSSRDLKVIAGPISAAFSPFDHKVVIRKGLVYNYDFPEYGAMNPGAFGDIQASSLDATDIVARTDIRLLKPSVKNIHVPYTQAVSGYEMWKNNSGRPLQETAPFGCKIEVEPLRATNCAYGHIPISIDIPDAAFVRSSESPTILEVSCTVADCIYSADFGGSLTLQYKANREGHCPVHSHSTTAVLKEATTHVTATGSITLHFSTSSPQANFIVSLCGKKTTCNAECKPPADHIIGEPHKVDQEFQAAVSKTSWNWLLALFGGASSLIVVGLIVLVCSSMLINTRR

SEQ ID NO: 110 - protein, 439 aa, Sindbis virus; identical to SEQ ID NO: 78

SEQ ID NO: 111 - protein, 441 aa, Eastern equine encephalitis virus
YEHTAVMPNKVGIPYKALVERPGYAPVHLQIQLVNTRIIPSTNLEYITCKYKTKVPSPVVKCCGATQCTSKPHPDYQCQVFTGVYPFMWGGAYCFCDTENTQMSEAYVERSEECSIDHAKAYKVHTGTVQAMVNITYGSVSWRSADVYVNGETPAKIGDAKLIIGPLSSAWSPFDNKVVVHGHEVYNYDFPEYGTGKAGSFGDLQSRTSTSNDLYANTNLKLQRPQAGIVHTPFTQAPSGFERWKRDKGAPLNDVAPFGCSIALEPLRAENCAVGSIPISIDIPDAAFTRISETPTVSDLECKITECTYASDFGGIATVAYKSSKAGNCPIHSPSGVAVIKENDVTLAESGSFTFHFSTANIHPAFKLQVCTSAVTCKGDCKPPKDHIVDYPAQHTESFTSAISATAWSWLKVLVGGTSAFIVLGLIATAVVALVLFFHRH

SEQ ID NO: 112 - protein, 442 aa, Venezuelan equine encephalitis virus
YEHATTMPSQAGISYNTIVNRAGYAPLPISITPTKIKLIPTVNLEYVTCHYKTGMDSPAIKCCGSQECTPTNRPDEQCKVFTGVYPFMWGGAYCFCDTENTQVSKAYVMKSDDCLADHAEAYKAHTASVQAFLNITVGEHSIVTTVYVNGETPVNFNGVKLTAGPLSTAWTPFDRKIVQYAGEIYNYDFPEYGAGQPGAFGDIQSRTVSSSDLYANTNLVLQRPKAGAIHVPYTQAPSGFEQWKKDKAPSLKFTAPFGCEIYTNPIRAENCAVGSIPLAFDIPDALFTRVSETPTLSAAECTLNECVYSSDFGGIATVKYSASKSGKCAVHVPSGTATLKEAAVELTEQGSATIHFSTANIHPEFRLQICTSYVTCKGDCHPPKDHIVTHPQYHAQTFTAAVSKTAWTWLTSLLGGSAVIIIIGLVLATIVAMYVLTNQKHN

SEQ ID NO: 113 - protein, 438 aa, Ross River virus
YEHTATIPNVVGFPYKAHIERNXFSPMTLQLEVVXXSLEPTLNLEYITCEYKTVVPSPFIKCCGTSECSSKEQPDYQCKVYTGVYPFMWGGAYCFCDSENTQLSEAYVDRSDVCKHDHALAYKAHTASLKATIRISYGTINQTTEAFVNGEHAVNVGGSKFIFGPISTAWSPFDNKIVVYKDDVYNQDFPPYGSGQPGRFGDIQSRTVESKDLYANTALKLSRPSPGVVHVPYTQTPSGFKYWLKEKGSSLNTKAPFGCKIKTNPVRAMDCAVGSIPVSMDIPDSAFTRVVDAPAVTDLSCQVAVCTHSSDFGXVATLSYKTDKPGKCAVHSHSNVATLQEATVDVKEDGKVTVHFSXXSASPAFKVSVCDAKTTCTAACEPPKDHIVPYGASHNNQVFPDMSGTAMTWVQRMASGLGGLALIAVVVLVLVTCITMRR

SEQ ID NO: 114 - protein, 436 aa, Mayaro virus
YEHTAIIPNQVGFPYKAHVAREGYSPLTLQMQVIETSLEPTLNLEYITCDYKTKVPSPYVKCCGTAECRTQDKPEYKCAVFTGVYPFMWGGAYCFCDSENTQMSEAYVERADVCKHDHAAAYRAHTASLRAKIKVTYGTVNQTVEAYVNGDHAVTIAGTKFIFGPVSTPWTPFDTKILVYKGELYNQDFPRYGAGQPGRFGDIQSRTLDSRDLYANTGLKLARPAAGNIHVPYTQTPSGFKTWQKDRDSPLNAKAPFGCIIQTNPVRAMNCAVGNIPVSMDIADSAFTRLTDAPVISELTCTVSTCTHSSDFGGIAVLSYKVEKSGRCDIHSHSNVAVLQEVSIETEGRSVIHFSTASASPSFVVSVCSSRATCTAKCEPPKDHVVTYPANHNGVTLPDLSSTAMTWAQHLAGGVGLLIALAVLILVIVTCVTLRR

SEQ ID NO: 115 - protein, 438 aa, Chikungunya virus
YEHVTVIPNTVGVPYKTLVNRPGYSPMVLEMELLSVTLEPTLSLDYITCEYKTVIPSPYVKCCGTAECKDKNLPDYSCKVFTGVYPFMWGGAYCFCDAENTQLSEAHVEKSESCKTEFASAYRAHTASASAKLRVLYQGNNITVTAYANGDHAVTVKDAKFIVGPMSSAWTPFDNKIVVYKGDVYNMDYPPFGAGRPGQFGDIQSRTPESKDVYANTQLVLQRPAAGTVHVPYSQAPSGFKYWLKERGASLQHTAPFGCQIATNPVRAMNCAVGNMPISIDIPDAAFTRVVDAPSLTDMSCEVPACTHSSDFGGVAIIKYAVSKKGKCAVHSMTNAVTIREAEIEVEGNSQLQISFSTALASAEFRVQVCSTQVHCAAECHPPKDHIVNYPASHTTLGVQDISATAMSWVQKITGGVGLVVAVAALILIVVLCVSFSR

SEQ ID NO: 116 - protein, 423 aa, Western equine encephalitis virus
SITDDFTLTSPYLGFCPYCRHSAPCFSPIKIENVWDESDDGSIRIQVSAQFGYNQAGTADVTKFRYMSYDHDHDIKEDSMEKLAISTSGPCRRLGHKGYFLLAQCPPGDSVTVSITSGASENSCTVEKKIRRKFVGREEYLFPPVHGKLVKCHVYDHLKETSAGYITMHRPGPHAYKSYLEEASGEVYIKPPSGKNVTYECKCGDYSTGIVSTRTKMNGCTKAKQCIAYKRDQTKWVFNSPDLIRHTDHSVQGKLHIPFRLTPTVCPVPLAHTPTVTKWFKGITLHLTATRPTLLTTRKLGLRADATAEWITGTTSRNFSVGREGLEYVWGNHEPVRVWAQESAPGDPHGWPHEIIIHYYHRHPVYTVIVLCGVALAILVGTASSAACIAKARRDCLTPYALAPNATVPTALAVLCCIRPTNA

SEQ ID NO: 117 - protein, 423 aa, Sindbis virus
SVIDDFTLTSPYLGTCSYCHHTVPCFSPVKIEQVWDEADDNTIRIQTSAQFGYDQSGAASANKYRYMSLKQDHTVKEGTMDDIKISTSGPCRRLSYKGYFLLAKCPPGDSVTVSIVSSNSATSCTLARKIKPKFVGREKYDLPPVHGKKIPCTVYDRLKETTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANA

SEQ ID NO: 118 - protein, 420 aa, Eastern equine encephalitis virus
DLDTHFTQYKLARPYIADCPNCGHSRCDSPIAIEEVRGDAHAGVIRIQTSAMFGLKTDGVDLAYMSFMNGKTQKSIKIDNLHVRTSAPCSLVSHHGYYILAQCPPGDTVTVGFHDGPNRHTCTVAHKVEFRPVGREKYRHPPEHGVELPCNRYTHKRADQGHYVEMHQPGLVADHSLLSIHSAKVKITVPSGAQVKYYCKCPDVREGTTSSDYTTTCTDVKQCRAYLIDNKKWVYNSGRLPRGEGDTFKGKLHVPFVPVKAKCIATLAPEPLVEHKHRTLILHLYPDHPTLLTTRSLGSDANPTRQWIERPTTVNFTVTGEGLEYTWGNHPPKRVWAQESGEGNPHGWPHEVVVYYYNRYPLTTIIGLCTCVAIIMVSCVTSVWLLCRTRNLCITPYKLAPNAQVPILLALLCCIKPTRA

SEQ ID NO: 119 - protein, 423 aa, Venezuelan equine encephalitis virus
STEELFKEYKLTRPYMARCIRCAVGSCHSPIAIEAVKSDGHDGYVRLQTSSQYGLDSSGNLKGRTMRYDMHGTIEEIPLHQVSLHTSRPCHIVDGHGYFLLARCPAGDSITMEFKKGSVTHSCSVPYEVKFNPVGRELYTHPPEHGAEQACQVYAHDAQNRGAYVEMHLPGSEVDSSLISLSGSSVTVTPPVGTSALVKCKCGGTKISETINKAKQFSQCTKKEQCRAYRLQNDKWVYNSDKLPKAAGATLKGKLHVPFLLADGKCTVPLAPEPMITFGFRSVSLKLHPKNPTYLTTRQLADEPHYTHELISEPAVRNFTVTEKGWEFVWGNHPPKRFWAQETAPGNPHGLPHEVITHYYHRYPMSTILGLSICAAIVTVSVAASTWLFCKSRVSCLTPYRLTPNARMPLCLAVLCCARTARA

SEQ ID NO: 120 - protein, 422 aa, Ross River virus
SVIEHFNVYKATRPYLAXCADCGDGYFCYSPVAIEKIRDEASDGMLKIQVSAQIGLDKAGTHAHTKMRYMAGHDVQESKRDSLRVYTSAACSIHGTMGHFIVAHCPPGDYLKXSFEDANSHVKACKVQYKHDPLPVGREKFVVRPHFGVELPCTSYQLTTAPTDEEIDMHTPPDIPDRTLLSQTAGNVKITAGGRTIRYNCTCGRDNVGTTSTDKTINTCKIDQCHAAVTSHDKWXFTSPFVPRADQTARKGKVHVPFPLTNVTCRVPLARAPDVTYGKKEVTLRLHPDHPTXFSYRSLGAVPHPYEEWVDKFSERIIPVTEEGIEYQWGNNPPVRLWAQLTTEGKPHGWPHEIIQYYYGLYPAATIAAVSGASLMALLTLAATCCMLATARRKCLTPYALTPGAVVPLTLGLLXCAPRANA

SEQ ID NO: 121 - protein, 422 aa, Mayaro virus
STANHFNAYKLTRPYVAYCADCGMGHSCHSPAMIENIQADATDGTLKIQFASQIGLTKTDTHDHTKIRYAEGHDIAEAARSTLKVHSSSECTVTGTMGHFILAKCPPGERISVSFVDSKNEHRTCRIAYHHEQRLIGRERFTVRPHHGIELPCTTYQLTTAETSEEIDMHMPPDIPDRTILSQQSGNVKITVNGRTVRYSSSCGSQAVGTTTTDKTINSCTVDKCQAYVTSHTKWQFNSPFVPRRMQAERKGKVHIPFPLINTTCRVPLAPEALVRSGKREATLSLHPIHPTLLSYRTFGAERVFDEQWITAQTEVTIPVPVEGVEYQWGNHKPQRFVVALTTEGKAHGWPHEIIEYYYGLHPTTTIVVVIRVSVVVLLSFAASVYMCVVARTKCLTPYALTPGAVVPVTIGVLCCAPKAHA

SEQ ID NO: 122 - protein, 404 aa, Chikungunya virus
PYLAHCPDCGEGHSCHSPVALERIRNEATDGTLKIQVSLQIGIGTDDSHDWTKLRYMDNHIPADAGRAGLFVRTSAPCTITGTMGHFILARCPKGETLTVGFTDSRKISHSCTHPFHHDPPVIGREKFHSRPQHGKELPCSTYVQSNAATAEEIEVHMPPDTPDRTLLSQQSGNVKITVNSQTVRYKCNCGGSNEGLITTDKVINNCKVDQCHAAVTNHKKWQYNSPLVPRNAELGDRKGKIHIPFPLANVTCMVPKARNPTVTYGKNQVIMLLYPDHPTLLSYRSMGEEPNYQEEWVTHKKEVVLTVPTEGLEVTWGNNEPYKYWPQLSANGTAHGHPHEIILYYYELYPTMTVVVVSVASFILLSMVGMAVGMCMCARRRCITPYELTPGATVPFLLSLICC

SEQ ID NO: 123 - protein, 427 aa, Asarcornis scutulata
LQPLLCMLLTRKLFSFLCSWLSVPADASNPNSVVVSVLNISATLGSQAVLPCKSYRMVWTQDRLNDRQRVVHWDVYSTYYGDNKMERLCDMYSAGDQRVYSSYNQGRIFMPQNAFTDGNFSLVIKDVAESDGGIYSCNLHHHYCHLYETVKIQLDVTKKAKAAKEYWDGEKAVIVALEGSTVMLPCVNRNQIWTERHSEEEQQVVHWDRQPPGVPHDRADRLIDLYASGERRSYGPLFIRQKMNITDTAFALGDFSLRISELESADEGTYSCHLHHHYCGLHERRIYQVFVTEPVREKKVVNLTTHNTAPAVDPNVVRGHNVINVIIPEGRIHFFQQLGYVLATLLLFIVLLIIVVLITRKRRQRGYEYNVKKYGEKDVNLKEFTVDTTDLTQYRSEDIRLDYKNNILKEKAEQARTFPAKNIDLDK

SEQ ID NO: 124 - protein, 436 aa, Passer montanus
MEQLLKLLLWQILLQQSSVCSYSVPADASNPASVVVSVLNISAALGSQAVLPCKSHRMVWTQDRLNDRQRVVHWDLLSSYYGDGRMERLCDMYSAGDQRVYSSYNQGRILMPENAFADGNFSLVIKGVAESDAGTYSCNLHHHYCHLYETVKIQLVVAKRGEEASEYWDGEKPVLVAPEGSTATLPCVNRQHIWTERHSEEEQQVVHWDRQPPGVPQDRADRLVDLYASGERRSYGPQFLRQKMNVTRGAFALGDFSLRISRLESADEGTYSCHLHHHYCGLHERRIYHVAVTEPERERKVVNISTHAVVPAADPNVVRGHNVINVIIPESRMHFFQQLGYILATLLLFLVLLIVVVLVTRRRRQRGYEYNVKKYGEKDVNLKEFTVDTTDLTPHKSEDIRLDYKNNILKEKAEQARSFPAKNIDLDKDFRKEYCK

SEQ ID NO: 125 - protein, 437 aa, Centrocercus urophasianus
MEQLAKLLLWQLLLQQSSVVYLYSVPADASNPDSVVVSVLNISATRGSQAVLPCKSYRMVWTQDRLNDRQRVVHWDVYSTYYGDNKMERLCDMYSAGDQRVYSSYNQGRILMPQNAFTDGNFSLVIKDVAESDAGIYSCNLHHHYCHLYETVKIQLDITKKAKAAKEYWDGEKAVIVALEGSTVMLPCVNRNHIWTERHSEEEQQVVHWDRQPPGVPHDRADRLIDLYASGERRSYGPLFIRQKMNITDTAFALGDFSLRISELENADEGTYSCHLHHHYCGLHERRIYQVFVTEPVREKKVVNLTTHNTAPAVDPNVVRGHNVINVIIPESRIHFFQQLGYVLATLLLFIVLLIIVVFITRKRRQRGYEYNVKKYGEKDVNLKEFTVDTTDLTQYKSEDIRLDYKNNILKEKAEQARSFPAKNIDLDKDFRKEYCK

SEQ ID NO: 126 - protein, 437 aa, Gallus gallus
MEQLAKLLLWQLLLQQSSVVYLYSVPADASNPDSVVVSVLNISATRGSQAVLPCKSYRMVWTQDRLNDRQRVVHWDVYSTYYGDNKMERLCDMYSAGNQRVYSSYNQGRILMPQNAFTDGNFSLVIKDVAESDAGVYSCNLHHHYCHLYETVKIQLDITKKAKAAKEYWDGEKAVIVALEGSTVMLPCVNRNHIWTERHSEEEQQVVHWDRQPPGVPHDRADRLIDLYASGERRSYGPLFIRQKMNITDTAFALGDFSLRISELENADEGTYSCHLHHHYCGLHERRIYQVFVTEPVREKKVVNLTTHNTAPAIDPNVVRGHNVINVIIPESRIHFFQQLGYVLATLLLFVVLLIIVVFITRKRRQRGYEYNVKKYGEKDVNLKEFTVDTTDLTQYKSEDIRLDYKNNILKEKAEQARSFPAKNIDLDKDFRKEYCK

SEQ ID NO: 127 - protein, 442 aa, Mus musculus
MELLSRVLLWKLLLLQSSAVLSSGPSGTAAASNSLVSESVVSLAAGTQAVLRCQSPRMVWTQDRLHDRQRVVHWDLSGGPGSQRRRLVDMYSAGEQRVYEPRDRDRLLLSPSAFHDGNFSLLIRAVERGDEGVYTCNLHHHYCHLDESLAVRLEVTEDPLLSRAYWDGEKEVLVVAHGAPALMTCINRAHVWTDRHLEEAQQVVHWDRQLPGVSHDRADRLLDLYASGERRAYGPPFLRDRVSVNTNAFARGDFSLRIDELERADEGIYSCHLHHHYCGLHERRVFHLQVTEPAFEPPARASPGNGSGHSSAPSPDPTLTRGHSIINVIVPEDHTHFFQQLGYVLATLLLFILLLITVVLATRHRHSGGCKTSDKKAGKSKGKDVNMVEFAVATRDQAPYRTEDIQLDYKNNILKERAELAHSPLPAKDVDLDKEFRKEYCK

SEQ ID NO: 128 - protein, 442 aa, Homo sapiens; identical to SEQ ID NO: 27

SEQ ID NO: 129 - protein, 420 aa, Mus musculus
SGPSGTAAASNSLVSESVVSLAAGTQAVLRCQSPRMVWTQDRLHDRQRVVHWDLSGGPGSQRRRLVDMYSAGEQRVYEPRDRDRLLLSPSAFHDGNFSLLIRAVERGDEGVYTCNLHHHYCHLDESLAVRLEVTEDPLLSRAYWDGEKEVLVVAHGAPALMTCINRAHVWTDRHLEEAQQVVHWDRQLPGVSHDRADRLLDLYASGERRAYGPPFLRDRVSVNTNAFARGDFSLRIDELERADEGIYSCHLHHHYCGLHERRVFHLQVTEPAFEPPARASPGNGSGHSSAPSPDPTLTRGHSIINVIVPEDHTHFFQQLGYVLATLLLFILLLITVVLATRHRHSGGCKTSDKKAGKSKGKDVNMVEFAVATRDQAPYRTEDIQLDYKNNILKERAELAHSPLPAKDVDLDKEFRKEYCK

SEQ ID NO: 130 - protein, 406 aa, Asarcornis scutulata
SVPADASNPNSVVVSVLNISATLGSQAVLPCKSYRMVWTQDRLNDRQRVVHWDVYSTYYGDNKMERLCDMYSAGDQRVYSSYNQGRIFMPQNAFTDGNFSLVIKDVAESDGGIYSCNLHHHYCHLYETVKIQLDVTKKAKAAKEYWDGEKAVIVALEGSTVMLPCVNRNQIWTERHSEEEQQVVHWDRQPPGVPHDRADRLIDLYASGERRSYGPLFIRQKMNITDTAFALGDFSLRISELESADEGTYSCHLHHHYCGLHERRIYQVFVTEPVREKKVVNLTTHNTAPAVDPNVVRGHNVINVIIPEGRIHFFQQLGYVLATLLLFIVLLIIVVLITRKRRQRGYEYNVKKYGEKDVNLKEFTVDTTDLTQYRSEDIRLDYKNNILKEKAEQARTFPAKNIDLDK

SEQ ID NO: 131 - protein, 414 aa, Synthetic construct
SGPSGTAAASSSLVSESVVSLAAGTQAVLRCQSPRMVWTQDRLNDRQRVVHWDVYSTYYGDNKMERLCDMYSAGDQRVYSSYNQGRIFMPQNAFTDGNFSLVIKDVAESDGGIYSCNLHHHYCHLYETVKIQLDVTKKAKAAKEYWDGEKAVIVALEGSTVMLPCVNRNQIWTERHLEEAQQVVHWDRQLPGVSHDRADRLLDLYASGERRAYGPPFLRDRVSVNTNAFARGDFSLRIDELERADEGIYSCHLHHHYCGLHERRVFHLQVTEPVREKKVVNLTTHNTAPAVDHNVVRGHNVINVIIPEGRIHFFQQLGYVLATLLLFIVLLIIVVLITRKRRQRGYEYNVKKYGEKDVNLKEFTVDTTDLTQYRSEDIRLDYKNNILKEKAEQARTFPAKNIDLDKDFRKEYCK

SEQ ID NO: 132 - protein, 406 aa, Synthetic construct
SNPNSVVVSVLNISATLGSQAVLPCKSYRMVWTQDRLHDRQRVVHWDLSGGPGSQRRRLVDMYSAGEQRVYEPRDRDRLLLSPSAFHDGNFSLLIRAVDRGDEGVYTCNLHHHYCHLDESLAVRLEVTEDPLLSRAYWDGEKEVLVVAHGAPALMTCINRAHVWTDRHSEEEQQVVHWDRQPPGVPHDRADRLIDLYASGERRSYGPLFIRQKMNITDTAFALGDFSLRISELESADEGTYSCHLHHHYCGLHERRIYQVFVTEPVREKKVVNLTTHNTAPAVDHNVVRGHNVINVIIPEGRIHFFQQLGYVLATLLLFIVLLIIVVLITRKRRQRGYEYNVKKYGEKDVNLKEFTVDTTDLTQYRSEDIRLDYKNNILKEKAEQARTFPAKNIDLDKDFRKEYCK

SEQ ID NO: 133 - protein, 408 aa, Synthetic construct
SNPNSVVVSVLNISATLGSQAVLPCKSYRMVWTQDRLNDRQRVVHWDVYSTYYGDNKMERLCDMYSAGDQRVYSRRNQGRIFMPQNAFTDGNFSLVIKDVAESDGGIYSCNLHHHYCHLYETVKIQLDVTKKAKAAKEYWDGEKAVIVALEGSTVMLPCVNRNQIWTERHSEEEQQVVHWDRQPPGVPHDRADRLIDLYASGERRSYGPLFIRQKMNITDTAFALGDFSLRISELESADEGTYSCHLHHHYCGLHERRIYQVFVTEPVREKKVVNLTTHNTAPAVDHNVVRGHNVINVIIPEGRIHFFQQLGYVLATLLLFIVLLIIVVLITRKRRQRGYEYNVKKYGEKDVNLKEFTVDTTDLTQYRSEDIRLDYKNNILKEKAEQARTFPAKNIDLDKDFRKEYCK

SEQ ID NO: 134 - protein, 417 aa, Synthetic construct
SNPNSVVVSVLNISATLGSQAVLPCKSYRMVWTQDRLNDRQRVVHWDVYSTYYGDNKMERLCDMYSAGDQRVGEQRVGEQRLGEQRVGRIFMPQNAFTDGNFSLVIKDVAESDGGIYSCNLHHHYCHLYETVKIQLDVTKKAKAAKEYWDGEKAVIVALEGSTVMLPCVNRNQIWTERHSEEEQQVVHWDRQPPGVPHDRADRLIDLYASGERRSYGPLFIRQKMNITDTAFALGDFSLRISELESADEGTYSCHLHHHYCGLHERRIYQVFVTEPVREKKVVNLTTHNTAPAVDHNVVRGHNVINVIIPEGRIHFFQQLGYVLATLLLFIVLLIIVVLITRKRRQRGYEYNVKKYGEKDVNLKEFTVDTTDLTQYRSEDIRLDYKNNILKEKAEQARTFPAKNIDLDKDFRKEYCK

SEQ ID NO: 135 - protein, 408 aa, Synthetic construct
SNPNSVVVSVLNISATLGSQAVLPCKSYRMVWTQDRLNDRQRVVHWDVYSTYYGDNKMERLCDMYSAGDQRVYSSYNQGRIFMPQNAFRRGNFSLVIKDVAESDGGIYSCNLHHHYCHLYETVKIQLDVTKKAKAAKEYWDGEKAVIVALEGSTVMLPCVNRNQIWTERHSEEEQQVVHWDRQPPGVPHDRADRLIDLYASGERRSYGPLFIRQKMNITDTAFALGDFSLRISELESADEGTYSCHLHHHYCGLHERRIYQVFVTEPVREKKVVNLTTHNTAPAVDHNVVRGHNVINVIIPEGRIHFFQQLGYVLATLLLFIVLLIIVVLITRKRRQRGYEYNVKKYGEKDVNLKEFTVDTTDLTQYRSEDIRLDYKNNILKEKAEQARTFPAKNIDLDKDFRKEYCK

SEQ ID NO: 136 - protein, 423 aa, Synthetic construct
SNPNSVVVSVLNISATLGSQAVLPCKSYRMVWTQDRLNDRQRVVHWDVYSTYYGDGEQRVGEQRLGEQRVNKMERLCDMYSAGDQRVYSSYNQGRIFMPQNAFTDGNFSLVIKDVAESDGGIYSCNLHHHYCHLYETVKIQLDVTKKAKAAKEYWDGEKAVIVALEGSTVMLPCVNRNQIWTERHSEEEQQVVHWDRQPPGVPHDRADRLIDLYASGERRSYGPLFIRQKMNITDTAFALGDFSLRISELESADEGTYSCHLHHHYCGLHERRIYQVFVTEPVREKKVVNLTTHNTAPAVDHNVVRGHNVINVIIPEGRIHFFQQLGYVLATLLLFIVLLIIVVLITRKRRQRGYEYNVKKYGEKDVNLKEFTVDTTDLTQYRSEDIRLDYKNNILKEKAEQARTFPAKNIDLDKDFRKEYCK

SEQ ID NO: 137 - protein, 408 aa, Synthetic construct
SNPNSVVVSVLNISATLGSQAVLPCKSYRMVWTQDRLNDRQRVVHWDVYSTYYGDNKMERLCDMYSAGDQRVYSSYNQGRIFMPQNAFTDGNFSLVIKDVAESDGGIYSCNLHHHYCHLYETVKIQLDVTKKAKAAKEYWDGEKAVIVALEGSTVMLPCVNRNQIWTERHSEEEQQVVHWDRQPPGVPHDRADRLIDLYASGERRSYGPRRIRQKMNITDTAFALGDFSLRISELESADEGTYSCHLHHHYCGLHERRIYQVFVTEPVREKKVVNLTTHNTAPAVDHNVVRGHNVINVIIPEGRIHFFQQLGYVLATLLLFIVLLIIVVLITRKRRQRGYEYNVKKYGEKDVNLKEFTVDTTDLTQYRSEDIRLDYKNNILKEKAEQARTFPAKNIDLDKDFRKEYCK

SEQ ID NO: 138 - protein, 417 aa, Synthetic construct
SNPNSVVVSVLNISATLGSQAVLPCKSYRMVWTQDRLNDRQRVVHWDVYSTYYGDNKMERLCDMYSAGDQRVYSSYNQGRIFMPQNAFTDGNFSLVIKDVAESDGGIYSCNLHHHYCHLYETVKIQLDVTKKAKAAKEYWDGEKAVIVALEGSTVMLPCVNRNQIWTERHSEEEQQVVHWDRQPPGVPHDRADRLIDLYASGERRSYGGEQRVGEQRLGEQRVKMNITDTAFALGDFSLRISELESADEGTYSCHLHHHYCGLHERRIYQVFVTEPVREKKVVNLTTHNTAPAVDHNVVRGHNVINVIIPEGRIHFFQQLGYVLATLLLFIVLLIIVVLITRKRRQRGYEYNVKKYGEKDVNLKEFTVDTTDLTQYRSEDIRLDYKNNILKEKAEQARTFPAKNIDLDKDFRKEYCK

SEQ ID NO: 139 - protein, 408 aa, Synthetic construct
SNPNSVVVSVLNISATLGSQAVLPCKSYRMVWTQDRLNDRQRVVHWDVYSTYYGDNKMERLCDMYSAGDQRVYSSYNQGRIFMPQNAFTDGNFSLVIKDVAESDGGIYSCNLHHHYCHLYETVKIQLDVTKKAKAAKEYWDGEKAVIVALEGSTVMLPCVNRNQIWTERHSEEEQQVVHWDRQPPGVPHDRADRLIDLYASGERRSYGPLFIRQKMNITDTAFRRGDFSLRISELESADEGTYSCHLHHHYCGLHERRIYQVFVTEPVREKKVVNLTTHNTAPAVDHNVVRGHNVINVIIPEGRIHFFQQLGYVLATLLLFIVLLIIVVLITRKRRQRGYEYNVKKYGEKDVNLKEFTVDTTDLTQYRSEDIRLDYKNNILKEKAEQARTFPAKNIDLDKDFRKEYCK

SEQ ID NO: 140 - protein, 423 aa, Synthetic construct
SNPNSVVVSVLNISATLGSQAVLPCKSYRMVWTQDRLNDRQRVVHWDVYSTYYGDNKMERLCDMYSAGDQRVYSSYNQGRIFMPQNAFTDGNFSLVIKDVAESDGGIYSCNLHHHYCHLYETVKIQLDVTKKAKAAKEYWDGEKAVIVALEGSTVMLPCVNRNQIWTERHSEEEQQVVHWDRQPPGVPHGEQRVGEQRLGEQRVDRADRLIDLYASGERRSYGPLFIRQKMNITDTAFALGDFSLRISELESADEGTYSCHLHHHYCGLHERRIYQVFVTEPVREKKVVNLTTHNTAPAVDHNVVRGHNVINVIIPEGRIHFFQQLGYVLATLLLFIVLLIIVVLITRKRRQRGYEYNVKKYGEKDVNLKEFTVDTTDLTQYRSEDIRLDYKNNILKEKAEQARTFPAKNIDLDKDFRKEYCK

SEQ ID NO: 141 - protein, 132 aa, Asarcornis scutulata
DRQRVVHWDVYSTYYGDNKMERLCDMYSAGDQRVYSSYNQGRIFMPQNAFTDGNFSLVIKDVAESDGGIYSCNLHHHYCHLYETVKIQLDVTKKAKAAKEYWDGEKAVIVALEGSTVMLPCVNRNQIWTERH

SEQ ID NO: 142 - protein, 132 aa, Alligator mississippiensis
DRQRVVHWDLYSNYYGDYKSERLCDMYSAGEQRVYSSYNKGRIFMPENAFTDGNFSLVIKDLAESDKGIYSCNLHHHYCHLYKTVKIQLDVTKKAKAVKNYWDGEKEVLVALRGSTVVLSCVNRDHIWTERH

SEQ ID NO: 143 - protein, 132 aa, Crocodylus porosus
DRQRVVHWDLYSNYYGDYKSERLCDMYSAGEQRVYSSYNKGRIFMPENAFTDGNFSLVIKDLAESDKGIYSCNLHHHYCHLYKTVKIQLDITKKAKAVKNYWDGEKEVLVALRGSTVVLSCVNRDHIWTERH

SEQ ID NO: 144 - protein, 132 aa, Chelonia mydas
DRQRVVHWDLYSNPEGDYKAERLCDMYSAGEQRVYSSYNQGRIAMPQNAFADGNFSLVIKDVAESDQGTYSCNLHHHYCHLYETVKIQLEVTEKAREAKRYWDGEKAVIVALKDSTVVLPCVNRNQIWTERH

SEQ ID NO: 145 - protein, 132 aa, Chelydra serpentina
DRQRVVHWDLYSNQDGDYKAERLCDMYSAGEQRVYSNYNQGRIAMPQNAFADGNFSLVIKDVAESDQGTYSCNLHHHYCHLYETVKIQLEVTKKAREAKRYWDGEKAVIVALKDSTVVLPCVNRNQIWTERH

SEQ ID NO: 146 - protein, 130 aa, Latimeria chalumnae
DRQRVVHWDLYHATGNMIERVFDMFSAGDQRIYNSYNQRRMFMAQNAFSEGNFSLVIKDVSLTDRGIYSCNLHHHYCHLYESIQVQLNVTKSVKKAKISWDGEKVVIVALSGSTVVLPCVNRNQVWTERY

SEQ ID NO: 147 - protein, 129 aa, Sceloporus undulatus
DRQRVVHWDLYRSQLGAERLCDMYSAGDQRVYSGYNQGRIFMPENAFVDGNFSLVVKDVAESDQGIYSCNLHHHYCHLYETVQIQLEVTKRAREVHSYWDGEKPVVVALKGSTVVLPCINRNQVWTERH

SEQ ID NO: 148 - protein, 131 aa, Microcaecilia unicolor
DRQRVVHWDLYSSDGSYKAERLVDMYSAGEQRIYSSYNQGRVFMPETAFVDGNFSLVIKGIADRDQGIYSCNLHHHYCHLYKTEKIQLDVTYTAEEEKMYWDGEKIVMVAVAGSTVMLPCENRNHLWTERH

SEQ ID NO: 149 - protein, 131 aa, Erpetoichthys calabaricus
DRQRVVHWDLYSTGRDYKVERLLDMFSAGDQRIYNSYNQGRMSMISTAFADGNFSLIIKDVAPSDKGIYSCNLHHHYCHLYESTKMQLNITKSARKERRYWDGDKVVVMALVGSTVLLPCVNRRQVWTERN

SEQ ID NO: 150 - protein, 129 aa, Varanus komodoensis
DRQRVVHWDLDRSGQVTERLCDMYSAGEQRIYSSYNEGRISMPENAFIDGNFSLVIKDVAESDQGVYSCNLHHHYCHLYEREQIQLDVTKKAKEVERYWDGEKVVIVALKGSTVVLSCINRNQVWTERH

SEQ ID NO: 151 - protein, 129 aa, Pantherophis guttatus
DRQRVVHWDLYRSQQDVERLCDMYSAGDRRVYHTYNQGRISMPENAFTEGNFSLMVQDVAESDQGIYSCNLHHHYCHLYETVKLQLEVTEEAEEVQRYWDGEKVVIVALKGSAVILPCINRNQVWTGRD

SEQ ID NO: 152 - protein, 131 aa, Polypterus bichir
DRQRVVHWDLYSTGREYKVERLLDMFSAGDQRIYNSYNQGRMSMISTAFADGNFSLIIKDVASSDKGIYSCNLHHHYCHLYESTKVQLNITKSARKERRYWDGDKVVVMALVGSTVLLPCVNRRQVWTERN

SEQ ID NO: 153 - protein, 131 aa, Cyprinus carpio
DRQRVAHWDLLRSGPDHAMERVIDMFSAGDQRIYNGYNQGRIYMPKTAFKDGNFSLVIKDVAMSDKGIYSCNLHHHYCHLYESIKVQLNVTKSARKERRFWDGQKAVFVVLVGSTVVLPCINRRPIWTEGN

SEQ ID NO: 154 - protein, 131 aa, Carcharodon carcharias
DRQRVAHWDWFSISGEYKVDRVLDMFSAGDQRVYNSYNQGRVMMSTSAFSSGNFSLVIKDVRTNDRGIYSCNLHHHYCHLYETVKIQLNITRSAKKVRKFWDGEKAVMVALVGTTVVLPCENHNHIWTERH

SEQ ID NO: 155 - protein, 130 aa, Monodelphis domestica
DRQRVVHWDLSGGPGSTPRRLLDMYSAGEQRVYASRDHNRLLLAPSAFQDGNFSLIIRSVEEQDEGLYTCNLHHHYCHLYETLAIRLQVTDDPMEAGAHWDGEKEVLVVERGFPALLTCVNRAQVWTERH

SEQ ID NO: 156 - protein, 131 aa, Neogale vison
DRQRVVHWDLSGRPDGGPARRLVDMYSAGEQRVYEPRDRGRLLLPLSAFHDGNFSLLIRAVDEADAGLYTCNLHHHYCHLYESLAVRLEVTDNPREAGAHWDGEKEVLVAEHGAPALLTCVNRAHVWTDRH

SEQ ID NO: 157 - protein, 132 aa, Eleutherodactylus coqui
IDRQRVVHWDLYSSQGQYRGERLLDMFSVGEQRIYSGYNKGRIMVSKSAFQDGNFSLVIKGVSTSDQGLYSCNLHHHYCHLDETVRVQLNITKSERKVKMYWDGEKAVVVALVGSNVMLPCENWNHIWTDRH

SEQ ID NO: 158 - protein, 131 aa, Bufo bufo
DRQRVVHWDLYSSQGVYRGERLLDMFSAGDQRIYSGYNQGRIMVSNSAFQSGNFSLVIKAVSMSDQGLYSCNLHHHYCHLDETVRVQLNITKSERRVKMYWDGEKAVVVALVGSNVVLPCENWNHIWTDRH

SEQ ID NO: 159 - protein, 130 aa, Castor canadensis
DRQRVVHWDLSSGSGSPGRRLVDMYSAGEQRVYEPHDHDRLLLSPSAFHDGNFSLLIRAVGDSDEGLYTCNLHHHYCHLYESLAIRLEVTDDPRAARAYWDGEKEVLVAARGAPALLTCVNRAHVWTDRH

SEQ ID NO: 160 - protein, 130 aa, Trichosurus vulpecula
DRQRVVHWDLSGGPSGPARRLLDMYSAGGQRIYEARDEGRLLLAPTAFQDGNFSLIIHSVEEQDEGLYTCNLHHHYCHLYETLAIRLEVTDDPLAAGAHWDGEKEVLVVERGSPALLTCVNRAQVWTERH

SEQ ID NO: 161 - protein, 131 aa, Rousettus aegyptiacus
DRQRVVHWDVSGRRAGGPAKRLVDMYSAGEQRVYEPRDRGRLQLPPSAFQEGNFSLLIRAVEETDGGLYTCNLHHHYCHLYESLAIRLEVTDNPRAAGAHWDGEKEVLVAERGAPALLTCVNRAHVWTDRH

SEQ ID NO: 162 - protein, 130 aa, Mus musculus
DRQRVVHWDLSGGPGSQRRRLVDMYSAGEQRVYEPRDRDRLLLSPSAFHDGNFSLLIRAVERGDEGVYTCNLHHHYCHLDESLAVRLEVTEDPLLSRAYWDGEKEVLVVAHGAPALMTCINRAHVWTDRH

SEQ ID NO: 163 - protein, 20 aa, Synthetic construct
MARSVTLVFLVLVSLTGLYA

SEQ ID NO: 164 - protein, 8 aa, Synthetic construct
DYKDDDDK

SEQ ID NO: 165 - DNA, 30 bp, Synthetic construct
  source 1..30: mol_type = other DNA
gcacttgatgtaattctccttggaatttgc

SEQ ID NO: 166 - DNA, 27 bp, Synthetic construct
  source 1..27: mol_type = other DNA
ctcaagcctcagacagtggttcaaagt

SEQ ID NO: 167 - DNA, 29 bp, Synthetic construct
  source 1..29: mol_type = other DNA
ggtggaaaataacatatagacaaacgcac

SEQ ID NO: 168 - DNA, 24 bp, Synthetic construct
  source 1..24: mol_type = other DNA
tgaacatcactgatactgcctttg

SEQ ID NO: 169 - DNA, 25 bp, Synthetic construct
  source 1..25: mol_type = other DNA
tcaggggagaatatttatgccacaa

SEQ ID NO: 170 - DNA, 25 bp, Synthetic construct
  source 1..25: mol_type = other DNA
tgaatataaccgatactgctttcgc

SEQ ID NO: 171 - DNA, 20 bp, Synthetic construct
  source 1..20: mol_type = other DNA
caggggaggatactgatgcc

SEQ ID NO: 172 - DNA, 19 bp, Synthetic construct
  source 1..19: mol_type = other DNA
tgggcccctctttatccga

SEQ ID NO: 173 - DNA, 24 bp, Synthetic construct
  source 1..24: mol_type = other DNA
gtggagtcatactggaacatgtag

SEQ ID NO: 174 - DNA, 19 bp, Synthetic construct
  source 1..19: mol_type = other DNA
aatggtgaaggtcggtgtg

SEQ ID NO: 175 - DNA, 20 bp, Synthetic construct
  source 1..20: mol_type = other DNA
tgcaaatggcagccctggtg

SEQ ID NO: 176 - DNA, 20 bp, Synthetic construct
  source 1..20: mol_type = other DNA
acagctcctacaaccaaggg

SEQ ID NO: 177 - DNA, 21 bp, Synthetic construct
  source 1..21: mol_type = other DNA
ctgaaaaaggaaggagttgag

SEQ ID NO: 178 - DNA, 20 bp, Synthetic construct
  source 1..20: mol_type = other DNA
aagatgaaaggaaaggcgtt

SEQ ID NO: 179 - DNA, 21 bp, Synthetic construct
  source 1..21: mol_type = other DNA
aagatcatcgacgcagtcatc

SEQ ID NO: 180 - DNA, 21 bp, Synthetic construct
  source 1..21: mol_type = other DNA
gctgtggaagtaaccgaatct

SEQ ID NO: 181 - DNA, 25 bp, Synthetic construct
  source 1..25: mol_type = other DNA
ccaccttacttctgcggcggattta

SEQ ID NO: 182 - DNA, 18 bp, Synthetic construct
  source 1..18: mol_type = other DNA
agttggctgtttgccctt

SEQ ID NO: 183 - DNA, 20 bp, Synthetic construct
  source 1..20: mol_type = other DNA
cgtgtgctagtcagcatcat

SEQ ID NO: 184 - DNA, 24 bp, Synthetic construct
  source 1..24: mol_type = other DNA
taattaatagcgacgaggcgccgc

SEQ ID NO: 185 - DNA, 25 bp, Synthetic construct
  source 1..25: mol_type = other DNA
atgcttttacagatggtaacttcag